(19) United States
(12) Patent Application Publication
(10) Pub. No.: US 2009/0270363 A1
(43) Pub. Date: Oct. 29, 2009

(54) 2-Aryl- and 2-Heteroarylthiazolyl Compounds, Methods for Their Preparation and Use Thereof

(75) Inventors: Nan Zhang, Bayside, NY (US); Semiramis Ayral-Kaloustian, Tarrytown, NY (US); Tarek Suhayl Mansour, New City, NY (US); Thai Hiep Nguyen, Fair Lawn, NJ (US); Chuansheng Niu, Cheshire, CT (US); Edward Christian Rosfjord, Oakland, NJ (US); Ronald Suayan, Hoboken, NJ (US); Hwei-Ru Tsou, New City, NY (US)

Correspondence Address: WYETH;PATENT LAW GROUP, 5 GIRALDA FARMS, MADISON, NJ, 07940, US

(73) Assignee: Wyeth, Madison, NJ (US)

(21) Appl. No.: 12/412,506

(22) Filed: Mar. 27, 2009

Related U.S. Application Data
(60) Provisional application No. 61/039,893, filed on Mar. 27, 2008.

(51) Int. Cl. A61K 31/428; C07D 277/66; C07D 417/10; A61K 31/506; A61K 31/397; A61P 35/00

(52) U.S. Cl. 514/210.21; 548/178; 544/297; 514/367; 514/275

(57) ABSTRACT

The present invention discloses fused bicyclic 2-aryl- or 2-heteroarylthiazolyl compounds and their pharmaceutically acceptable salts and esters thereof, which are useful for inhibiting the growth of cancerous cells, inhibiting human breast carcinoma tumor growth in particular and to treat diseases or disorders associated with securin, including elevated securin levels.

### CROSS-REFERENCE TO RELATED APPLICATION

This application claims the benefit under 35 U.S.C. §119(e) to U.S. Provisional Application Ser. No. 61/039,893, filed Mar. 27, 2008, which is hereby incorporated by reference in their entirety.

### FIELD OF THE INVENTION

The present invention relates to compounds that prevent, inhibit and/or terminate growth of transformed and cancerous cell lines during tumorigenesis. More specifically, the present invention is directed to certain fused bicyclic 2-substituted aryl- and heteroarylthiazolyl compounds and their pharmaceutically acceptable salts. The invention is also directed to methods for preparing fused bicyclic 2-aryl- and 2-heteroarylthiazolyl compounds, including their pharmaceutically acceptable salts, methods for preparing pharmaceutical compositions and formulations that include fused bicyclic 2-aryl- and 2-heteroarylthiazolyl compounds and methods for treating diseases associated with securin activity and inhibiting abnormal growth of certain cell types using fused bicyclic 2-aryl- and 2-heteroarylthiazolyl compounds.

### BACKGROUND OF THE INVENTION

Cancer is characterized by the uncontrolled rapid growth of abnormal cells, which spread to other tissues and organs through the lymphatic system or blood stream. Tumorigenesis in mammals is a multi-step process in which accumulation of genetic alterations drives the progressive transformation of normal cells to tumor cells. Genetic instability is required to generate the multiple mutations underlying cancer. An estrogen induced transforming gene securin is implicated in functional mechanisms related to cell-cycle control and tumorigenesis. It has been shown, for example, that securin is overexpressed in breast cancer and is associated with metastatic tumors, tumor spread and lymph node invasion. Anti-cancer agents are used to treat and control the growth of these cancerous cells by inhibiting, preventing and/or destroying the cancerous cells.

Certain fused bicyclic thiazoles substituted at the 2-position are known in the prior art and their corresponding uses as a pesticide, a sedative, an anti-inflammatory or an antipyretic. For example, Japanese Patent No. JP 10-017569, discloses certain fused bicyclic 2-phenylthiazole compounds, where the cyclic ring fused to the thiazolyl ring is unsubstituted and methods for using the compounds as 5 HT3 agonists. There is a need to identify and characterize new fused bicyclic 2-substituted aryl- and heteroarylthiazolyl compounds having a variety of substituents on the 2-aryl ring or the 2-heteroaryl ring and on the cyclic ring fused to the thiazolyl ring, that inhibit growth of tumor cells. Little is known regarding how various functional groups substituted on the 2-aryl or 2-heteroaryl ring and on the cyclic ring fused to the thiazolyl ring, in addition to the effects of cyclic groups fused to the thiazolyl ring, influence structure-activity relationships (SAR). The fused bicyclic 2-aryl- or 2-heteroarylthiazolyl compounds of the present invention fulfill this unmet need and are useful for inhibiting the growth of cancerous cells, inhibiting human breast carcinoma tumor growth in particular and to treat diseases or disorders associated with securin activity, including elevated securin levels.

### SUMMARY OF THE INVENTION

The present invention provides fused bicyclic 2-aryl- or 2-heteroarylthiazolyl compounds of formula I:

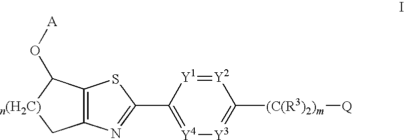

and pharmaceutically acceptable salts thereof
wherein,
A is H, C₁-C₃ alkyl, or acetyl;

### Q is —NR¹R², —NR³N(R³)₂, —NR³OR³, or —OH;

R¹ and R² are each independently H or C₁-C₃ alkyl, or R¹ and R² join together with the nitrogen atom to which each is attached, forming a 4 to 6 membered saturated heterocyclic ring comprising heteroatoms selected from 1-2 nitrogen atoms, 0-1 oxygen atom and 0-1 sulfur atom, said ring optionally substituted with one or more of R⁴;
R³ at each occurrence, is independently H or C₁-C₃ alkyl;
R⁴ is C₁-C₃ alkyl, —N(R³)₂, or —OH;
Y¹, Y², Y³, and Y⁴ are the same or different, and are each independently N or CR⁵, or two R⁵ groups on adjacent carbon atoms join together, with the carbon atoms which they are bonded, to form a 9 to 10 membered bicyclic aryl ring or bicyclic heteroaryl ring, said ring comprising members selected from CR⁵ and N;
R⁵ is independently H or is independently selected from C₁-C₃ alkyl, F, Cl, Br, I, CF₃, NO₂, —NR¹R², —CHO, —CONHAr, —C(R³)₂OR³, —C(R³)₂O[C(R³)₂]Ar, —C(R³)₂NR¹R², —C(R³)₂NR³[C(R³)₂]₂NR¹R², —CO₂R⁶, —SOR⁶, and —SO₂R⁶, where Ar is phenyl, 2-pyridyl, 3-pyridyl, 4-pyridyl, 2-thienyl, 3-thienyl, 2-furyl, or 3-furyl, optionally substituted with one or more of R⁴;
R⁶ is independently H or is independently selected from C₁-C₈ alkyl, C₂-C₈ alkenyl, and C₂-C₈ alkynyl, each optionally substituted with —NR¹R², —OR³, C₄-C₆ cycloalkyl, a saturated heterocyclic ring comprising heteroatoms selected from 0-1 nitrogen atom, 0-1 oxygen atom and 0-1 sulfur atom, or —COCH₃, said C₄-C₆ cycloalkyl, optionally substituted with R⁴; or a saturated heterocyclic ring comprising heteroatoms selected from 0-1 nitrogen atom, 0-1 oxygen atom and 0-1 sulfur atom, and optionally substituted with one or more of R⁴;
m is 0 or 1; and n is an integer from 1 to 3.

The present invention also provides esters of fused bicyclic 2-aryl- or 2-heteroarylthiazolyl compounds of formula I.

The present invention provides a pharmaceutical composition comprising: a fused bicyclic 2-aryl- or 2-heteroarylthiazolyl compound of formula I, including a pharmaceutically acceptable salt thereof, and one or more pharmaceutically acceptable carriers.

The present invention also includes a method for preparing a fused bicyclic 2-aryl- or 2-heteroarylthiazolyl compound of formula I or a pharmaceutically acceptable salt thereof, comprising the steps of:

(a) reacting a substituted arylnitrile compound of formula 1:

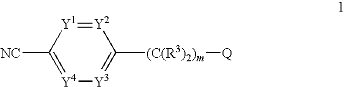

with phosphorous pentasulfide, thereby forming a substituted thioamide compound of formula 2:

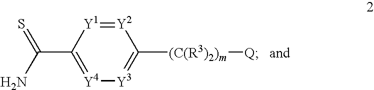

(b) reacting the substituted thioamide compound with an epoxyketone of formula 3:

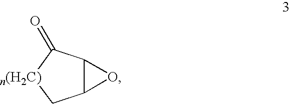

thereby forming the fused bicyclic 2-aryl- or 2-heteroarylthiazolyl compound of formula I.

The present invention also includes a method for preparing a fused bicyclic 2-aryl- or 2-heteroarylthiazolyl compound of formula I or a pharmaceutically acceptable salt thereof, comprising the steps of:

(a) reacting a 4-bromoarylnitrile compound of formula 4:

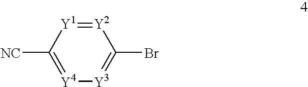

with phosphorous pentasulfide, thereby forming a 4-bromoarylthioamide compound of formula 5:

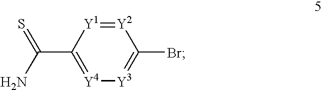

(b) reacting the thioamide compound of step (a) with an epoxyketone of formula 3:

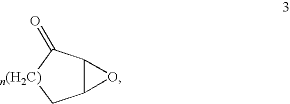

thereby forming an intermediate 2-arylthiazolyl compound of formula 6:

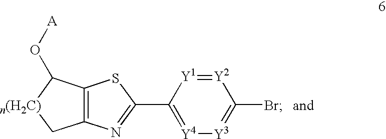

(c) treating the intermediate compound of formula 6 formed in step (b) with alkyl or cyclic alkyl amines, thereby forming the 2-aryl- or 2-heteroarylthiazolyl compound of formula 1.

Accordingly, the invention provides a method of inhibiting the growth of cancerous cells or tumors comprising the step of: administering to a patient in need a pharmaceutically effective amount of a 2-aryl- or 2-heteroarylthiazolyl compound of formula I. The invention also provides a method of treating a disease associated with securin activity comprising the step of administering to a subject in need a therapeutically effective amount of one or more 2-aryl- or 2-heteroarylthiazolyl compounds of formula I and pharmaceutically acceptable salts thereof. In one embodiment, securin activity is associated with a human breast carcinoma. In other embodiments, securin activity is associated with one or more cancers selected from: leukemia, brain cancer, lung cancer, colon cancer, thyroid cancer, ovarian cancer, renal cancer and prostate cancer. Other embodiments of the invention are found in the following detailed description.

### BRIEF DESCRIPTION OF THE DRAWINGS

FIG. 1 is a plot comparing the effectiveness of Examples 164 and 166 versus Herceptin at controlling tumor growth.

FIG. 2 is a plot demonstrating the effectiveness of Examples 164, 166 and 167 versus Vincristine at controlling tumor growth.

### DETAILED DESCRIPTION OF THE INVENTION

The term “alkyl” refers to the radical of saturated aliphatic groups of 1 to 8 carbon atoms, including straight-chain alkyl groups, branched-chain alkyl groups, cycloalkyl (alicyclic) groups, alkyl substituted cycloalkyl groups, and cycloalkyl substituted alkyl groups. In one embodiment, a straight chain or branched chain alkyl has 6 or fewer carbon atoms in its backbone. The term “alkyl” can be used alone or as part of a chemical name, such as “alkylamine”. The terms “alkenyl” and “alkynyl” refer to unsaturated aliphatic groups analogous in length and possible substitution to the alkyls described above, but which contain at least one double or triple carbon-carbon bond, respectively. The term “alkoxy” as used herein refers to an alkyl group, as defined above, having an oxygen radical attached thereto. Representative alkoxyl groups include methoxy, ethoxy, propyloxy, tert-butoxy and the like. The term alkoxy can be used alone or as part of a chemical name. Alkoxy also means a group —OR, wherein R is an alkyl, alkenyl, or alkynyl group which can optionally be substituted with one or more functional groups. Hydroxy means —OH. Carbonyl means carbon bonded to oxygen with a double bond, C═O. Acetyl means the functional group, —C(═O)CH₃. Amino means the —NH₂ group or a substituted amino group, —NR¹R².

The term “aryl”, as used herein, whether used alone or as part of another group, is defined as a substituted or unsubstituted aromatic hydrocarbon ring group having 6 to about 50 carbon atoms (unless explicitly specified otherwise) with from about 6 to about 10 atoms being preferred. The “aryl” group can have a single ring or multiple condensed rings. The term “aryl” includes, but is not limited to phenyl, α-naphthyl, β-naphthyl, biphenyl, anthryl, tetrahydronaphthyl, fluorenyl, indanyl, biphenylenyl, and acenaphthenyl. Specifically included within the definition of “aryl” are those aromatic groups that are optionally substituted. For example, in representative embodiments of the present invention, the, “aryl” groups are optionally substituted with from 1 to 5 substituents selected from: H, C₁-C₃ alkyl, F, Cl, Br, I, CF₃, NO₂, —NR¹R², —CHO, —CONHAr, —C(R³)₂OR³, —C(R³)₂O[C(R³)₂]Ar, —C(R³)₂NR¹R², —C(R³)₂NR³[C(R³)₂]₂NR¹R², —CO₂R⁶, —SOR⁶, or —SO₂R⁶, where Ar is phenyl, 2-pyridyl, 3-pyridyl, 4-pyridyl, 2-thienyl, 3-thienyl, 2-furyl, or 3-furyl, optionally substituted with C₁-C₃ alkyl, —N(R³)₂, and —OH.

The term “heteroaryl” as used herein is defined as a substituted or unsubstituted aromatic heterocyclic ring system (monocyclic or bicyclic). Heteroaryl groups can have, for example, from about 3 to about 50 carbon atoms and 1-8 heteroatoms (unless explicitly specified otherwise) with from about 4 to about 10 carbon atoms and 1-4 heteroatoms being preferred. In some embodiments, heteroaryl groups are aromatic heterocyclic rings systems having about 4 to about 14 ring atoms including carbon atoms and 1, 2, 3, or 4 heteroatoms selected from oxygen, nitrogen or sulfur. Bicyclic aromatic heteroaryl groups include phenyl, pyridine, pyrimidine or pyridizine rings that are (a) fused to a 6-membered aromatic (unsaturated) heterocyclic ring having one nitrogen atom; (b) fused to a 5- or 6-membered aromatic (unsaturated) heterocyclic ring having two nitrogen atoms; (c) fused to a 5-membered aromatic (unsaturated) heterocyclic ring having one nitrogen atom together with either one oxygen or one sulfur atom; or (d) fused to a 5-membered aromatic (unsaturated) heterocyclic ring having one heteroatom selected from O, N or S. Specifically included within the definition of “heteroaryl” are those aromatic groups that are optionally substituted. Accordingly, the heteroaryl groups described herein include both unsubstituted or substituted groups. Suitable examples of monocyclic and bicyclic heteroaryl groups are selected from: furan, thiophene, indole, azaindole, oxazole, thiazole, isoxazole, isothiazole, imidazole, N-methylimidazole, pyridine, pyrimidine, pyrazine, pyrrole, N-methylpyrrole, pyrazole, N-methylpyrazole, 1,3,4-oxadiazole, 1,2,4-triazole, 1-methyl-1,2,4-triazole, 1H-tetrazole, 1-methyltetrazole, benzoxazole, benzothiazole, benzofuran, benzisoxazole, benzimidazole, N-methylbenzimidazole, azabenzimidazole, indazole, quinazoline, quinoline, and isoquinoline. In representative embodiments of the present invention, the, “heteroaryl” groups are optionally substituted with 1 to 5 substituents selected from: H, C₁-C₃ alkyl, F, Cl, Br, I, CF₃, NO₂, —NR¹R², —CHO, —CONHAr, —C(R³)₂OR³, —C(R³)₂O[C(R³)₂]Ar, —C(R³)₂NR¹R², —C(R³)₂NR³[C(R³)₂]₂NR¹R², —CO₂R⁶, —SOR⁶, or —SO₂R⁶, where Ar is phenyl, 2-pyridyl, 3-pyridyl, 4-pyridyl, 2-thienyl, 3-thienyl, 2-furyl, or 3-furyl, optionally substituted with C₁-C₃ alkyl, —N(R³)₂, and —OH.

The term “heterocycle”, as used herein, whether used alone or as part of another group, refers to a stable 3 to about 10-member ring containing carbons atoms and from 1 to 4 heteroatoms selected from the group consisting of nitrogen, oxygen, and sulfur. A heterocycle of this invention can be either a monocyclic or bicyclic ring system, and can be either saturated, unsaturated, or partially saturated. A heterocycle can be optionally fused to a phenyl ring. Suitable examples of heterocycles include, but are not limited to, aziridinyl, azetidinyl, 1,4-dioxanyl, hexahydroazepinyl, piperazinyl, piperidinyl, pyrrolidinyl, morpholinyl, thiomorpholinyl, dihydrobenzimidazolyl, dihydrobenzofuranyl, dihydrobenzothienyl, dihydrobenzoxazolyl, dihydrofuranyl, dihydroimidazolyl, dihydroindolyl, dihydroisooxazolyl, dihydroisothiazolyl, dihydrooxadiazolyl, dihydrooxazolyl, dihydropyrrazinyl, dihydropyrazolyl, dihydropyridinyl, dihydropyrimidinyl, dihydropyrrolyl, dihydroquinolinyl, dihydrotetrazolyl, dihydrothiadiazolyl, dihydrothiazolyl, dihydrothienyl, dihydrotriazolyl, dihydroazetidinyl, dihydro-1,4-dioxanyl, tetrahydrofuranyl, tetrahydrothienyl, tetrahydroquinolinyl, and tetrahydroisoquinolinyl. Preferred heterocycle moieties include: (a) 6-membered saturated, partially unsaturated, or unsaturated heterocycles containing 1-2 nitrogens, optionally fused to a phenyl ring; (b) 5-membered saturated, partially saturated, or unsaturated heterocycles containing 1-3 nitrogen, oxygen, or sulfur atoms, optionally fused to a phenyl ring; (c) saturated, partially unsaturated, or unsaturated bicyclic heterocycles containing 1-4 nitrogen, oxygen, or sulfur atoms; (d) carbazole, dibenzofuran, and dibenzothiophene. Specifically included in the definition of “heterocycle” are those heterocycles that are optionally substituted with one to four substituents selected from: H, C₁-C₃ alkyl, F, Cl, Br, I, CF₃, NO₂, —NR¹R², —CHO, —CONHAr, —C(R³)₂OR³, —C(R³)₂O[C(R³)₂]Ar, —C(R³)₂NR¹R², —C(R³)₂NR³[C(R³)₂]₂NR¹R², —CO₂R⁶, —SOR⁶, or —SO₂R⁶, where Ar is phenyl, 2-pyridyl, 3-pyridyl, 4-pyridyl, 2-thienyl, 3-thienyl, 2-furyl, or 3-furyl, optionally substituted with C₁-C₃ alkyl, —N(R³)₂, and —OH.

The term “halogen” refers to an atom of fluorine, chlorine, bromine, or iodine. As used herein, the term “substituted” is contemplated to include all permissible substituents of organic compounds. Typically, suitable substituents of organic compounds include acyclic and cyclic, branched and unbranched, carbocyclic and heterocyclic, aromatic and nonaromatic substituents of organic compounds as well as inorganic substituents such as halogen or amino. The substituents can be one or more and the same or different for appropriate organic compounds. For purposes of this invention, the heteroatoms such as nitrogen may have hydrogen substituents, halogen substituents and/or any suitable substituents of organic compounds described herein which satisfy the valencies of the heteroatoms. This invention is not intended to be limited in any manner by the suitable substituents of organic compounds.

Accordingly, present invention provides fused bicyclic 2-aryl- or 2-heteroarylthiazolyl compounds of formula I:

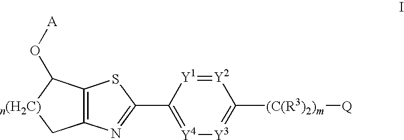

and pharmaceutically acceptable salts thereof
wherein,
A is H, C₁-C₃ alkyl, or acetyl;

### Q is —NR¹R², —NR³N(R³)₂, —NR³OR³ or —OH;

R¹ and R² are each independently H or C₁-C₃ alkyl, or R¹ and R² join together with the nitrogen atom to which each is attached, forming a 4 to 6 membered saturated heterocyclic ring comprising heteroatoms selected from 1-2 nitrogen atoms, 0-1 oxygen atom and 0-1 sulfur atom, said ring optionally substituted with one or more of R⁴;
R³ at each occurrence, is independently H or C₁-C₃ alkyl;
R⁴ is C₁-C₃ alkyl, —N(R³)₂, or —OH;
Y¹, Y², Y³, and Y⁴ are the same or different, and are each independently N or CR⁵, or two R⁵ groups on adjacent carbon atoms join together, with the carbon atoms which they are bonded, to form a 9 to 10 membered bicyclic aryl ring or bicyclic heteroaryl ring, said ring comprising members selected from CR⁵ and N;
R⁵ is independently H or is independently selected from C₁-C₃ alkyl, F, Cl, Br, I, CF₃, NO₂, —NR¹R², —CHO, —CONHAr, —C(R³)₂OR³, —C(R³)₂O[C(R³)₂]Ar, —C(R³)₂NR¹R², —C(R³)₂NR³[C(R³)₂]₂NR¹R², —CO₂R⁶, —SOR⁶, and —SO₂R⁶, where Ar is phenyl, 2-pyridyl, 3-pyridyl, 4-pyridyl, 2-thienyl, 3-thienyl, 2-furyl, or 3-furyl, optionally substituted with one or more of R⁴;
R⁶ is independently H or is independently selected from C₁-C₈ alkyl, C₂-C₈ alkenyl, and C₂-C₈ alkynyl, each optionally substituted with —NR¹R², —OR³, C₄-C₆ cycloalkyl, a saturated heterocyclic ring comprising heteroatoms selected from 0-1 nitrogen atom, 0-1 oxygen atom and 0-1 sulfur atom, or —COCH₃, said C₄-C₆ cycloalkyl, optionally substituted with R⁴; or a saturated heterocyclic ring comprising heteroatoms selected from 0-1 nitrogen atom, 0-1 oxygen atom and 0-1 sulfur atom, and optionally substituted with one or more of R⁴;
m is 0 or 1; and n is an integer from 1 to 3.

Suitable examples of cyclic rings fused to thiazolyl rings in accordance with the invention include, but are not limited to, 4,5-dihydro-4H-cyclopenta[d][1,3]thiazolyl, 5,6-dihydro-4H-cyclopenta[d][1,3]thiazolyl, 4,5,6,7-tetrahydro-1,3-benzothiazolyl, 5,6,7,8-tetrahydro-4H-cyclohepta[d][1,3]thiazolyl and 5,6,7,8-tetrahydro-4H-cycloocta[d][1,3]thiazolyl. According to one embodiment, the cycloalkyl ring, fused to the thiazole ring at the 1,3-position, is substituted by functional groups selected from H, C₁-C₃ alkoxy, and O-acetyl.

According to one embodiment, the ring formed by Y¹, Y², Y³, and Y⁴ that is covalently bonded to the thiazole ring at the 2-position, is an aryl or heteroaryl ring of 6 atoms selected from: phenyl, pyridinyl, pyrimidinyl, and pyrazinyl. According to a separate embodiment, Y¹ and Y² or Y³ and Y⁴ are CR⁵, the two R⁵ groups on adjacent carbon atoms join together, with the carbon atoms which they are bonded, to form a 9 to 10 membered bicyclic aryl ring or bicyclic heteroaryl ring, said ring selected from: napthyl, indenyl, indolyl, benzoxazolyl, benzothiazolyl, benzofuranyl, benzisoxazolyl, benzimidazolyl, N-methylbenzimidazolyl, azabenzimidazolyl, indazolyl, quinazolinyl, quinolinyl, and isoquinolinyl.

According to one embodiment, a phenyl group is covalently bonded to the thiazole ring at the 2-position. The phenyl group is substituted with 1 to 4 functional groups that are selected from: C₁-C₃ alkyl, F, Cl, Br, I, CF₃, NO₂, —NR¹R², —CHO, —CONHAr, —C(R³)₂OR³, —C(R³)₂O[C(R³)₂]Ar, —C(R³)₂NR¹R², —C(R³)₂NR³[C(R³)₂]₂NR¹R², —CO₂R⁶, —SOR⁶, and —SO₂R⁶, wherein Ar is phenyl, 2-pyridyl, 3-pyridyl, 4-pyridyl, 2-thienyl, 3-thienyl, 2-furyl, or 3-furyl, optionally substituted with one or more of R⁴.

According to a separate embodiment, a pyridinyl group is covalently bonded to the thiazole ring at the 2-position. The pyridinyl group can be substituted at positions 2-5. The pyridinyl group is substituted with 1 to 3 functional groups that are selected from C₁-C₃ alkyl, F, Cl, Br, I, CF₃, NO₂, —NR¹R², —CHO, —CONHAr, —C(R³)₂OR³, —C(R³)₂O[C(R³)₂]Ar, —C(R³)₂NR¹R², —C(R³)₂NR³[C(R³)₂]₂NR¹R², —CO₂R⁶, —SOR⁶, and —SO₂R⁶, wherein Ar is phenyl, 2-pyridyl, 3-pyridyl, 4-pyridyl, 2-thienyl, 3-thienyl, 2-furyl, or 3-furyl, optionally substituted with one or more of R⁴.

According to a separate embodiment, an indazolyl group is covalently bonded to the thiazole ring at the 2-position. The indazolyl group is substituted with 1 to 3 functional groups at any acceptable position of the indazolyl ring, including the N-atom of the indazolyl ring, that are selected from C₁-C₃ alkyl, F, Cl, Br, I, CF₃, NO₂, —NR¹R², —CHO, —CONHAr, —C(R³)₂O R³, —C(R³)₂O[C(R³)₂]Ar, —C(R³)₂NR¹R², —C(R³)₂NR³[C(R³)₂]₂NR¹R², —CO₂R⁶, —SOR⁶, and —SO₂R⁶, wherein Ar is phenyl, 2-pyridyl, 3-pyridyl, 4-pyridyl, 2-thienyl, 3-thienyl, 2-furyl, or 3-furyl, optionally substituted with one or more of R⁴.

Suitable examples of fused bicyclic 2-aryl- or 2-heteroarylthiazolyl compounds of formula I include, but are not limited to, compounds selected from:

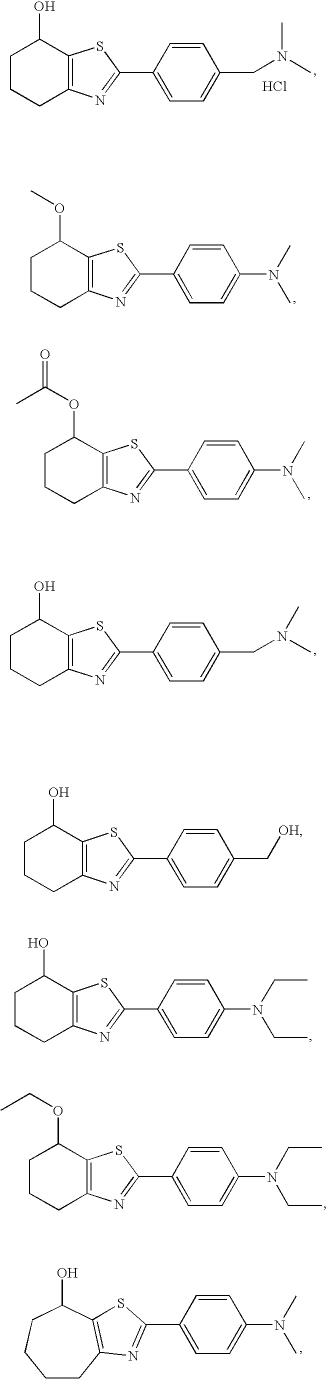

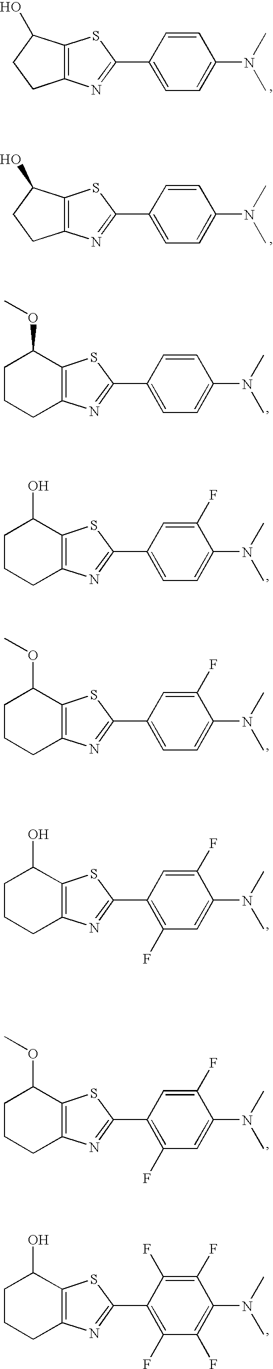

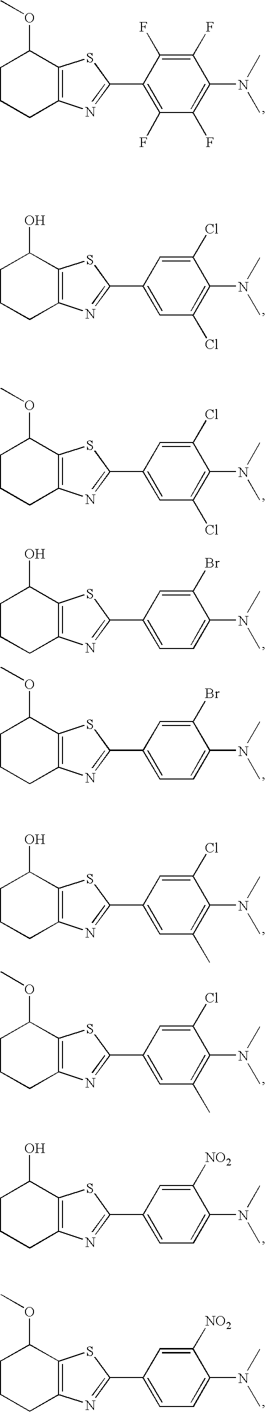

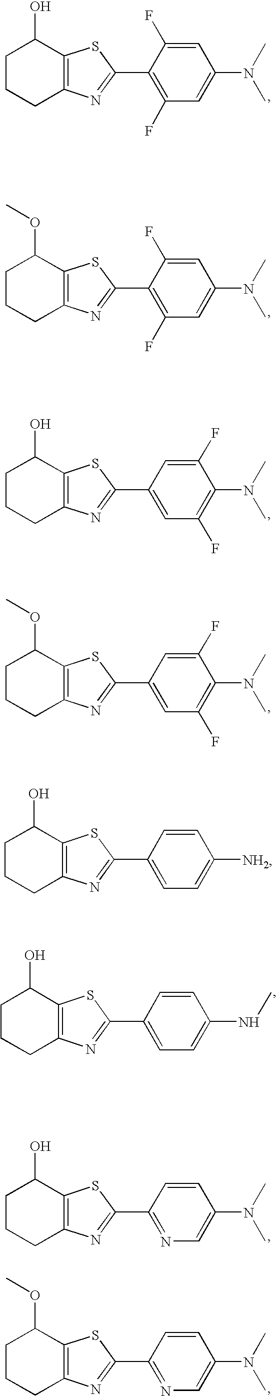

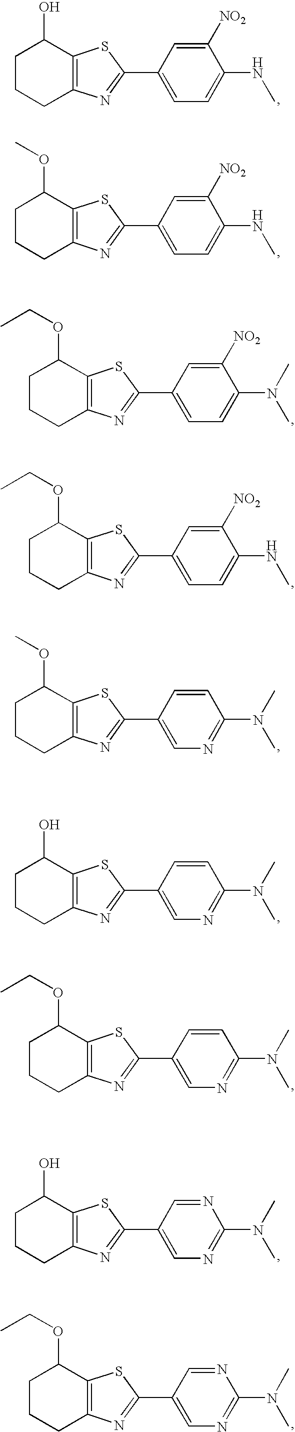

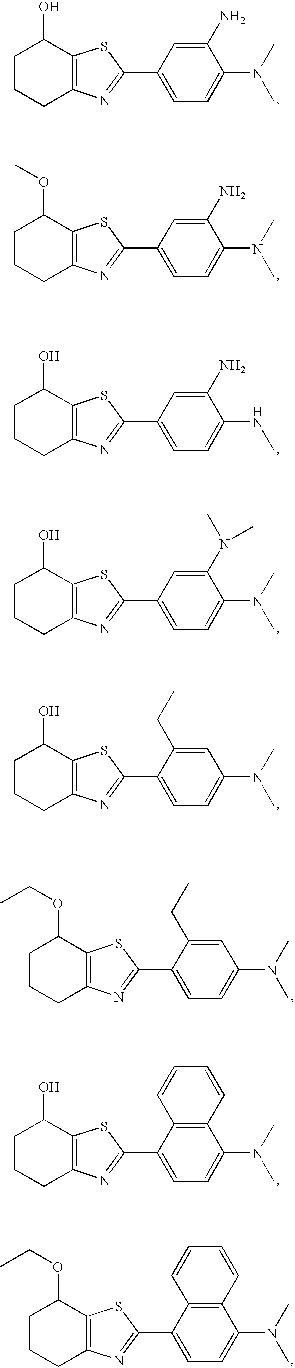

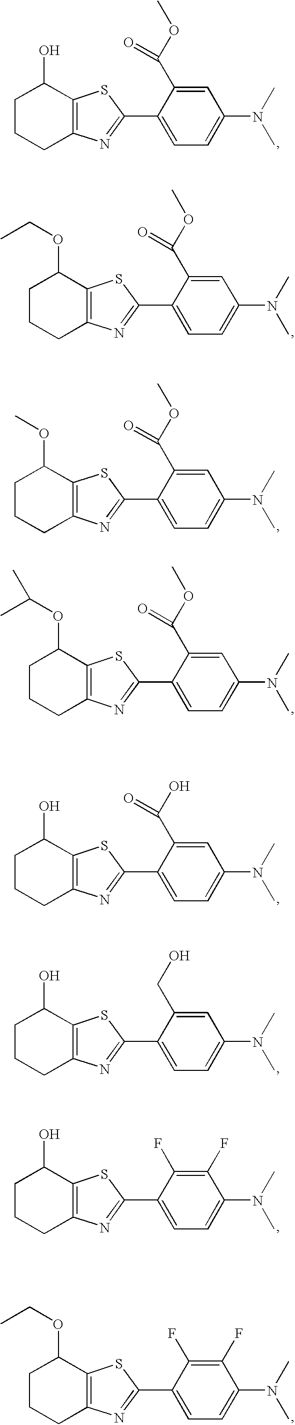

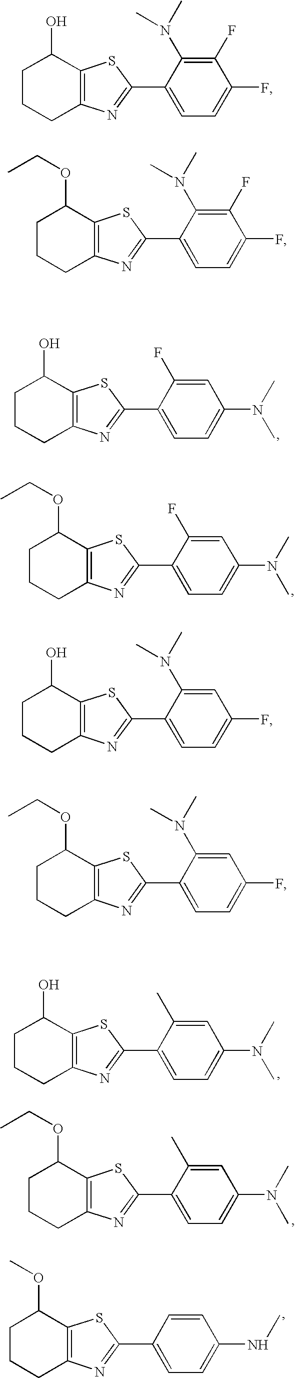

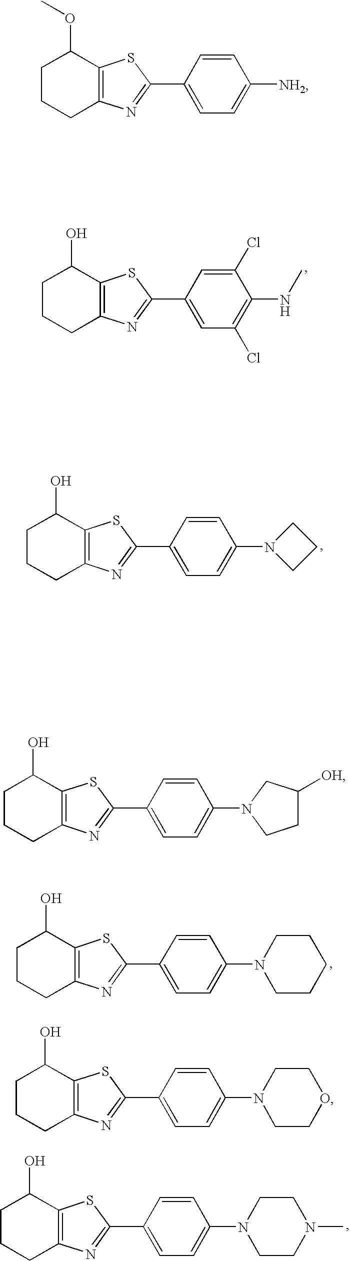

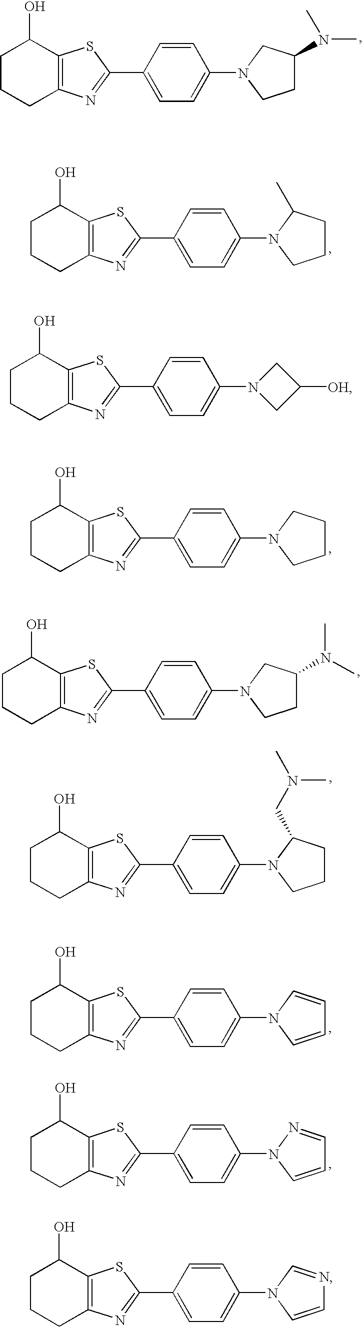

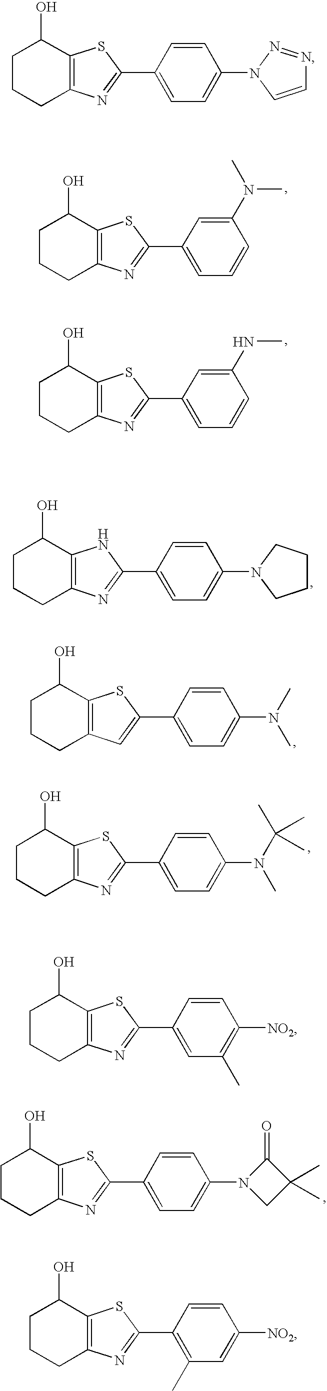

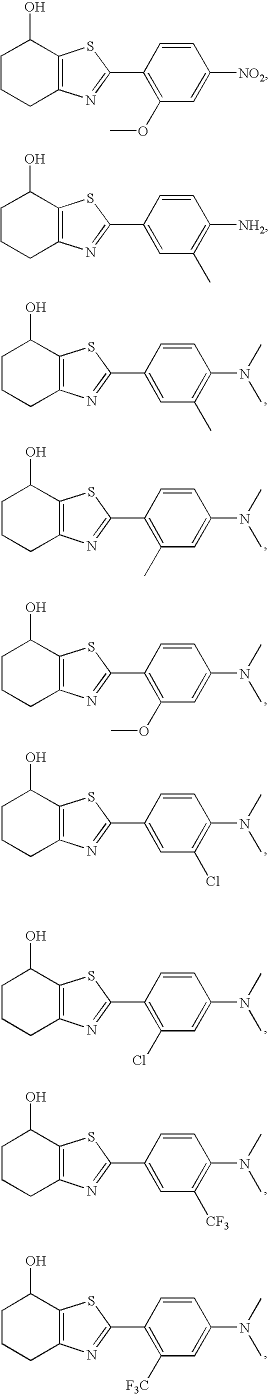

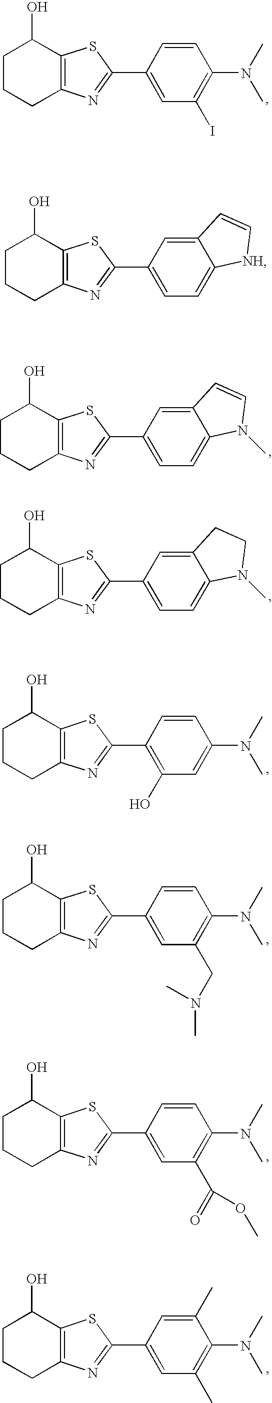

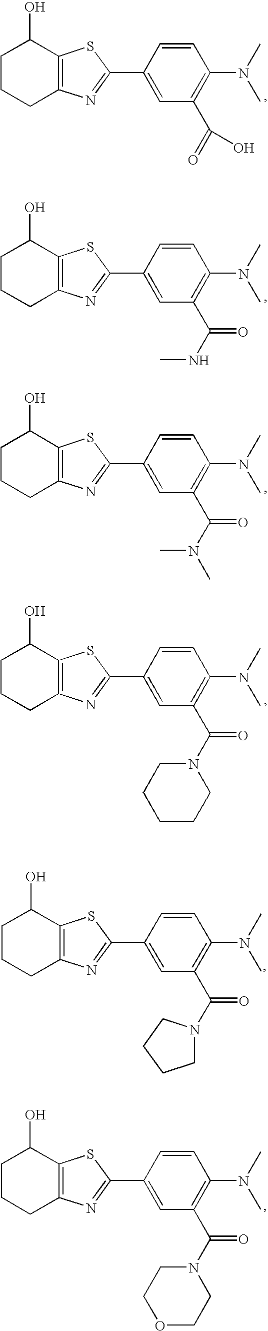

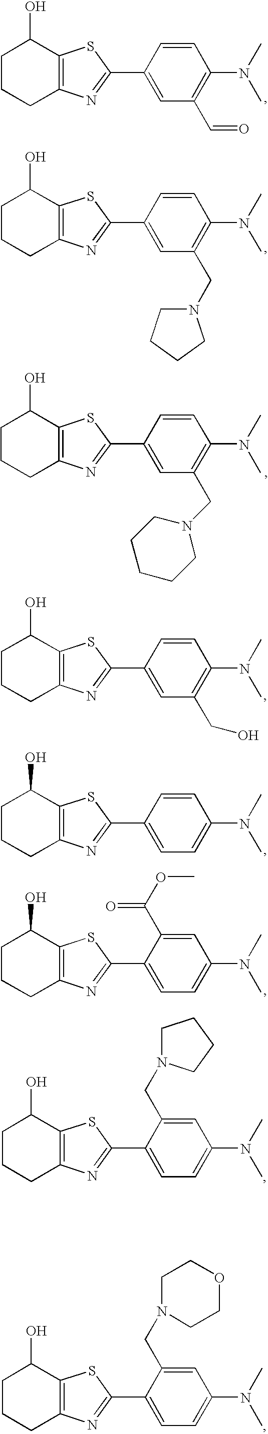

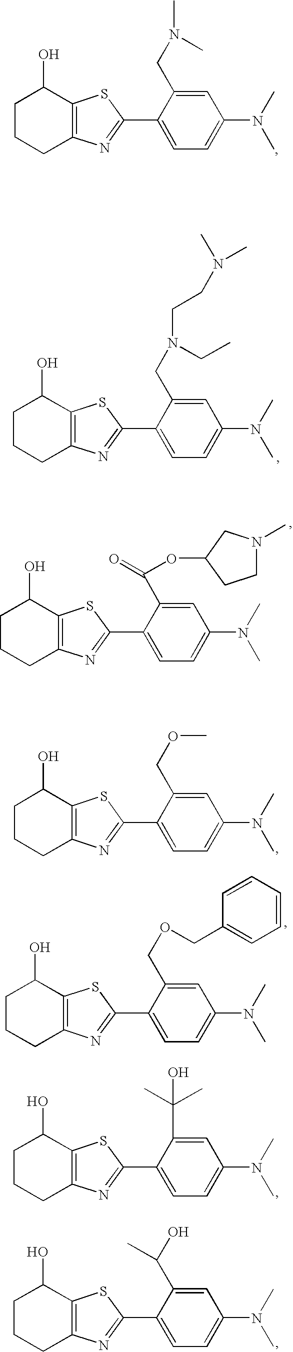

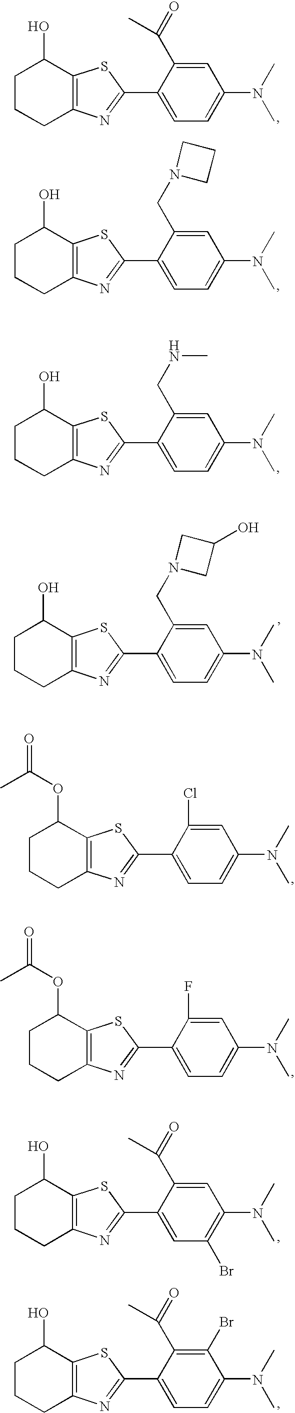

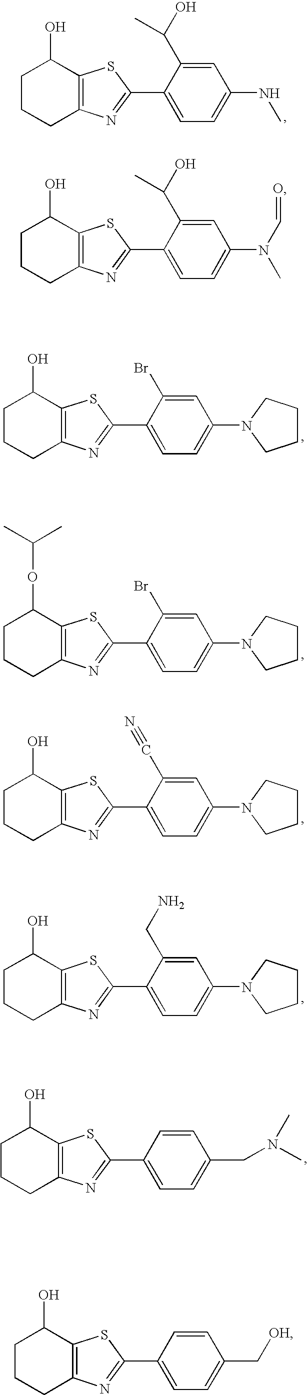

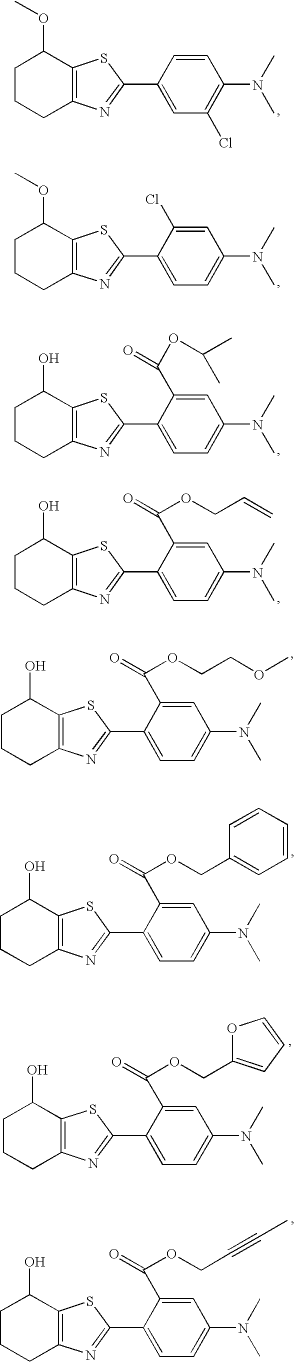

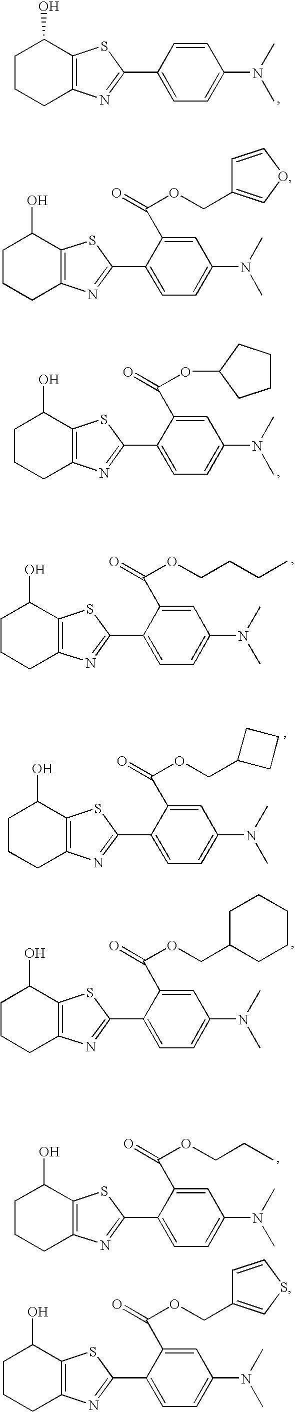

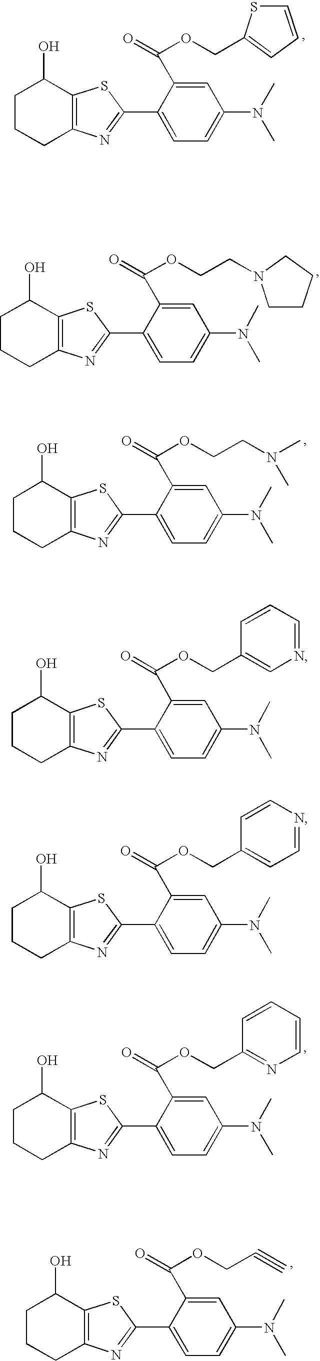

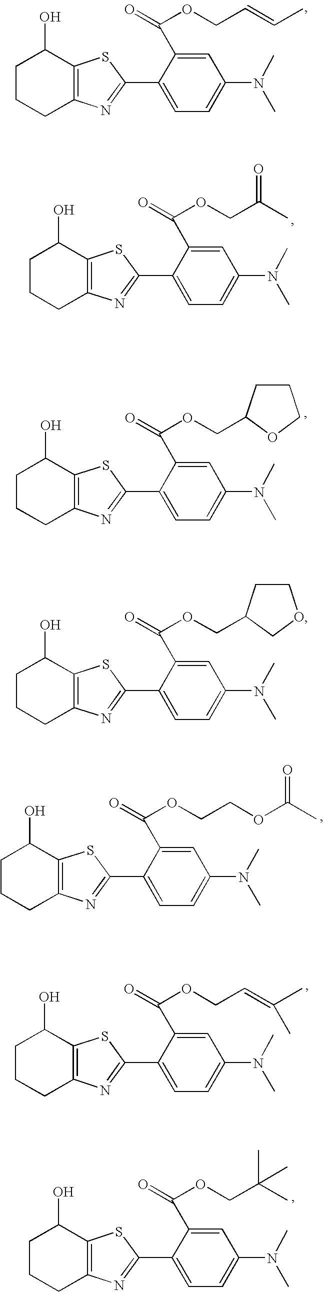

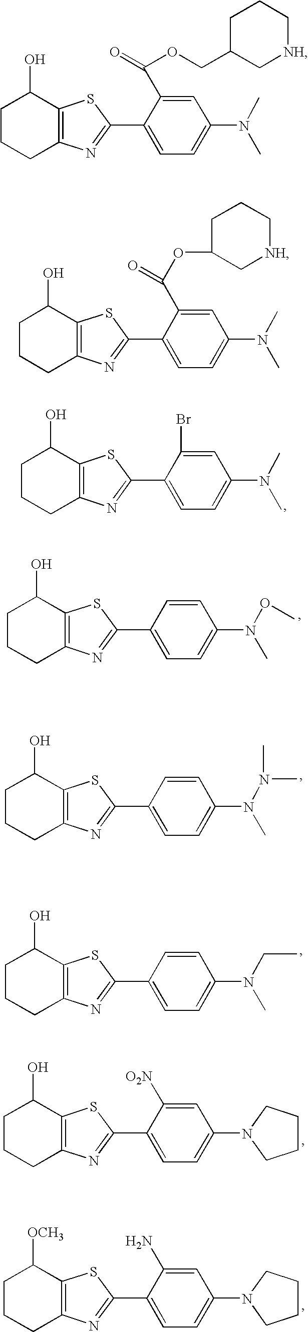

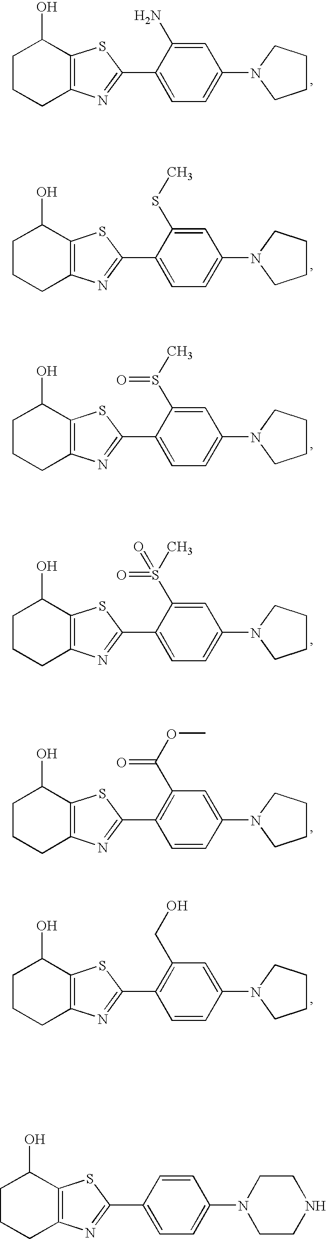

and pharmaceutically acceptable salts thereof.

Compound names corresponding to the structures of fused bicyclic 2-aryl- or 2-heteroarylthiazolyl compounds of formula I include, but are not limited to, compounds selected from: N-[4-(7-methoxy-4,5,6,7-tetrahydro-1,3-benzothiazol-2-yl)phenyl]-N,N-dimethylamine, 2-[4-(dimethylamino)phenyl]-4,5,6,7-tetrahydro-1,3-benzothiazol-7-yl acetate, N-[4-(7-ethoxy-4,5,6,7-tetrahydro-1,3-benzothiazol-2-yl)phenyl]-N,N-diethylamine, 2-[4-(diethylamino)phenyl]-4,5,6,7-tetrahydro-1,3-benzothiazol-7-ol, N-{4-[(7R)-7-methoxy-4,5,6,7-tetrahydro-1,3-benzothiazol-2-yl]phenyl}-N,N-dimethylamine, 2-(4-aminophenyl)-4,5,6,7-tetrahydro-1,3-benzothiazol-7-ol, 2-[4-(methylamino)phenyl]-4,5,6,7-tetrahydro-1,3-benzothiazol-7-ol, 2-[4-(dimethylamino)-3-fluorophenyl]-4,5,6,7-tetrahydro-1,3-benzothiazol-7-ol, N-[2-fluoro-4-(7-methoxy-4,5,6,7-tetrahydro-1,3-benzothiazol-2-yl)phenyl]-N,N-dimethylamine, 2-[4-(dimethylamino)-2,5-difluorophenyl]-4,5,6,7-tetrahydro-1,3-benzothiazol-7-ol, N-[2,5-difluoro-4-(7-methoxy-4,5,6,7-tetrahydro-1,3-benzothiazol-2-yl)phenyl]-N,N-dimethylamine, 2-[4-(dimethylamino)-2,3,5,6-tetrafluorophenyl]-4,5,6,7-tetrahydro-1,3-benzothiazol-7-ol, N,N-dimethyl-N-[2,3,5,6-tetrafluoro-4-(7-methoxy-4,5,6,7-tetrahydro-1,3-benzo-thiazol-2-yl)phenyl]amine, 2-[3,5-dichloro-4-(dimethylamino)phenyl]-4,5,6,7-tetrahydro-1,3-benzothiazol-7-ol, N-[2,6-dichloro-4-(7-methoxy-4,5,6,7-tetrahydro-1,3-benzothiazol-2-yl)phenyl]-N,N-dimethylamine, 2-[3-bromo-4-(dimethylamino)-phenyl]-4,5,6,7-tetrahydro-1,3-benzothiazol-7-ol, N-[2-bromo-4-(7-methoxy-4,5,6,7-tetrahydro-1,3-benzothiazol-2-yl)phenyl]-N,N-dimethylamine, 2-[3-chloro-4-(dimethyl-amino)-5-methylphenyl]-4,5,6,7-tetrahydro-1,3-benzothiazol-7-ol, N-[2-chloro-4-(7-methoxy-4,5,6,7-tetrahydro-1,3-benzothiazol-2-yl)-6-methylphenyl]-N,N-dimethylamine, 2-[4-(dimethylamino)-3-nitrophenyl]-4,5,6,7-tetrahydro-1,3-benzo-thiazol-7-ol, N-[4-(7-methoxy-4,5,6,7-tetrahydro-1,3-benzothiazol-2-yl)-2-nitrophenyl]-N,N-dimethylamine, 2-[4-(dimethylamino)-2,6-difluorophenyl]-4,5,6,7-tetrahydro-1,3-benzothiazol-7-ol, [3,5-difluoro-4-(7-methoxy-4,5,6,7-tetrahydro-1,3-benzothiazol-2-yl)phenyl]dimethylamine, 2-[4-(dimethylamino)-3,5-difluorophenyl]-4,5,6,7-tetrahydro-1,3-benzothiazol-7-ol, [2,6-difluoro-4-(7-methoxy-4,5,6,7-tetrahydro-1,3-benzothiazol-2-yl)phenyl]dimethylamine, 2-[3,5-Dichloro-4-(methylamino)phenyl]-4,5,6,7-tetrahydro-1,3-benzothiazol-7-ol, [6-(7-methoxy-4,5,6,7-tetrahedro-benzothiazol-2-yl)-pyridin-3-yl]-dimethylamine, 2-(5-Dimethylamino-pyridine-2-yl)-4,5,6,7-tetrahedro-benzothiazol-7-ol, [4-(7-Methoxy-4,5,6,7-tetrahydro-1,3-benzothiazol-2-yl)phenyl]-amine, 2-[4-(methylamino)-3-nitrophenyl]-4,5,6,7-tetrahydro-1,3-benzothiazol-7-ol, N-[4-(7-methoxy-4,5,6,7-tetrahydro-1,3-benzothiazol-2-yl)-2-nitrophenyl]-N-methyl-amine, [4-(7-ethoxy-4,5,6,7-tetrahydro-1,3-benzothiazol-2-yl)-2-nitrophenyl]-dimethylamine, [4-(7-ethoxy-4,5,6,7-tetrahydro-1,3-benzothiazol-2-yl)-2-nitrophenyl]-methylamine, N-[4-(7-methoxy-4,5,6,7-tetrahydro-1,3-benzothiazol-2-yl)phenyl]-N-methylamine, 5-(7-Methoxy-4,5,6,7-tetrahydro-1,3-benzothiazol-2-yl)-N,N-dimethyl-pyridin-2-amine, 2-[6-(dimethylamino)pyridin-3-yl]-4,5,6,7-tetrahydro-1,3-benzo-thiazol-7-ol, 5-(7-Ethoxy-4,5,6,7-tetrahydro-1,3-benzothiazol-2-yl)-N,N-dimethyl-pyridin-2-amine, 2-[4-(dimethylamino)-2-fluorophenyl]-4,5,6,7-tetrahydro-1,3-benzothiazol-7-ol, [4-(7-ethoxy-4,5,6,7-tetrahydro-1,3-benzothiazol-2-yl)-3-fluorophenyl]dimethylamine, 2-[4-(dimethylamino)-2,3-difluorophenyl]-4,5,6,7-tetrahydro-1,3-benzothiazol-7-ol, [4-(7-ethoxy-4,5,6,7-tetrahydro-1,3-benzothiazol-2-yl)-2,3-difluorophenyl]dimethylamine, 2-[4-(dimethylamino)-2-methylphenyl]-4,5,6,7-tetrahydro-1,3-benzothiazol-7-ol, [4-(7-ethoxy-4,5,6,7-tetrahydro-1,3-benzothiazol-2-yl)-3-methylphenyl]dimethylamine, 2-[4-(dimethylamino)-2-ethylphenyl]-4,5,6,7-tetrahydro-1,3-benzothiazol-7-ol, [4-(7-ethoxy-4,5,6,7-tetrahydro-1,3-benzothiazol-2-yl)-3-ethylphenyl]dimethylamine, 2-[3-amino-4-(dimethylamino)phenyl]-4,5,6,7-tetrahydro-1,3-benzothiazol-7-ol, 4-(7-methoxy-4,5,6,7-tetrahydro-1,3-benzothiazol-2-yl)-N1,N1-dimethylbenzene-1,2-diamine, 2-[3,4-bis(dimethylamino)phenyl]-4,5,6,7-tetrahydro-1,3-benzothiazol-7-ol, 2-[3-amino-4-(methylamino)phenyl]-4,5,6,7-tetrahydro-1,3-benzothiazol-7-ol, 2-[2-(dimethylamino)pyrimidin-5-yl]-4,5,6,7-tetrahydro-1,3-benzothiazol-7-ol, 5-(7-ethoxy-4,5,6,7-tetrahydro-1,3-benzothiazol-2-yl)-N,N-dimethylpyrimidin-2-amine, methyl 5-(dimethylamino)-2-(7-hydroxy-4,5,6,7-tetrahydro-1,3-benzothiazol-2-yl)benzoate, methyl 5-(dimethylamino)-2-(7-ethoxy-4,5,6,7-tetrahydro-1,3-benzothiazol-2-yl)benzoate, methyl 5-(dimethylamino)-2-(7-methoxy-4,5,6,7-tetrahydro-1,3-benzothiazol-2-yl)benzoate, methyl 5-(dimethylamino)-2-(7-isopropoxy-4,5,6,7-tetrahydro-1,3-benzothiazol-2-yl)benzoate, 5-(dimethylamino)-2-(7-hydroxy-4,5,6,7-tetrahydro-1,3-benzothiazol-2-yl)benzoic acid, 2-[4-(dimethylamino)-2-(hydroxymethyl)phenyl]-4,5,6,7-tetrahydro-1,3-benzothiazol-7-ol, 2-[4-(dimethylamino)-2-(pyrrolidin-1-ylmethyl)phenyl]-4,5,6,7-tetrahydro-1,3-benzothiazol-7-ol, 2-[4-(dimethylamino)-2-(morpholin-4-ylmethyl)phenyl]-4,5,6,7-tetrahydro-1,3-benzothiazol-7-ol, 2-{4-(dimethylamino)-2-[(dimethylamino)methyl]phenyl}-4,5,6,7-tetrahydro-1,3-benzothiazol-7-ol, 2-(4-(dimethylamino)-2-{[[2-(dimethylamino)ethyl]-(ethyl)amino]methyl}phenyl)-4,5,6,7-tetrahydro-1,3-benzothiazol-7-ol, 1-methylpyrrolidin-3-yl 5-(dimethylamino)-2-(7-hydroxy-4,5,6,7-tetrahydro-1,3-benzothiazol-2-yl)benzoate, 2-[4-(Dimethylamino)-2-(methoxymethyl)phenyl]-4,5,6,7-tetrahydro-1,3-benzothiazol-7-ol, 2-[2-[(Benzyloxy)-methyl]-4-(dimethylamino)phenyl]-4,5,6,7-tetrahydro-1,3-benzothiazol-7-ol, 3-[(benzyloxy)methyl]-4-(7-isopropoxy-4,5,6,7-tetrahydro-1,3-benzothiazol-2-yl)-N,N-dimethylaniline, 2-[4-(dimethylamino)-2-(1-hydroxy-1-methylethyl)phenyl]-4,5,6,7-tetrahydro-1,3-benzothiazol-7-ol, 2-[4-(dimethylamino)-2-(1-hydroxyethyl)phenyl]-4,5,6,7-tetrahydro-1,3-benzothiazol-7-ol, 1-[5-(Dimethylamino)-2-(7-hydroxy-4,5,6,7-tetrahydro-1,3-benzothiazol-2-yl)phenyl]ethanone, 2-[2-(azetidin-1-ylmethyl)-4-(dimethylamino)phenyl]-4,5,6,7-tetrahydro-1,3-benzothiazol-7-ol, 2-{4-(dimethyl-amino)-2-[(methylamino)methyl]phenyl}-4,5,6,7-tetrahydro-1,3-benzothiazol-7-ol, 2-{4-(dimethylamino)-2-[(3-hydroxyazetidin-1-yl)methyl]phenyl}-4,5,6,7-tetrahydro-1,3-benzothiazol-7-ol, 2-[2-chloro-4-(dimethylamino)phenyl]-4,5,6,7-tetrahydro-1,3-benzothiazol-7-yl acetate, 2-[4-(dimethylamino)-2-fluorophenyl]-4,5,6,7-tetrahydro-1,3-benzothiazol-7-yl acetate, 1-[4-bromo-5-(dimethylamino)-2-(7-hydroxy-4,5,6,7-tetrahydro-1,3-benzothiazol-2-yl)phenyl]ethanone, 1-[2-bromo-3-(dimethylamino)-6-(7-hydroxy-4,5,6,7-tetrahydro-1,3-benzothiazol-2-yl)phenyl]ethanone, 2-[2-(1-hydroxyethyl)-4-(methylamino)phenyl]-4,5,6,7-tetrahydro-1,3-benzothiazol-7-ol, 2-(2-bromo-4-pyrrolidin-1-ylphenyl)-4,5,6,7-tetrahydro-1,3-benzothiazol-7-ol, 2-(2-bromo-4-pyrrolidin-1-ylphenyl)-7-isopropoxy-4,5,6,7-tetrahydro-1,3-benzothiazole, 2-(7-hydroxy-4,5,6,7-tetrahydro-1,3-benzothiazol-2-yl)-5-pyrrolidin-1-ylbenzonitrile, 2-[2-(aminomethyl)-4-pyrrolidin-1-yl phenyl]-4,5,6,7-tetrahydro-1,3-benzothiazol-7-ol, 2-{4-[(dimethylamino)methyl]phenyl}-4,5,6,7-tetrahydro-1,3-benzothiazol-7-ol, 2-[4-(hydroxylmethyl)phenyl]-4,5,6,7-tetrahydro-1,3-benzothiazol-7-ol, 2-(4-amino-3-methylphenyl)-4,5,6,7-tetrahydro-1,3-benzothiazol-7-ol, 2-[4-(dimethylamino)-3-methylphenyl]-4,5,6,7-tetrahydro-1,3-benzothiazol-7-ol, [2-chloro-4-(7-methoxy-4,5,6,7-tetrahydro-1,3-benzothiazol-2-yl)phenyl]-dimethylamine, 2-[3-chloro-4-(dimethylamino)phenyl]-4,5,6,7-tetrahydro-1,3-benzothiazol-7-ol, 3-chloro-4-(7-methoxy-4,5,6,7-tetrahydro-1,3-benzothiazol-2-yl)phenyl]dimethylamine, 2-[2-chloro-4-(dimethylamino)phenyl]-4,5,6,7-tetrahydro-1,3-benzothiazol-7-ol, 2-[4-(dimethylamino)-2-methylphenyl]-4,5,6,7-tetrahydro-1,3-benzothiazol-7-ol, 2-[4-(dimethylamino)-2-methoxyphenyl]-4,5,6,7-tetrahydro-1,3-benzothiazol-7-ol, 2-[4-(dimethylamino)-3-iodophenyl]-4,5,6,7-tetrahydro-1,3-benzothiazol-7-ol, 2-[4-(dimethylamino)-2-(trifluoromethyl)phenyl]-4,5,6,7-tetrahydro-1,3-benzothiazol-7-ol, 2-[4-(dimethylamino)-3-(trifluoromethyl)phenyl]-4,5,6,7-tetrahydro-1,3-benzothiazol-7-ol, (7R)-2-[4-(dimethylamino)-2-methyl phenyl]-4,5,6,7-tetrahydro-1,3-benzothiazol-7-ol, (7S)-2-[4-(dimethylamino)-2-methylphenyl]-4,5,6,7-tetrahydro-1,3-benzothiazol-7-ol, 2-[4-(dimethylamino)-2-hydroxyphenyl]-4,5,6,7-tetrahydro-1,3-benzothiazol-7-ol, 2-[4-(dimethylamino)-3,5-dimethylphenyl]-4,5,6,7-tetrahydro-1,3-benzothiazol-7-ol, methyl 2-(dimethylamino)-5-(7-hydroxy-4,5,6,7-tetrahydro-1,3-benzothiazol-2-yl)benzoate, methyl 2-(dimethylamino)-5-(7-methoxy-4,5,6,7-tetrahydro-1,3-benzothiazol-2-yl)benzoate, 2-(dimethylamino)-5-(7-hydroxy-4,5,6,7-tetrahydro-1,3-benzothiazol-2-yl)benzoic acid, 2-[4-(dimethylamino)-3-(hydroxymethyl)phenyl]-4,5,6,7-tetrahydro-1,3-benzothiazol-7-ol, 2-{4-(dimethylamino)-3-[(dimethylamino)methyl]phenyl}-4,5,6,7-tetrahydro-1,3-benzothiazol-7-ol, 2-[4-(dimethylamino)-3-(pyrrolidin-1-ylmethyl)phenyl]-4,5,6,7-tetrahydro-1,3-benzothiazol-7-ol, 2-[4-(dimethylamino)-3-(piperidin-1-ylmethyl)phenyl]-4,5,6,7-tetrahydro-1,3-benzothiazol-7-ol, 2-(dimethylamino)-5-(7-hydroxy-4,5,6,7-tetrahydro-1,3-benzothiazol-2-yl)benzaldehyde, isopropyl 5-(dimethylamino)-2-(7-hydroxy-4,5,6,7-tetrahydro-1,3-benzothiazol-2-yl)benzoate, allyl 5-(dimethylamino)-2-(7-hydroxy-4,5,6,7-tetrahydro-1,3-benzothiazol-2-yl)benzoate, 2-methoxyethyl 5-(dimethyl-amino)-2-(7-hydroxy-4,5,6,7-tetrahydro-1,3-benzothiazol-2-yl)benzoate, benzyl 5-(dimethylamino)-2-(7-hydroxy-4,5,6,7-tetrahydro-1,3-benzothiazol-2-yl)benzoate, 2-furylmethyl 5-(dimethylamino)-2-(7-hydroxy-4,5,6,7-tetrahydro-1,3-benzothiazol-2-yl)benzoate, 3-furylmethyl 5-(dimethylamino)-2-(7-hydroxy-4,5,6,7-tetrahydro-1,3-benzothiazol-2-yl)benzoate, cyclopentyl 5-(dimethylamino)-2-(7-hydroxy-4,5,6,7-tetrahydro-1,3-benzothiazol-2-yl)benzoate, butyl 5-(dimethylamino)-2-(7-hydroxy-4,5,6,7-tetrahydro-1,3-benzothiazol-2-yl)benzoate, cyclobutylmethyl 5-(dimethyl-amino)-2-(7-hydroxy-4,5,6,7-tetrahydro-1,3-benzothiazol-2-yl)benzoate, cyclohexyl methyl 5-(dimethylamino)-2-(7-hydroxy-4,5,6,7-tetrahydro-1,3-benzo-thiazol-2-yl)benzoate, propyl 5-(dimethylamino)-2-(7-hydroxy-4,5,6,7-tetrahydro-1,3-benzothiazol-2-yl)benzoate, 3-thienylmethyl 5-(dimethylamino)-2-(7-hydroxy-4,5,6,7-tetrahydro-1,3-benzothiazol-2-yl)benzoate, 2-thienylmethyl 5-(dimethylamino)-2-(7-hydroxy-4,5,6,7-tetrahydro-1,3-benzothiazol-2-yl)benzoate, 2-pyrrolidin-1-ylethyl 5-(dimethylamino)-2-(7-hydroxy-4,5,6,7-tetrahydro-1,3-benzothiazol-2-yl)benzoate, 2-(dimethylamino)ethyl 5-(dimethylamino)-2-(7-hydroxy-4,5,6,7-tetrahydro-1,3-benzothiazol-2-yl)benzoate, pyridin-3-ylmethyl 5-(dimethylamino)-2-(7-hydroxy-4,5,6,7-tetrahydro-1,3-benzothiazol-2-yl)benzoate, pyridin-4-ylmethyl 5-(dimethylamino)-2-(7-hydroxy-4,5,6,7-tetrahydro-1,3-benzothiazol-2-yl)benzoate, pyridin-2-ylmethyl 5-(dimethylamino)-2-(7-hydroxy-4,5,6,7-tetrahydro-1,3-benzo-thiazol-2-yl)benzoate, prop-2-yn-1-yl 5-(dimethylamino)-2-(7-hydroxy-4,5,6,7-tetrahydro-1,3-benzothiazol-2-yl)benzoate, (2E)-but-2-en-1-yl 5-(dimethylamino)-2-(7-hydroxy-4,5,6,7-tetrahydro-1,3-benzothiazol-2-yl)benzoate, 2-oxopropyl 5-(dimethylamino)-2-(7-hydroxy-4,5,6,7-tetrahydro-1,3-benzothiazol-2-yl)benzoate, tetrahydrofuran-2-ylmethyl 5-(dimethylamino)-2-(7-hydroxy-4,5,6,7-tetrahydro-1,3-benzothiazol-2-yl)benzoate, but-2-yn-1-yl 5-(dimethylamino)-2-(7-hydroxy-4,5,6,7-tetrahydro-1,3-benzothiazol-2-yl)benzoate, tetrahydrofuran-3-ylmethyl 5-(dimethylamino)-2-(7-hydroxy-4,5,6,7-tetrahydro-1,3-benzothiazol-2-yl)benzoate, 2-(acetyloxy)ethyl 5-(dimethylamino)-2-(7-hydroxy-4,5,6,7-tetrahydro-1,3-benzothiazol-2-yl)benzoate, 3-methyl but-2-en-1-yl 5-(dimethylamino)-2-(7-hydroxy-4,5,6,7-tetrahydro-1,3-benzothiazol-2-yl)benzoate and 2,2-dimethylpropyl 5-(dimethylamino)-2-(7-hydroxy-4,5,6,7-tetrahydro-1,3-benzothiazol-2-yl)benzoate, piperidin-3-ylmethyl 5-(dimethylamino)-2-(7-hydroxy-4,5,6,7-tetrahydro-1,3-benzothiazol-2-yl)benzoate, piperidin-3-yl 5-(dimethylamino)-2-(7-hydroxy-4,5,6,7-tetrahydro-1,3-benzothiazol-2-yl)benzoate, 2-[2-bromo-4-(dimethylamino)phenyl]-4,5,6,7-tetrahydro-1,3-benzothiazol-7-ol, 2-{4-[methoxy(methyl)amino]phenyl}-4,5,6,7-tetrahydro-1,3-benzothiazol-7-ol, 2-[4-(trimethylhydrazino)phenyl]-4,5,6,7-tetrahydro-1,3-benzothiazol-7-ol, 2-(4-azetidin-1-ylphenyl)-4,5,6,7-tetrahydro-1,3-benzothiazol-7-ol, 2-[4-(3-Hydroxypyrrolidin-1-yl)phenyl]-4,5,6,7-tetrahydro-1,3-benzothiazol-7-ol, 2-(4-Piperidin-1-ylphenyl)-4,5,6,7-tetrahydro-1,3-benzothiazol-7-ol, 2-(4-Morpholin-4-ylphenyl)-4,5,6,7-tetrahydro-1,3-benzothiazol-7-ol, 2-[4-(4-methylpiperazin-1-yl)phenyl]-4,5,6,7-tetrahydro-1,3-benzothiazol-7-ol, 2-{4-[(3S)-3-(Dimethyl-amino)pyrrolidin-1-yl]phenyl}-4,5,6,7-tetrahydro-1,3-benzothiazol-7-ol, 2-[4-(2-Methylpyrrolidin-1-yl)phenyl]-4,5,6,7-tetrahydro-1,3-benzothiazol-7-ol, 2-{4-[ethyl(methyl)amino]phenyl}-4,5,6,7-tetrahydro-1,3-benzothiazol-7-ol, 2-(2-nitro-4-pyrrolidin-1-ylphenyl)-4,5,6,7-tetrahydro-1,3-benzothiazol-7-ol, 2-(7-methoxy-4,5,6,7-tetrahydro-1,3-benzothiazol-2-yl)-5-pyrrolidin-1-ylaniline, 2-(7-methoxy-4,5,6,7-tetrahydro-1,3-benzothiazol-2-yl)-5-pyrrolidin-1-ylaniline, 2-(2-amino-4-pyrrolidin-1-ylphenyl)-4,5,6,7-tetrahydro-1,3-benzothiazol-7-ol, 2-[2-(methylthio)-4-pyrrolidin-1-ylphenyl]-4,5,6,7-tetrahydro-1,3-benzothiazol-7-ol, 2-[2-(Methylsulfinyl)-4-pyrrolidin-1-ylphenyl]-4,5,6,7-tetrahydro-1,3-benzothiazol-7-ol and 2-[2-(Methylsulfonyl)-4-pyrrolidin-1-ylphenyl]-4,5,6,7-tetrahydro-1,3-benzothiazol-7-ol, 2-[4-(3-hydroxy-azetidin-1-yl)-phenyl]-4,5,6,7-tetrahydro-benzothiazol-7-ol, 2-(4-pyrrolidin-1-yl-phenyl)-4,5,6,7-tetrahydro-benzothiazol-7-ol, 2-[4-(3-dimethylamino-pyrrolidin-1-yl)-phenyl]-4,5,6,7-tetrahydro-benzothiazol-7-ol, 2-[4-(2-dimethylaminomethyl-pyrrolidin-1-yl)-phenyl]-4,5,6,7-tetrahydro-benzothiazol-7-ol, 2-(7-Hydroxy-4,5,6,7-tetrahydro-benzothiazol-2-yl)-5-pyrrolidin-1-yl-benzoic acid methyl ester, 2-(2-Hydroxymethyl-4-pyrrolidin-1-yl-phenyl)-4,5,6,7-tetrahydro-benzothiazol-7-ol, 2-(4-Piperazin-1-yl-phenyl)-4,5,6,7-tetrahydrobenzothiazol-7-ol, (7R)-2-[4-(dimethylamino)phenyl]-4,5,6,7-tetrahydro-1,3-benzothiazol-7-ol, methyl 5-(dimethylamino)-2-[(7R)-7-hydroxy-4,5,6,7-tetrahydro-1,3-benzothiazol-2-yl]benzoate, 2-[4-(dimethylamino)phenyl]-4,5,6,7-tetrahydro-1,3-benzothiazol-7-ol, (7S)-2-[4-(dimethylamino)phenyl]-4,5,6,7-tetrahydro-1,3-benzothiazol-7-ol, 2-[4-(dimethylamino)-1-naphthyl]-4,5,6,7-tetrahydro-1,3-benzothiazol-7-ol, [4-(7-ethoxy-4,5,6,7-tetrahydro-1,3-benzothiazol-2-yl)-1-naphthyl]dimethylamine and pharmaceutically acceptable salts thereof.

Where present, fused bicyclic 2-aryl- or 2-heteroarylthiazolyl compounds of formula I and corresponding pharmaceutically acceptable salts or esters thereof include isomers either individually or as a mixture, such as enantiomers, diastereomers, and positional isomers. “Pharmaceutically acceptable salts and esters” refers to salts and esters that are pharmaceutically acceptable and have the desired pharmacological properties. Such salts include, for example, salts that can be formed where acidic protons present in the compounds are capable of reacting with inorganic or organic bases. Suitable inorganic salts include, for example, those formed with the alkali metals or alkaline earth metals, e.g. sodium and potassium, magnesium, calcium, and aluminum. Suitable organic salts include, for example, those formed with organic bases such as the amine bases, e.g. ethanolamine, diethanolamine, triethanolamine, tromethamine, N-methylglucamine, and the like. Pharmaceutically acceptable salts can also include acid addition salts formed from the reaction of basic moieties, such as amines, in the parent compound with inorganic acids (e.g. hydrochloric and hydrobromic acids) and organic acids (e.g. acetic acid, citric acid, maleic acid, and the alkane- and arene-sulfonic acids such as methanesulfonic acid and benzenesulfonic acid).

Pharmaceutically acceptable esters include esters formed from carboxy, sulfonyloxy, and phosphonoxy groups present in the compounds, e.g. C₁₋₆alkyl esters. When there are two acidic groups present, a pharmaceutically acceptable salt or ester can be a mono-acid-mono-salt or ester or a di-salt or ester; and similarly where there are more than two acidic groups present, some or all of such groups can be salified or esterified. Compounds named in this invention can be present in unsalified or unesterified form, or in salified and/or esterified form, and the naming of such compounds is intended to include both the original (unsalified and unesterified) compound and its pharmaceutically acceptable salts and esters. Also, certain compounds named in this invention can be present in more than one stereoisomeric form, and the naming of such compounds is intended to include all single stereoisomers and all mixtures (whether racemic or otherwise) of such stereoisomers.

Pharmaceutically acceptable salts of fused bicyclic 2-aryl- or 2-heteroarylthiazolyl compounds of formula I with an acidic moiety may be formed from organic and inorganic bases. For example with alkali metals or alkaline earth metals such as sodium, potassium, lithium, calcium, or magnesium or organic bases and N-tetraalkylammonium salts such as N-tetrabutylammonium salts. Similarly, when a compound of this invention contains a basic moiety, salts may be formed from organic and inorganic acids. For example salts may be formed from acids: acetic, propionic, lactic, citric, tartaric, succinic, fumaric, maleic, malonic, mandelic, malic, phthalic, hydrochloric, hydrobromic, phosphoric, nitric, sulfuric, methanesulfonic, naphthalenesulfonic, benzenesulfonic, toluenesulfonic, camphorsulfonic, and similarly known acceptable acids when a compound of this invention contains a basic functional group. Other suitable examples of pharmaceutically acceptable salts include, but are not limited, to sulfate; citrate, acetate; oxalate; chloride; bromide; iodide; nitrate; bisulfate; phosphate; acid phosphate; isonicotinate; lactate; salicylate; acid citrate; tartrate; oleate; tannate; pantothenate; bitartrate; ascorbate; succinate; maleate; gentisinate; fumarate; gluconate; glucaronate; saccharate; formate; benzoate; glutamate; methanesulfonate; ethanesulfonate; benzenesulfonate; p-toluenesulfonate; pamoate (i.e., 1,1′-methylene-bis-(2-hydroxy-3-naphthoate)); and salts of fatty acids such as caproate, laurate, myristate, palmitate, stearate, oleate, linoleate, and linolenate salts. The compounds can also be used in the form of esters, carbamates and other conventional ester forms, also referred to herein as prodrug forms, which when administered in such form, convert to the active moiety in-vivo. Exemplary ester forms of the compounds of this invention include, but are not limited to, straight chain alkyl esters having from 1 to 6 carbon atoms or branched chain alkyl groups containing 1 to 6 carbon atoms, including methyl, ethyl, propyl, butyl, 2-methylpropyl and 1,1-dimethylethyl esters, cycloalkyl esters, alkylaryl esters, benzyl esters, and the like.

The fused bicyclic 2-aryl- or 2-heteroarylthiazolyl compounds of formula I are prepared from: (a) commercially available starting materials (b) known starting materials which may be prepared as described in literature procedures or (c) new intermediates described in the schemes and experimental procedures herein. Reactions are performed in a solvent appropriate to the reagents and materials employed and suitable for the transformation being effected. It is understood by those skilled in the art of organic synthesis that the various functionalities present on the molecule is consistent with the chemical transformation proposed.

One method for preparing a fused bicyclic 2-aryl- or 2-heteroarylthiazolyl compound of formula I and pharmaceutically acceptable salts thereof is to react an appropriately substituted arylnitrile compound of formula 1 with phosphorous pentasulfide, which yields a corresponding substituted thioamide compound of formula 2. The resulting substituted thioamide compound of formula 2 is then treated with an epoxyketone of formula 3. The product of the reaction is the fused bicyclic 2-aryl- or 2-heteroarylthiazolyl compound of formula I, or its pharmaceutically acceptable salt. The reaction is summarized in Scheme 1.

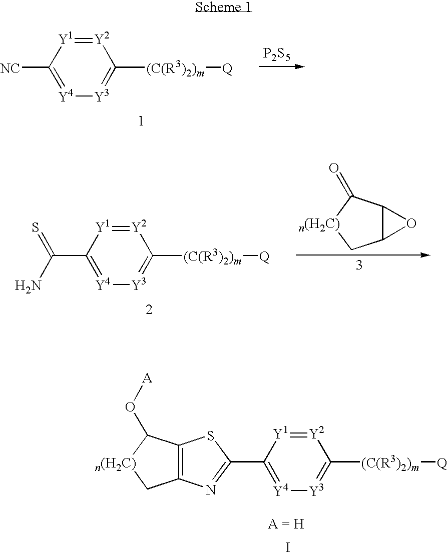

Another method used to synthesize a fused bicyclic 2-aryl- or 2-heteroarylthiazolyl compound of formula I and pharmaceutically acceptable salts thereof, comprises the steps of:

(a) reacting a substituted 4-bromoarylnitrile compound of formula 4:

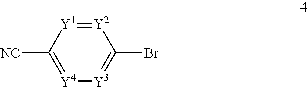

with phosphorous pentasulfide, thereby forming a 4-bromoarylthioamide compound of formula 5:

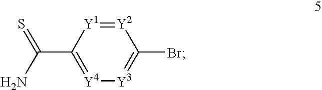

(b) reacting the 4-bromoarylthioamide compound of formula 5 in step (a) with an epoxyketone of the formula 3:

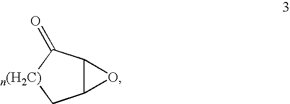

thereby forming an intermediate 2-aryl- or 2-heteroarylthiazolyl compound of formula 6:

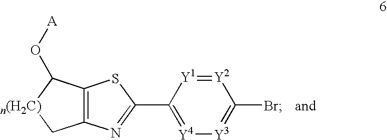

(c) treating the intermediate 2-aryl- or 2-heteroarylthiazolyl compound of formula 6 in step (b) with alkyl or cyclic alkyl amines, thereby forming the fused bicyclic 2-aryl- or 2-heteroarylthiazolyl compound of formula I or a pharmaceutically acceptable salt thereof, as summarized in Scheme 2.

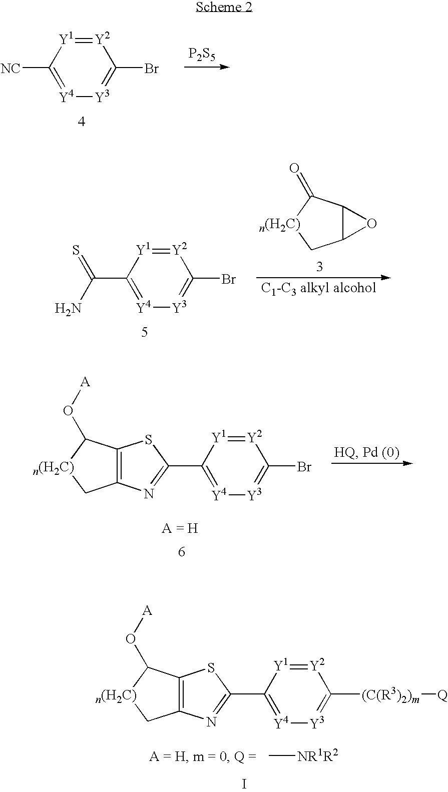

According to one embodiment, the fused bicyclic 2-aryl- or 2-heteroarylthiazolyl compound of formula I that is formed may be further treated with a C₁-C₃ alkyl halide in the presence of a base, forming a derivative of the fused bicyclic 2-aryl- or 2-heteroarylthiazolyl compound of formula I or its pharmaceutically acceptable salt, as summarized in Scheme 3.

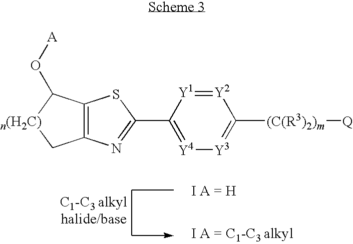

According to a separate embodiment, the fused bicyclic 2-aryl- or 2-heteroarylthiazolyl compound of formula I that is formed may be further treated with acetic anhydride in the presence of a base, forming a derivative, namely an ester of the fused bicyclic 2-aryl- or 2-heteroarylthiazolyl compound of formula I or its pharmaceutically acceptable salt, as summarized in Scheme 4.

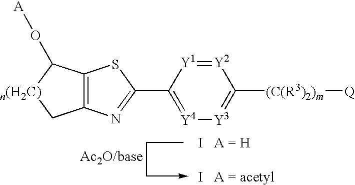

In the synthesis reactions, as summarized in Schemes 3 and 4, a base is used as a catalyst. Suitable examples of bases include, but are not limited to, 4-dimethylamino pyridine, triethylamine, pyridine, sodium carbonate, potassium carbonate, potassium hydroxide, and mixtures thereof.

The present invention accordingly provides a pharmaceutical composition, which comprises an effective amount of a fused bicyclic 2-aryl- or 2-heteroarylthiazolyl compound of the invention in combination or association with a pharmaceutically acceptable carrier. Suitable examples of pharmaceutical carriers used in accordance with the present invention include, but are not limited to, excipients, diluents, fillers, disintegrants, lubricants and other agents that can function as a carrier. The term “pharmaceutically acceptable excipient” means an excipient that is useful in preparing a pharmaceutical composition that is generally safe, non-toxic, and desirable, and includes excipients that are acceptable for veterinary use as well as for human pharmaceutical use. Such excipients can be solid, liquid, semisolid, or, in the case of an aerosol composition, gaseous. Pharmaceutical compositions are prepared in accordance with acceptable pharmaceutical procedures, such as described in Remingtons Pharmaceutical Sciences, 17th edition, ed. Alfonoso R. Gennaro, Mack Publishing Company, Easton, Pa. (1985). Pharmaceutically acceptable carriers are those that are compatible with the other ingredients in the formulation and biologically acceptable. As used herein, the term “effective amount” refers to the amount of active compound or pharmaceutical agent that elicits the biological or medicinal response in a tissue, system, animal, individual or human that is being sought by a researcher, veterinarian, medical doctor or other clinician, which includes one or more of the following: (1) preventing the disease; for example, preventing a disease, condition or disorder in an individual that may be predisposed to the disease, condition or disorder but does not yet experience or display the pathology or symptomatology of the disease; (2) inhibiting the disease; for example, inhibiting a disease, condition or disorder in an individual that is experiencing or displaying the pathology or symptomatology of the disease, condition or disorder (i.e., arresting or slowing further development of the pathology and/or symptomatology); and (3) ameliorating the disease; for example, ameliorating a disease, condition or disorder in an individual that is experiencing or displaying the pathology or symptomatology of the disease, condition or disorder (i.e., reversing the pathology and/or symptomatology).

The term “treating” or “treatment” refers to any indicia of success in amelioration of an injury, pathology, or condition, including any objective or subjective parameter such as abatement; remission; diminishing of symptoms or making the injury, pathology, or condition more tolerable to the patient; slowing the rate of degeneration or decline; making the final point of degeneration less debilitating; or improving a subject's physical or mental well-being. The treatment or amelioration of symptoms can be based on objective or subjective parameters; including the results of a physical examination, neurological examination, and/or psychiatric evaluation. “Treating” or “treatment of a securin related disorder” includes preventing the onset of symptoms in a subject that may be predisposed to a securin related disorder but does not yet experience or exhibit symptoms of the disorder (prophylactic treatment), inhibiting the symptoms of the disorder (slowing or arresting its development), providing relief from the symptoms or side-effects of the disorder (including palliative treatment), and/or relieving the symptoms of the disorder (causing regression). Accordingly, the term “treating” includes the administration of the compounds or agents of the present invention to a subject to prevent or delay, to alleviate, or to arrest or inhibit development of the symptoms or conditions associated with securin related disorders, e.g., tumor growth associated with cancer. A skilled medical practitioner will know how to use standard methods to determine whether a patient is suffering from a disease associated with enhanced levels and/or activity of securin, e.g., by examining the patient and determining whether the patient is suffering from a disease known to be associated with elevated securin levels or activity or by assaying for securin levels in blood plasma or tissue of the individual suspected of suffering from a securin related disease and comparing securin levels in the blood plasma or tissue of the individual suspected of suffering from a securin related disease to securin levels in the blood plasma or tissue of a healthy individual. Increased securin levels are indicative of disease. Accordingly, the present invention provides, inter alia, methods of administering a compound of the present invention to a subject and determining levels of securin in the subject. The level of securin in the subject can be determined before and/or after administration of the compound.

In healthy individuals, securin is found at low levels in the plasma, but it is elevated in many securin related disorders, including, for example, breast cancer (J. A. Bernal, et al. Nature Genetics, Vol. 32, pp. 306-311, 2002 and S. Ogbagabriel, et al. Mod. Path. Vol. 18, pp. 985-990, 2005).

The term “securin related disorder or disease associated with securin activity” refers to any disease or condition that is associated with increased or enhanced expression or activity of securin or increased or enhanced expression or activity of a gene encoding securin. Examples of such increased activity or expression can include one or more of the following: activity of the protein or expression of the gene encoding the protein is increased above the level of that in normal subjects; activity of the protein or expression of the gene encoding the protein is in an organ, tissue or cell where it is not normally detected in normal subjects (i.e. spatial distribution of the protein or expression of the gene encoding the protein is altered); activity of the protein or expression of the gene encoding the protein is increased when activity of the protein or expression of the gene encoding the protein is present in an organ, tissue or cell for a longer period than in a normal subjects (i.e., duration of activity of the protein or expression of the gene encoding the protein is increased). A normal or healthy subject is a subject not suffering from a securin related disorder or disease.

“Inhibitors,” “activators,” and “modulators” of expression or of activity are used to refer to inhibitory, activating, or modulating molecules, respectively, identified using in-vitro and in-vivo assays for expression or activity. Inhibitors of the present invention are compositions that, inhibit expression of securin or bind to, partially or totally block stimulation, decrease, prevent, delay activation, inactivate, desensitize, or down regulate the activity of securin. Samples or assays comprising securin can be treated with a composition of the present invention and compared to control samples without a composition of the present invention. Control samples (untreated with compositions of the present invention) can be assigned a relative activity value of 100%. In certain embodiments, inhibition of securin is achieved when the activity value relative to the control is about 80% or less, optionally 50% or 25, 10%, 5% or 1%.

The terms “pharmaceutically acceptable”, “physiologically tolerable” and grammatical variations thereof, as they refer to compositions, carriers, diluents and reagents, are used interchangeably and represent that the materials are capable of administration to or upon a human without the production of undesirable physiological effects such as nausea, dizziness, gastric upset and the like which would be to a degree that would prohibit administration of the compound.

A “therapeutically effective amount” or “pharmaceutically effective amount” means the amount that, when administered to a subject, produces effects for which it is administered. For example, a “therapeutically effective amount,” when administered to a subject to inhibit securin activity, is sufficient to inhibit securin activity. A “therapeutically effective amount,” when administered to a subject for treating a disease, is sufficient to effect treatment for that disease.

Except when noted, the terms “subject” or “patient” are used interchangeably and refer to mammals such as human patients and non-human primates, as well as experimental animals such as rabbits, rats, and mice, and other animals. Accordingly, the term “subject” or “patient” as used herein means any mammalian patient or subject to which the compounds of the invention can be administered. In an exemplary embodiment of the present invention, to identify subject patients for treatment according to the methods of the invention, accepted screening methods are employed to determine risk factors associated with a targeted or suspected disease or condition or to determine the status of an existing disease or condition in a subject. These screening methods include, for example, conventional work-ups to determine risk factors that are associated with the targeted or suspected disease or condition. These and other routine methods allow the clinician to select patients in need of therapy using the methods and formulations of the present invention.

The present invention provides fused bicyclic 2-aryl- or 2-heteroarylthiazolyl compounds of formula I as pharmaceuticals. In a preferred embodiment, fused bicyclic 2-aryl- or 2-heteroarylthiazolyl compounds of formula I are formulated as pharmaceuticals to treat diseases associated with increased securin activity, e.g., by inhibiting growth of cancerous cell lines, including but limited to for example, the growth of human breast carcinoma in a subject.

In general, the fused bicyclic 2-aryl- or 2-heteroarylthiazolyl compounds of formula I can be administered as pharmaceutical compositions by any method known in the art for administering therapeutic drugs including oral, buccal, topical, systemic (e.g., transdermal, intranasal, or by suppository), or parenteral (e.g., intramuscular, subcutaneous, or intravenous injection). Compositions can take the form of tablets, pills, capsules, semisolids, powders, sustained release formulations, solutions, suspensions, emulsions, syrups, elixirs, aerosols, or any other appropriate compositions; and comprise at least one compound of this invention in combination with at least one pharmaceutically acceptable excipient. Suitable excipients are well known to persons of ordinary skill in the art, and they, and the methods of formulating the compositions, can be found in such standard references as Alfonso A R: Remington's Pharmaceutical Sciences, 17th ed., Mack Publishing Company, Easton Pa., 1985. Suitable liquid carriers, especially for injectable solutions, include water, aqueous saline solution, aqueous dextrose solution, and glycols. In some embodiments of the present invention, the fused bicyclic 2-arylthiazolyl compounds of formula I suitable for use in the practice of this invention will be administered either singly or in combination with at least one other compound of this invention. The fused bicyclic 2-aryl- or 2-heteroarylthiazolyl compounds of formula I suitable for use in the practice of the present invention can also be administered with at least one other conventional therapeutic agent for the disease being treated. Compounds of the invention may preferably be orally administered, for example, with an inert diluent or with an assimilable edible carrier, or they may be enclosed in hard or soft shell gelatin capsules, or they may be compressed into tablets or they may be incorporated directly with the food of the diet. For oral therapeutic administration, these active compounds may be incorporated with excipients and used in the form of ingestible tablets, buccal tablets, troches, capsules, elixirs, suspensions, syrups, wafers and the like. Such compositions and preparations should contain at least 0.1% of active compound. The percentage of the compositions and preparations may, of course, be varied and may conveniently be between about 2% to about 60% of the weight of the unit. The amount of active compound in such therapeutically useful compositions is such that a suitable dosage will be obtained. Preferred compositions or preparations according to the present invention are prepared so that an oral dosage unit form contains between 10 and 1000 mg of active compound.

Aqueous suspensions of the invention can contain a fused bicyclic 2-aryl- or 2-heteroarylthiazolyl compound of formula I in admixture with excipients suitable for the manufacture of aqueous suspensions. Such excipients can include a suspending agent, such as sodium carboxymethylcellulose, methylcellulose, hydroxypropylmethylcellulose, sodium alginate, polyvinylpyrrolidone, gum tragacanth and gum acacia, and dispersing or wetting agents such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethylene oxycetanol), a condensation product of ethylene oxide with a partial ester derived from a fatty acid and a hexitol (e.g., polyoxyethylene sorbitol mono-oleate), or a condensation product of ethylene oxide with a partial ester derived from fatty acid and a hexitol anhydride (e.g., polyoxyethylene sorbitan mono-oleate). The aqueous suspension can also contain one or more preservatives such as ethyl or n-propyl p-hydroxybenzoate, one or more coloring agents, one or more flavoring agents, and one or more sweetening agents, such as sucrose, aspartame or saccharin. Formulations can be adjusted for osmolarity.

Oil suspensions can be formulated by suspending a fused bicyclic 2-aryl- or 2-heteroarylthiazolyl compound of formula I in a vegetable oil, such as arachis oil, olive oil, sesame oil or coconut oil, or in a mineral oil such as liquid paraffin; or a mixture of these. The oil suspensions can contain a thickening agent, such as beeswax, hard paraffin or cetyl alcohol. Sweetening agents can be added to provide a palatable oral preparation, such as glycerol, sorbitol or sucrose. These formulations can be preserved by the addition of an antioxidant such as ascorbic acid. As an example of an injectable oil vehicle, see Minto, J. Pharmacol. Exp. Ther. 281:93-102, 1997. The pharmaceutical formulations of the invention can also be in the form of oil-in-water emulsions. The oily phase can be a vegetable oil or a mineral oil, described above, or a mixture of these. Suitable emulsifying agents include naturally-occurring gums, such as gum acacia and gum tragacanth, naturally occurring phosphatides, such as soybean lecithin, esters or partial esters derived from fatty acids and hexitol anhydrides, such as sorbitan mono-oleate, and condensation products of these partial esters with ethylene oxide, such as polyoxyethylene sorbitan mono-oleate. The emulsion can also contain sweetening agents and flavoring agents, as in the formulation of syrups and elixirs. Such formulations can also contain a demulcent, a preservative, or a coloring agent. Under ordinary conditions of storage and use, these preparations contain a preservative to prevent the growth or microorganisms.

The compound of choice, alone or in combination with other suitable components, can be made into aerosol formulations (i.e., they can be “nebulized”) to be administered via inhalation. Aerosol formulations can be placed into pressurized acceptable propellants, such as dichlorodifluoromethane, propane, nitrogen, and the like.

Formulations suitable for parenteral administration, such as, for example, by intraarticular (in the joints), intravenous, intramuscular, intradermal, intraperitoneal, and subcutaneous routes, include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. Among the acceptable vehicles and solvents that can be employed are water and Ringer's solution, an isotonic sodium chloride. In addition, sterile fixed oils can conventionally be employed as a solvent or suspending medium. For this purpose any bland fixed oil can be employed including synthetic mono- or diglycerides. In addition, fatty acids such as oleic acid can likewise be used in the preparation of injectables. These solutions are sterile and generally free of undesirable matter. Where the compounds are sufficiently soluble they can be dissolved directly in normal saline with or without the use of suitable organic solvents, such as propylene glycol or polyethylene glycol. Dispersions of the finely divided compounds can be made-up in aqueous starch or sodium carboxymethyl cellulose solution, or in suitable oil, such as arachis oil. These formulations can be sterilized by conventional, well-known sterilization techniques. The formulations can contain pharmaceutically acceptable auxiliary substances as required to approximate physiological conditions such as pH adjusting and buffering agents, toxicity adjusting agents, e.g., sodium acetate, sodium chloride, potassium chloride, calcium chloride, sodium lactate and the like. The concentration of the fused bicyclic 2-aryl- or 2-heteroarylthiazolyl compound of formula I in these formulations can vary widely, and will be selected primarily based on fluid volumes, viscosities, body weight, and the like, in accordance with the particular mode of administration selected and the patient's needs. For IV administration, the formulation can be a sterile injectable preparation, such as a sterile injectable aqueous or oleaginous suspension. This suspension can be formulated according to the known art using those suitable dispersing or wetting agents and suspending agents. The sterile injectable preparation can also be a sterile injectable solution or suspension in a nontoxic parenterally-acceptable diluent or solvent, such as a solution of 1,3-butanediol. The formulations of fused bicyclic 2-aryl- or 2-heteroarylthiazolyl compounds of formula I can be presented in unit-dose or multi-dose sealed containers, such as ampules and vials. Injection solutions and suspensions can be prepared from sterile powders, granules, and tablets of the kind previously described.

The fused bicyclic 2-aryl- or 2-heteroarylthiazolyl compounds of this invention can be administered orally. The amount of a compound of the present invention in the composition can vary widely depending on the type of composition, size of a unit dosage, kind of excipients, and other factors well known to those of ordinary skill in the art. In general, the final composition can comprise from, for example, 0.000001 percent by weight (% w) to 10% w of the fused bicyclic 2-aryl- or 2-heteroarylthiazolyl compound of formula I, preferably 0.00001% w to 1% w, with the remainder being the excipient or excipients.

Pharmaceutical formulations for oral administration can be formulated using pharmaceutically acceptable carriers well known in the art in dosages suitable for oral administration. Such carriers enable the pharmaceutical formulations to be formulated in unit dosage forms as tablets, pills, powder, dragees, capsules, liquids, lozenges, gels, syrups, slurries, suspensions, etc. suitable for ingestion by the patient. Formulations suitable for oral administration can consist of (a) liquid solutions, such as an effective amount of the packaged nucleic acid suspended in diluents, such as water, saline or polyethylene glycol (e.g. PEG 400); (b) capsules, sachets or tablets, each containing a predetermined amount of the active ingredient, as liquids, solids, granules or gelatin; (c) suspensions in an appropriate liquid; and (d) suitable emulsions.

Pharmaceutical preparations for oral use can be obtained through combination of the compounds of the present invention with a solid excipient, optionally grinding a resulting mixture, and processing the mixture of granules, after adding suitable additional compounds, if desired, to obtain tablets or dragee cores. Suitable solid excipients are carbohydrate or protein fillers and include, but are not limited to sugars, including lactose, sucrose, mannitol, or sorbitol; starch from corn, wheat, rice, potato, or other plants; cellulose such as methyl cellulose, hydroxymethyl cellulose, hydroxypropylmethyl-cellulose or sodium carboxymethylcellulose; and gums including arabic and tragacanth; as well as proteins such as gelatin and collagen. If desired, disintegrating or solubilizing agents can be added, such as the cross-linked polyvinyl pyrrolidone, agar, alginic acid, or a salt thereof, such as sodium alginate. Tablet forms can include one or more of lactose, sucrose, mannitol, sorbitol, calcium phosphates, corn starch, potato starch, microcrystalline cellulose, gelatin, colloidal silicon dioxide, talc, magnesium stearate, stearic acid, and other excipients, colorants, fillers, binders, diluents, buffering agents, moistening agents, preservatives, flavoring agents, dyes, disintegrating agents, and pharmaceutically compatible carriers. Lozenge forms can comprise the active ingredient in a flavor, e.g., sucrose, as well as pastilles comprising the active ingredient in an inert base, such as gelatin and glycerin or sucrose and acacia emulsions, gels, and the like containing, in addition to the active ingredient, carriers known in the art.

The fused bicyclic 2-aryl- or 2-heteroarylthiazolyl compound of the present invention can also be administered in the form of suppositories for rectal administration of the drug. These formulations can be prepared by mixing the drug with a suitable non-irritating excipient, which is solid at ordinary temperatures but liquid at the rectal temperatures and will therefore melt in the rectum to release the drug. Such materials are cocoa butter and polyethylene glycols.

The compounds of the present invention can also be administered by intranasal, intraocular, intravaginal, and intrarectal routes including suppositories, insufflation, powders and aerosol formulations (for examples of steroid inhalants, see Rohatagi, J. Clin. Pharmacol. 35:1187-1193, 1995; Tjwa, Ann. Allergy Asthma Immunol. 75:107-111, 1995).

The fused bicyclic 2-aryl- or 2-heteroarylthiazolyl compound of formula I can be delivered transdermally, by a topical route, formulated as applicator sticks, solutions, suspensions, emulsions, gels, creams, ointments, pastes, jellies, paints, powders, and aerosols.

Encapsulating materials can also be employed with the compounds of the present invention and the term “composition” can include the active ingredient in combination with an encapsulating material as a formulation, with or without other carriers. For example, the compounds of the present invention can also be delivered as microspheres for slow release in the body. In one embodiment, microspheres can be administered via intradermal injection of drug-containing microspheres, which slowly release subcutaneously (see Rao, J. Biomater Sci. Polym. Ed. 7:623-645, 1995; as biodegradable and injectable gel formulations (see, e.g., Gao, Pharm. Res. 12:857-863, 1995); or, as microspheres for oral administration (see, e.g., Eyles, J. Pharm. Pharmacol. 49:669-674, 1997). Both transdermal and intradermal routes afford constant delivery for weeks or months. Cachets can also be used in the delivery of the compounds of the present invention, e.g., anti-atherosclerotic medicaments.

In another embodiment, the compounds of the present invention can be delivered by the use of liposomes which fuse with the cellular membrane or are endocytosed, i.e., by employing ligands attached to the liposome, or attached directly to the oligonucleotide, that bind to surface membrane protein receptors of the cell resulting in endocytosis. By using liposomes, particularly where the liposome surface carries ligands specific for target cells, or are otherwise preferentially directed to a specific organ, one can focus the delivery of the compound into the target cells in vivo. (See, e.g., Al-Muhammed, J. Microencapsul. 13:293-306, 1996; Chonn, Curr. Opin. Biotechnol. 6:698-708, 1995; Ostro, Am. J. Hosp. Pharm. 46:1576-1587, 1989). In other cases, the preferred preparation can be a lyophilized powder which may contain, for example, any or all of the following: 1 mM-50 mM histidine, 0.1%-2% sucrose, 2%-7% mannitol, at a pH range of 4.5 to 5.5, that is combined with buffer prior to use.

A pharmaceutical composition of the invention can optionally contain, in addition to a fused bicyclic 2-aryl- or 2-heteroarylthiazolyl compound of formula I, at least one other therapeutic agent useful in the treatment of a disease or condition associated with increased securin activity. The pharmaceutical compositions are generally formulated as sterile, substantially isotonic and in full compliance with all Good Manufacturing Practice (GMP) regulations of the U.S. Food and Drug Administration. The pharmaceutical compositions of the present invention may comprise combining fused bicyclic 2-aryl- or 2-heteroarylthiazolyl compounds of formula I with one or more other kinase-inhibiting compounds or chemotherapeutic agents. Chemotherapeutic agents include, but are not limited to exemestane, formestane, anastrozole, letrozole, fadrozole, taxane and derivatives such as paclitaxel or docetaxel, encapsulated taxanes, CPT-11, camptothecin derivatives, anthracycline glycosides, e.g., doxorubicin, idarubicin, epirubicin, etoposide, navelbine, vinblastine, carboplatin, cisplatin, estramustine, celecoxib, tamoxifen, raloxifen, Sugen SU-5416, Sugen SU-6668, and Herceptin. Methods of administrating a pharmaceutical composition in accordance with the invention are not specifically restricted, and can be administered in various preparations depending on the age, sex, and symptoms of the patient. For example, tablets, pills, solutions, suspensions, emulsions, granules and capsules may be orally administered. Injection preparations may be administered individually or mixed with injection transfusions such as glucose solutions and amino acid solutions intravenously. If necessary, the injection preparations are administered singly intramuscularly, intracutaneously, subcutaneously or intraperitoneally. Suppositories may be administered into the rectum. The dosage of a pharmaceutical composition according to the present invention will depend on the method of use, the age, sex, and condition of the patient.

The present invention provides methods of inhibiting securin activity in a subject for the treatment of diseases and conditions associated with increased securin activity using a fused bicyclic 2-aryl- or 2-heteroarylthiazolyl compound of formula I. In an exemplary embodiment of the present invention, a skilled practitioner will treat a subject having a disease associated with elevated securin levels and/or activity with the compounds of the present invention.

For treatment purposes, the compositions or compounds disclosed herein can be administered to the subject in a single bolus delivery, via continuous delivery (e.g., continuous transdermal, mucosal, or intravenous delivery) over an extended time period, or in a repeated administration protocol (e.g., by an hourly, daily or weekly, repeated administration protocol). The pharmaceutical formulations of the present invention can be administered, for example, one or more times daily, 3 times per week, or weekly. In an exemplary embodiment of the present invention, the pharmaceutical formulations of the present invention are orally administered once or twice daily.

In this context, a therapeutically effective dosage of the biologically active agent(s) can include repeated doses within a prolonged treatment regimen that will yield clinically significant results to alleviate one or more symptoms or detectable conditions associated with increased securin activity. Determination of effective dosages in this context is typically based on animal model studies followed up by human clinical trials and is guided by determining effective dosages and administration protocols that significantly reduce the occurrence or severity of targeted exposure symptoms or conditions in the subject. Suitable models in this regard include, for example, murine, rat, porcine, feline, non-human primate, and other accepted animal model subjects known in the art. Alternatively, effective dosages can be determined using in-vitro models (e.g., immunologic and histopathologic assays). Using such models, only ordinary calculations and adjustments are typically required to determine an appropriate concentration and dose to administer a therapeutically effective amount of the biologically active agent(s) (e.g., amounts that are intranasally effective, transdermally effective, intravenously effective, or intramuscularly effective to elicit a desired response). In alternative embodiments, an “effective amount” or “therapeutically effective dose” of the biologically active agent(s) will simply inhibit or enhance one or more selected biological activity(ies) correlated with a disease or condition, as set forth above, for either therapeutic or diagnostic purposes.

The actual dosage of biologically active agents will of course vary according to factors such as the extent of exposure and particular status of the subject (e.g., the subject's age, size, fitness, extent of symptoms, susceptibility factors, etc), time and route of administration, as well as other drugs or treatments being administered concurrently. Dosage regimens can be adjusted to provide an optimum prophylactic or therapeutic response. By “therapeutically effective dose” herein is meant a dose that produces effects for which it is administered. More specifically, a therapeutically effective dose of the compound(s) of the invention preferably alleviates symptoms, complications, or biochemical indicia of diseases associated with increased securin activity. The exact dose will depend on the purpose of the treatment, and will be ascertainable by one skilled in the art using known techniques (see, e.g., Lieberman, Pharmaceutical Dosage Forms (Vols. 1-3, 1992); Lloyd, 1999, The Art, Science, and Technology of Pharmaceutical Compounding; and Pickar, 1999, Dosage Calculations). A therapeutically effective dose is also one in which any toxic or detrimental side effects of the active agent is outweighed in clinical terms by therapeutically beneficial effects. It is to be further noted that for each particular subject, specific dosage regimens should be evaluated and adjusted over time according to the individual need and professional judgment of the person administering or supervising the administration of the compound.

In an exemplary embodiment of the present invention, unit dosage forms of the compounds are prepared for standard administration regimens. In this way, the composition can be subdivided readily into smaller doses at the physicians direction. For example, unit dosages can be made up in packeted powders, vials or ampoules and preferably in capsule or tablet form. The active compound present in these unit dosage forms of the composition can be present in an amount of, for example, from about one gram to about fifteen grams or more, for single or multiple daily administration, according to the particular need of the patient. By initiating the treatment regimen with a minimal daily dose of about one gram, the blood levels of securin and the patients symptomatic relief analysis can be used to determine whether a larger or smaller dose is indicated. Effective administration of the compounds of this invention can be given at an oral dose of from, for example about 0.1 mg/kg/day to about 1,000 mg/kg/day. Preferably, administration will be from about 10/mg/kg/day to about 600 mg/kg/day, more preferably from about 25 to about 200 mg/kg/day, and even more preferably from about 50 mg/kg/day to about 100 mg/kg/day.

In certain embodiments, the present invention is directed to prodrugs of fused bicyclic 2-aryl- or 2-heteroarylthiazolyl compounds of formula I. The term “prodrug,” as used herein, means a compound that is convertible in vivo by metabolic means (e.g. by hydrolysis) to a compound of formula 1 or 2. Various forms of prodrugs are known in the art such as those discussed in, for example, Bundgaard, (ed.), Design of Prodrugs, Elsevier (1985); Widder, et al. (ed.), Methods in Enzymology, vol. 4, Academic Press (1985); Krogsgaard-Larsen, et al., (ed). “Design and Application of Prodrugs, Textbook of Drug Design and Development, Chapter 5, 113-191 (1991), Bundgaard, et al., Journal of Drug Delivery Reviews, 8:1-38 (1992), Bundgaard, J. of Pharmaceutical Sciences, 77:285 et seq. (1988); and Higuchi and Stella (eds.) Prodrugs as Novel Drug Delivery Systems, American Chemical Society (1975).

After a pharmaceutical comprising a fused bicyclic 2-aryl- or 2-heteroarylthiazolyl compound of formula I has been formulated in a suitable carrier, it can be placed in an appropriate container and labeled for treatment of a securin related disorder, e.g., breast cancer. Additionally, another pharmaceutical comprising at least one other therapeutic agent useful in the treatment of the securin related disorder can be placed in the container as well and labeled for treatment of the indicated disease. Alternatively, a single pharmaceutical comprising a fused bicyclic 2-aryl- or 2-heteroarylthiazolyl compound of formula I and at least one other therapeutic agent useful in the treatment of a securin related disorder can be placed in an appropriate container and labeled for treatment. For administration of pharmaceuticals comprising a fused bicyclic 2-aryl- or 2-heteroarylthiazolyl compound of formula I and of pharmaceuticals comprising, in a single pharmaceutical, a fused bicyclic 2-aryl- or 2-heteroarylthiazolyl compound of formula I and at least one other therapeutic agent useful in the treatment of a securin related disorder, such labeling would include, for example, instructions concerning the amount, frequency and method of administration. Similarly, for administration of multiple pharmaceuticals provided in the container, such labeling would include, for example, instructions concerning the amount, frequency and method of administration of each pharmaceutical.

Based on the results of standard pharmacological test procedures described herein, the fused bicyclic 2-aryl- or 2-heteroarylthiazolyl compounds of formula I are useful as agents for treating, inhibiting or controlling the growth of cancerous tumor cells and associated diseases in a mammal in need thereof. The compounds of the invention are useful as agents for treating, inhibiting or controlling the growth of cancerous tumor cells and associated diseases in a mammal. In the case of cancer treatment, it is believed that many neoplasias such as leukemia, lung cancer, colon cancer, thyroid cancer, ovarian cancer, renal cancer, prostate cancer and breast cancers may be treated by effectively administering effective amounts of a fused bicyclic 2-aryl- or 2-heteroarylthiazolyl compound of formula I. Suitable examples of cancers for treatment using methods provided herein include carcinoma, sarcoma, lymphoma, or leukemia. The term “carcinoma” refers to a benign or malignant epithelial tumor and includes, but is not limited to, breast carcinoma, prostate carcinoma, non-small lung carcinoma, colon carcinoma, melanoma carcinoma, ovarian carcinoma, or renal carcinoma. A preferred subject or mammalian host benefiting from treatment using one or more compounds of the invention is a human.

Selected fused bicyclic 2-aryl- or 2-heteroarylthiazolyl compounds of formula I were tested for their activity in a securin parental cell line (HCT116 FB) and a securin knockout (KO) cell line. Certain fused bicyclic 2-aryl- or 2-heteroarylthiazolyl compounds of formula I inhibited the growth of the securin knockout cells but not securin parental cells, which were characterized by high selectivity ratios (IC₅₀ HCT116 FB/IC₅₀ securin KO), as summarized in Table I. For example, fused bicyclic 2-aryl- or 2-heteroarylthiazolyl compounds of Examples 45 and 164 exhibited high selectivity ratios. The in-vivo activity of selected fused bicyclic 2-aryl- or 2-heteroarylthiazolyl compounds of formula I was studied in cells growing as xenografts in athymic (nude) mice. The effect of Examples 164, 166 and 167 was studied in xenografts of a human breast carcinoma cell line (MDA-MB-361).

Examples of fused bicyclic 2-aryl- or 2-heteroarylthiazolyl compounds of formula I were evaluated in several standard pharmacological test procedures that showed that the compounds of this invention possess significant activity in reducing the volume of tumor growth in-vivo. Based on the activity shown in the standard pharmacological test procedures, the compounds of this invention are therefore useful as anti-cancer agents. Associated cancers are selected from the group consisting of breast, colon, lung, prostate, melanoma, epidermal, leukemia, kidney, bladder, mouth, larynx, esophagus, stomach, ovary, pancreas, liver, skin and brain. In particular, the compounds of this invention possess an effect similar to HKI-272. The test procedures used and results obtained are shown below. Having described the invention, the invention is further illustrated by the following non-limiting examples.

### EXAMPLES

The syntheses of Examples 1-169 are described in Examples.

### Example 1

### Preparation of N-[4-(7-methoxy-4,5,6,7-tetrahydro-1,3-benzothiazol-2-yl)phenyl]-N,N-dimethylamine

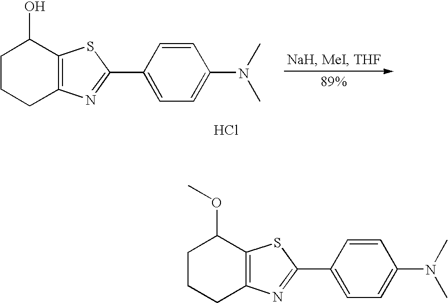

To suspension of 7-hydroxy-2-(4-N,N-dimethylaminophenyl)-4,5,6,7-tetrahydrobenzothiazole hydrochloride (93 mg, 0.3 mmol) and NaH (24 mg, 0.6 mmol) in 2 mL of anhydrous tetrahydrofuran (THF) was added MeI (64 mg, 0.45 mmol). The reaction mixture was stirred at room temperature for 3 hours, then quenched with 2 mL of sat. NaHCO₃ solution and was extracted with ethyl acetate (EtOAc). The combined organic layers were dried using anhydrous Na₂SO₄, separated by silica gel column (hexane/EtOAc=3:1) to give 77 mg (89%) of N-[4-(7-methoxy-4,5,6,7-tetrahydro-1,3-benzothiazol-2-yl)phenyl]-N,N-dimethylamine as a yellow solid. MS: m/z 289 (M+H).

### Example 2

### Preparation of 2-[4-(dimethylamino)phenyl]-4,5,6,7-tetrahydro-1,3-benzothiazol-7-yl acetate

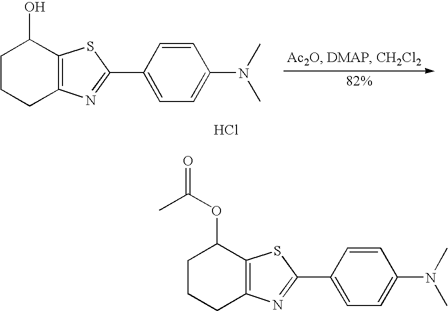

To suspension of 7-hydroxy-2-(4-N,N-dimethylaminophenyl)-4,5,6,7-tetrahydrobenzothiazole hydrochloride (93 mg, 0.3 mmol) and DMAP (55 mg, 0.45 mmol) in 2 mL of CH₂Cl₂ was added anhydrous acetic anhydride Ac₂O (46 mg, 0.45 mmol). The reaction mixture was stirred at room temperature for 3 hours, then quenched with 2 mL of saturated NaHCO₃ solution and was extracted with CH₂Cl₂. The combined organic layers were dried using Na₂SO₄, separated by silica gel column (hexane/EtOAc=3:1) to give 78 mg (82%) of 2-[4-(dimethylamino)phenyl]-4,5,6,7-tetrahydro-1,3-benzothiazol-7-yl acetate as a yellow solid. MS: m/z 317 (M+H).

### Example 3

### Preparation of N-[4-(7-ethoxy-4,5,6,7-tetrahydro-1,3-benzothiazol-2-yl)phenyl]-N,N-diethylamine

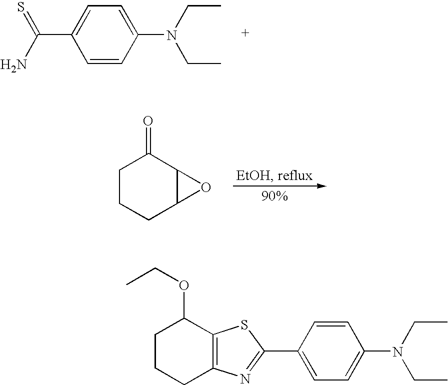

A solution of 7-oxabicyclo[4.1.0]heptan-2-one (160 mg, 1.43 mmol) and 4-(diethylamino)thiobenzamide (208 mg, 1.0 mmol) in anhydrous EtOH (5 mL) was refluxed for 40 hours then concentrated. The residue was separated by flash chromatography, eluting with CH₂Cl₂-CH₃CN=20:1, providing 297 mg (90%) of N-[4-(7-ethoxy-4,5,6,7-tetrahydro-1,3-benzothiazol-2-yl)phenyl]-N,N-diethylamine as a yellow solid. MS: m/z 331 (M+H).

### Example 4

### Preparation of 2-[4-(diethylamino)phenyl]-4,5,6,7-tetrahydro-1,3-benzothiazol-7-ol

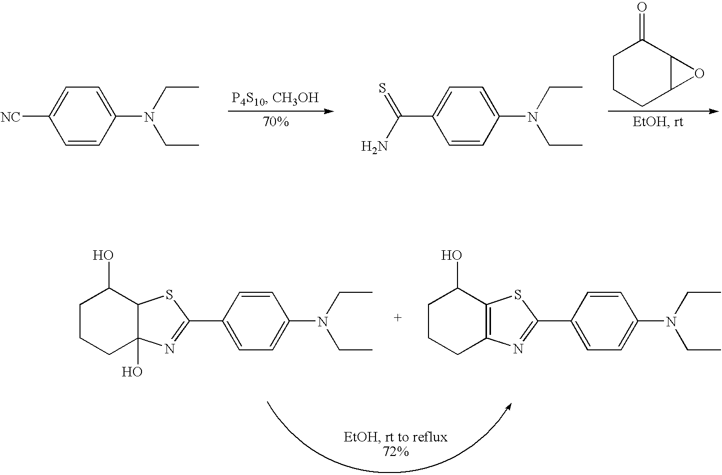

### Step A. 4-(diethylamino)thiobenzamide

A flask was placed with 4-(diethylamino)benzonitrile (731 mg, 5.0 mmol) and P₄S₁₀ (578 mg, 1.3 mmol). To this was added CH₃OH (1.0 mL). The reaction mixture was stirred at room temperature for 24 hours and additional P₄S₁₀ (200 mg) was added. The reaction mixture was stirred at room temperature for at least 48 hours. The solvent was removed and the residue was separated by flash chromatography, eluting with CH₂Cl₂-CH₃CN=15:1, providing 725 mg (70%) of 4-(diethylamino)thiobenzamide as a yellow solid. MS: m/z 209 (M+H).

### Step B. 2-[4-(diethylamino)phenyl]-4,5,6,7-tetrahydro-1,3-benzothiazol-7-ol

A solution of 7-oxabicyclo[4.1.0]heptan-2-one (134 mg, 1.2 mmol) and 4-(diethylamino)thiobenzamide (208 mg, 1.0 mmol) in anhydrous EtOH (5 mL) was stirred at room temperature for 3 days, then refluxed for 2 hours and then concentrated. The residue was separated by flash chromatography (CH₂Cl₂—CH₃CN=10:1), providing 217 mg (72%) of 2-[4-(diethylamino)phenyl]-4,5,6,7-tetrahydro-1,3-benzothiazol-7-ol as a yellow solid. MS: m/z 303 (M+H).

### Examples 5 and 6

### Preparation of 2-[4-(dimethylamino)phenyl]-5,6-dihydro-4H-cyclopenta[d][1,3]thiazol-6-ol (Example 5) and (6R)-2-[4-(dimethylamino)phenyl]-5,6-dihydro-4H-cyclopenta[d][1,3]thiazol-6-ol (Example 6)

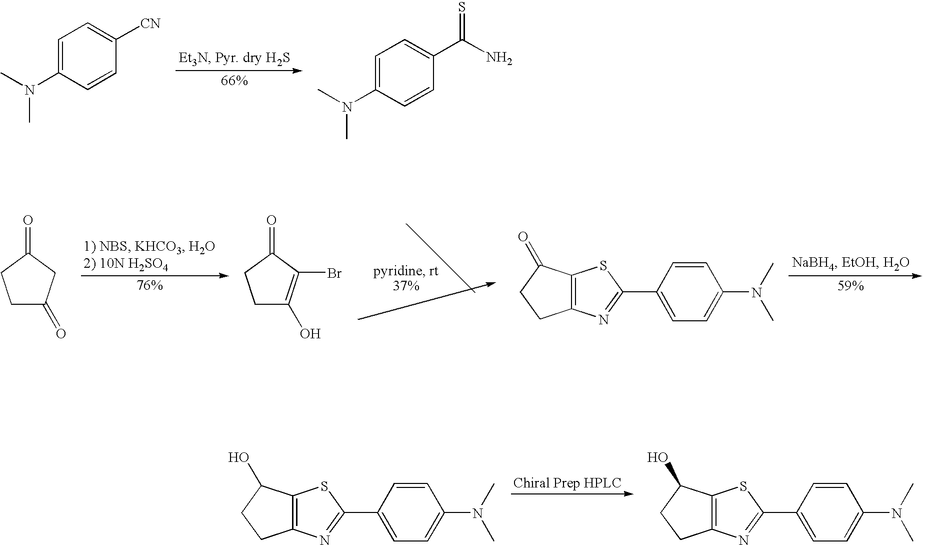

### Step A. 4-(dimethylamino)thiobenzamide

A solution of 4-(dimethylamino)benzonitrile (731 mg, 5.0 mmol) in pyridine (35 mL) containing triethylamine (15 mL) was cooled in an ice bath and saturated with H₂S gas. The reaction bottle was then sealed and stirred at room temperature for 3 days. The bottle was opened, and the solvent was evaporated under reduced pressure. The residue was added to H₂O, then extracted with EtOAc. The combined extracts were washed with H₂O, dried using Na₂SO₄, then filtered and concentrated. The resulting solid was dried in vacuo to provide 597 mg (66%) of 4-(dimethylamino)thiobenzamide as a yellow-brown solid. MS: m/z 181 (M+H).

### Step B. 2-bromo-3-hydroxycyclopent-2-en-1-one

A suspension of 1,3-cyclopentanedione (981 mg, 10 mmol) in H₂O (7 mL) was added a solution of KHCO₃ (1.101 g, 11 mmol) in H₂O (8 mL). CO gas evolved during the addition and the reaction mixture turned into a dark green homogenous solution. To this was added N-bromosuccinimide (NBS, 1.958 g, 11 mmol) in portions. The resulting brown solution was stirred for 2 hours at room temperature, then acidified with 10N H₂SO₄. A yellow solid precipitated out during the acidification. The slurry was stirred for 30 minutes, then filtered. The yellow solid was washed with H₂O, dried in vacuo to give 1.34 g (76%) of 2-bromo-3-hydroxycyclopent-2-en-1-one as a yellow solid. MS: m/z 176 (M−H).

### Step C. 2-[4-(dimethylamino)phenyl]-4,5-dihydro-6H-cyclopenta[d][1,3]thiazol-6-one

A reaction mixture of 4-(dimethylamino)thiobenzamide (540 mg, 3.0 mmol) and 2-bromo-3-hydroxycyclopent-2-en-1-one (531 mg, 3.0 mmol) in anhydrous pyridine (10 mL) was stirred for 24 hours at room temperature. The solvent was removed under reduced pressure. The residue was separated by flash chromatography (CH₂Cl₂—CH₃CN=4:1 and 2:1) to give 290 mg (37%) of 2-[4-(dimethylamino)phenyl]-4,5-dihydro-6H-cyclopenta[d][1,3]thiazol-6-one as a yellow solid. MS: m/z 259 (M+H).

### Step D. 2-[4-(dimethylamino)phenyl]-5,6-dihydro-4H-cyclopenta[d][1,3]thiazol-6-ol

The ketone compound 2-[4-(dimethylamino)phenyl]-4,5-dihydro-6H-cyclopenta[d][1,3]thiazol-6-one (26 mg, 0.1 mmol) and NaBH₄ (38 mg, 1.0 mmol) in a mixture of EtOH (2 mL) and H₂O (0.6 mL) was stirred at room temperature for 24 hours. H₂O (3 mL) was added to the mixture and the mixture was then extracted with CH₂Cl₂. The combined extracts were washed with H₂O, dried using Na₂SO₄, then filtered and concentrated. The residue was separated on silica gel column (CH₂Cl₂—CH₃CN=4:1) to give 16 mg (59%) of 2-[4-(dimethylamino)phenyl]-5,6-dihydro-4H-cyclopenta[d][1,3]thiazol-6-ol as a yellow solid. MS: m/z 261 (M+H).

### Step E. (6R)-2-[4-(dimethylamino)phenyl]-5,6-dihydro-4H-cyclopenta[d][1,3]thiazol-6-ol

2-[4-(Dimethylamino)phenyl]-5,6-dihydro-4H-cyclopenta[d][1,3]thiazol-6-ol was subjected to the chiral prep HPLC separation, providing (6R)-2-[4-(dimethylamino)phenyl]-5,6-dihydro-4H-cyclopenta[d][1,3]thiazol-6-ol as a yellow solid. MS: m/z 261 (M+H).

### Example 7

### Preparation of 2-[4-(dimethylamino)phenyl]-5,6,7,8-tetrahydro-4H-cyclohepta[d][1,3]thiazol-8-ol

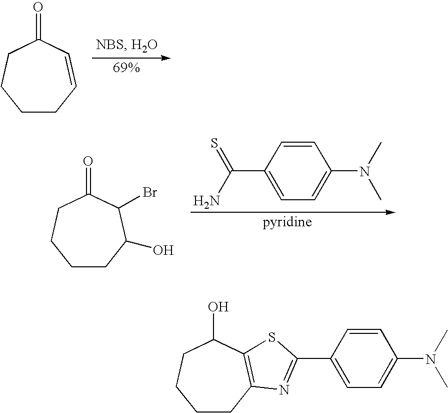

### Step A. 2-bromo-3-hydroxycycloheptanone

A suspension of 2-cyclohepten-1-one (551 mg, 5.0 mmol) and NBS (979 mg, 5.5 mmol) in H₂O was stirred at room temperature for at least 48 hours. The reaction mixture was extracted with CH₂Cl₂. The combined extracts were dried over Na₂SO₄, then filtered and concentrated. The residue was separated by flash chromatography, eluting with CH₂Cl₂-CH₃CN=30:1, to give 710 mg (69%) of 2-bromo-3-hydroxycycloheptanone as a colorless oil. MS: m/z 207 (M+H).

### Step B. 2-[4-(dimethylamino)phenyl]-5,6,7,8-tetrahydro-4H-cyclohepta[d][1,3]thiazol-8-ol

The reaction mixture of 2-bromo-3-hydroxycycloheptanone (260 mg, 1.26 mmol) and 4-(dimethylamino)thiobenzamide (145 mg, 0.8 mmol) in pyridine (3 mL) was stirred for 6 days. The solvent was removed under reduced pressure. The residue was separated by flash chromatography, eluting with CH₂Cl₂—CH₃CN=10:1, providing 81 mg (35%) of 2-[4-(dimethylamino)phenyl]-5,6,7,8-tetrahydro-4H-cyclohepta[d][1,3]thiazol-8-ol as a yellow solid. MS: m/z 289 (M+H).

### Example 8

### Preparation of N-{4-[(7R)-7-methoxy-4,5,6,7-tetrahydro-1,3-benzothiazol-2-yl]phenyl}-N,N-dimethylamine

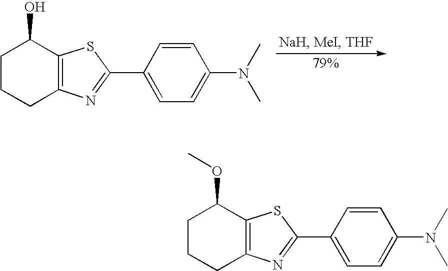

A solution of (7R)-2-[4-(dimethylamino)phenyl]-4,5,6,7-tetrahydro-1,3-benzothiazol-7-ol (40 mg, 0.146 mmol) and NaH (9 mg, 0.22 mmol) in 2 mL of anhydrous THF was added MeI (31 mg, 0.22 mmol). The reaction mixture was stirred at room temperature for 3 hours, quenched with 2 mL of sat. NaHCO₃ solution, extracted with EtOAc. The combined organic layers were dried over Na₂SO₄, separated by silica gel column (hexane/EtOAc=3:1) to give 33 mg (79%) of N-{4-[(7R)-7-methoxy-4,5,6,7-tetrahydro-1,3-benzothiazol-2-yl]phenyl}-N,N-dimethylamine as a yellow solid. MS: m/z 289 (M+H).

### Examples 9 and 10

### Preparation of metabolites 2-(4-aminophenyl)-4,5,6,7-tetrahydro-1,3-benzothiazol-7-ol and 2-[4-(methylamino)phenyl]-4,5,6,7-tetrahydro-1,3-benzothiazol-7-ol

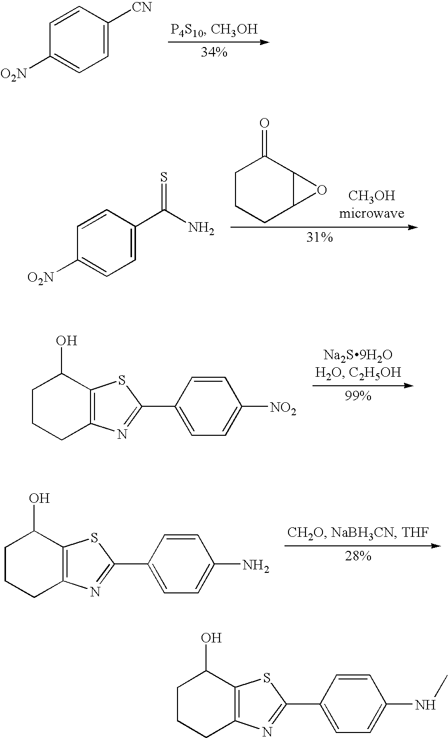

Step A. 4-Nitrobenzenecarbothioamide was prepared by following General Procedure II.

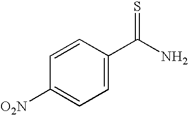

4-nitrobenzenecarbothioamide, MS: m/z 183 (M+H)

Step B. 2-(4-nitrophenyl)-4,5,6,7-tetrahydro-1,3-benzothiazol-7-ol was prepared by following General Procedure III.

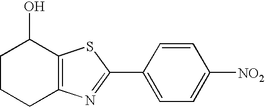

2-(4-nitrophenyl)-4,5,6,7-tetrahydro-1,3-benzothiazol-7-ol, MS: m/z 277 (M+H)

### Step C. 2-(4-Aminophenyl)-4,5,6,7-tetrahydro-1,3-benzothiazol-7-ol

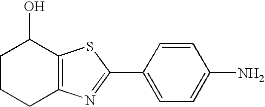

2-(4-aminophenyl)-4,5,6,7-tetrahydro-1,3-benzothiazol-7-ol, MS: m/z 247 (M+H)

A suspension of 2-(4-nitrophenyl)-4,5,6,7-tetrahydro-1,3-benzothiazol-7-ol (90 mg, 0.33 mmol) in a 1:1 mixture of H₂O/C₂H₅OH (4 mL) was added Na₂S.9H₂O (392 mg, 1.63 mmol). The mixture was stirred at room temperature for 40 hours, then concentrated to dryness. The residue was added to H₂O and neutralized with 1.0 N citric acid to pH 6-7, then filtered. The solid residue was washed with H₂O and dried by suction in air overnight, providing 79 mg (99%) of 2-(4-aminophenyl)-4,5,6,7-tetrahydro-1,3-benzothiazol-7-ol as a yellow solid. MS: m/z 247 (M+H).

### Step D. 2-[4-(Methylamino)phenyl]-4,5,6,7-tetrahydro-1,3-benzothiazol-7-ol

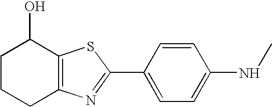

2-[4-(methylamino)phenyl]-4,5,6,7-tetrahydro-1,3-benzothiazol-7-ol, MS: m/z 261 (M+H)

To a solution of 2-(4-aminophenyl)-4,5,6,7-tetrahydro-1,3-benzothiazol-7-ol (37 mg, 0.15 mmol) in THF (1 mL) was added 37% CH₂O (8 μL, 0.1 mmol), followed by NaBH₃CN (10 mg, 0.15 mmol). The reaction mixture was stirred in an ice bath for 30 minutes, then additional CH₂O (6 μL, 0.07 mmol) was added. After stirring for additional 30 minutes, the mixture was concentrated, then separated by silica gel column (CH₂Cl₂/THF=6:1) to give 11 mg (28%) of 2-[4-(methylamino)phenyl]-4,5,6,7-tetrahydro-1,3-benzothiazol-7-ol as a yellow solid. MS: m/z 261 (M+H).

### Examples 11-28

Synthesis of Examples 11-28 were prepared following General Procedures I-III.

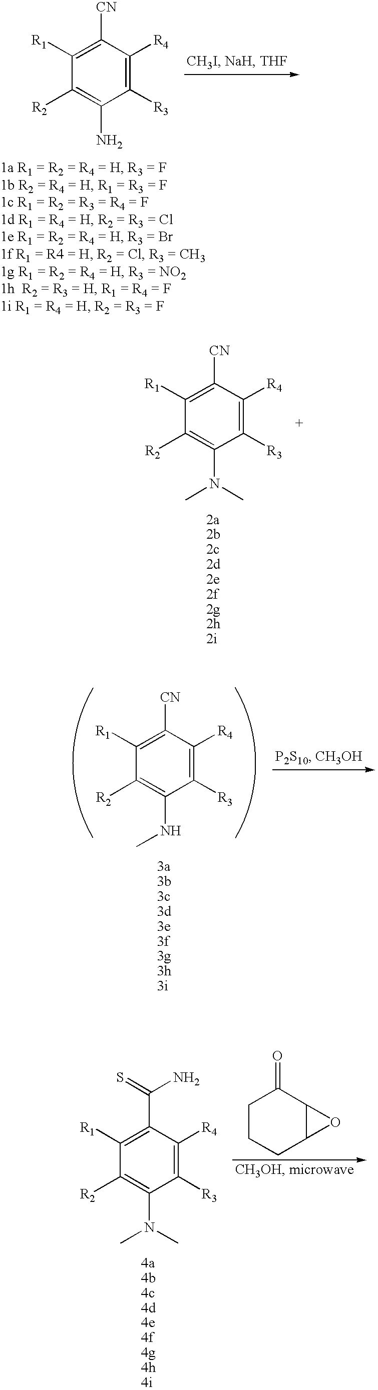

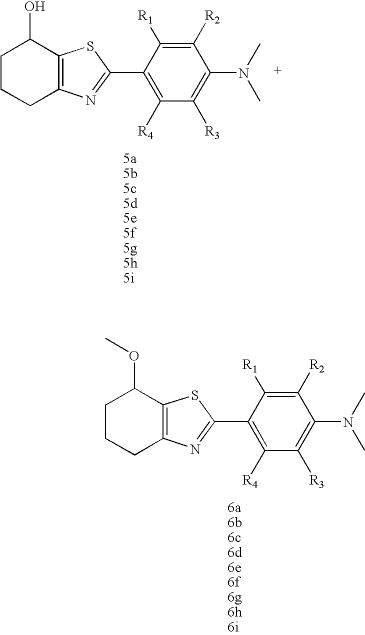

### General Procedure I

### Preparation of Dimethylamino Compounds 2a-i

To mixture of 1a-i (1 mmol) and NaH (2.6 mmol) in anhydrous THF (4 mL) was added CH₃I (2.6 mmol). The reaction mixture was stirred at room temperature for 3-12 hours, monitoring by TLC. The reaction was quenched by adding small amount of CH₃OH, then H₂O, the mixture was then concentrated and extracted three times with EtOAc. The combined extracts were washed with H₂O, then dried over Na₂SO₄. Separation and purification (hexane/EtOAc=10:1 or 10:2) gave the following compounds.

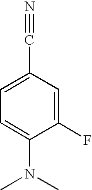

4-Dimethylamino-3-fluoro-benzonitrile, MS: m/z 165 (M+H)

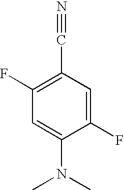

4-Dimethylamino-3,5-difluoro-benzonitrile, MS: m/z 183 (M+H)

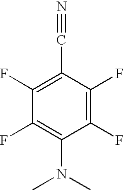

4-(dimethylamino)-2,3,5,6-tetrafluorobenzonitrile, MS: m/z 219 (M+H)

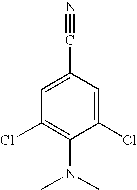

3,5-dichloro-4-(dimethylamino)benzonitrile, MS: m/z 216 (M+H)

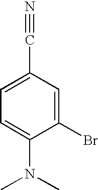

3-bromo-4-(dimethylamino)benzonitrile, MS: m/z 226 (M+H)

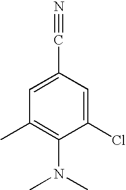

3-chloro-4-(dimethylamino)-5-methylbenzonitrile, MS: m/z 195 (M+H)

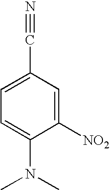

4-(dimethylamino)-3-nitrobenzonitrile, MS: m/z 192 (M+H)

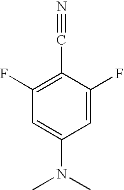

4-(dimethylamino)-2,6-difluorobenzonitrile, MS: m/z 183 (M+H)

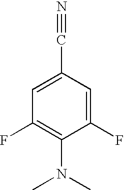

4-Dimethylamino-3,5-difluoro-benzonitrile, MS: m/z 183 (M+H)

### General Procedure II

### Preparation of Thioamides 4a-i

To a mixture of 2a-i (1 mmol) and P₄S₁₀ (0.5 mmol) was added CH₃OH (1 mL). The reaction mixture was stirred at room temperature for 16-24 hours. To this was added hexane (30 mL) or a 1:1 mixture of hexane/ether. The reaction mixture was refluxed for 1 hour, then cooled to room temperature and filtered. The solid residue was washed with a 1:1 mixture of hexane/CH₂Cl₂ several times, then dried in vacuum to give the following compounds (4b, 4c, 4e, 4g, 4h, 4i). Compounds 4a, 4d and 4f were subjected to silica gel column separation, eluting with CH₂Cl₂/THF=30:1 or 50:1).

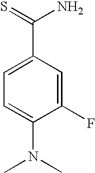

4-(dimethylamino)-3-fluorobenzenecarbothioamide, MS: m/z 199 (M+H)

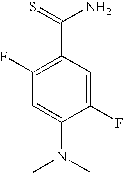

4-(dimethylamino)-2,5-difluorobenzenecarbothioamide, MS: m/z 217 (M+H)

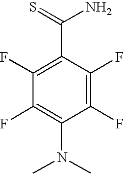

4-(dimethylamino)-2,3,5,6-tetrafluorobenzenecarbothioamide, MS: m/z 253 (M+H)

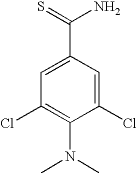

3,5-dichloro-4-(dimethylamino)benzenecarbothioamide, MS: m/z 250 (M+H)

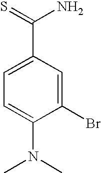

3-bromo-4-(dimethylamino)benzenecarbothioamide, MS: m/z 260 (M+H)

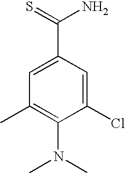

3-chloro-4-(dimethylamino)-5-methylbenzenecarbothioamide, MS: m/z 229 (M+H)

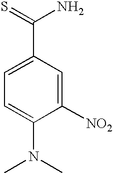

4-(dimethylamino)-3-nitrobenzenecarbothioamide, MS: m/z 226 (M+H)

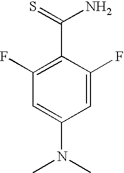

4-(dimethylamino)-2,6-difluorobenzenecarbothioamide, MS: m/z 217 (M+H)

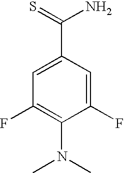

4-Dimethylamino-3,5-difluoro-thiobenzamide, MS: m/z 217 (M+H)

### General Procedure III

### Cycloaddition Reaction

Thioamide 4a-i (0.5 mmol) in CH₃OH (3 mL) was added 7-oxabicyclo[4.1.0]heptan-2-one (1.0 mmol). The reaction was carried out under microwave at 60° C.-100° C. for 40 minutes to 1 hour, monitoring by LC/MS. Alternatively, the reaction mixture was refluxed in C₂H₅OH for 3 hours, then concentrated. The residue was separated by silica gel column (CH₂Cl₂/THF=30:1), providing the following compounds 5a-i and 6a-i.

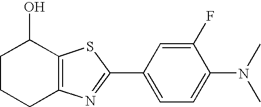

2-[4-(dimethylamino)-3-fluorophenyl]-4,5,6,7-tetrahydro-1,3-benzothiazol-7-ol (Example 17), MS: m/z 293 (M+H)

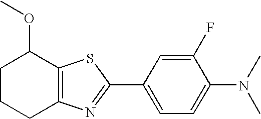

N-[2-fluoro-4-(7-methoxy-4,5,6,7-tetrahydro-1,3-benzothiazol-2-yl)phenyl]-N,N-dimethylamine (Example 18), MS: m/z 307 (M+H)

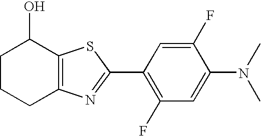

2-[4-(dimethylamino)-2,5-difluorophenyl]-4,5,6,7-tetrahydro-1,3-benzothiazol-7-ol (Example 11), MS: m/z 311 (M+H)

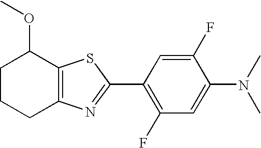

N-[2,5-difluoro-4-(7-methoxy-4,5,6,7-tetrahydro-1,3-benzothiazol-2-yl)phenyl]-N,N-dimethylamine (Example 12), MS: m/z 325 (M+H)

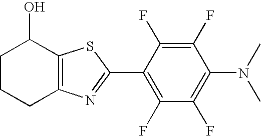

2-[4-(dimethylamino)-2,3,5,6-tetrafluorophenyl]-4,5,6,7-tetrahydro-1,3-benzothiazol-7-ol (Example 15), MS: m/z 347 (M+H)

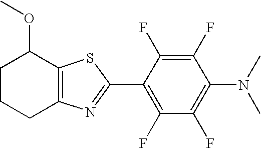

N,N-dimethyl-N-[2,3,5,6-tetrafluoro-4-(7-methoxy-4,5,6,7-tetrahydro-1,3-benzothiazol-2-yl)phenyl]amine (Example 16), MS: m/z 361 (M+H)

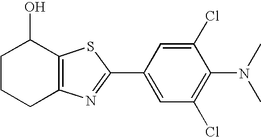

2-[3,5-dichloro-4-(dimethylamino)phenyl]-4,5,6,7-tetrahydro-1,3-benzothiazol-7-ol (Example 13), MS: m/z 344 (M+H)

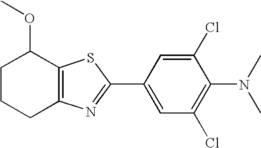

N-[2,6-dichloro-4-(7-methoxy-4,5,6,7-tetrahydro-1,3-benzothiazol-2-yl)phenyl]-N,N-dimethylamine (Example 14), MS: m/z 358 (M+H)

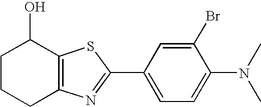

2-[3-bromo-4-(dimethylamino)phenyl]-4,5,6,7-tetrahydro-1,3-benzothiazol-7-ol (Example 21), MS: m/z 354 (M+H)

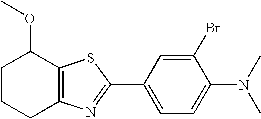

N-[2-bromo-4-(7-methoxy-4,5,6,7-tetrahydro-1,3-benzothiazol-2-yl)phenyl]-N,N-dimethylamine (Example 22), MS: m/z 368 (M+H)

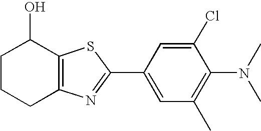

2-[3-chloro-4-(dimethylamino)-5-methylphenyl]-4,5,6,7-tetrahydro-1,3-benzothiazol-7-ol (Example 19), MS: m/z 323 (M+H)

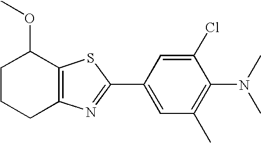

N-[2-chloro-4-(7-methoxy-4,5,6,7-tetrahydro-1,3-benzothiazol-2-yl)-6-methylphenyl]-N,N-dimethylamine (Example 20), MS: m/z 337 (M+H)

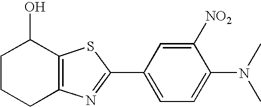

2-[4-(dimethylamino)-3-nitrophenyl]-4,5,6,7-tetrahydro-1,3-benzothiazol-7-ol (Example 24), MS: m/z 320 (M+H)

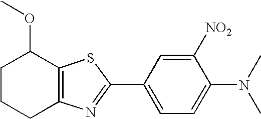

N-[4-(7-methoxy-4,5,6,7-tetrahydro-1,3-benzothiazol-2-yl)-2-nitrophenyl]-N,N-dimethylamine (Example 23), MS: m/z 334 (M+H)

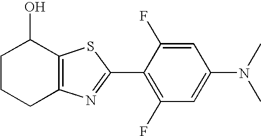

2-[4-(dimethylamino)-2,6-difluorophenyl]-4,5,6,7-tetrahydro-1,3-benzothiazol-7-ol (Example 25), MS: m/z 311 (M+H)

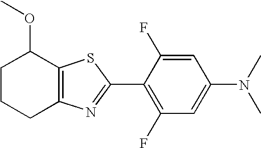

[3,5-difluoro-4-(7-methoxy-4,5,6,7-tetrahydro-1,3-benzothiazol-2-yl)phenyl]dimethylamine (Example 26), MS: m/z 325 (M+H)

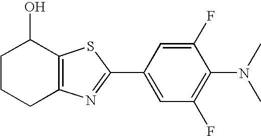

2-[4-(dimethylamino)-3,5-difluorophenyl]-4,5,6,7-tetrahydro-1,3-benzothiazol-7-ol (Example 27), MS: m/z 311 (M+H)

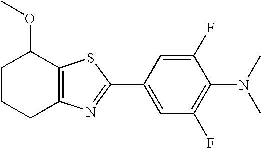

[2,6-difluoro-4-(7-methoxy-4,5,6,7-tetrahydro-1,3-benzothiazol-2-yl)phenyl]dimethylamine (Example 28), MS: m/z 325 (M+H)

### Example 29

### Preparation of 2-[3,5-Dichloro-4-(methylamino)phenyl]-4,5,6,7-tetrahydro-1,3-benzothiazol-7-ol

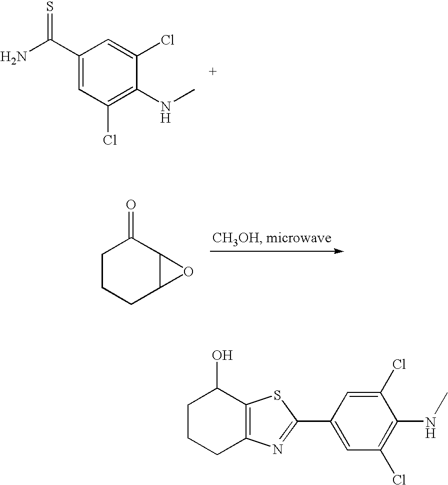

2-[3,5-Dichloro-4-(methylamino)phenyl]-4,5,6,7-tetrahydro-1,3-benzothiazol-7-ol was prepared by following the General Procedure III. MS: m/z 330 (M+H).

### Examples 30 and 31

### Preparation of [6-(7-methoxy-4,5,6,7-tetrahedro-benzothiazol-2-yl)-pyridin-3-yl]-dimethyl-amine (Example 30) and 2-(5-Dimethylamino-pyridine-2-yl)-4,5,6,7-tetrahedro-benzothiazol-7-ol (Example 31)

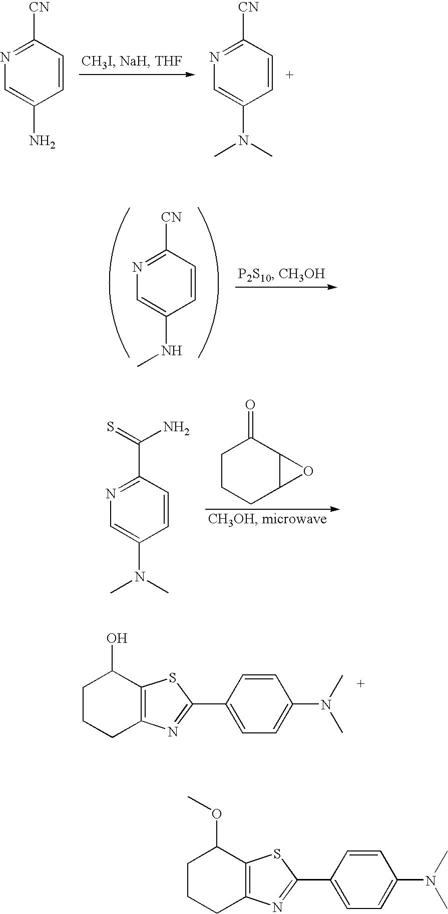

Step A. 5-Dimethylamino-pyridine-2-carbonitrile was prepared by following General Procedure I.

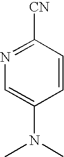

5-Dimethylamino-pyridine-2-carbonitrile, MS: m/z 148 (M+H)

Step B. 5-Dimethylamino-pyridine-2-carbothioic amide was prepared by following General Procedure II.

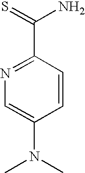

5-Dimethylamino-pyridine-2-carbothioic acid amide, MS: m/z 182 (M+H)

Step C. [6-(7-methoxy-4,5,6,7-tetrahedro-benzothiazol-2-yl)-pyridin-3-yl]-dimethyl-amine (Example 30) and 2-(5-Dimethylamino-pyridine-2-yl)-4,5,6,7-tetrahedro-benzothiazol-7-ol (Example 31) were prepared by following General Procedure III.

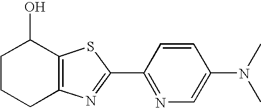

2-(5-Dimethylamino-pyridin-2-yl)-4,5,6,7-tetrahydro-benzothiazol-7-ol, MS: m/z 276 (M+H)

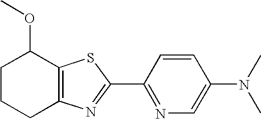

[6-(7-Methoxy-4,5,6,7-tetrahydro-benzothiazol-2-yl)-pyridin-3-yl]-dimethyl-amine, MS: m/z 290 (M+H)

### Example 32

### Preparation of [4-(7-Methoxy-4,5,6,7-tetrahydro-1,3-benzothiazol-2-yl)phenyl]amine

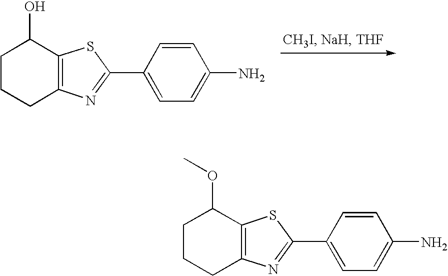

[4-(7-Methoxy-4,5,6,7-tetrahydro-1,3-benzothiazol-2-yl)phenyl]amine was prepared by following the previous procedure described in Example 1. MS: m/z 261 (M+H).

### Examples 33-36

### Preparation of 2-[4-(methylamino)-3-nitrophenyl]-4,5,6,7-tetrahydro-1,3-benzothiazol-7-ol (Example 33), N-[4-(7-methoxy-4,5,6,7-tetrahydro-1,3-benzothiazol-2-yl)-2-nitrophenyl]-N-methylamine (Example 34), [4-(7-ethoxy-4,5,6,7-tetrahydro-1,3-benzothiazol-2-yl)-2-nitrophenyl]dimethylamine (Example 35) and [4-(7-ethoxy-4,5,6,7-tetrahydro-1,3-benzothiazol-2-yl)-2-nitrophenyl]methylamine (Example 36)

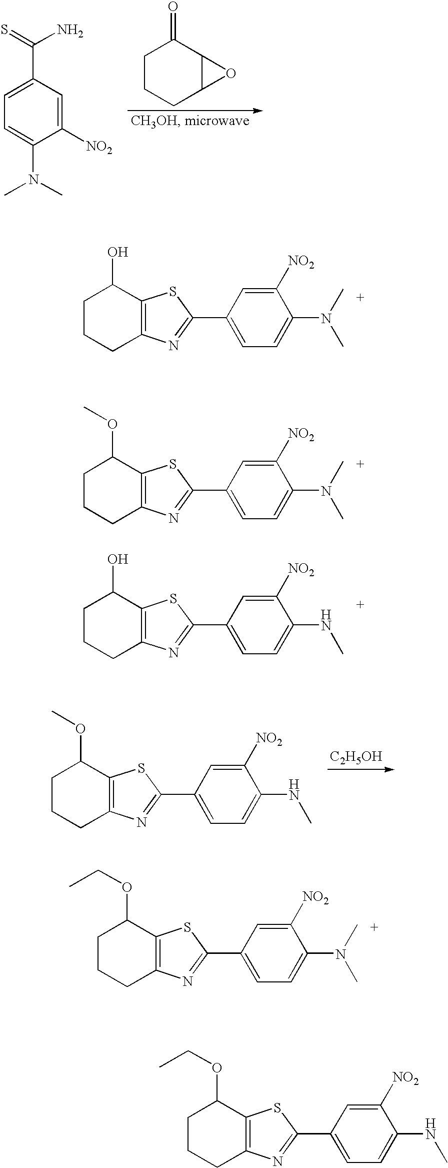

Examples 33-36 were prepared following General Procedure III. The compound 4-(dimethylamino)-3-nitrobenzenecarbothioamide (113 mg, 0.5 mmol) in CH₃OH (2 mL) was added to 7-oxabicyclo[4.1.0]heptan-2-one (0.1 mL, 1.0 mmol). The reaction was irradiated with microwave at 100° C. for 40 minutes, then concentrated. The residue was separated by silica gel column (CH₂Cl₂/THF=30:1), providing four products (see Example 11-28) as well as 2-[4-(methylamino)-3-nitrophenyl]-4,5,6,7-tetrahydro-1,3-benzothiazol-7-ol (Example 33) and N-[4-(7-methoxy-4,5,6,7-tetrahydro-1,3-benzothiazol-2-yl)-2-nitrophenyl]-N-methylamine (Example 34). When using C₂H₅OH as solvent, ethyl ether [4-(7-ethoxy-4,5,6,7-tetrahydro-1,3-benzothiazol-2-yl)-2-nitrophenyl]dimethylamine (Example 35) and [4-(7-ethoxy-4,5,6,7-tetrahydro-1,3-benzothiazol-2-yl)-2-nitrophenyl]methylamine (Example 36) were obtained.

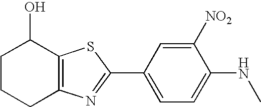

2-[4-(methylamino)-3-nitrophenyl]-4,5,6,7-tetrahydro-1,3-benzothiazol-7-ol, MS: m/z 306 (M+H)

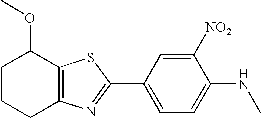

N-[4-(7-methoxy-4,5,6,7-tetrahydro-1,3-benzothiazol-2-yl)-2-nitrophenyl]-N-methylamine, MS: m/z 320 (M+H)

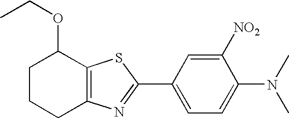

[4-(7-ethoxy-4,5,6,7-tetrahydro-1,3-benzothiazol-2-yl)-2-nitrophenyl]dimethylamine, MS: m/z 348 (M+H)

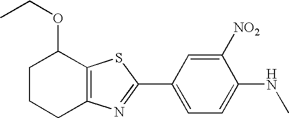

4-(7-ethoxy-4,5,6,7-tetrahydro-1,3-benzothiazol-2-yl)-2-nitrophenyl]methylamine, MS: m/z 334 (M+H)

### Example 37

### Preparation of N-[4-(7-methoxy-4,5,6,7-tetrahydro-1,3-benzothiazol-2-yl)phenyl]-N-methylamine

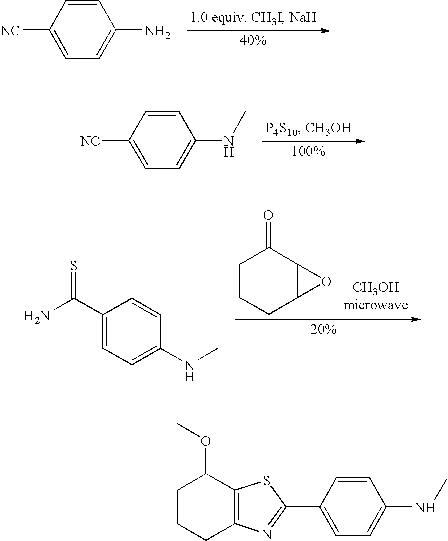

Step A. 4-(Isocyano-phenyl)-methylamine was prepared by following General Procedure I.

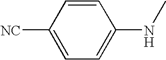

(4-Isocyano-phenyl)-methyl-amine, MS: m/z 133 (M+H)

Step B. 4-Metamino-thiobenzamide was prepared by following General Procedure II.

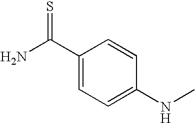

4-Methylamino-thiobenzamide, MS: m/z 167 (M+H)

Step C. N-[4-(7-methoxy-4,5,6,7-tetrahydro-1,3-benzothiazol-2-yl)phenyl]-N-methylamine was prepared by following General Procedure III.

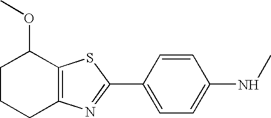

N-[4-(7-methoxy-4,5,6,7-tetrahydro-1,3-benzothiazol-2-yl)phenyl]-N-methylamine, MS: m/z 275 (M+H)

### Examples 38-40

### Preparation of 5-(7-Methoxy-4,5,6,7-tetrahydro-1,3-benzothiazol-2-yl)-N,N-dimethylpyridin-2-amine (Example 38), 2-[6-(dimethylamino)pyridin-3-yl]-4,5,6,7-tetrahydro-1,3-benzothiazol-7-ol (Example 39) and 5-(7-Ethoxy-4,5,6,7-tetrahydro-1,3-benzothiazol-2-yl)-N,N-dimethylpyridin-2-amine (Example 40)

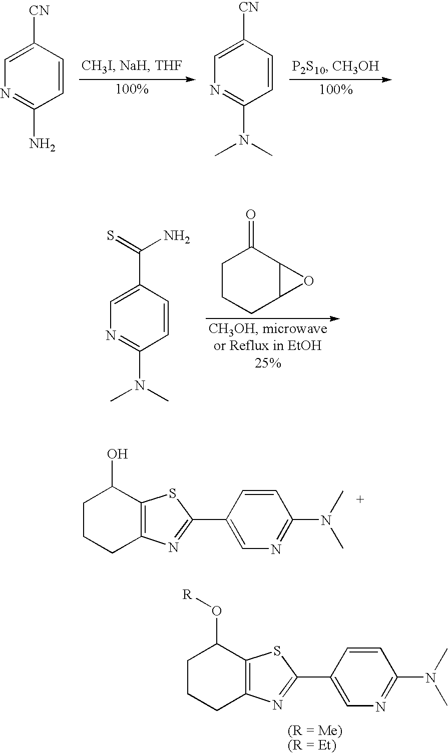

Step A. 6-(Dimethylamino)nicotinonitrile was prepared by following General Procedure I.

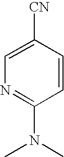

6-(dimethylamino)nicotinonitrile, MS: m/z 148 (M+H)

Step B. 6-(Dimethylamino)pyridine-3-carbothioamide was prepared by following General Procedure II.

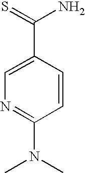

6-(dimethylamino)pyridine-3-carbothioamide, MS: m/z 182 (M+H)

Step C. 5-(7-Methoxy-4,5,6,7-tetrahydro-1,3-benzothiazol-2-yl)-N,N-dimethylpyridin-2-amine (Example 38) and 2-[6-(dimethylamino)pyridin-3-yl]-4,5,6,7-tetrahydro-1,3-benzothiazol-7-ol (example 39) were prepared by following General Procedure III. 5-(7-Ethoxy-4,5,6,7-tetrahydro-1,3-benzothiazol-2-yl)-N,N-dimethylpyridin-2-amine (Example 40) was prepared in the same way using ethanol as the solvent.

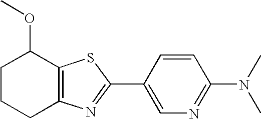

5-(7-methoxy-4,5,6,7-tetrahydro-1,3-benzothiazol-2-yl)-N,N-dimethylpyridin-2-amine, MS: m/z 290 (M+H)

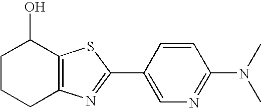

2-[6-(dimethylamino)pyridin-3-yl]-4,5,6,7-tetrahydro-1,3-benzothiazol-7-ol, MS: m/z 276 (M+H)

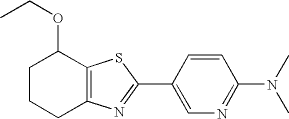

5-(7-ethoxy-4,5,6,7-tetrahydro-1,3-benzothiazol-2-yl)-N,N-dimethylpyridin-2-amine, MS: m/z 304 (M+H)

### Examples 41 and 42

### Preparation of 2-[4-(dimethylamino)-2-fluorophenyl]-4,5,6,7-tetrahydro-1,3-benzothiazol-7-ol (Example 41) and [4-(7-ethoxy-4,5,6,7-tetrahydro-1,3-benzothiazol-2-yl)-3-fluorophenyl]dimethylamine (Example 42)

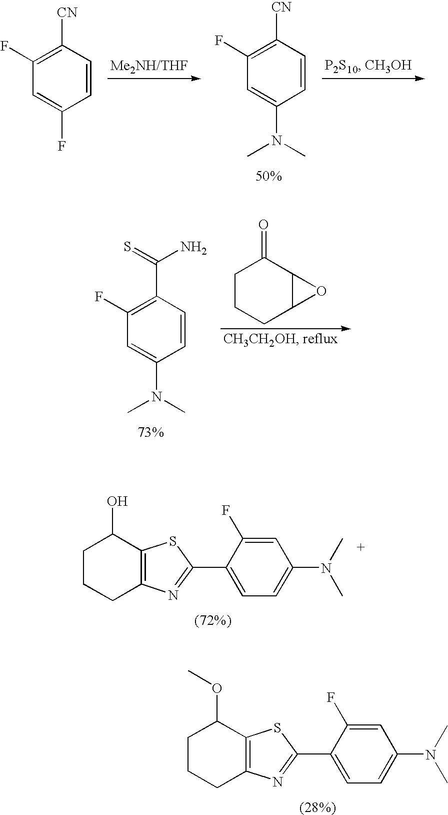

Step A. 4-Dimethylamino-2-fluoro-benzonitrile was prepared by following the procedure described earlier.

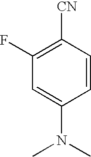

4-Dimethylamino-2-fluoro-benzonitrile, MS: m/z 165 (M+H)

Step B. 4-(dimethylamino)-2-fluorobenzenecarbothioamide was prepared by following General Procedure II.

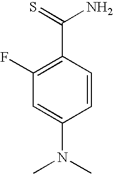

4-(dimethylamino)-2-fluorobenzenecarbothioamide, MS: m/z 199 (M+H)

Step C. 2-[4-(dimethylamino)-2-fluorophenyl]-4,5,6,7-tetrahydro-1,3-benzothiazol-7-ol (Example 41) and [4-(7-ethoxy-4,5,6,7-tetrahydro-1,3-benzothiazol-2-yl)-3-fluorophenyl]dimethylamine (Example 42) were prepared by following General Procedure III.

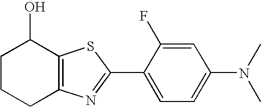

2-[4-(dimethylamino)-2-fluorophenyl]-4,5,6,7-tetrahydro-1,3-benzothiazol-7-ol, MS: m/z 293 (M+H)

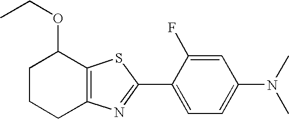

[4-(7-ethoxy-4,5,6,7-tetrahydro-1,3-benzothiazol-2-yl)-3-fluorophenyl]dimethylamine, MS: m/z 321 (M+H)

### Examples 43 and 44

### Preparation of 2-[4-(dimethylamino)-2,3-difluorophenyl]-4,5,6,7-tetrahydro-1,3-benzothiazol-7-ol (Example 43) and [4-(7-ethoxy-4,5,6,7-tetrahydro-1,3-benzothiazol-2-yl)-2,3-difluorophenyl]dimethylamine (Example 44)

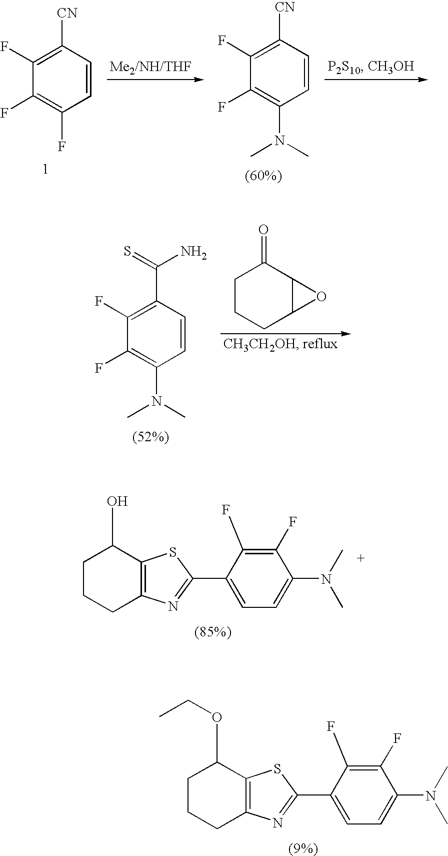

### Step A. 4-(dimethylamino)-2,3-difluorobenzonitrile

A mixture of 2,3,4-trifluoro benzonitrile (1.686 g, 10.73 mmol) in dimethylamine (2M solution in THF, 16.1 mL, 32.2 mmol) was stirred at room temperature for 4 hours, quenched with saturated NaHCO₃ solution, then extracted with ether three times. The combined extracts were washed with H₂O, brine, then dried over Na₂SO₄, filtered and concentrated. The residue was separated by silica gel column, eluting with hexane/EtOAc=3:1, to give 1.164 g (60%) of 4-(dimethylamino)-2,3-difluorobenzonitrile as a white solid.

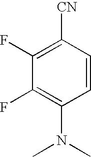

4-(dimethylamino)-2,3-difluorobenzonitrile, MS: m/z 183 (M+H)

Step B. 4-(dimethylamino)-2,3-difluorobenzenecarbothioamide was prepared by following General Procedure II.

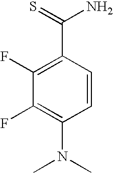

4-(dimethylamino)-2,3-difluorobenzenecarbothioamide, MS: m/z 217 (M+H)

Step C. The compounds 2-[4-(dimethylamino)-2,3-difluorophenyl]-4,5,6,7-tetrahydro-1,3-benzothiazol-7-ol (Example 43) and [4-(7-ethoxy-4,5,6,7-tetrahydro-1,3-benzothiazol-2-yl)-2,3-difluorophenyl]dimethylamine (Example 44) were prepared by following General Procedure III.

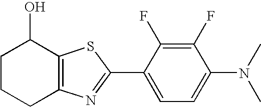

2-[4-(dimethylamino)-2,3-difluorophenyl]-4,5,6,7-tetrahydro-1,3-benzothiazol-7-ol, MS: m/z 311 (M+H)

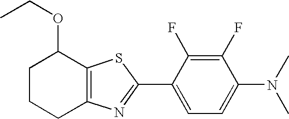

[4-(7-ethoxy-4,5,6,7-tetrahydro-1,3-benzothiazol-2-yl)-2,3-difluorophenyl]dimethylamine, MS: m/z 339 (M+H)

### Examples 45 and 46

### Preparation of 2-[4-(dimethylamino)-2-methylphenyl]-4,5,6,7-tetrahydro-1,3-benzothiazol-7-ol (Example 45) and [4-(7-ethoxy-4,5,6,7-tetrahydro-1,3-benzothiazol-2-yl)-3-methylphenyl]dimethylamine (Example 46)

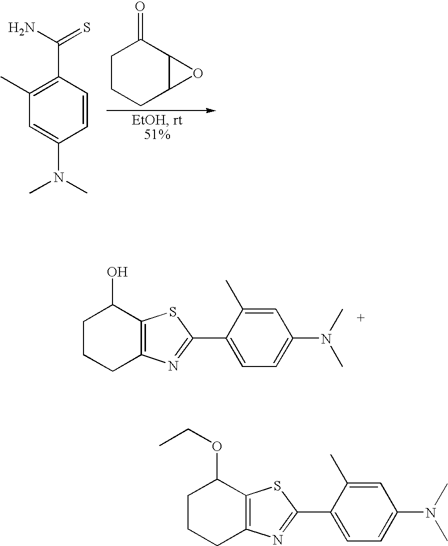

The compounds 2-[4-(dimethylamino)-2-methyl phenyl]-4,5,6,7-tetrahydro-1,3-benzothiazol-7-ol (Example 45) and [4-(7-ethoxy-4,5,6,7-tetrahydro-1,3-benzothiazol-2-yl)-3-methylphenyl]dimethylamine (Example 46) were prepared by following General Procedure III.

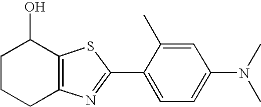

2-[4-(dimethylamino)-2-methylphenyl]-4,5,6,7-tetrahydro-1,3-benzothiazol-7-ol, MS: m/z 289 (M+H)

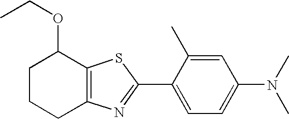

[4-(7-ethoxy-4,5,6,7-tetrahydro-1,3-benzothiazol-2-yl)-3-methylphenyl]dimethylamine, MS: m/z 317 (M+H)

### Examples 47 and 48

### Preparation of 2-[4-(dimethylamino)-2-ethyl phenyl]-4,5,6,7-tetrahydro-1,3-benzothiazol-7-ol (Example 47) and [4-(7-ethoxy-4,5,6,7-tetrahydro-1,3-benzothiazol-2-yl)-3-ethylphenyl]dimethylamine (Example 48)

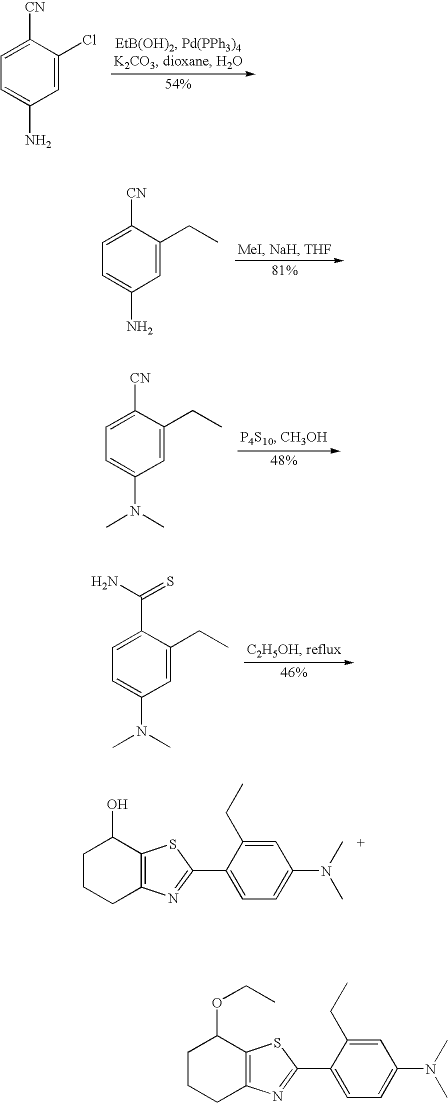

### Step A. Procedure for preparation of 4-amino-2-ethylbenzonitrile

A mixture of 4-amino-2-chlorobenzonitrile (1.53 g, 10 mmol), EtB(OH)₂ (739 mg, 10 mmol), Pd(PPh₃)₄ (578 mg, 0.5 mmol) and a 2.0 M K₂CO₃ solution (15 ml, 30 mmol) in dioxane (30 ml) was refluxed for 40 hours, then concentrated. The residue was added to H₂O, then extracted with EtOAc. The combined extracts were washed with brine, dried over Na₂SO₄, then filtered and concentrated. The residue was separated by flash chromatography (hexane/EtOAc=2:1) to give 794 mg (54%) of 4-amino-2-ethylbenzonitrile as a pale-yellow solid.

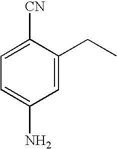

4-amino-2-ethylbenzonitrile, MS: m/z 147 (M+H)

Step B. 4-(dimethylamino)-2-ethylbenzonitrile was prepared by following General Procedure I.

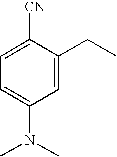

4-(dimethylamino)-2-ethylbenzonitrile, MS: m/z 175 (M+H)

Step C. 4-(dimethylamino)-2-ethylbenzenecarbothioamide was prepared by following General Procedure II.

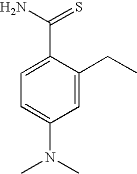

4-(dimethylamino)-2-ethylbenzenecarbothioamide, MS: m/z 209 (M+H)

Step D. 2-[4-(dimethylamino)-2-ethylphenyl]-4,5,6,7-tetrahydro-1,3-benzothiazol-7-ol (Example 47) and [4-(7-ethoxy-4,5,6,7-tetrahydro-1,3-benzothiazol-2-yl)-3-ethylphenyl]dimethylamine (Example 48) were prepared by following General Procedure III.

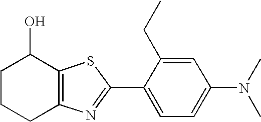

2-[4-(dimethylamino)-2-ethylphenyl]-4,5,6,7-tetrahydro-1,3-benzothiazol-7-ol, MS: m/z 303 (M+H)

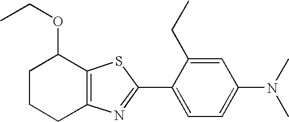

[4-(7-ethoxy-4,5,6,7-tetrahydro-1,3-benzothiazol-2-yl)-3-ethylphenyl]dimethylamine, MS: m/z 331 (M+H)

### Examples 49-52

### Preparation of 2-[3-amino-4-(dimethylamino)phenyl]-4,5,6,7-tetrahydro-1,3-benzothiazol-7-ol (Example 49), 4-(7-methoxy-4,5,6,7-tetrahydro-1,3-benzothiazol-2-yl)-N1,N1-dimethylbenzene-1,2-diamine (Example 50), 2-[3,4-bis(dimethylamino)phenyl]-4,5,6,7-tetrahydro-1,3-benzothiazol-7-ol (Example 51) and 2-[3-amino-4-(methylamino)phenyl]-4,5,6,7-tetrahydro-1,3-benzothiazol-7-ol (Example 52)

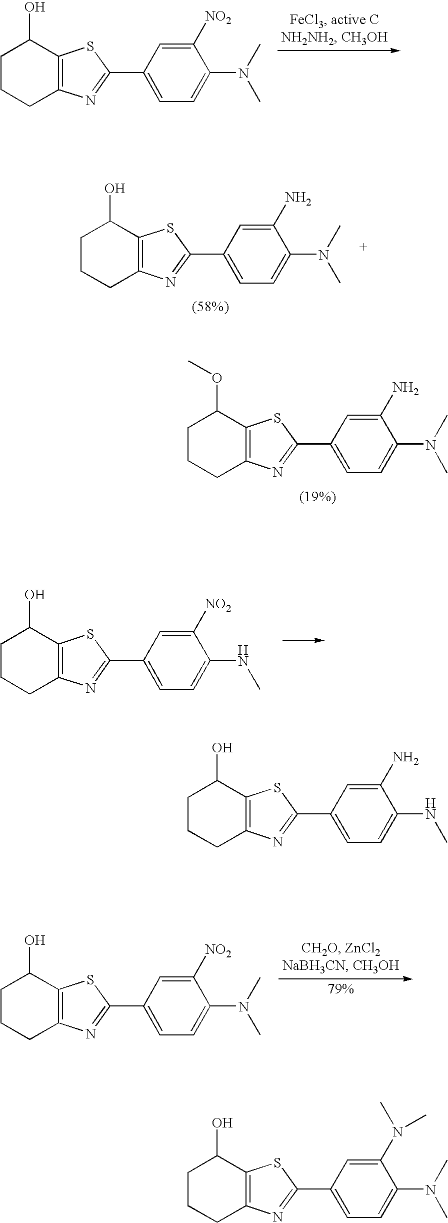

### Step A. Procedure for Preparation of 2-[3-amino-4-(dimethylamino)phenyl]-4,5,6,7-tetrahydro-1,3-benzothiazol-7-ol (Example 49), 4-(7-methoxy-4,5,6,7-tetrahydro-1,3-benzothiazol-2-yl)-N1,N1-dimethylbenzene-1,2-diamine (Example 50) and 2-[3-amino-4-(methylamino)phenyl]-4,5,6,7-tetrahydro-1,3-benzothiazol-7-ol (Example 52)

A mixture of 2-[4-(dimethylamino)-3-nitrophenyl]-4,5,6,7-tetrahydro-1,3-benzothiazol-7-ol (230 mg), FeCl₃.6H₂O (24 mg) and activated carbon (12 mg) in anhydrous CH₃OH (20 ml) was heated to reflux, then NH₂NH₂ (1.5 mL) was added. The reaction mixture was refluxed for 4 hours, cooled to room temperature, then stirred at room temperature overnight, and then concentrated. The residue was separated by flash chromatography (CH₂Cl₂/THF=10:1) to give 121 mg of 2-[3-amino-4-(dimethylamino)phenyl]-4,5,6,7-tetrahydro-1,3-benzothiazol-7-ol as a yellow solid, 41 mg of 4-(7-methoxy-4,5,6,7-tetrahydro-1,3-benzothiazol-2-yl)-N1,N1-dimethylbenzene-1,2-diamine as a yellow solid. Using the same procedure, 2-[3-amino-4-(methylamino)phenyl]-4,5,6,7-tetrahydro-1,3-benzothiazol-7-ol was prepared as a yellow solid.

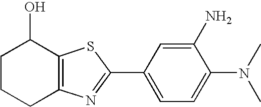

2-[3-amino-4-(dimethylamino)phenyl]-4,5,6,7-tetrahydro-1,3-benzothiazol-7-ol, MS: m/z 290 (M+H)

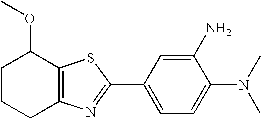

4-(7-methoxy-4,5,6,7-tetrahydro-1,3-benzothiazol-2-yl)-N1,N1-dimethylbenzene-1,2-diamine, MS: m/z 304 (M+H)

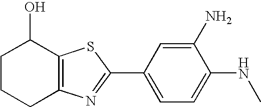

2-[3-amino-4-(methylamino)phenyl]-4,5,6,7-tetrahydro-1,3-benzothiazol-7-ol, MS: m/z 276 (M+H)

### Step B. Procedure for Preparation of 2-[3,4-bis(dimethylamino)phenyl]-4,5,6,7-tetrahydro-1,3-benzothiazol-7-ol (Example 51)

A solution of 2-[3-amino-4-(dimethylamino)phenyl]-4,5,6,7-tetrahydro-1,3-benzothiazol-7-ol (90 mg, 0.31 mmol) and CH₂O (37% in H₂O, 80 μL, 0.99 mmol) in CH₃OH (2 mL) was added to a mixture of NaBH₃CN (21 mg, 0.31 mmol) and ZnCl₂ (21 mg, 0.16 mmol) in CH₃OH (2 mL). The reaction mixture was stirred at room temperature overnight, then concentrated and separated by silica gel column (hexane/EtOAc=2:1) to give 78 mg (79%) of 2-[3,4-bis(dimethylamino)phenyl]-4,5,6,7-tetrahydro-1,3-benzothiazol-7-ol as a yellow sticky oil.

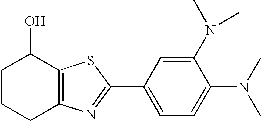

2-[3,4-bis(dimethylamino)phenyl]-4,5,6,7-tetrahydro-1,3-benzothiazol-7-ol, MS: m/z 318 (M+H)

### Examples 53 and 54

### Preparation of 2-[2-(dimethylamino)pyrimidin-5-yl]-4,5,6,7-tetrahydro-1,3-benzothiazol-7-ol (Example 53) and 5-(7-ethoxy-4,5,6,7-tetrahydro-1,3-benzothiazol-2-yl)-N,N-dimethylpyrimidin-2-amine (Example 54)

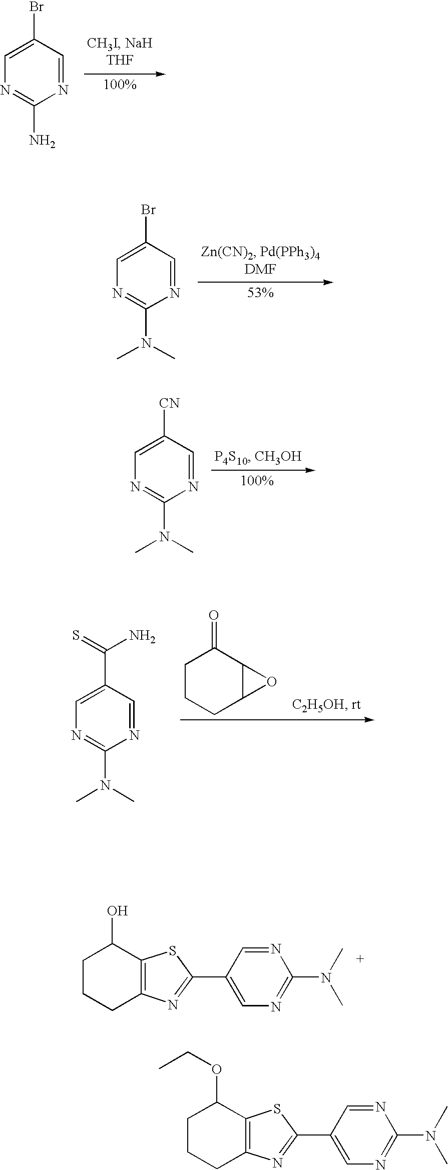

Step A. (5-Bromo-pyrimidin-2-yl)-dimethyl-amine was prepared by following the General Procedure I.

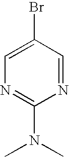

5-Bromo-pyrimidin-2-yl)-dimethyl-amine, MS: m/z 203 (M+H)

### Step B. 2-(dimethylamino)pyrimidine-5-carbonitrile

The compound (5-Bromo-pyrimidin-2-yl)-dimethyl-amine (202 mg, 1.0 mmol), Zn(CN)₂ (82 mg, 0.7 mmol) and Pd(PPh₃)₄ (58 mg, 0.05 mmol) in anhydrous DMF (1.0 ml) was heated to 86° C. (oil bath temperature) for 4 hours, then cooled to room temperature, diluted with toluene (5 ml), washed twice with diluted NH₄OH solution, brine and dried over Na₂SO₄, then filtered and concentrated. The residue was separated by flash chromatography (CH₂Cl₂/THF=40:1) to give 79 mg (53%) of the desired product as a white solid.

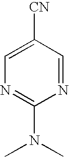

2-(dimethylamino)pyrimidine-5-carbonitrile, MS: m/z 149 (M+H)

Step C. 2-(dimethylamino)pyrimidine-5-carbothioamide was prepared by following General Procedure II.

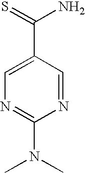

2-(dimethylamino)pyrimidine-5-carbothioamide, MS: m/z 183 (M+H)

Step D. The compounds 2-[2-(dimethylamino)pyrimidin-5-yl]-4,5,6,7-tetrahydro-1,3-benzothiazol-7-ol (Example 53) and 5-(7-ethoxy-4,5,6,7-tetrahydro-1,3-benzothiazol-2-yl)-N,N-dimethylpyrimidin-2-amine (Example 54) were prepared by following the General Procedure III.

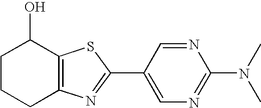

2-[2-(dimethylamino)pyrimidin-5-yl]-4,5,6,7-tetrahydro-1,3-benzothiazol-7-ol, MS: m/z 277 (M+H)

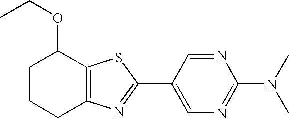

5-(7-ethoxy-4,5,6,7-tetrahydro-1,3-benzothiazol-2-yl)-N,N-dimethylpyrimidin-2-amine, MS: m/z 305 (M+H)

### Example 55-58

### Preparation of Methyl 5-(dimethylamino)-2-(7-hydroxy-4,5,6,7-tetrahydro-1,3-benzothiazol-2-yl)benzoate (Example 55), methyl 5-(dimethylamino)-2-(7-ethoxy-4,5,6,7-tetrahydro-1,3-benzothiazol-2-yl)benzoate (Example 56), methyl 5-(dimethylamino)-2-(7-methoxy-4,5,6,7-tetrahydro-1,3-benzothiazol-2-yl)benzoate (Example 57) and methyl 5-(dimethylamino)-2-(7-isopropoxy-4,5,6,7-tetrahydro-1,3-benzothiazol-2-yl)benzoate (Example 58)

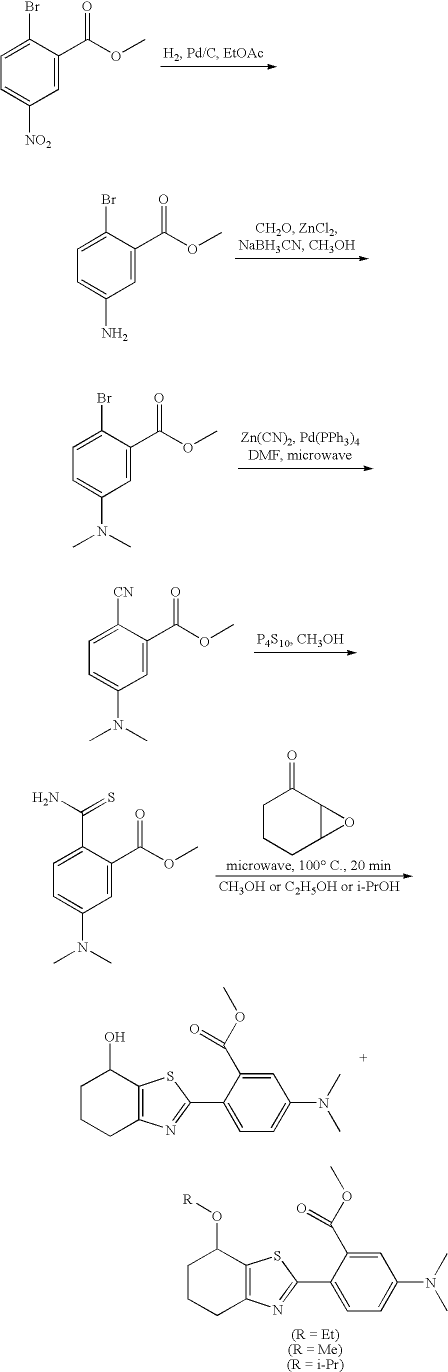

### Step A. Preparation of 5-amino-2-bromo-benzoic acid methyl ester

Methyl 2-bromo-5-nitrobenzoate (5.2 g, 20 mmol) dissolved in 100 mL of EtOAc was hydrogenated over 10% Pd/C (wet, 1.0 g) at 30 psi pressure of H₂ overnight, filtered and then concentrated to give 4.11 g (89%) of 5-amino-2-bromo-benzoic acid methyl ester as a brown sticky oil.

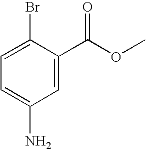

5-Amino-2-bromo-benzoic acid methyl ester, MS: m/z 231 (M+H)

### Step B. Preparation of methyl 2-bromo-5-(dimethylamino)benzoate

To a stirred solution of 5-amino-2-bromo-benzoic acid methyl ester (3.30 g, 14.35 mmol) in CH₃OH (45 mL) containing CH₂O (3.6 mL, 45.0 mmol) at room temperature was added a solution of NaBH₃CN (945 mg, 14.35 mmol) and ZnCl₂ (990 mg, 7.2 mmol) in CH₃OH (45 mL). The reaction mixture was stirred at room temperature overnight, then concentrated. The residue was taken up in 0.1 N NaOH solution, extracted with EtOAc several times. The combined extracts were washed with H₂O, brine, dried over dried Na₂SO₄, then filtered and concentrated. The residue was separated by silica gel column (hexane/EtOAc=3:1) to give 2.991 g (92%) of methyl 2-bromo-5-(dimethylamino)benzoate as a yellow oil based on the partial recovery of (412 mg).

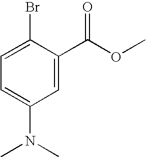

methyl 2-bromo-5-(dimethylamino)benzoate, MS: m/z 259 (M+H)

### Step C. Preparation of methyl 2-cyano-5-(dimethylamino)benzoate

Methyl 2-bromo-5-(dimethylamino)benzoate (774 mg, 3.0 mmol), Zn(CN)₂ (387 mg, 3.3 mmol) and Pd(PPh₃)₄ (173 mg. 0.15 mmol) in DMF (3 mL) was microwaved at 140° C. for 30 minutes. To this mixture was added 2N NH₄OH solution (20 mL), which was then extracted with EtOAc twice. The combined extracts were washed with 2N NH₄OH solution, H₂O, brine, dried over Na₂SO₄, then filtered and concentrated. The residue was separated by silica gel column (CH₂Cl₂/THF=40:1) to give 588 mg (96%) of methyl 2-cyano-5-(dimethylamino)benzoate as a yellow solid.

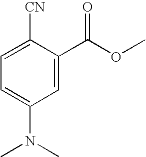

methyl 2-cyano-5-(dimethylamino)benzoate, MS: m/z 205 (M+H)

Step D. Methyl 2-(aminocarbonothioyl)-5-(dimethylamino)benzoate was prepared by following General Procedure II.

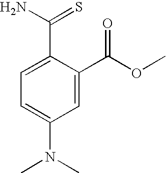

methyl 2-(aminocarbonothioyl)-5-(dimethylamino)benzoate, MS: m/z 239 (M+H)

Step E. Methyl 5-(dimethylamino)-2-(7-hydroxy-4,5,6,7-tetrahydro-1,3-benzothiazol-2-yl)benzoate, methyl 5-(dimethylamino)-2-(7-ethoxy-4,5,6,7-tetrahydro-1,3-benzothiazol-2-yl)benzoate, methyl 5-(dimethylamino)-2-(7-methoxy-4,5,6,7-tetrahydro-1,3-benzothiazol-2-yl)benzoate and methyl 5-(dimethylamino)-2-(7-isopropoxy-4,5,6,7-tetrahydro-1,3-benzothiazol-2-yl)benzoate were prepared by following General Procedure III.

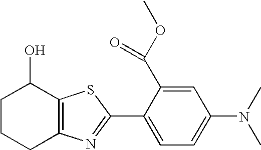

methyl 5-(dimethylamino)-2-(7-hydroxy-4,5,6,7-tetrahydro-1,3-benzothiazol-2-yl)benzoate MS: m/z 333 (M+H)

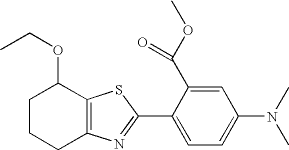

methyl 5-(dimethylamino)-2-(7-ethoxy-4,5,6,7-tetrahydro-1,3-benzothiazol-2-yl)benzoate, MS: m/z 361 (M+H)

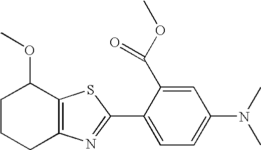

methyl 5-(dimethylamino)-2-(7-methoxy-4,5,6,7-tetrahydro-1,3-benzothiazol-2-yl)benzoate, MS: m/z 347 (M+H)

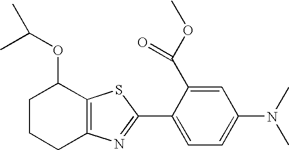

methyl 5-(dimethylamino)-2-(7-isopropoxy-4,5,6,7-tetrahydro-1,3-benzothiazol-2-yl)benzoate, MS: m/z 375 (M+H)

### Examples 59 and 60

### Preparation of 5-(dimethylamino)-2-(7-hydroxy-4,5,6,7-tetrahydro-1,3-benzothiazol-2-yl)benzoic acid (Example 59) and 2-[4-(dimethylamino)-2-(hydroxymethyl)phenyl]-4,5,6,7-tetrahydro-1,3-benzothiazol-7-ol (Example 60)

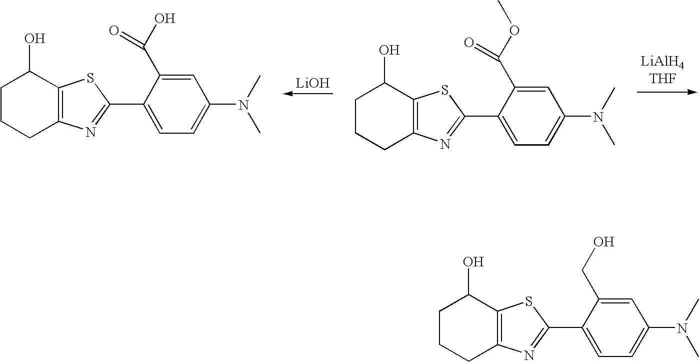

Methyl 5-(dimethylamino)-2-(7-hydroxy-4,5,6,7-tetrahydro-1,3-benzothiazol-2-yl)benzoate (104 mg, 0.313 mmol) dissolved in THF (1.0 mL) was added 1.0M LiOH solution (1.3 mL). The reaction mixture was microwaved at 100° C. for 1 hour, then neutralized with 1.0M citric acid solution to pH 5-6, and then extracted with CH₂Cl₂. The combined extracts were washed with H₂O, brine, dried over Na₂SO₄, filtered and then concentrated to give 80 mg (80%) of 5-(dimethylamino)-2-(7-hydroxy-4,5,6,7-tetrahydro-1,3-benzothiazol-2-yl)benzoic acid as a yellow solid.

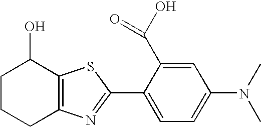

5-(dimethylamino)-2-(7-hydroxy-4,5,6,7-tetrahydro-1,3-benzothiazol-2-yl)benzoic acid, MS: m/z 319 (M+H)

Methyl 5-(dimethylamino)-2-(7-hydroxy-4,5,6,7-tetrahydro-1,3-benzothiazol-2-yl)benzoate (60 mg, 0.18 mmol) and LiAlH₄ (50 mg) in THF (1.0 mL) was stirred at room temperature for 5 hours, then quenched with sat. NH₄Cl solution, extracted with CH₂Cl₂. Separation by CombiFlash (grading elution with hexane/EtOAc) gave 20 mg (36%) of 2-[4-(dimethylamino)-2-(hydroxymethyl)phenyl]-4,5,6,7-tetrahydro-1,3-benzothiazol-7-ol as a yellow solid.

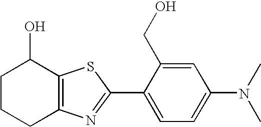

2-[4-(dimethylamino)-2-(hydroxymethyl)phenyl]-4,5,6,7-tetrahydro-1,3-benzothiazol-7-ol, MS: m/z 305 (M+H)

### Examples 61 and 62

The amides were prepared as follows:

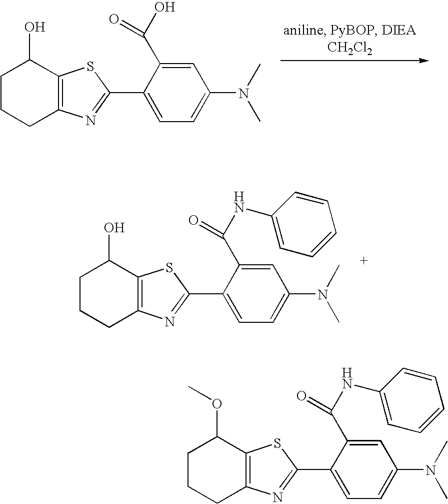

A mixture of 5-(dimethylamino)-2-(7-hydroxy-4,5,6,7-tetrahydro-1,3-benzothiazol-2-yl)benzoic acid (64 mg, 0.2 mmol), PyBOP (135 mg, 0.26 mmol) in CH₂Cl₂ (2.5 mL) was added diisopropylethylamine (80 μL, 0.46 mmol) and aniline (18 μL, 0.2 mmol). The reaction mixture was stirred at room temperature for 48 hours, then concentrated. The residue was dissolved in CH₃OH and separated by HPLC. The fraction was co-evaporated with CH₃OH. The residue was re-separated by silica gel column (hexane/EtOAc=1:1), providing 6 mg (Example 61) and 10 mg (Example 62).

Example 61: yellow solid; MS: m/z 394 (M+H); Example 62: yellow film; MS: m/z 408 (M+H).

### Examples 63-66

### Preparation of 2-[4-(dimethylamino)-2-(pyrrolidin-1-ylmethyl)phenyl]-4,5,6,7-tetrahydro-1,3-benzothiazol-7-ol (Example 63), 2-[4-(dimethylamino)-2-(morpholin-4-ylmethyl)phenyl]-4,5,6,7-tetrahydro-1,3-benzothiazol-7-ol (Example 64), 2-{4-(dimethylamino)-2-[(dimethylamino)methyl]phenyl}-4,5,6,7-tetrahydro-1,3-benzothiazol-7-ol (Example 65) and 2-(4-(dimethylamino)-2-{[[2-(dimethylamino)ethyl](ethyl)amino]methyl}phenyl)-4,5,6,7-tetrahydro-1,3-benzothiazol-7-ol (Example 66)

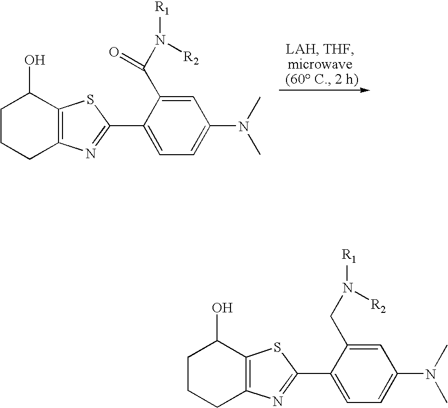

The amides were prepared by following the procedure described in Examples 61-62.

General Procedure of preparation of amino analogs. A solution of amide (0.16 mmol) in anhydrous THF (2 mL) was added lithium aluminum hydride, LAH (0.25 mL, 1.0 M in THF available from Aldrich) under N₂. The reaction mixture was subjected to a microwave-assisted reaction at 60° C. for 1.5-2 hours, then cooled to room temperature and quenched with a few drops of H₂O, then concentrated and separated either by HPLC or silica gel column (EtOAc/TEA=20:1 or EtOAc/MeOH=10:1).

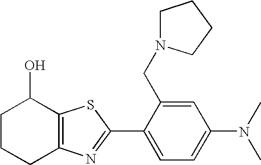

2-[4-(dimethylamino)-2-(pyrrolidin-1-ylmethyl)phenyl]-4,5,6,7-tetrahydro-1,3-benzothiazol-7-ol, (Example 63): 62% as a yellow solid; MS: m/z 358 (M+H)

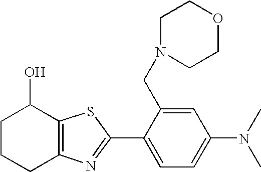

2-[4-(dimethylamino)-2-(morpholin-4-ylmethyl)phenyl]-4,5,6,7-tetrahydro-1,3-benzothiazol-7-ol, (Example 64): 61% as a yellow solid; MS: m/z 374 (M+H)

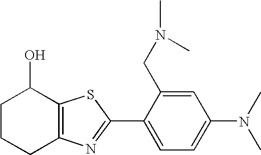

2-{4-(dimethylamino)-2-[(dimethylamino)methyl]phenyl}-4,5,6,7-tetrahydro-1,3-benzothiazol-7-ol, (Example 65): 44% as a yellow solid; MS: m/z 332 (M+H)

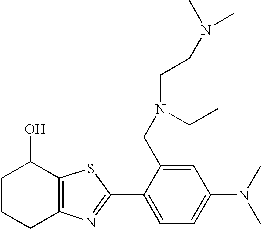

2-(4-(dimethylamino)-2-{[[2-(dimethylamino)ethyl](ethyl)amino]methyl}phenyl)-4,5,6,7-tetrahydro-1,3-benzothiazol-7-ol, (Example 66): 35% as a yellow gum; MS: m/z 403 (M+H)

### Example 67

The benzaldehyde derivative was prepared from the benzoic acid derivative as follows:

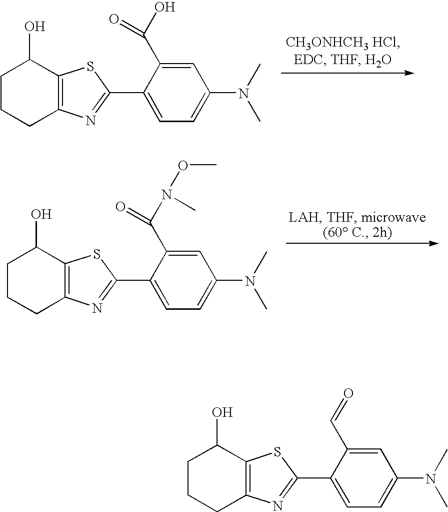

A mixture of 5-(dimethylamino)-2-(7-hydroxy-4,5,6,7-tetrahydro-1,3-benzothiazol-2-yl)benzoic acid (80 mg, 0.25 mmol) and N,O-dimethylhydroxylamine hydrochloride (41 mg, 0.425 mmol) in THF (0.5 mL) and H₂O (0.5 mL) was slowly added a solution of N-(3-dimethylaminopropyl)-N′-ethylcarbodiimide hydrochloride (120 mg, 0.625 mmol) in H₂O (1.8 mL). The reaction mixture was stirred at pH 4.5 at room temperature for 3 hours. To this was added brine (2 mL) and extracted with CH₂Cl₂. The combined organic layers were washed with H₂O, brine, then over dried Na₂SO₄. The crude mixture was separated by flash chromatography (CH₂Cl₂:CH₃CN=11:9), providing 50 mg (55%) of the amide compound as a yellow solid.

A solution of the amide compound (37 mg, 0.1 mmol) in anhydrous THF (1 mL) was added a 1.0 M solution of LiAlH₄/THF (0.2 mL, 0.2 mmol). The microwave-assisted reduction was carried out by following the procedure described in Example 63-66. Separation by flash chromatography (hexane:EtOAc=1:1) gave 11 mg (36%) of the product as a yellow film, MS: m/z 303 (M+H).

### Example 68

### Preparation of 1-methylpyrrolidin-3-yl 5-(dimethylamino)-2-(7-hydroxy-4,5,6,7-tetrahydro-1,3-benzothiazol-2-yl)benzoate

The product was prepared following the procedure described in Example 61-62.

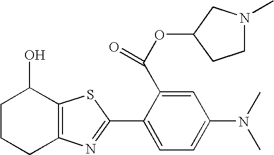

1-methylpyrrolidin-3-yl 5-(dimethylamino)-2-(7-hydroxy-4,5,6,7-tetrahydro-1,3-benzothiazol-2-yl)benzoate, MS: m/z 402 (M+H)

### Example 69

### Preparation of 2-[4-(Dimethylamino)-2-(methoxymethyl)phenyl]-4,5,6,7-tetrahydro-1,3-benzothiazol-7-ol

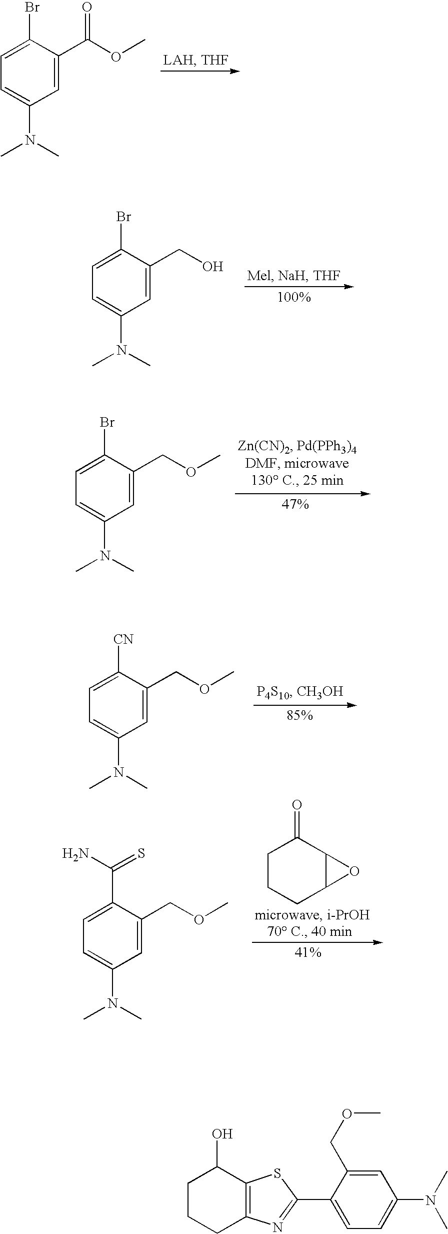

### Step A. Preparation of [2-bromo-5-(dimethylamino)phenyl]methanol

A solution of methyl 2-bromo-5-(dimethylamino)benzoate (4.35 g, 16.86 mmol) in anhydrous THF (60 mL) was added a 1.0M solution of LiAlH₄/THF (21.92 mL, 21.92 mmol). The reaction mixture was stirred at room temperature for 3 hours, then quenched with H₂O, and then extracted with EtOAc. The combined extracts were washed with H₂O, brine and dried over anhydrous Na₂SO₄. Separation by flash chromatography (CH₂Cl₂:THF=30:1), provided the desired product methyl 2-bromo-5-(dimethylamino)benzyl alcohol as a pale-yellow solid.

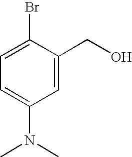

[2-bromo-5-(dimethylamino)phenyl]methanol, MS: m/z 231 (M+H)

### Step B. Preparation of 4-bromo-3-(methoxymethyl)-N,N-dimethylaniline

A suspension of [2-bromo-5-(dimethylamino)phenyl]methanol (184 mg, 0.8 mmol) and NaH (38 mg, 0.96 mmol) in anhydrous THF was added CH₃I (136 mg, 0.96 mmol). The reaction mixture was stirred at room temperature for 2 hours, quenched with H₂O, and then extracted with EtOAc. The combined extracts were washed with H₂O, brine and then dried over anhydrous Na₂SO₄. After filtration, the solvent was evaporated to give 4-bromo-3-(methoxymethyl)-N,N-dimethylaniline in quantitative yield as a yellow oil.

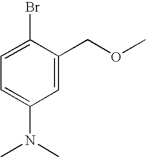

4-bromo-3-(methoxymethyl)-N,N-dimethylaniline, MS: m/z 245 (M+H)

### Step C. Preparation of 4-(dimethylamino)-2-(methoxymethyl)benzonitrile

4-(Dimethylamino)-2-(methoxymethyl)benzonitrile was prepared by following the Step C in Examples 55-58. Separation by CombiFlash (hexane/EtOAc gradient) gave 47% of 4-(dimethylamino)-2-(methoxymethyl)benzonitrile as a pale-yellow oil.

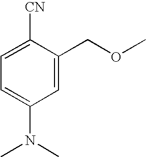

4-(dimethylamino)-2-(methoxymethyl)benzonitrile, MS: m/z 191 (M+H)

Step D. 4-(Dimethylamino)-2-(methoxymethyl)benzenecarbothioamide was prepared by following General procedure II. Separation by silica gel column (hexane:EtOAc, 1:1) gave 85% of 4-(dimethylamino)-2-(methoxymethyl)-benzenecarbothioamide as a dark yellow solid.

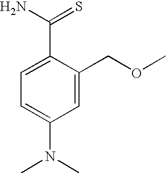

4-(dimethylamino)-2-(methoxymethyl)benzenecarbothioamide, MS: m/z 225 (M+H)

Step E. 2-[4-(Dimethylamino)-2-(methoxymethyl)phenyl]-4,5,6,7-tetrahydro-1,3-benzothiazol-7-ol was prepared by following General procedure III, providing 41% of 2-[4-(Dimethylamino)-2-(methoxymethyl)phenyl]-4,5,6,7-tetrahydro-1,3-benzothiazol-7-ol as a yellow solid.

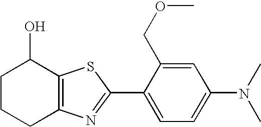

2-[4-(dimethylamino)-2-(methoxymethyl)phenyl]-4,5,6,7-tetrahydro-1,3-benzothiazol-7-ol, MS: m/z 319 (M+H)

### Examples 70 and 71

### Preparation of 2-[2-[(Benzyloxy)methyl]-4-(dimethylamino)phenyl]-4,5,6,7-tetrahydro-1,3-benzothiazol-7-ol (Example 70) and 3-[(benzyloxy)methyl]-4-(7-isopropoxy-4,5,6,7-tetrahydro-1,3-benzothiazol-2-yl)-N,N-dimethylaniline (Example 71)

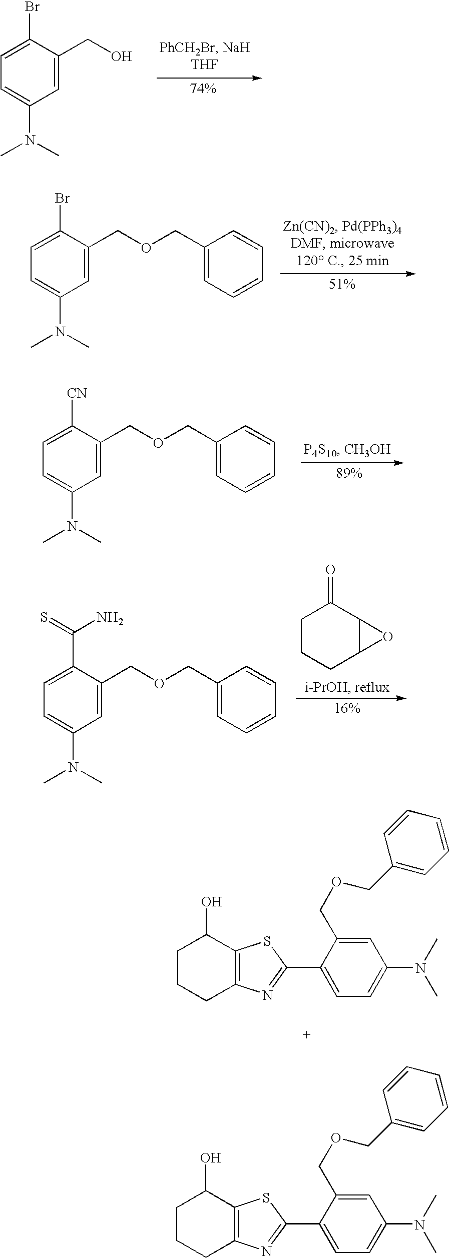

### Step A. Preparation of 3-[(benzyloxy)methyl]-4-bromo-N,N-dimethylaniline

Following Step A in Example 69, a reaction mixture of [2-bromo-5-(dimethylamino)phenyl]methanol (184 mg, 0.8 mmol), NaH (38 mg, 0.96 mmol) and benzyl bromide (164 mg, 0.96 mmol) in anhydrous THF (5 mL) was stirred at room temperature for 6 hours. Separation by CombiFlash (hexane/EtOAc gradient) give 190 mg (74%) of 3-[(benzyloxy)methyl]-4-bromo-N,N-dimethylaniline as a pale-yellow oil.

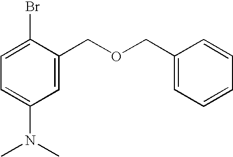

3-[(benzyloxy)methyl]-4-bromo-N,N-dimethylaniline, MS: m/z 321 (M+H)

### Step B. Preparation of 2-[(benzyloxy)methyl]-4-(dimethylamino)benzonitrile

2-[(Benzyloxy)methyl]-4-(dimethylamino)benzonitrile was prepared by following Step C in Examples 55-58. Separation by CombiFlash (hexane/EtOAc gradient) gave 51% of 2-[(benzyloxy)methyl]-4-(dimethylamino)benzonitrile as a pale-yellow sticky oil.

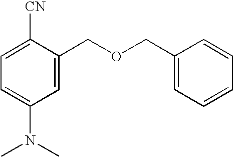

2-[(benzyloxy)methyl]-4-(dimethylamino)benzonitrile, MS: m/z 267 (M+H)

Step C. 2-[(Benzyloxy)methyl]-4-(dimethylamino)benzenecarbothioamide was prepared by following the General procedure II. Separation by CombiFlash (hexane/EtOAc gradient) gave 89% of 2-[(Benzyloxy)methyl]-4-(dimethylamino)benzenecarbothioamide as a yellow solid.

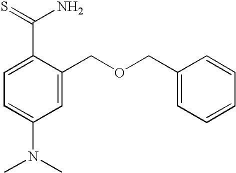

2-[(benzyloxy)methyl]-4-(dimethylamino)benzenecarbothioamide, MS: m/z 301 (M+H)

Step D. 2-[2-[(Benzyloxy)methyl]-4-(dimethylamino)phenyl]-4,5,6,7-tetrahydro-1,3-benzothiazol-7-ol and 3-[(benzyloxy)methyl]-4-(7-isopropoxy-4,5,6,7-tetrahydro-1,3-benzothiazol-2-yl)-N,N-dimethylaniline were prepared by following General procedure III

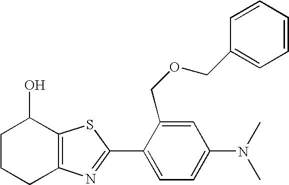

2-[2-[(benzyloxy)methyl]-4-(dimethylamino)phenyl]-4,5,6,7-tetrahydro-1,3-benzothiazol-7-ol, 16% as a yellow gum; MS: m/z 395 (M+H)

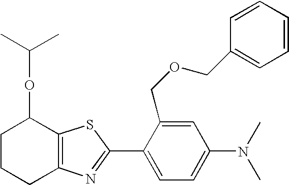

3-[(benzyloxy)methyl]-4-(7-isopropoxy-4,5,6,7-tetrahydro-1,3-benzothiazol-2-yl)-N,N-dimethylaniline, 10% as a yellow sticky oil; MS: m/z 437 (M+H)

### Example 72

### Preparation of 2-[4-(dimethylamino)-2-(1-hydroxy-1-methylethyl)phenyl]-4,5,6,7-tetrahydro-1,3-benzothiazol-7-ol

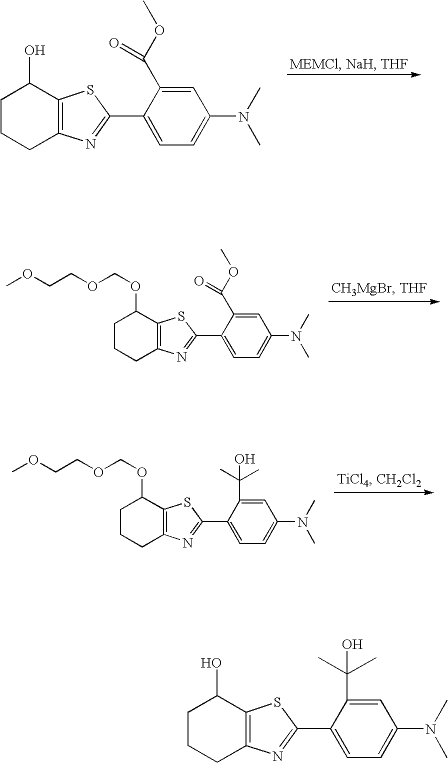

### Step A. Preparation of methyl 5-(dimethylamino)-2-{7-[(2-methoxyethoxy)methoxy]-4,5,6,7-tetrahydro-1,3-benzothiazol-2-yl}benzoate

To a solution of 5-(dimethylamino)-2-(7-hydroxy-4,5,6,7-tetrahydro-1,3-benzothiazol-2-yl)benzoic acid methyl ester (2.324 g, 7.0 mmol) in anhydrous THF (20 mL) cooled in an ice bath was added NaH (560 mg, 14.0 mmol) followed by the addition of MEMCI (1.2 mL, 10.5 mmol). The reaction mixture was stirred at 0° C., then gradually warmed up to room temperature for 8 hours, quenched with H₂O, and then extracted with EtOAc. The combined extracts were washed with H₂O, brine and dried over Na₂SO₄, filtered and then concentrated to give methyl 5-(dimethylamino)-2-{7-[(2-methoxyethoxy)methoxy]-4,5,6,7-tetrahydro-1,3-benzothiazol-2-yl}benzoate in quantitative yield as a yellow sticky oil which is pure enough for the use in next steps. If purification is needed, use CH₂Cl₂/THF=30:1 as the eluent.

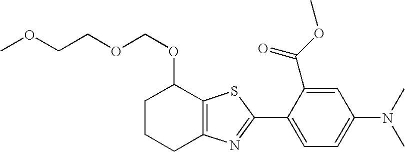

Methyl 5-(dimethylamino)-2-{7-[(2-methoxyethoxy)methoxy]-4,5,6,7-tetrahydro-1,3-benzothiazol-2-yl}benzoate, MS: m/z 421 (M+H)

### Step B. Preparation of 2-(5-(dimethylamino)-2-{7-[(2-methoxyethoxy)methoxy]-4,5,6,7-tetrahydro-1,3-benzothiazol-2-yl}phenyl)propan-2-ol

To a solution of methyl 5-(dimethylamino)-2-{7-[(2-methoxyethoxy)methoxy]-4,5,6,7-tetrahydro-1,3-benzothiazol-2-yl}benzoate (294 mg, 0.7 mmol) in anhydrous THF (2 mL) cooled in an ice bath was added a 1.4M solution of CH3MgBr/toluene/THF. The reaction mixture was stirred at 0° C., then gradually warmed up to room temperature overnight, quenched with 1.0M citric acid solution, and then extracted with EtOAc. The combined extracts were washed with H₂O, brine and dried over Na₂SO₄, filtered and concentrated to give 2-(5-(dimethylamino)-2-{7-[(2-methoxyethoxy)methoxy]-4,5,6,7-tetrahydro-1,3-benzothiazol-2-yl}phenyl)propan-2-ol in quantitative yield as a yellow sticky oil.

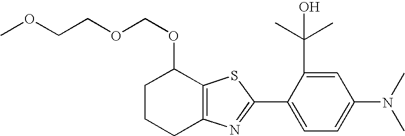

2-(5-(dimethylamino)-2-{7-[(2-methoxyethoxy)methoxy]-4,5,6,7-tetrahydro-1,3-benzothiazol-2-yl}phenyl)propan-2-ol, MS: m/z 421 (M+H)

### Step C. Deprotection to Remove MEM Protecting Group for Preparation of 2-[4-(dimethylamino)-2-(1-hydroxy-1-methylethyl)phenyl]-4,5,6,7-tetrahydro-1,3-benzothiazol-7-ol

To a solution of 2-(5-(dimethylamino)-2-{7-[(2-methoxyethoxy)methoxy]-4,5,6,7-tetrahydro-1,3-benzothiazol-2-yl}phenyl)propan-2-ol (84 mg, 0.2 mmol) in anhydrous CH₂Cl₂ (1 mL) at 0° C. was added a 1.0M solution of TiCl₄/CH₂Cl₂ (1.0 mL, 1.0 mmol). The reaction mixture was stirred at 0° C. for 5 hours, then quenched with concentrated NH₄OH and H₂O, then extracted with CH₂Cl₂. The combined organic layers were washed with H₂O, brine and dried over Na₂SO₄, filtered and then concentrated. The residue was separated by flash chromatography (CH₂Cl₂:THF=30:2), providing 37 mg (56%) of 2-[4-(dimethylamino)-2-(1-hydroxy-1-methylethyl)phenyl]-4,5,6,7-tetrahydro-1,3-benzothiazol-7-ol as a yellow solid.

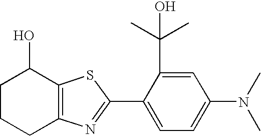

2-[4-(dimethylamino)-2-(1-hydroxy-1-methylethyl)phenyl]-4,5,6,7-tetrahydro-1,3-benzothiazol-7-ol, MS: m/z 333 (M+H)

### Example 73

### Preparation of 2-[4-(dimethylamino)-2-(1-hydroxyethyl)phenyl]-4,5,6,7-tetrahydro-1,3-benzothiazol-7-ol

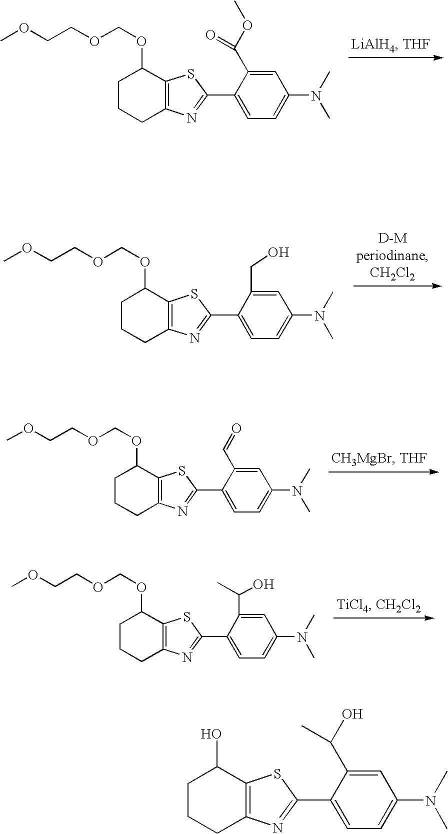

### Step A. Preparation of (5-(dimethylamino)-2-{7-[(2-methoxyethoxy)methoxy]-4,5,6,7-tetrahydro-1,3-benzothiazol-2-yl}phenyl)methanol

To a solution of ester (772 mg, 1.84 mmol) in anhydrous THF (10 mL) cooled in an ice bath was added LiAlH₄ (1.0 M/THF, 1.8 mL, 1.8 mmol). The reaction mixture was stirred at 0° C. for 2 hours, then quenched with H₂O, and then filtered to remove the insoluble materials. The filtrate was extracted with EtOAc. The combined extracts were washed with H₂O, brine and dried over Na₂SO₄, filtered and then concentrated. The residue was dried in vacuum to give quantitative yield of (5-(dimethylamino)-2-{7-[(2-methoxyethoxy)methoxy]-4,5,6,7-tetrahydro-1,3-benzothiazol-2-yl}phenyl)methanol as a yellow sticky oil, which was pure enough for the next step reaction.

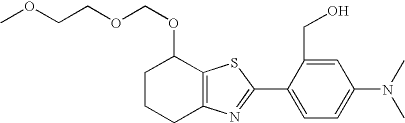

(5-(dimethylamino)-2-{7-[(2-methoxyethoxy)methoxy]-4,5,6,7-tetrahydro-1,3-benzothiazol-2-yl}phenyl)methanol, MS: m/z 393 (M+H)

### Step B. Preparation of 5-(dimethylamino)-2-{7-[(2-methoxyethoxy)methoxy]-4,5,6,7-tetrahydro-1,3-benzothiazol-2-yl}benzaldehyde

A solution of (5-(dimethylamino)-2-{7-[(2-methoxyethoxy)methoxy]-4,5,6,7-tetrahydro-1,3-benzothiazol-2-yl}phenyl)methanol (1.18 g, 3.0 mmol) in CH₂Cl₂ (35 mL) cooled in an ice bath was added Dess-Martin periodinane (1.43 g, 3.3 mmol). The reaction mixture was stirred at 0° C. for 3 hours, then quenched with H₂O and 1.0N NaOH solution (20 mL). After stirring at 0° C. for 10 minutes, the organic layer was separated, and the aqueous layer was extracted with CH₂Cl₂ twice. The combined organic layers were washed with H₂O, brine, dried over Na₂SO₄, filtered and then concentrated. The residue was dried in vacuum to give quantitative yield of crude 5-(dimethylamino)-2-{7-[(2-methoxyethoxy)methoxy]-4,5,6,7-tetrahydro-1,3-benzothiazol-2-yl}benzaldehyde as a yellow gum or purified by silica gel column (CH₂Cl₂-THF=30:1) if necessary.

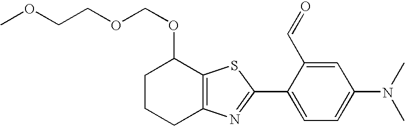

5-(dimethylamino)-2-{7-[(2-methoxyethoxy)methoxy]-4,5,6,7-tetrahydro-1,3-benzothiazol-2-yl}benzaldehyde, MS: m/z 391 (M+H)

### Step C. Preparation of 1-(5-(dimethylamino)-2-{7-[(2-methoxyethoxy)methoxy]-4,5,6,7-tetrahydro-1,3-benzothiazol-2-yl}phenyl)ethanol

A solution of 5-(dimethylamino)-2-{7-[(2-methoxyethoxy)methoxy]-4,5,6,7-tetrahydro-1,3-benzothiazol-2-yl}benzaldehyde (66 mg, 0.17 mmol) in anhydrous THF (1.0 mL) cooled in an ice bath was added to 1.4M CH₃MgBr/toluene/THF (0.15 mL, 0.21 mmol). The reaction mixture was stirred at 0° C. for 7 hours, then quenched with 1.0N citric acid solution and extracted with EtOAc. The combined organic layers were washed with H₂O, brine, dried over Na₂SO₄, filtered and then concentrated. The residue was purified by silica gel column (CH₂Cl₂-THF=30:2) to give quantitative yield of 1-(5-(dimethylamino)-2-{7-[(2-methoxyethoxy)methoxy]-4,5,6,7-tetrahydro-1,3-benzothiazol-2-yl}phenyl)ethanol as a yellow sticky oil.

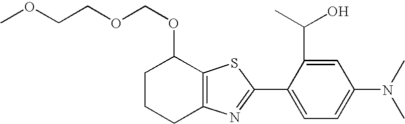

1-(5-(dimethylamino)-2-{7-[(2-methoxyethoxy)methoxy]-4,5,6,7-tetrahydro-1,3-benzothiazol-2-yl}phenyl)ethanol, MS: m/z 407 (M+H)

### Step D. Preparation of 2-[4-(dimethylamino)-2-(1-hydroxyethyl)phenyl]-4,5,6,7-tetrahydro-1,3-benzothiazol-7-ol

2-[4-(Dimethylamino)-2-(1-hydroxyethyl)phenyl]-4,5,6,7-tetrahydro-1,3-benzothiazol-7-ol was prepared in 47% as a yellow gum by following the procedure described in Step C, Example 72.

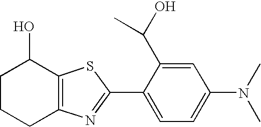

2-[4-(dimethylamino)-2-(1-hydroxyethyl)phenyl]-4,5,6,7-tetrahydro-1,3-benzothiazol-7-ol, MS: m/z 319 (M+H)

### Example 74

### Preparation of 1-[5-(Dimethylamino)-2-(7-hydroxy-4,5,6,7-tetrahydro-1,3-benzothiazol-2-yl)phenyl]ethanone

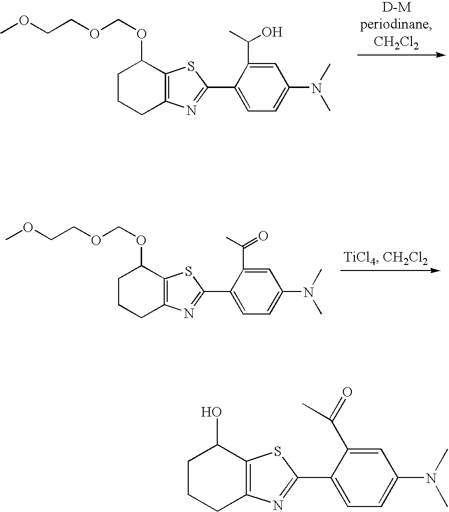

### Step A. Preparation of 1-(5-(dimethylamino)-2-{7-[(2-methoxyethoxy)methoxy]-4,5,6,7-tetrahydro-1,3-benzothiazol-2-yl}phenyl)ethanone

1-(5-(Dimethylamino)-2-{7-[(2-methoxyethoxy)methoxy]-4,5,6,7-tetrahydro-1,3-benzothiazol-2-yl}phenyl)ethanone was prepared in 29% yield as a yellow gum by following the procedure described in Step B, Example 73.

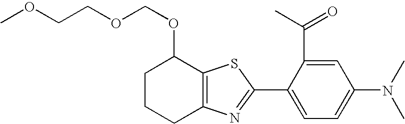

1-(5-(dimethylamino)-2-{7-[(2-methoxyethoxy)methoxy]-4,5,6,7-tetrahydro-1,3-benzothiazol-2-yl}phenyl)ethanone, MS: m/z 405 (M+H)

### Step B. Preparation of 1-[5-(dimethylamino)-2-(7-hydroxy-4,5,6,7-tetrahydro-1,3-benzothiazol-2-yl)phenyl]ethanone

The compound 1-[5-(Dimethylamino)-2-(7-hydroxy-4,5,6,7-tetrahydro-1,3-benzothiazol-2-yl)phenyl]ethanone was prepared in 27% yield as a yellow solid by following the procedure described in Step C, Example 72.

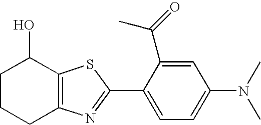

1-[5-(dimethylamino)-2-(7-hydroxy-4,5,6,7-tetrahydro-1,3-benzothiazol-2-yl)phenyl]ethanone, MS: m/z 317 (M+H)

### Examples 75-77

### Preparation of 2-[2-(azetidin-1-ylmethyl)-4-(dimethylamino)phenyl]-4,5,6,7-tetrahydro-1,3-benzothiazol-7-ol (Example 75), 2-{4-(dimethylamino)-2-[(methylamino)methyl]phenyl}-4,5,6,7-tetrahydro-1,3-benzothiazol-7-ol (Example 76) and 2-{4-(dimethylamino)-2-[(3-hydroxyazetidin-1-yl)methyl]phenyl}-4,5,6,7-tetrahydro-1,3-benzothiazol-7-ol (Example 77)

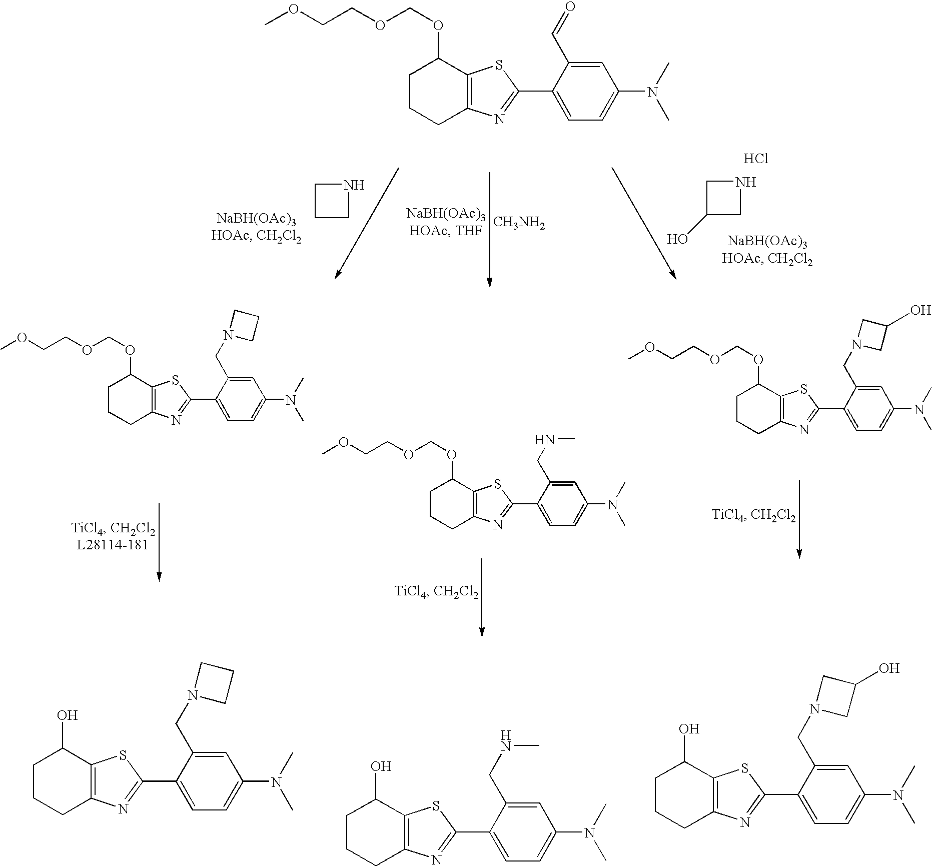

Step A. Reductive Amination

To a solution of 5-(dimethylamino)-2-{7-[(2-methoxyethoxy)methoxy]-4,5,6,7-tetrahydro-1,3-benzothiazol-2-yl}benzaldehyde (69 mg, 0.18 mmol), azetidine (60 μL, 0.88 mmol) in CH₂Cl₂ (3 mL) cooled in an ice bath was added NaBH(OAc)₃ (187 mg, 0.88 mmol) followed by the addition of glacial acetic acid, HOAc (1 drop). After removal of the ice bath, the reaction mixture was stirred at room temperature for 7 hours, then quenched with sat. NaHCO₃ solution, and then extracted with CH₂Cl₂. The combined extracts were dried over Na₂SO₄, filtered and then concentrated. The residue was separated by HPLC (mobile phase A: 0.02% TFA/H₂O; mobile phase B: 0.02% TFA/acetonitrile) to give 69 mg (90%) of 3-(azetidin-1-ylmethyl)-4-{7-[(2-methoxyethoxy)methoxy]-4,5,6,7-tetrahydro-1,3-benzothiazol-2-yl}-N,N-dimethylaniline as a yellow gum.

Following the same procedure, the aldehyde reacted with CH₃NH₂ for 5 days, providing 34% of 4-{7-[(2-methoxyethoxy)methoxy]-4,5,6,7-tetrahydro-1,3-benzothiazol-2-yl}-N,N-dimethyl-3-[(methylamino)methyl]aniline as a yellow film.

Following the same procedure, the aldehyde reacted with 3-hydroxylazetidine hydrochloride for 24 hours, providing a 55% yield of 1-(5-(dimethylamino)-2-{7-[(2-methoxyethoxy)methoxy]-4,5,6,7-tetrahydro-1,3-benzothiazol-2-yl}benzyl)azetidin-3-ol as a yellow gum.

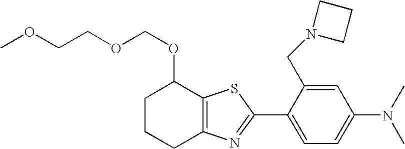

3-(azetidin-1-ylmethyl)-4-{7-[(2-methoxyethoxy)methoxy]-4,5,6,7-tetrahydro-1,3-benzothiazol-2-yl}-N,N-dimethylaniline, MS: m/z 432 (M+H)

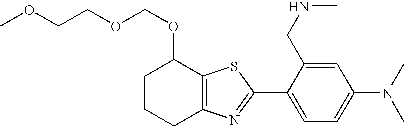

4-{7-[(2-methoxyethoxy)methoxy]-4,5,6,7-tetrahydro-1,3-benzothiazol-2-yl}-N,N-dimethyl-3-[(methylamino)methyl]aniline, MS: m/z 406 (M+H)

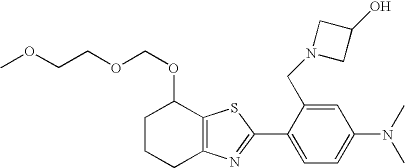

1-(5-(dimethylamino)-2-{7-[(2-methoxyethoxy)methoxy]-4,5,6,7-tetrahydro-1,3-benzothiazol-2-yl}benzyl)azetidin-3-ol, MS: m/z 448 (M+H)

Step B. Deprotection

A solution of 3-(azetidin-1-ylmethyl)-4-{7-[(2-methoxyethoxy)methoxy]-4,5,6,7-tetrahydro-1,3-benzothiazol-2-yl}-N,N-dimethylaniline (45 mg, 0.104 mmol) in CH₂Cl₂ (1.0 mL) cooled in an ice bath was added TiCl₄ solution (1.0 M/CH₂Cl₂, 1.04 mL, 1.04 mmol). The dark reaction mixture was stirred at 0° C. for 3 hours, then quenched with conc. NH₄OH and H₂O, passed through a pad of Celite, and then extracted with CH₂Cl₂. The combined extracts were dried over Na₂SO₄, filtered and then concentrated. The residue was separated by HPLC (mobile phase A: 0.02% TFA/H₂O; mobile phase B: 0.02% TFA/acetonitrile) to give 7 mg of 2-[2-(azetidin-1-ylmethyl)-4-(dimethylamino)phenyl]-4,5,6,7-tetrahydro-1,3-benzothiazol-7-ol as a yellow film.

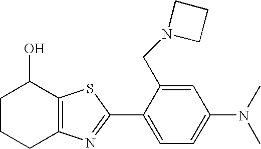

2-[2-(azetidin-1-ylmethyl)-4-(dimethylamino)phenyl]-4,5,6,7-tetrahydro-1,3-benzothiazol-7-ol, MS: m/z 344 (M+H)

Deprotection of 4-{7-[(2-methoxyethoxy)methoxy]-4,5,6,7-tetrahydro-1,3-benzothiazol-2-yl}-N,N-dimethyl-3-[(methylamino)methyl]anilinem in a similar manner provided 2-{4-(dimethylamino)-2-[(methylamino)methyl]phenyl}-4,5,6,7-tetrahydro-1,3-benzothiazol-7-ol

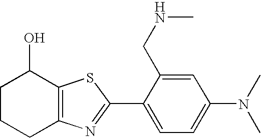

2-{4-(dimethylamino)-2-[(methylamino)methyl]phenyl}-4,5,6,7-tetrahydro-1,3-benzothiazol-7-ol, MS: m/z 318 (M+H)

Deprotection of 1-(5-(dimethylamino)-2-{7-[(2-methoxyethoxy)methoxy]-4,5,6,7-tetrahydro-1,3-benzothiazol-2-yl}benzyl)azetidin-3-ol in a similar manner provided 2-{4-(dimethylamino)-2-[(3-hydroxyazetidin-1-yl)methyl]phenyl}-4,5,6,7-tetrahydro-1,3-benzothiazol-7-ol

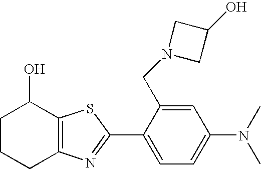

2-{4-(dimethylamino)-2-[(3-hydroxyazetidin-1-yl)methyl]phenyl}-4,5,6,7-tetrahydro-1,3-benzothiazol-7-ol, MS: m/z 360 (M+H)

### Examples 78 and 79

### Preparation of 2-[2-chloro-4-(dimethylamino)phenyl]-4,5,6,7-tetrahydro-1,3-benzothiazol-7-yl acetate (Example 78) and 2-[4-(dimethylamino)-2-fluorophenyl]-4,5,6,7-tetrahydro-1,3-benzothiazol-7-yl acetate (Example 79)

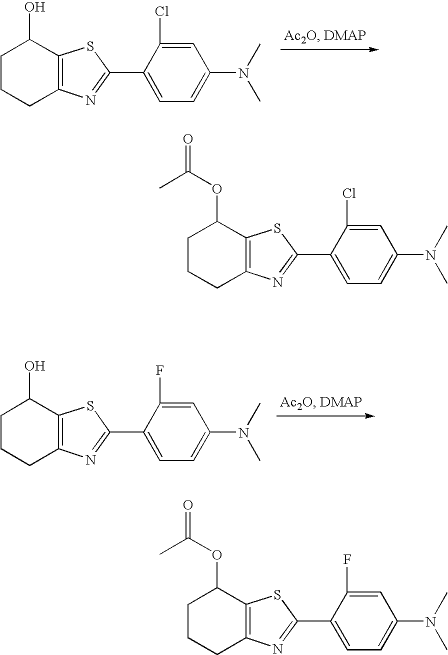

A reaction mixture of 2-[2-chloro-4-(dimethylamino)phenyl]-4,5,6,7-tetrahydro-1,3-benzothiazol-7-ol (30 mg, 0.1 mmol), acetic anhydride (14 μL, 0.15 mmol) and 4-dimethylaminopyridine (18 mg, 0.15 mmol) in CH₂Cl₂ (1 mL) was stirred at room temperature for 4 hours. The resulting mixture was separated by CombiFlash (hexane/EtOAc, gradient), providing 27 mg (77%) of 2-[2-chloro-4-(dimethylamino)phenyl]-4,5,6,7-tetrahydro-1,3-benzothiazol-7-yl acetate as a pale yellow solid.

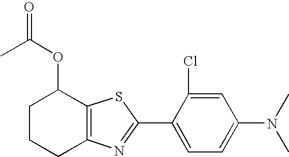

2-[2-chloro-4-(dimethylamino)phenyl]-4,5,6,7-tetrahydro-1,3-benzothiazol-7-yl acetate, MS: m/z 351 (M+H).

Following the same procedure, 2-[4-(dimethylamino)-2-fluorophenyl]-4,5,6,7-tetrahydro-1,3-benzothiazol-7-yl acetate was prepared in 64% as a pale yellow solid.

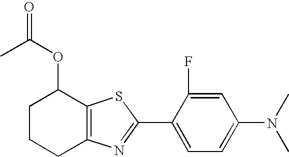

2-[4-(dimethylamino)-2-fluorophenyl]-4,5,6,7-tetrahydro-1,3-benzothiazol-7-yl acetate, MS: m/z 335 (M+H)

### Examples 80 and 81

### Preparation of 1-[4-bromo-5-(dimethylamino)-2-(7-hydroxy-4,5,6,7-tetrahydro-1,3-benzothiazol-2-yl)phenyl]ethanone and 1-[2-bromo-3-(dimethylamino)-6-(7-hydroxy-4,5,6,7-tetrahydro-1,3-benzothiazol-2-yl)phenyl]ethanone

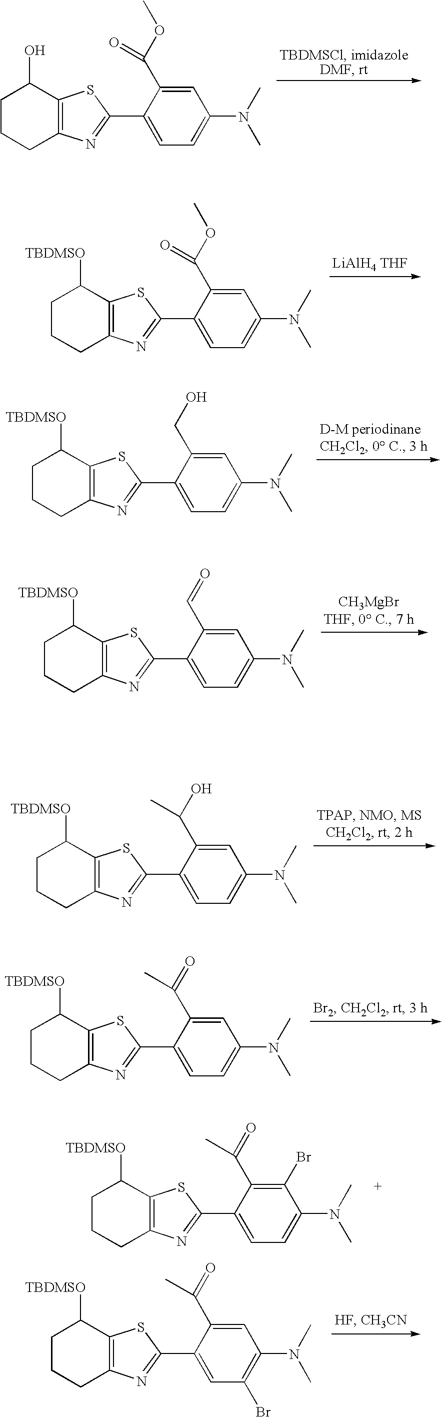

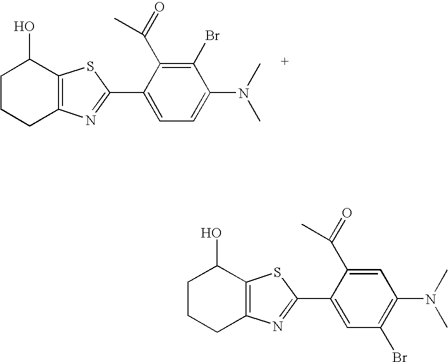

### Step A. Preparation of methyl 2-(7-{[tert-butyl(dimethyl)silyl]oxy}-4,5,6,7-tetrahydro-1,3-benzothiazol-2-yl)-5-(dimethylamino)benzoate

To a mixture of 5-(dimethylamino)-2-(7-hydroxy-4,5,6,7-tetrahydro-1,3-benzothiazol-2-yl)benzoic acid methyl ester (2.09 g, 6.3 mmol) and imidazole (0.953 g, 14.0 mmol) in anhydrous DMF (25 mL) cooled in an ice bath was added tert-butyldimethylsilyl chloride (TBDMSCI, 1.06 g, 7.0 mmol). The reaction mixture was stirred at 0° C. for 30 minutes under N₂, and then at room temperature for 17 hours. The reaction mixture was added H₂O (200 mL), extracted with EtOAc (3×100 mL). The combined organic layers were washed with H₂O, brine, dried over Na₂SO₄, filtered and then concentrated to give 2.734 g (97%) of methyl 2-(7-{[tert-butyl(dimethyl)silyl]oxy}-4,5,6,7-tetrahydro-1,3-benzothiazol-2-yl)-5-(dimethylamino)benzoate as a yellow solid. This compound is pure enough and used in the next step without further purification.

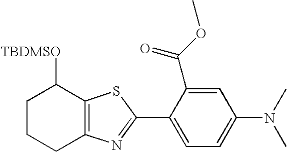

methyl 2-(7-{[tert-butyl(dimethyl)silyl]oxy}-4,5,6,7-tetrahydro-1,3-benzothiazol-2-yl)-5-(dimethylamino)benzoate, MS: m/z 447 (M+H)

### Step B. Preparation of [2-(7-{[tert-butyl(dimethyl)silyl]oxy}-4,5,6,7-tetrahydro-1,3-benzothiazol-2-yl)-5-(dimethylamino)phenyl]methanol

This compound was prepared by following the procedure described in Step A, Example 73.

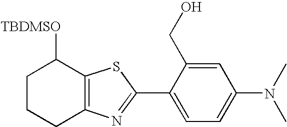

[2-(7-{[tert-butyl(dimethyl)silyl]oxy}-4,5,6,7-tetrahydro-1,3-benzothiazol-2-yl)-5-(dimethylamino)phenyl]methanol, MS: m/z 419 (M+H)

### Step C. Preparation 2-(7-{[tert-butyl(dimethyl)silyl]oxy}-4,5,6,7-tetrahydro-1,3-benzothiazol-2-yl)-5-(dimethylamino)benzaldehyde

This compound was prepared by following the procedure described in Step B, Example 73.

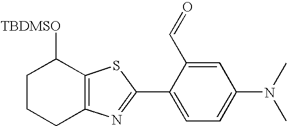

2-(7-{[tert-butyl(dimethyl)silyl]oxy}-4,5,6,7-tetrahydro-1,3-benzothiazol-2-yl)-5-(dimethylamino)benzaldehyde, MS: m/z 417 (M+H)

### Step D. Preparation 1-[2-(7-{[tert-butyl(dimethyl)silyl]oxy}-4,5,6,7-tetrahydro-1,3-benzothiazol-2-yl)-5-(dimethylamino)phenyl]ethanol

This compound was prepared by following the procedure described in Step C, Example 73.

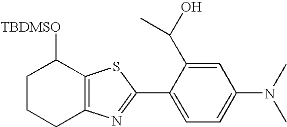

1-[2-(7-{[tert-butyl(dimethyl)silyl]oxy}-4,5,6,7-tetrahydro-1,3-benzothiazol-2-yl)-5-(dimethylamino)phenyl]ethanol, MS: m/z 433 (M+H)

### Step E. Preparation of 1-[2-(7-{[tert-butyl(dimethyl)silyl]oxy}-4,5,6,7-tetrahydro-1,3-benzothiazol-2-yl)-5-(dimethylamino)phenyl]ethanone

A mixture of the alcohol (812 mg, 1.88 mmol) and 4-methylmorpholine N-oxide (NMO, 330 mg, 2.82 mmol) and 4 Å molecular sieves (900 mg) in anhydrous CH₂Cl₂ (20 mL) was stirred at room temperature for 5 minutes. To this was added tetrapropylammonium perruthenate (TPAP, 90 mg, 0.26 mmol). The reaction mixture was stirred at room temperature for 2 hours, then filtered through a pad of Celite. The filtrate was concentrated. The residue was separated by CombiFlash (hexane/EtOAc, gradient), providing 543 mg (67%) of 1-[2-(7-{[tert-butyl(dimethyl)silyl]oxy}-4,5,6,7-tetrahydro-1,3-benzothiazol-2-yl)-5-(dimethylamino)phenyl]ethanone as a yellow gum.

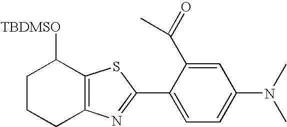

1-[2-(7-{[tert-butyl(dimethyl)silyl]oxy}-4,5,6,7-tetrahydro-1,3-benzothiazol-2-yl)-5-(dimethylamino)phenyl]ethanone, MS: m/z 431 (M+H)

### Step F. Preparation of 1-[4-bromo-2-(7-{[tert-butyl(dimethyl)silyl]oxy}-4,5,6,7-tetrahydro-1,3-benzothiazol-2-yl)-5-(dimethylamino)phenyl]ethanone and 1-[2-bromo-6-(7-{[tert-butyl(dimethyl)silyl]oxy}-4,5,6,7-tetrahydro-1,3-benzothiazol-2-yl)-3-(dimethylamino)phenyl]ethanone

To a solution of 1-[2-(7-{[tert-butyl(dimethyl)silyl]oxy}-4,5,6,7-tetrahydro-1,3-benzothiazol-2-yl)-5-(dimethylamino)phenyl]ethanone (43 mg, 0.10 mmol) in anhydrous CH₂Cl₂ (1 mL) was added bromine (6 μL, 0.11 mmol). The reaction mixture was stirred at room temperature for 2 hours, then, diluted with CH₂Cl₂ (3 mL). The reaction mixture was washed with saturated NaHCO₃ solution, H₂O, 3N NaHSO₃ solution and H₂O sequentially, and then dried over Na₂SO₄. After being filtered and concentrated, the residue was separated by CombiFlash (hexane/EtOAc, gradient), providing 33 mg (64%) of 1-[4-bromo-2-(7-{[tert-butyl(dimethyl)silyl]oxy}-4,5,6,7-tetrahydro-1,3-benzothiazol-2-yl)-5-(dimethylamino)phenyl]ethanone as a yellow sticky oil and 3 mg (6%) of 1-[2-bromo-6-(7-{[tert-butyl(dimethyl)silyl]oxy}-4,5,6,7-tetrahydro-1,3-benzothiazol-2-yl)-3-(dimethylamino)phenyl]ethanone as a yellow sticky oil.

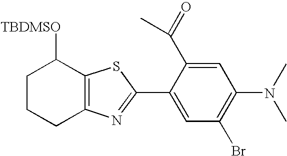

1-[4-bromo-2-(7-{[tert-butyl(dimethyl)silyl]oxy}-4,5,6,7-tetrahydro-1,3-benzothiazol-2-yl)-5-(dimethylamino)phenyl]ethanone, MS: m/z 510 (M+H)

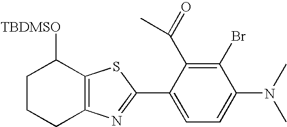

1-[2-bromo-6-(7-{[tert-butyl(dimethyl)silyl]oxy}-4,5,6,7-tetrahydro-1,3-benzothiazol-2-yl)-3-(dimethylamino)phenyl]ethanone, MS: m/z 510 (M+H)

### Step G. Preparation of 1-[4-bromo-5-(dimethylamino)-2-(7-hydroxy-4,5,6,7-tetrahydro-1,3-benzothiazol-2-yl)phenyl]ethanone and 1-[2-bromo-3-(dimethylamino)-6-(7-hydroxy-4,5,6,7-tetrahydro-1,3-benzothiazol-2-yl)phenyl]ethanone

To a solution of 1-[2-bromo-6-(7-{[tert-butyl(dimethyl)silyl]oxy}-4,5,6,7-tetrahydro-1,3-benzothiazol-2-yl)-3-(dimethylamino)phenyl]ethanone (86 mg, 0.20 mmol) in anhydrous CH₂Cl₂ (2 mL) was treated with bromine (13 μL, 0.26 mmol), providing a mixture of 1-[4-bromo-5-(dimethylamino)-2-(7-hydroxy-4,5,6,7-tetrahydro-1,3-benzothiazol-2-yl)phenyl]ethanone and 1-[2-bromo-3-(dimethylamino)-6-(7-hydroxy-4,5,6,7-tetrahydro-1,3-benzothiazol-2-yl)phenyl]ethanone by following the procedure described above. The mixture was used in the next reaction without further purification.

The above mixture was dissolved in CH₃CN (2 mL) and added a solution of 48 wt % HF in H₂O (17 μL, 0.4 mmol). The reaction mixture was stirred at room temperature for 2 hours. Additional HF solution (50 μL) was added. The reaction mixture was stirred at room temperature for additional 2 hours. Additional HF solution (150 μL) was added. The reaction mixture was stirred at room temperature for additional 2 hours, then neutralized to pH 8 with saturated NaHCO₃ solution, and then extracted with EtOAc. The combined organic layers were washed with H₂O, brine and then dried over Na₂SO₄. After the residue was filtered and concentrated, the residue was separated by flash chromatography (CH₂Cl₂:THF=30:1), providing 65 mg (82%) of 1-[4-bromo-5-(dimethylamino)-2-(7-hydroxy-4,5,6,7-tetrahydro-1,3-benzothiazol-2-yl)phenyl]ethanone as a yellow sticky oil and 13 mg (16%) of 1-[2-bromo-3-(dimethylamino)-6-(7-hydroxy-4,5,6,7-tetrahydro-1,3-benzothiazol-2-yl)phenyl]ethanone as a yellow sticky oil.

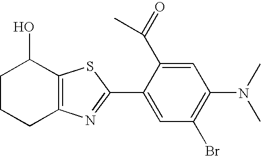

1-[4-bromo-5-(dimethylamino)-2-(7-hydroxy-4,5,6,7-tetrahydro-1,3-benzothiazol-2-yl)phenyl]ethanone, MS: m/z 396 (M+H)

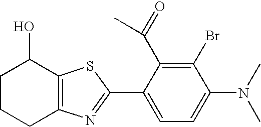

1-[2-bromo-3-(dimethylamino)-6-(7-hydroxy-4,5,6,7-tetrahydro-1,3-benzothiazol-2-yl)phenyl]ethanone, MS: m/z 396 (M+H)

### Example 82

### Preparation of 2-[2-(1-hydroxyethyl)-4-(methylamino)phenyl]-4,5,6,7-tetrahydro-1,3-benzothiazol-7-ol

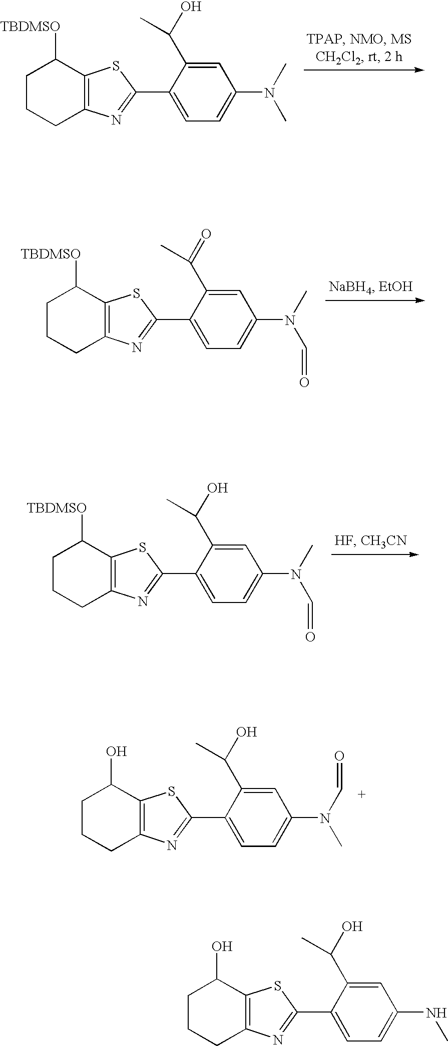

### Step A. Preparation of N-[3-acetyl-4-(7-{[tert-butyl(dimethyl)silyl]oxy}-4,5,6,7-tetrahydro-1,3-benzothiazol-2-yl)phenyl]-N-methylformamide

N-[3-Acetyl-4-(7-{[tert-butyl(dimethyl)silyl]oxy}-4,5,6,7-tetrahydro-1,3-benzothiazol-2-yl)phenyl]-N-methylformamide was obtained from Step E, Example 80-81 as a by-product (87 mg, 10% yield as a yellow gum).

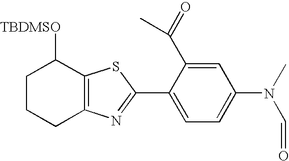

N-[3-acetyl-4-(7-{[tert-butyl(dimethyl)silyl]oxy}-4,5,6,7-tetrahydro-1,3-benzothiazol-2-yl)phenyl]-N-methylformamide, MS: m/z 445 (M+H)

### Step B. Preparation of N-[4-(7-{[tert-butyl(dimethyl)silyl]oxy}-4,5,6,7-tetrahydro-1,3-benzothiazol-2-yl)-3-(1-hydroxyethyl)phenyl]-N-methylformamide

A reaction mixture of N-[3-Acetyl-4-(7-{[tert-butyl(dimethyl)silyl]oxy}-4,5,6,7-tetrahydro-1,3-benzothiazol-2-yl)phenyl]-N-methylformamide (66 mg, 0.15 mmol) and NaBH₄ (5.6 mg, 0.15 mmol) in CH₃CH₂OH (1 mL) was stirred at room temperature for 2 hours, then diluted with 5 mL of CH₂Cl₂. The organic layer was washed with saturated NaHCO₃ solution, H₂O, brine and then dried over Na₂SO₄. The filtration and concentration gave N-[4-(7-{[tert-butyl(dimethyl)silyl]oxy}-4,5,6,7-tetrahydro-1,3-benzothiazol-2-yl)-3-(1-hydroxyethyl)phenyl]-N-methylformamide as a yellow sticky oil.

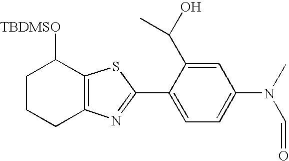

N-[4-(7-{[tert-butyl(dimethyl)silyl]oxy}-4,5,6,7-tetrahydro-1,3-benzothiazol-2-yl)-3-(1-hydroxyethyl)phenyl]-N-methylformamide, MS: m/z 447 (M+H)

### Step C. Preparation of 2-[2-(1-hydroxyethyl)-4-(methylamino)phenyl]-4,5,6,7-tetrahydro-1,3-benzothiazol-7-ol

N-[4-(7-{[tert-butyl(dimethyl)silyl]oxy}-4,5,6,7-tetrahydro-1,3-benzothiazol-2-yl)-3-(1-hydroxyethyl)phenyl]-N-methylformamide prepared above was dissolved in CH₃CN (2 mL) and added a solution of 48 wt % HF in H₂O (20 μL). The reaction mixture was stirred at room temperature for 2 hours. Additional HF solution (150 μL) was added. The reaction mixture was stirred at room temperature for additional 2 hours, then neutralized to pH 8 with saturated NaHCO₃ solution, and then extracted with EtOAc. The combined organic layers were washed with H₂O, brine and then dried over Na₂SO₄. After the residue filtered and concentrated, it was separated by CombiFlash (hexane/EtOAc gradient), providing 16 mg (35% in two steps) of 2-[2-(1-hydroxyethyl)-4-(methylamino)phenyl]-4,5,6,7-tetrahydro-1,3-benzothiazol-7-ol as a yellow solid and 20 mg (40%) of N-[3-(1-hydroxyethyl)-4-(7-hydroxy-4,5,6,7-tetrahydro-1,3-benzothiazol-2-yl)phenyl]-N-methylformamide as a yellow sticky oil.

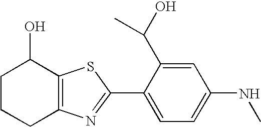

2-[2-(1-hydroxyethyl)-4-(methylamino)phenyl]-4,5,6,7-tetrahydro-1,3-benzothiazol-7-ol, MS: m/z 305 (M+H)

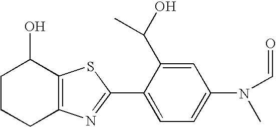

N-[3-(1-hydroxyethyl)-4-(7-hydroxy-4,5,6,7-tetrahydro-1,3-benzothiazol-2-yl)phenyl]-N-methylformamide, MS: m/z 333 (M+H)

### Examples 83-86

### Preparation of 2-(2-bromo-4-pyrrolidin-1-ylphenyl)-4,5,6,7-tetrahydro-1,3-benzothiazol-7-ol (Example 83), 2-(2-bromo-4-pyrrolidin-1-ylphenyl)-7-isopropoxy-4,5,6,7-tetrahydro-1,3-benzothiazole (Example 84), 2-(7-hydroxy-4,5,6,7-tetrahydro-1,3-benzothiazol-2-yl)-5-pyrrolidin-1-ylbenzonitrile (Example 85) and 2-[2-(aminomethyl)-4-pyrrolidin-1-ylphenyl]-4,5,6,7-tetrahydro-1,3-benzothiazol-7-ol (Example 86)

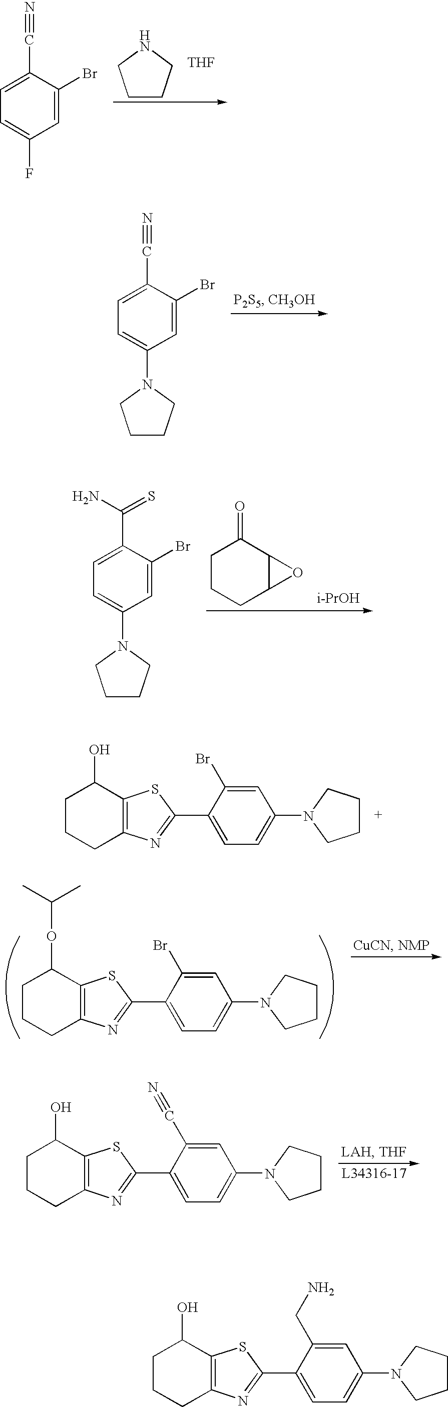

To a solution of 2-bromo-4-fluorobenzonitrile (200 mg, 1.0 mmol) in anhydrous THF (5 mL) was added pyrrolidine. The reaction mixture was stirred at room temperature for 4 hours. To this was added saturated NaHCO₃ solution and H₂O, then the mixture was extracted with EtOAc. The combined organic layers were washed with H₂O, brine, dried over Na₂SO₄, filtered and then concentrated to provide 222 mg (88%) of 2-bromo-4-pyrrolidin-1-ylbenzonitrile as a white solid.

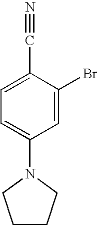

2-bromo-4-pyrrolidin-1-ylbenzonitrile, MS: m/z 252 (M+H)

### Step B. Preparation of 2-bromo-4-pyrrolidin-1-ylbenzenecarbothioamide

Following General Procedure II in Examples 11-28, 2-bromo-4-pyrrolidin-1-ylbenzonitrile, 128 mg, 0.5 mmol) was treated with P₄S₁₀ (445 mg, 1.0 mmol) in CH₃OH (2 mL) at room temperature for 20 hours, then separated by CombiFlash (hexane/EtOAc, gradient) to provide 120 mg (84%) of 2-Bromo-4-pyrrolidin-1-ylbenzenecarbothioamide as a yellow solid.

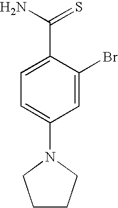

2-bromo-4-pyrrolidin-1-ylbenzenecarbothioamide, MS: m/z 286 (M+H)

### Step C. Preparation of 2-(2-bromo-4-pyrrolidin-1-ylphenyl)-4,5,6,7-tetrahydro-1,3-benzothiazol-7-ol and 2-(2-bromo-4-pyrrolidin-1-ylphenyl)-7-isopropoxy-4,5,6,7-tetrahydro-1,3-benzothiazole

Following General Procedure III in Examples 11-28, a mixture of 2-bromo-4-pyrrolidin-1-ylbenzenecarbothioamide (235 mg, 0.82 mmol) and 7-oxabicyclo[4.1.0]heptan-2-one (0.25 mL, 2.5 mmol) in 2-propanol (3 mL) was subjected to microwave-assisted reaction at 100° C. for 30 minutes. The reaction mixture was separated by CombiFlash (hexane/EtOAc, gradient) to provide 223 mg (71%) of 2-(2-bromo-4-pyrrolidin-1-ylphenyl)-4,5,6,7-tetrahydro-1,3-benzothiazol-7-ol as a yellow solid and 38 mg (11%) of 2-(2-bromo-4-pyrrolidin-1-ylphenyl)-7-isopropoxy-4,5,6,7-tetrahydro-1,3-benzothiazole as a yellow sticky oil.

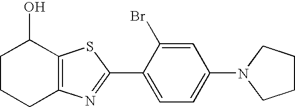

2-(2-bromo-4-pyrrolidin-1-ylphenyl)-4,5,6,7-tetrahydro-1,3-benzothiazol-7-ol, MS: m/z 380 (M+H)

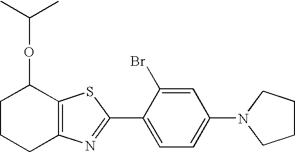

2-(2-bromo-4-pyrrolidin-1-ylphenyl)-7-isopropoxy-4,5,6,7-tetrahydro-1,3-benzothiazole, MS: m/z 422 (M+H)

### Step D. Preparation of 2-(7-hydroxy-4,5,6,7-tetrahydro-1,3-benzothiazol-2-yl)-5-pyrrolidin-1-ylbenzonitrile

A mixture of 2-(2-bromo-4-pyrrolidin-1-ylphenyl)-4,5,6,7-tetrahydro-1,3-benzothiazol-7-ol (60 mg, 0.16 mmol) and CuCN (21 mg, 0.24 mmol) in anhydrous N-methylpyrolidone, (0.8 mL) was subjected to microwave-assisted reaction at 80° C. for 20 minutes. To this mixture was added a 1:1 mixture of THF/EtOAc. The organic layer was washed with diluted NH₄OH solution, H₂O, brine, dried over Na₂SO₄, filtered and then concentrated. The residue was separated by flash chromatography (CH₂Cl₂:THF=30:1) to provide 2-(7-hydroxy-4,5,6,7-tetrahydro-1,3-benzothiazol-2-yl)-5-pyrrolidin-1-ylbenzonitrile as a yellow solid.

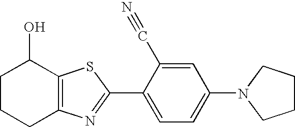

2-(7-hydroxy-4,5,6,7-tetrahydro-1,3-benzothiazol-2-yl)-5-pyrrolidin-1-ylbenzonitrile, MS: m/z 326 (M+H)

### Step E. Preparation of 2-[2-(aminomethyl)-4-pyrrolidin-1-ylphenyl]-4,5,6,7-tetrahydro-1,3-benzothiazol-7-ol

To a solution of 2-(7-hydroxy-4,5,6,7-tetrahydro-1,3-benzothiazol-2-yl)-5-pyrrolidin-1-ylbenzonitrile (43 mg, 0.13 mmol) in anhydrous THF (1.0 mL) was added 1.0M LiAlH₄/THF (0.15 mL, 0.15 mmol) and stirred at 0° C. for 4 hours. Additional LiAlH₄/THF (0.15 mL, 0.15 mmol) was added. The reaction mixture was stirred overnight from 0° C. to room temperature, then quenched with H₂O. The mixture was extracted with EtOAc. The combined organic layers were washed with H₂O, brine, dried over Na₂SO₄, filtered and then concentrated. The residue was separated by HPLC (0.02% TFA/H₂O, 0.02% TFA/CH₃CN) to provide 4 mg of 2-[2-(aminomethyl)-4-pyrrolidin-1-ylphenyl]-4,5,6,7-tetrahydro-1,3-benzothiazol-7-ol as a yellow solid.

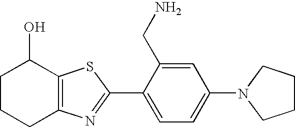

2-[2-(aminomethyl)-4-pyrrolidin-1-ylphenyl]-4,5,6,7-tetrahydro-1,3-benzothiazol-7-ol, MS: m/z 330 (M+H)

### Examples 87 and 88

### Preparation of 2-{4-[(dimethylamino)methyl]phenyl}-4,5,6,7-tetrahydro-1,3-benzothiazol-7-ol and 2-[4-(hydroxymethyl)phenyl]-4,5,6,7-tetrahydro-1,3-benzothiazol-7-ol

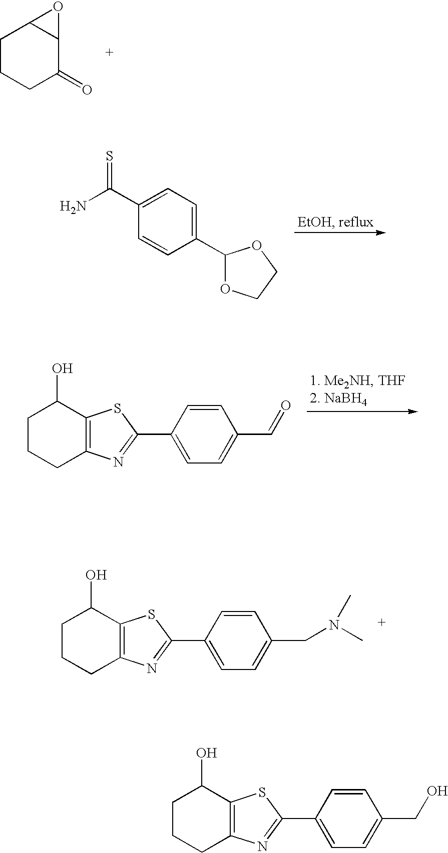

### Step A. 4-(7-hydroxy-4,5,6,7-tetrahydro-1,3-benzothiazol-2-yl)benzaldehyde

A solution of 7-oxabicyclo[4.1.0]heptan-2-one (112 mg, 1.0 mmol) and 4-(1,3-dioxolan-2-yl)benzene-1-carbothioamide (209 mg, 1.0 mmol) in anhydrous EtOH (5 mL) was refluxed for 12 hours, stirred at room temperature overnight, and then concentrated. The residue was separated by silica gel column (hexane/EtOAc=2:1) to give 79 mg (31%) of 4-(7-hydroxy-4,5,6,7-tetrahydro-1,3-benzothiazol-2-yl)benzaldehyde as a white solid.

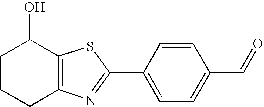

4-(7-hydroxy-4,5,6,7-tetrahydro-1,3-benzothiazol-2-yl)benzaldehyde, MS: m/z 260 (M+H).

### Step B. 2-{4-[(dimethylamino)methyl]phenyl}-4,5,6,7-tetrahydro-1,3-benzothiazol-7-ol and 2-[4-(hydroxymethyl)phenyl]-4,5,6,7-tetrahydro-1,3-benzothiazol-7-ol

A solution of 4-(7-hydroxy-4,5,6,7-tetrahydro-1,3-benzothiazol-2-yl)benzaldehyde (49 mg, 0.189 mmol) in 2 mL of anhydrous THF was added to dimethylamine (2.0 M/THF, 0.28 mL, 0.568 mmol, 3.0 equiv.). The reaction mixture was stirred at room temperature for 1 hour. NaBH₄ (14 mg, 0.378 mmol) was added. The reaction mixture was then stirred at room temperature overnight, quenched with H₂O, then extracted with EtOAc. The combined organic layers were dried over Na₂SO₄, filtered and then concentrated. The crude mixture was purified by preparative HPLC (mobile phase A: 0.02% TFA/CH₃CN; mobile phase B: 0.02% TFA/H₂O) to give 30 mg (55%) of 2-{4-[(dimethylamino)methyl]phenyl}-4,5,6,7-tetrahydro-1,3-benzothiazol-7-ol as a white solid. It also gave 5 mg (10%) of 2-[4-(hydroxymethyl)phenyl]-4,5,6,7-tetrahydro-1,3-benzothiazol-7-ol.

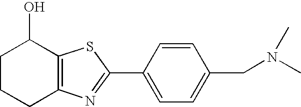

2-{4-[(dimethylamino)methyl]phenyl}-4,5,6,7-tetrahydro-1,3-benzothiazol-7-ol, MS: m/z 289 (M+H)

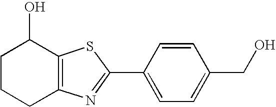

2-[4-(hydroxymethyl)phenyl]-4,5,6,7-tetrahydro-1,3-benzothiazol-7-ol, MS: m/z 262 (M+H)

### Examples 89 and 90

### Preparation of 2-(4-amino-3-methylphenyl)-4,5,6,7-tetrahydro-1,3-benzothiazol-7-ol (Example 89) and 2-[4-(dimethylamino)-3-methylphenyl]-4,5,6,7-tetrahydro-1,3-benzothiazol-7-ol (Example 90)

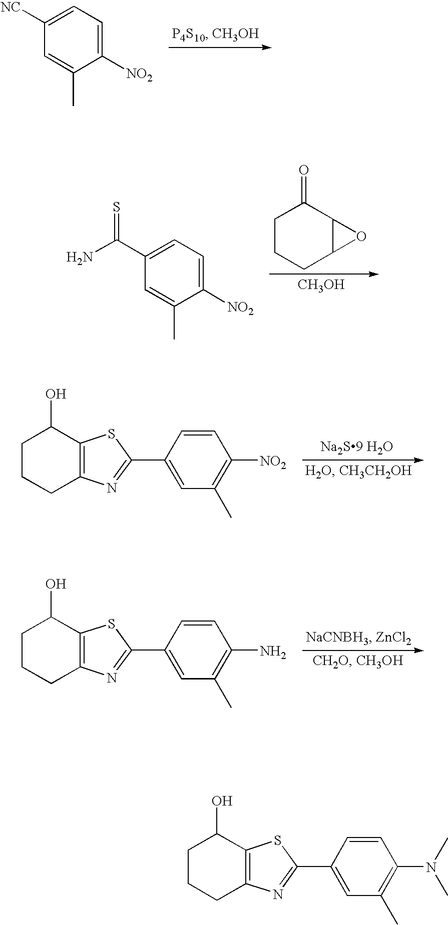

Step A: Following General Procedure II, 3-methyl-4-nitrobenzenecarbothioamide was prepared in 65% yield starting with commercially available 3-methyl-4-nitrobenzonitrile

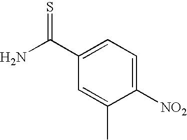

3-methyl-4-nitrobenzenecarbothioamide, MS: m/z 197 (M+H)

Step B: Following General Procedure III, 2-(3-methyl-4-nitrophenyl)-4,5,6,7-tetrahydro-1,3-benzothiazol-7-ol was prepared in 35% yield.

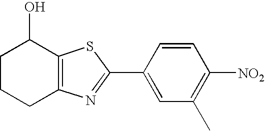

2-(3-methyl-4-nitrophenyl)-4,5,6,7-tetrahydro-1,3-benzothiazol-7-ol, MS: m/z 291 (M+H)

Step C. General Reduction Procedure Using Na₂S, Nitro compound (1.0 mmol) in a mixture of H₂O/C₂H₅OH (1:1, 12 mL) was added to Na₂S.9H₂O (5.0 mmol). The mixture was stirred at room temperature for one or two day, then concentrated to dryness. The residue was added to H₂O and neutralized with 1.0 N citric acid to pH 6-7, filtered. The solid residue was washed with H₂O and then dried by suction in air overnight, providing the desired amino compound, 2-(4-Amino-3-methylphenyl)-4,5,6,7-tetrahydro-1,3-benzothiazol-7-ol, in 94% yield.

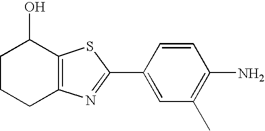

2-(4-amino-3-methylphenyl)-4,5,6,7-tetrahydro-1,3-benzothiazol-7-ol, MS: m/z 261 (M+H)

### Step D. Preparation of 2-[4-(dimethylamino)-3-methylphenyl]-4,5,6,7-tetrahydro-1,3-benzothiazol-7-ol

To a stirred solution of 2-(4-amino-3-methylphenyl)-4,5,6,7-tetrahydro-1,3-benzothiazol-7-ol (1.0 mmol) in CH₃OH (3 mL) containing 37% aqueous CH₂O (3.0 mmol) at room temperature was added a solution of NaBH₃CN (1.0 mmol) and ZnCl₂ (0.5 mmol) in CH₃OH (3 mL). The reaction mixture was stirred at room temperature overnight, then concentrated. The residue was taken up in 0.1 N NaOH solution, then extracted with EtOAc several times. The combined extracts were washed with H₂O, brine, dried over Na₂SO₄, filtered and then concentrated. The residue was separated by silica gel column (hexane/EtOAc=3:1) to give 2-[4-(dimethylamino)-3-methylphenyl]-4,5,6,7-tetrahydro-1,3-benzothiazol-7-ol as a yellow sticky oil.

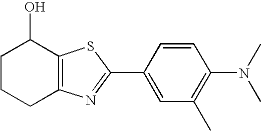

2-[4-(dimethylamino)-3-methylphenyl]-4,5,6,7-tetrahydro-1,3-benzothiazol-7-ol, MS: m/z 289 (M+H)

### Examples 91-94, 45, 91-100

### Preparation of [2-chloro-4-(7-methoxy-4,5,6,7-tetrahydro-1,3-benzothiazol-2-yl)phenyl]-dimethylamine (91), 2-[3-chloro-4-(dimethylamino)phenyl]-4,5,6,7-tetrahydro-1,3-benzothiazol-7-ol (92), 3-chloro-4-(7-methoxy-4,5,6,7-tetrahydro-1,3-benzothiazol-2-yl)phenyl]dimethylamine (93), 2-[2-chloro-4-(dimethylamino)phenyl]-4,5,6,7-tetrahydro-1,3-benzothiazol-7-ol (94), 2-[4-(dimethylamino)-2-methylphenyl]-4,5,6,7-tetrahydro-1,3-benzothiazol-7-ol (45), 2-[4-(dimethylamino)-2-methoxyphenyl]-4,5,6,7-tetrahydro-1,3-benzothiazol-7-ol (95), 2-[4-(dimethylamino)-3-iodophenyl]-4,5,6,7-tetrahydro-1,3-benzothiazol-7-ol (96), 2-[4-(dimethylamino)-2-(trifluoromethyl)phenyl]-4,5,6,7-tetrahydro-1,3-benzothiazol-7-ol (97), 2-[4-(dimethylamino)-3-(trifluoromethyl)phenyl]-4,5,6,7-tetrahydro-1,3-benzothiazol-7-ol (98), (7R)-2-[4-(dimethylamino)-2-methylphenyl]-4,5,6,7-tetrahydro-1,3-benzothiazol-7-ol (99) and (7S)-2-[4-(dimethylamino)-2-methylphenyl]-4,5,6,7-tetrahydro-1,3-benzothiazol-7-ol (100)

General synthetic procedure is outlined as follows:

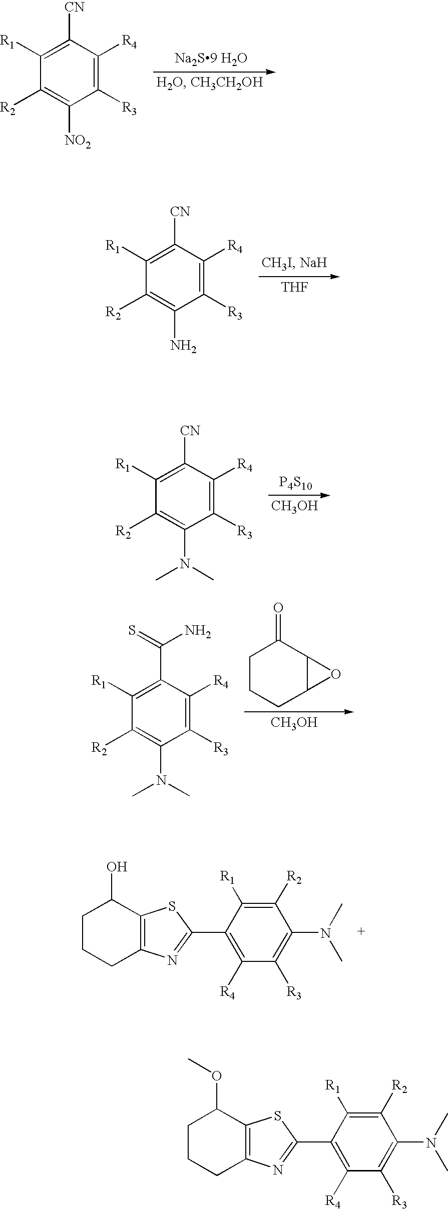

Step A. Following the General Reduction Procedure Using Na₂S, the following compounds were prepared:

Starting from 2-methoxy-4-nitrobenzonitrile, 4-amino-2-methoxybenzonitrile was prepared as a yellow solid.

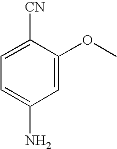

4-amino-2-methoxybenzonitrile, MS: m/z 149 (M+H)

Starting from 4-nitro-o-tolunitrile, 4-amino-2-methylbenzonitrile was prepared as a yellow solid.

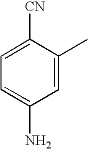

4-amino-2-methylbenzonitrile, MS: m/z 133 (M+H)

Step B: Following General Procedure I, the following compounds were prepared.

Starting from 4-amino-3-chlorobenzonitrile, 3-chloro-4-(dimethylamino)benzonitrile was prepared as a colorless oil.

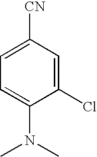

4-amino-2-methylbenzonitrile, MS: m/z 181 (M+H)

Starting from 4-amino-2-chlorobenzonitrile, 2-chloro-4-(dimethylamino)benzonitrile was prepared as a yellow solid.

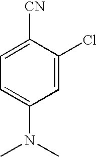

2-chloro-4-(dimethylamino)benzonitrile, MS: m/z 181 (M+H)

Starting from 4-cyano-3-trifluoromethylaniline, 4-(dimethylamino)-2-(trifluoromethyl)benzonitrile was prepared as a white solid.

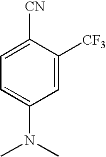

4-(dimethylamino)-2-(trifluoromethyl)benzonitrile, MS: m/z 215 (M+H)

Starting from 4-cyano-3-iodoaniline, 4-(dimethylamino)-3-iodobenzonitrile was prepared as a yellow oil.

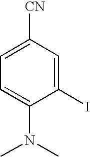

4-(dimethylamino)-3-iodobenzonitrile, MS: m/z 273 (M+H)

Starting from 4-amino-3-trifluoromethylbenzonitrile, 4-(dimethylamino)-3-(trifluoromethyl)benzonitrile was prepared as a white solid.

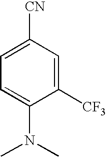

4-(dimethylamino)-3-(trifluoromethyl)benzonitrile, MS: m/z 215 (M+H)

Starting from 4-amino-2-methoxybenzonitrile, 4-(dimethylamino)-2-methoxybenzonitrile was prepared as a yellow solid.

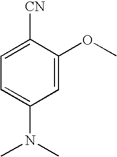

4-(dimethylamino)-2-methoxybenzonitrile, MS: m/z 177 (M+H)

Starting from 4-amino-2-methyl benzonitrile, 4-(dimethylamino)-2-methylbenzonitrile was prepared as a pale yellow solid.

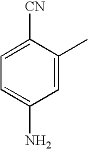

4-(dimethylamino)-2-methylbenzonitrile, MS: m/z 161 (M+H)

Step C: Following General Procedure II, the following compounds were prepared.

Starting from 3-chloro-4-(dimethylamino)benzonitrile, 3-chloro-4-(dimethylamino)benzenecarbothioamide was prepared as a pale green sticky oil.

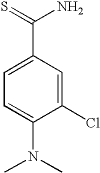

3-chloro-4-(dimethylamino)benzenecarbothioamide, MS: m/z 215 (M+H)

Starting from 2-chloro-4-(dimethylamino)benzonitrile, 2-chloro-4-(dimethylamino)benzenecarbothioamide was prepared as a yellow solid.

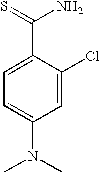

2-chloro-4-(dimethylamino)benzenecarbothioamide, MS: m/z 215 (M+H)

Starting from 4-(dimethylamino)-2-(trifluoromethyl)benzo-nitrile, 4-(dimethylamino)-2-(trifluoromethyl)benzenecarbothioamide was prepared as a yellow solid.

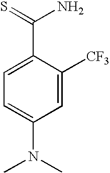

4-(dimethylamino)-2-(trifluoromethyl)benzenecarbothioamide, MS: m/z 249 (M+H)

Starting from 4-(dimethylamino)-3-iodobenzonitrile, 4-(dimethylamino)-3-iodobenzenecarbothioamide was prepared as a yellow solid.

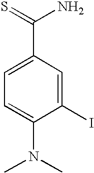

4-(dimethylamino)-3-iodobenzenecarbothioamide, MS: m/z 307 (M+H)

Starting from 4-(dimethylamino)-3-(trifluoromethyl)benzonitrile, 4-(dimethylamino)-3-(trifluoromethyl)benzenecarbothioamide was prepared as a white solid.

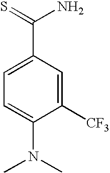

4-(dimethylamino)-3-(trifluoromethyl)benzenecarbothioamide, MS: m/z 249 (M+H)

Starting from 4-(dimethylamino)-2-methoxybenzonitrile, 4-(dimethylamino)-2-methoxybenzenecarbothioamide was prepared as a yellow solid.

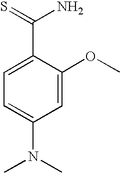

4-(dimethylamino)-2-methoxybenzenecarbothioamide, MS: m/z 211 (M+H)

Starting from 4-(dimethylamino)-2-methyl benzonitrile, 4-(dimethylamino)-2-methylbenzenecarbothioamide was prepared as a pale yellow solid.

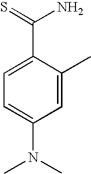

4-(dimethylamino)-2-methylbenzenecarbothioamide, MS: m/z 195 (M+H)

Step D: Following General Procedure III, the following compounds were prepared from the corresponding thioamides prepared in Step C.

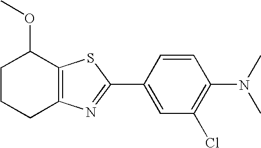

[2-chloro-4-(7-methoxy-4,5,6,7-tetrahydro-1,3-benzothiazol-2-yl)phenyl]-dimethylamine, yellow sticky oil, MS: m/z 323 (M+H)

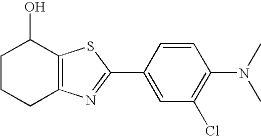

2-[3-chloro-4-(dimethylamino)phenyl]-4,5,6,7-tetrahydro-1,3-benzothiazol-7-ol, yellow foam, MS: m/z 309 (M+H)

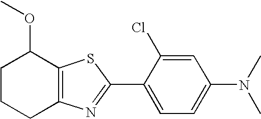

3-chloro-4-(7-methoxy-4,5,6,7-tetrahydro-1,3-benzothiazol-2-yl)phenyl-dimethylamine, yellow sticky oil, MS: m/z 323 (M+H)

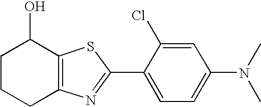

2-[2-chloro-4-(dimethylamino)phenyl]-4,5,6,7-tetrahydro-1,3-benzothiazol-7-ol, yellow foam, MS: m/z 309 (M+H)

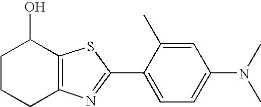

2-[4-(dimethylamino)-2-methylphenyl]-4,5,6,7-tetrahydro-1,3-benzothiazol-7-ol, yellow solid, MS: m/z 289 (M+H)

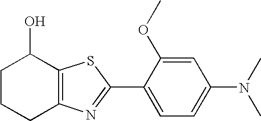

2-[4-(dimethylamino)-2-methoxyphenyl]-4,5,6,7-tetrahydro-1,3-benzothiazol-7-ol, yellow solid, MS: m/z 305 (M+H)

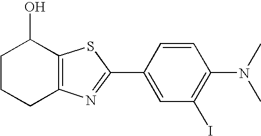

2-[4-(dimethylamino)-3-iodophenyl]-4,5,6,7-tetrahydro-1,3-benzothiazol-7-ol, yellow solid, MS: m/z 401 (M+H)

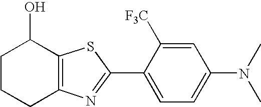

2-[4-(dimethylamino)-2-(trifluoromethyl)phenyl]-4,5,6,7-tetrahydro-1,3-benzothiazol-7-ol, yellow solid, MS: m/z 343 (M+H)

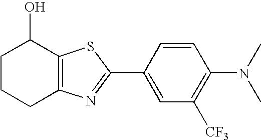

2-[4-(dimethylamino)-3-(trifluoromethyl)phenyl]-4,5,6,7-tetrahydro-1,3-benzothiazol-7-ol, yellow solid, MS: m/z 343 (M+H)

The compound, 2-[4-(dimethylamino)-2-methyl phenyl]-4,5,6,7-tetrahydro-1,3-benzothiazol-7-ol, was separated by chiral HPLC, providing (7R)-2-[4-(dimethylamino)-2-methylphenyl]-4,5,6,7-tetrahydro-1,3-benzothiazol-7-ol and (7S)-2-[4-(dimethylamino)-2-methylphenyl]-4,5,6,7-tetrahydro-1,3-benzothiazol-7-ol.

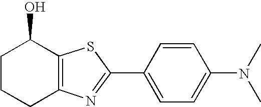

(7R)-2-[4-(dimethylamino)-2-methylphenyl]-4,5,6,7-tetrahydro-1,3-benzothiazol-7-ol, yellow solid, MS: m/z 289 (M+H)

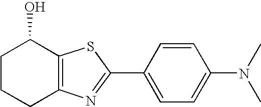

(7S)-2-[4-(dimethylamino)-2-methylphenyl]-4,5,6,7-tetrahydro-1,3-benzothiazol-7-ol, yellow solid, MS: m/z 289 (M+H)

### Example 101

### Preparation of 2-[4-(dimethylamino)-2-hydroxyphenyl]-4,5,6,7-tetrahydro-1,3-benzothiazol-7-ol

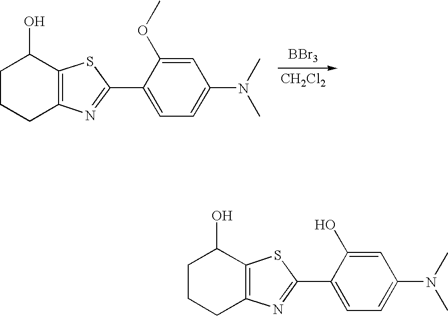

A solution of 2-[4-(dimethylamino)-2-methoxyphenyl]-4,5,6,7-tetrahydro-1,3-benzothiazol-7-ol (0.1 mmol) in anhydrous CH₂Cl₂ (1 mL) was treated with 1.0M boron tribromide in dichloromethane (0.2 mmol) at −78° C. The reaction mixture was stirred at −78° C. for 2 hours, and then at room temperature overnight. The reaction mixture was poured into H₂O, neutralized to pH 7 with saturated NaHCO₃ solution, then extracted with EtOAc. The mixture was purified by flash chromatography, providing 2-[4-(dimethylamino)-2-hydroxyphenyl]-4,5,6,7-tetrahydro-1,3-benzothiazol-7-ol as a yellow solid.

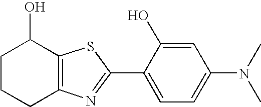

2-[4-(dimethylamino)-2-hydroxyphenyl]-4,5,6,7-tetrahydro-1,3-benzothiazol-7-ol, MS: m/z 291 (M+H)

### Example 102

### Preparation of 2-[4-(dimethylamino)-3,5-dimethylphenyl]-4,5,6,7-tetrahydro-1,3-benzothiazol-7-ol

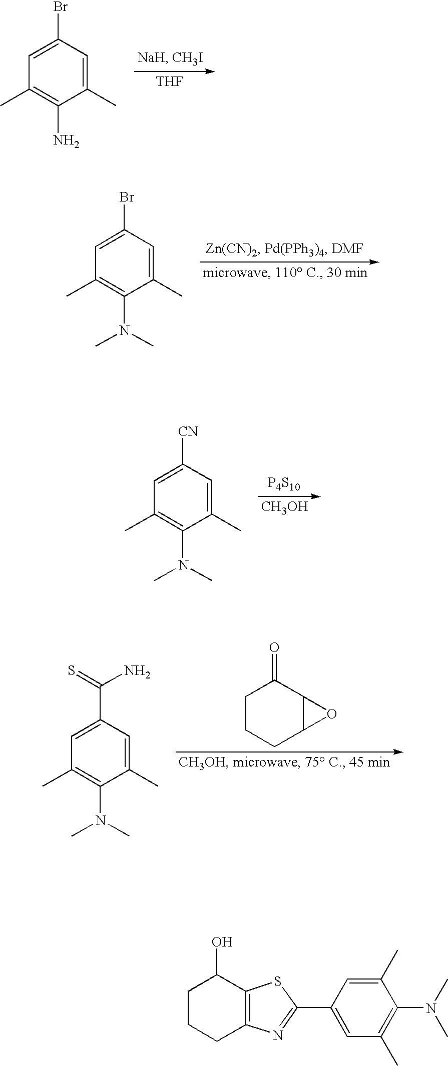

Step A: Following General Procedure I, (4-bromo-2,6-dimethylphenyl)dimethylamine was prepared as a colorless oil in 68% yield starting from 4-bromo-2,6-dimethylaniline.

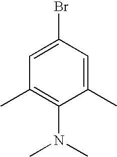

(4-bromo-2,6-dimethylphenyl)dimethylamine, MS: m/z 229 (M+H)

Step B: Following Step C in Example 55-58, 4-(dimethylamino)-3,5-dimethylbenzonitrile was prepared as a white solid in 94% yield.

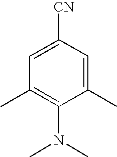

4-(dimethylamino)-3,5-dimethylbenzonitrile, MS: m/z 175 (M+H)

Step C: Following General Procedure II, 4-(dimethylamino)-3,5-dimethylbenzene-carbothioamide was prepared as a yellow solid in 60% yield.

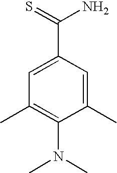

4-(dimethylamino)-3,5-dimethylbenzenecarbothioamide, MS: m/z 209 (M+H)

Step D: Following General Procedure III, 2-[4-(dimethylamino)-3,5-dimethylphenyl]-4,5,6,7-tetrahydro-1,3-benzothiazol-7-ol was prepared as a yellow solid in 78%.

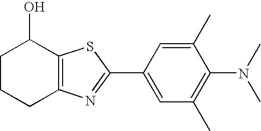

2-[4-(dimethylamino)-3,5-dimethylphenyl]-4,5,6,7-tetrahydro-1,3-benzothiazol-7-ol, MS: m/z 303 (M+H)

### Examples 103 and 104

### Preparation of methyl 2-(dimethylamino)-5-(7-hydroxy-4,5,6,7-tetrahydro-1,3-benzothiazol-2-yl)benzoate and methyl 2-(dimethylamino)-5-(7-methoxy-4,5,6,7-tetrahydro-1,3-benzothiazol-2-yl)benzoate

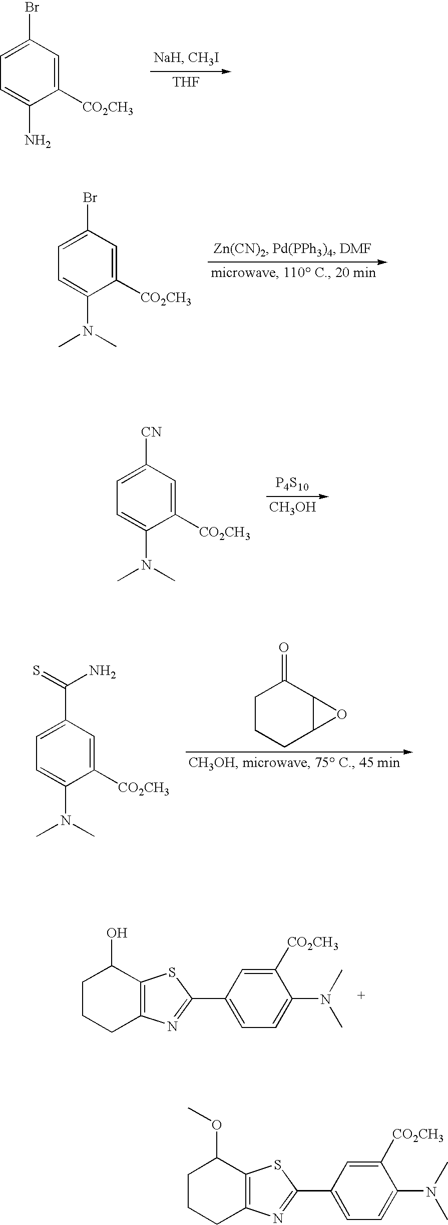

Step A: Following General Procedure I, methyl 5-bromo-2-(dimethylamino)benzoate was prepared as a white solid in 74% yield starting from methyl 2-amino-5-bromobenzoate.

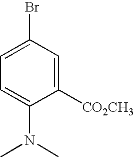

methyl 5-bromo-2-(dimethylamino)benzoate, MS: m/z 259 (M+H)

Step B: Following Step C in Example 55-58, methyl 5-cyano-2-(dimethylamino)benzoate was prepared as a white solid in 98% yield.

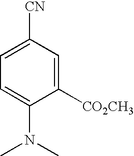

methyl 5-cyano-2-(dimethylamino)benzoate, MS: m/z 205 (M+H)

Step C: Following General Procedure II, methyl 5-(aminocarbonothioyl)-2-(dimethylamino)benzoate was prepared as a yellow solid in 76% yield.

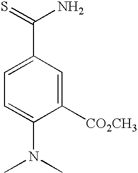

methyl 5-(aminocarbonothioyl)-2-(dimethylamino)benzoate, MS: m/z 239 (M+H)

Step D: Following the General Procedure III, methyl 2-(dimethylamino)-5-(7-hydroxy-4,5,6,7-tetrahydro-1,3-benzothiazol-2-yl)benzoate was prepared as a yellow solid in 74% yield, and methyl 2-(dimethylamino)-5-(7-methoxy-4,5,6,7-tetrahydro-1,3-benzothiazol-2-yl)benzoate was also separated as a colorless gel in 20% yield.

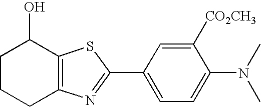

methyl 2-(dimethylamino)-5-(7-hydroxy-4,5,6,7-tetrahydro-1,3-benzothiazol-2-yl)benzoate, MS: m/z 333 (M+H)

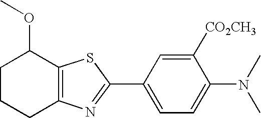

methyl 2-(dimethylamino)-5-(7-methoxy-4,5,6,7-tetrahydro-1,3-benzothiazol-2-yl)benzoate, MS: m/z 347 (M+H)

### Example 105

### Preparation of 2-(dimethylamino)-5-(7-hydroxy-4,5,6,7-tetrahydro-1,3-benzothiazol-2-yl)benzoic acid

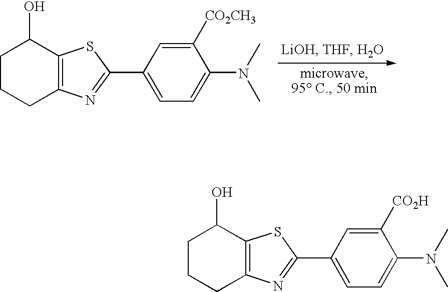

Following the procedure described in Example 59-60, 2-(dimethylamino)-5-(7-hydroxy-4,5,6,7-tetrahydro-1,3-benzothiazol-2-yl)benzoic acid was prepared as a yellow solid in 97% yield

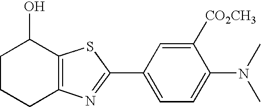

2-(dimethylamino)-5-(7-hydroxy-4,5,6,7-tetrahydro-1,3-benzothiazol-2-yl)benzoic acid, MS: m/z 319 (M+H)

### Example 106

### Preparation of 2-[4-(dimethylamino)-3-(hydroxymethyl)phenyl]-4,5,6,7-tetrahydro-1,3-benzothiazol-7-ol

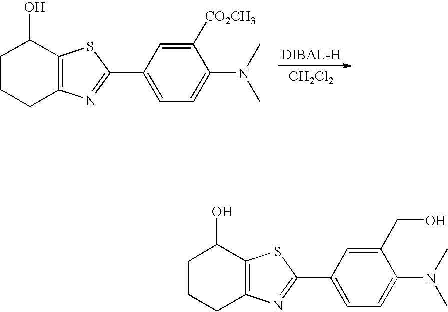

A solution of methyl 2-(dimethylamino)-5-(7-hydroxy-4,5,6,7-tetrahydro-1,3-benzothiazol-2-yl)benzoate (51 mg, 0.15 mmol) in anhydrous CH₂Cl₂ (2 mL) was treated with 1.0M diisobutylaluminum hydride (DIBAL-H) in dichloromethane (0.61 mL, 0.61 mmol). The reaction mixture was stirred at room temperature for 1 hour, then quenched with H₂O. To this mixture was added EtOAc and the mixture was then filtered through a pad of Celite. The organic layer was separated and dried over anhydrous Na₂SO₄, filtered and then concentrated to gave 40 mg (88%) of 2-[4-(dimethylamino)-3-(hydroxymethyl)phenyl]-4,5,6,7-tetrahydro-1,3-benzothiazol-7-ol as a yellow solid.

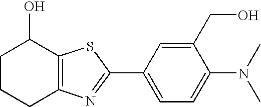

2-[4-(dimethylamino)-3-(hydroxymethyl)phenyl]-4,5,6,7-tetrahydro-1,3-benzothiazol-7-ol, MS: m/z 305 (M+H)

### Examples 107-109

### Preparation of 2-{4-(dimethylamino)-3-[(dimethylamino)methyl]phenyl}-4,5,6,7-tetrahydro-1,3-benzothiazol-7-ol, 2-[4-(dimethylamino)-3-(pyrrolidin-1-ylmethyl)phenyl]-4,5,6,7-tetrahydro-1,3-benzothiazol-7-ol and 2-[4-(dimethylamino)-3-(piperidin-1-ylmethyl)phenyl]-4,5,6,7-tetrahydro-1,3-benzothiazol-7-ol

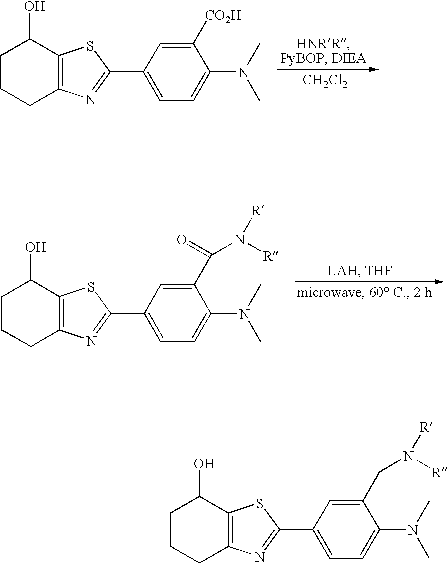

Step A. Preparation of amides. Following the procedure described in Examples 61-62, the following amides were prepared.

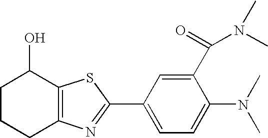

2-(dimethylamino)-5-(7-hydroxy-4,5,6,7-tetrahydro-1,3-benzothiazol-2-yl)-N,N-dimethylbenzamide, yellow solid, MS: m/z 346 (M+H)

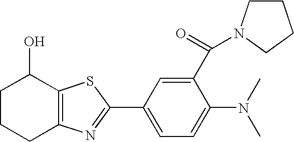

2-[4-(dimethylamino)-3-(pyrrolidin-1-ylcarbonyl)phenyl]-4,5,6,7-tetrahydro-1,3-benzothiazol-7-ol, yellow solid, MS: m/z 372 (M+H)

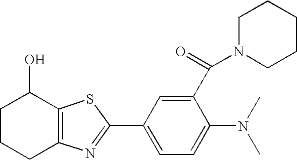

2-[4-(dimethylamino)-3-(piperidin-1-ylcarbonyl)phenyl]-4,5,6,7-tetrahydro-1,3-benzothiazol-7-ol, yellow solid, MS: m/z 386 (M+H)

Step B. Preparation of amines. Following the procedure described in Examples 63-66, the following amines were prepared.

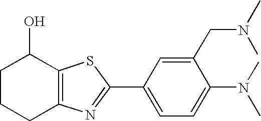

2-{4-(dimethylamino)-3-[(dimethylamino)methyl]phenyl}-4,5,6,7-tetrahydro-1,3-benzothiazol-7-ol, yellow solid, MS: m/z 332 (M+H)

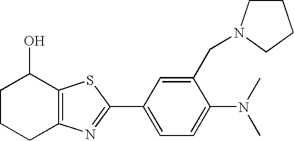

2-[4-(dimethylamino)-3-(pyrrolidin-1-ylmethyl)phenyl]-4,5,6,7-tetrahydro-1,3-benzothiazol-7-ol, yellow gum, MS: m/z 358 (M+H)

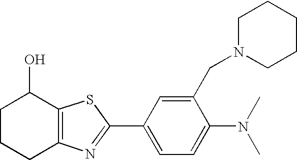

2-[4-(dimethylamino)-3-(piperidin-1-ylmethyl)phenyl]-4,5,6,7-tetrahydro-1,3-benzothiazol-7-ol, yellow gum, MS: m/z 372 (M+H)

### Example 110

### Preparation of 2-(dimethylamino)-5-(7-hydroxy-4,5,6,7-tetrahydro-1,3-benzothiazol-2-yl)benzaldehyde

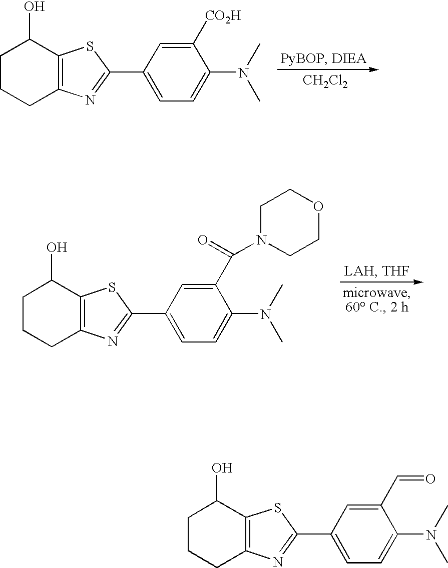

Step A. Following the procedure described in Examples 61-62, 2-[4-(dimethylamino)-3-(morpholin-4-ylcarbonyl)phenyl]-4,5,6,7-tetrahydro-1,3-benzothiazol-7-ol was prepared as a yellow solid.

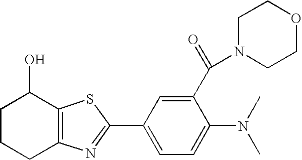

2-[4-(dimethylamino)-3-(morpholin-4-ylcarbonyl)phenyl]-4,5,6,7-tetrahydro-1,3-benzothiazol-7-ol, MS: m/z 388 (M+H)

Step B. Following the procedure described in Examples 63-66, 2-(dimethylamino)-5-(7-hydroxy-4,5,6,7-tetrahydro-1,3-benzothiazol-2-yl)benzaldehyde was prepared as a yellow solid.

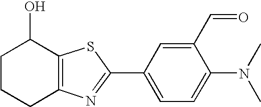

2-(dimethylamino)-5-(7-hydroxy-4,5,6,7-tetrahydro-1,3-benzothiazol-2-yl)benzaldehyde, MS: m/z 303 (M+H)

### Examples 111-137

### Preparation of isopropyl 5-(dimethylamino)-2-(7-hydroxy-4,5,6,7-tetrahydro-1,3-benzothiazol-2-yl)benzoate, allyl 5-(dimethylamino)-2-(7-hydroxy-4,5,6,7-tetrahydro-1,3-benzothiazol-2-yl)benzoate, 2-methoxyethyl 5-(dimethylamino)-2-(7-hydroxy-4,5,6,7-tetrahydro-1,3-benzothiazol-2-yl)benzoate, benzyl 5-(dimethylamino)-2-(7-hydroxy-4,5,6,7-tetrahydro-1,3-benzothiazol-2-yl)benzoate, 2-furylmethyl 5-(dimethylamino)-2-(7-hydroxy-4,5,6,7-tetrahydro-1,3-benzothiazol-2-yl)benzoate, 3-furylmethyl 5-(dimethylamino)-2-(7-hydroxy-4,5,6,7-tetrahydro-1,3-benzothiazol-2-yl)benzoate, cyclopentyl 5-(dimethylamino)-2-(7-hydroxy-4,5,6,7-tetrahydro-1,3-benzothiazol-2-yl)benzoate, butyl 5-(dimethylamino)-2-(7-hydroxy-4,5,6,7-tetrahydro-1,3-benzothiazol-2-yl)benzoate, cyclobutylmethyl 5-(dimethylamino)-2-(7-hydroxy-4,5,6,7-tetrahydro-1,3-benzothiazol-2-yl)benzoate, cyclohexyl methyl 5-(dimethylamino)-2-(7-hydroxy-4,5,6,7-tetrahydro-1,3-benzothiazol-2-yl)benzoate, propyl 5-(dimethylamino)-2-(7-hydroxy-4,5,6,7-tetrahydro-1,3-benzothiazol-2-yl)benzoate, 3-thienylmethyl 5-(dimethylamino)-2-(7-hydroxy-4,5,6,7-tetrahydro-1,3-benzothiazol-2-yl)benzoate, 2-thienylmethyl 5-(dimethylamino)-2-(7-hydroxy-4,5,6,7-tetrahydro-1,3-benzothiazol-2-yl)benzoate, 2-pyrrolidin-1-ylethyl 5-(dimethylamino)-2-(7-hydroxy-4,5,6,7-tetrahydro-1,3-benzothiazol-2-yl)benzoate, 2-(dimethylamino)ethyl 5-(dimethylamino)-2-(7-hydroxy-4,5,6,7-tetrahydro-1,3-benzothiazol-2-yl)benzoate, pyridin-3-ylmethyl 5-(dimethylamino)-2-(7-hydroxy-4,5,6,7-tetrahydro-1,3-benzothiazol-2-yl)benzoate, pyridin-4-ylmethyl 5-(dimethylamino)-2-(7-hydroxy-4,5,6,7-tetrahydro-1,3-benzothiazol-2-yl)benzoate, pyridin-2-ylmethyl 5-(dimethylamino)-2-(7-hydroxy-4,5,6,7-tetrahydro-1,3-benzothiazol-2-yl)benzoate, prop-2-yn-1-yl 5-(dimethylamino)-2-(7-hydroxy-4,5,6,7-tetrahydro-1,3-benzothiazol-2-yl)benzoate, (2E)-but-2-en-1-yl 5-(dimethylamino)-2-(7-hydroxy-4,5,6,7-tetrahydro-1,3-benzothiazol-2-yl)benzoate, 2-oxopropyl 5-(dimethylamino)-2-(7-hydroxy-4,5,6,7-tetrahydro-1,3-benzothiazol-2-yl)benzoate, tetrahydrofuran-2-ylmethyl 5-(dimethylamino)-2-(7-hydroxy-4,5,6,7-tetrahydro-1,3-benzothiazol-2-yl)benzoate, but-2-yn-1-yl 5-(dimethylamino)-2-(7-hydroxy-4,5,6,7-tetrahydro-1,3-benzothiazol-2-yl)benzoate, tetrahydrofuran-3-ylmethyl 5-(dimethylamino)-2-(7-hydroxy-4,5,6,7-tetrahydro-1,3-benzothiazol-2-yl)benzoate, 2-(acetyloxy)ethyl 5-(dimethylamino)-2-(7-hydroxy-4,5,6,7-tetrahydro-1,3-benzothiazol-2-yl)benzoate, 3-methyl but-2-en-1-yl 5-(dimethylamino)-2-(7-hydroxy-4,5,6,7-tetrahydro-1,3-benzothiazol-2-yl)benzoate and 2,2-dimethylpropyl 5-(dimethylamino)-2-(7-hydroxy-4,5,6,7-tetrahydro-1,3-benzothiazol-2-yl)benzoate

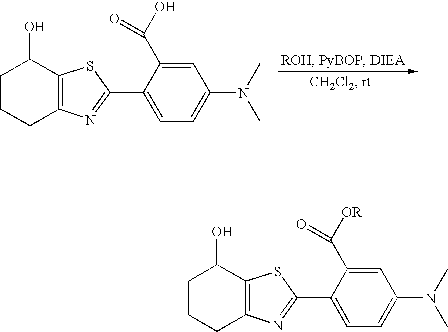

General Procedure of Esterification. A mixture of acid (1.0 mmol) and PyBOP (1.5 mmol) in CH₂Cl₂ (10 mL) was added diisopropylethylamine (3.0 mL) and the corresponding alcohol (1.5 mmol). The reaction mixture was stirred at room temperature, monitoring by TLC. After completion, the reaction mixture was concentrated and separated by HPLC. The following esters were prepared.

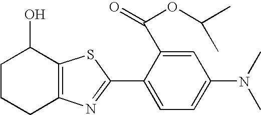

isopropyl 5-(dimethylamino)-2-(7-hydroxy-4,5,6,7-tetrahydro-1,3-benzothiazol-2-yl)benzoate, yellow gum, MS: m/z 361 (M+H)

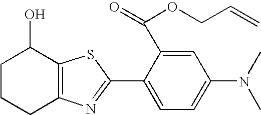

allyl 5-(dimethylamino)-2-(7-hydroxy-4,5,6,7-tetrahydro-1,3-benzothiazol-2-yl)benzoate, MS: m/z 359 (M+H)

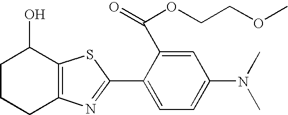

2-methoxyethyl 5-(dimethylamino)-2-(7-hydroxy-4,5,6,7-tetrahydro-1,3-benzothiazol-2-yl)benzoate, MS: m/z 377 (M+H)

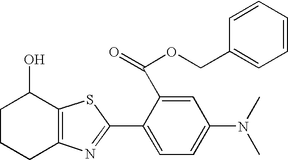

benzyl 5-(dimethylamino)-2-(7-hydroxy-4,5,6,7-tetrahydro-1,3-benzothiazol-2-yl)benzoate, MS: m/z 409 (M+H)

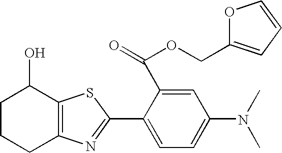

2-furylmethyl 5-(dimethylamino)-2-(7-hydroxy-4,5,6,7-tetrahydro-1,3-benzothiazol-2-yl)benzoate, MS: m/z 399 (M+H)

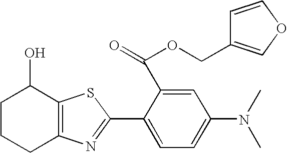

3-furylmethyl 5-(dimethylamino)-2-(7-hydroxy-4,5,6,7-tetrahydro-1,3-benzothiazol-2-yl)benzoate, MS: m/z 399 (M+H)

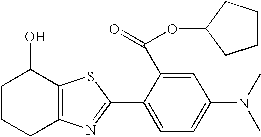

cyclopentyl 5-(dimethylamino)-2-(7-hydroxy-4,5,6,7-tetrahydro-1,3-benzothiazol-2-yl)benzoate, MS: m/z 387 (M+H)

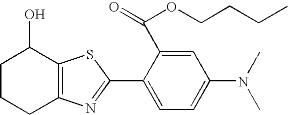

butyl 5-(dimethylamino)-2-(7-hydroxy-4,5,6,7-tetrahydro-1,3-benzothiazol-2-yl)benzoate, MS: m/z 375 (M+H)

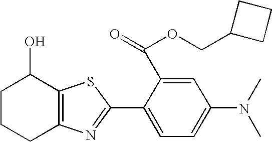

cyclobutylmethyl 5-(dimethylamino)-2-(7-hydroxy-4,5,6,7-tetrahydro-1,3-benzothiazol-2-yl)benzoate, MS: m/z 387 (M+H)

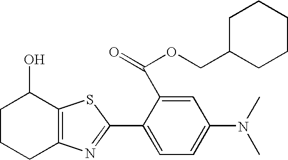

cyclohexylmethyl 5-(dimethylamino)-2-(7-hydroxy-4,5,6,7-tetrahydro-1,3-benzothiazol-2-yl)benzoate, MS: m/z 415 (M+H)

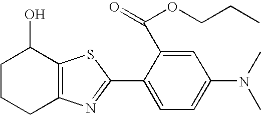

propyl 5-(dimethylamino)-2-(7-hydroxy-4,5,6,7-tetrahydro-1,3-benzothiazol-2-yl)benzoate, yellow gum, MS: m/z 361 (M+H)

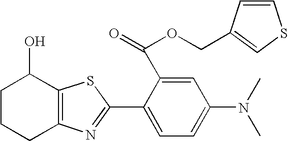

3-thienylmethyl 5-(dimethylamino)-2-(7-hydroxy-4,5,6,7-tetrahydro-1,3-benzothiazol-2-yl)benzoate, yellow solid, MS: m/z 415 (M+H)

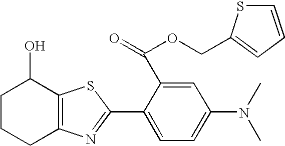

2-thienylmethyl 5-(dimethylamino)-2-(7-hydroxy-4,5,6,7-tetrahydro-1,3-benzothiazol-2-yl)benzoate, yellow solid, MS: m/z 415 (M+H)

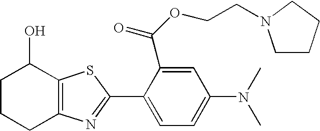

2-pyrrolidin-1-ylethyl 5-(dimethylamino)-2-(7-hydroxy-4,5,6,7-tetrahydro-1,3-benzothiazol-2-yl)benzoate, yellow gum, MS: m/z 416 (M+H)

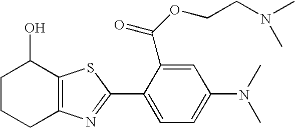

2-(dimethylamino)ethyl 5-(dimethylamino)-2-(7-hydroxy-4,5,6,7-tetrahydro-1,3-benzothiazol-2-yl)benzoate, yellow film, MS: m/z 390 (M+H)

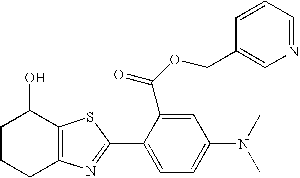

pyridin-3-ylmethyl 5-(dimethylamino)-2-(7-hydroxy-4,5,6,7-tetrahydro-1,3-benzothiazol-2-yl)benzoate, yellow gum, MS: m/z 410 (M+H)

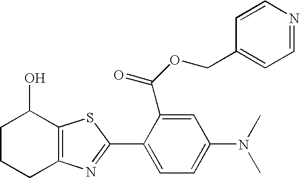

pyridin-4-ylmethyl 5-(dimethylamino)-2-(7-hydroxy-4,5,6,7-tetrahydro-1,3-benzothiazol-2-yl)benzoate, yellow gum, MS: m/z 410 (M+H)

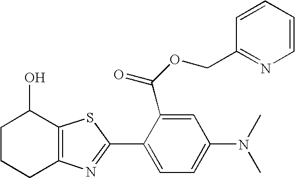

pyridin-2-ylmethyl 5-(dimethylamino)-2-(7-hydroxy-4,5,6,7-tetrahydro-1,3-benzothiazol-2-yl)benzoate, yellow gum, MS: m/z 410 (M+H)

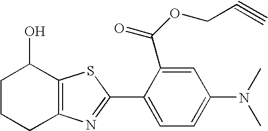

prop-2-yn-1-yl 5-(dimethylamino)-2-(7-hydroxy-4,5,6,7-tetrahydro-1,3-benzothiazol-2-yl)benzoate, yellow gum, MS: m/z 357 (M+H)

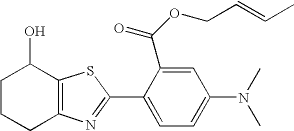

(2E)-but-2-en-1-yl 5-(dimethylamino)-2-(7-hydroxy-4,5,6,7-tetrahydro-1,3-benzothiazol-2-yl)benzoate, yellow gum, MS: m/z 373 (M+H)

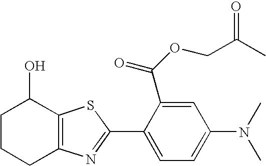

2-oxopropyl 5-(dimethylamino)-2-(7-hydroxy-4,5,6,7-tetrahydro-1,3-benzothiazol-2-yl)benzoate, yellow gum, MS: m/z 375 (M+H)

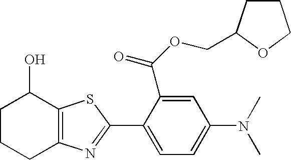

tetrahydrofuran-2-ylmethyl 5-(dimethylamino)-2-(7-hydroxy-4,5,6,7-tetrahydro-1,3-benzothiazol-2-yl)benzoate, yellow gum, MS: m/z 403 (M+H)

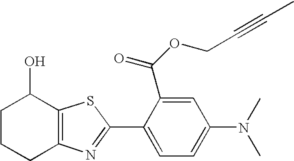

but-2-yn-1-yl 5-(dimethylamino)-2-(7-hydroxy-4,5,6,7-tetrahydro-1,3-benzothiazol-2-yl)benzoate, yellow gum, MS: m/z 371 (M+H)

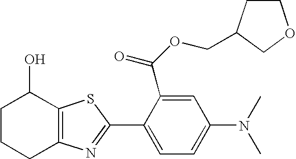

tetrahydrofuran-3-ylmethyl 5-(dimethylamino)-2-(7-hydroxy-4,5,6,7-tetrahydro-1,3-benzothiazol-2-yl)benzoate, yellow gum, MS: m/z 403 (M+H)

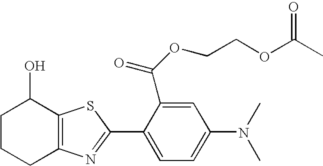

2-(acetyloxy)ethyl 5-(dimethylamino)-2-(7-hydroxy-4,5,6,7-tetrahydro-1,3-benzothiazol-2-yl)benzoate, yellow gum, MS: m/z 405 (M+H)

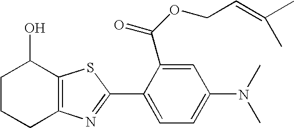

3-methyl but-2-en-1-yl 5-(dimethylamino)-2-(7-hydroxy-4,5,6,7-tetrahydro-1,3-benzothiazol-2-yl)benzoate, yellow gum, MS: m/z 387 (M+H)

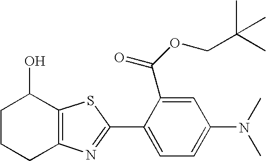

2,2-dimethylpropyl 5-(dimethylamino)-2-(7-hydroxy-4,5,6,7-tetrahydro-1,3-benzothiazol-2-yl)benzoate, yellow gum, MS: m/z 389 (M+H)

### Examples 138 and 139

### Preparation of piperidin-3-ylmethyl 5-(dimethylamino)-2-(7-hydroxy-4,5,6,7-tetrahydro-1,3-benzothiazol-2-yl)benzoate and piperidin-3-yl 5-(dimethylamino)-2-(7-hydroxy-4,5,6,7-tetrahydro-1,3-benzothiazol-2-yl)benzoate

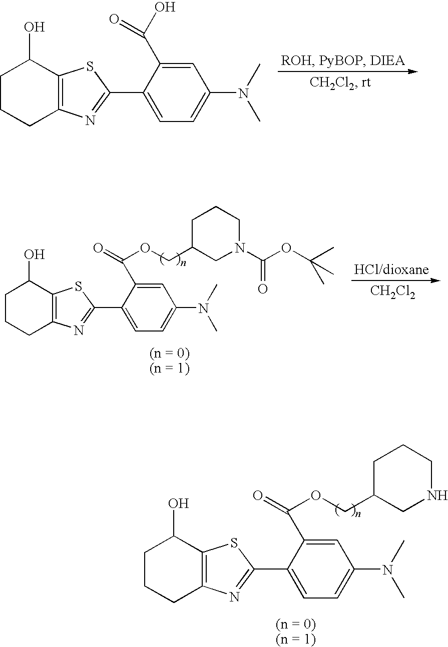

Step A. Following the General Procedure of Esterification described in Examples 111-137, tert-butyl 3-({[5-(dimethylamino)-2-(7-hydroxy-4,5,6,7-tetrahydro-1,3-benzothiazol-2-yl)benzoyl]oxy}methyl)piperidine-1-carboxylate was prepared, staring from N-Boc-3-hydroxymethylpiperidine.

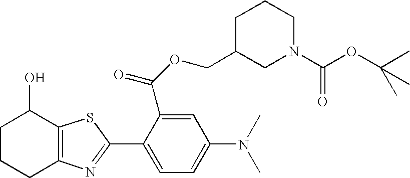

tert-butyl 3-({[5-(dimethylamino)-2-(7-hydroxy-4,5,6,7-tetrahydro-1,3-benzothiazol-2-yl)benzoyl]oxy}methyl)piperidine-1-carboxylate, yellow gum, MS: m/z 516 (M+H)

Tert-Butyl 3-{[5-(dimethylamino)-2-(7-hydroxy-4,5,6,7-tetrahydro-1,3-benzothiazol-2-yl)benzoyl]oxy}piperidine-1-carboxylate was prepared, starting from N-Boc-3-hydroxypiperidine. This compound was used in Step B without further purification.

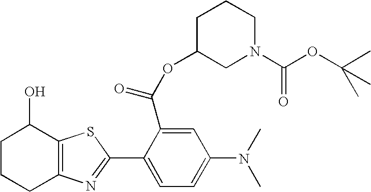

tert-butyl 3-{[5-(dimethylamino)-2-(7-hydroxy-4,5,6,7-tetrahydro-1,3-benzothiazol-2-yl)benzoyl]oxy}piperidine-1-carboxylate, MS: m/z 502 (M+H)

Step B. General Procedure of Deprotection to Remove BOC Protecting Group Using HCl/dioxane. A solution of the Boc-protected compound prepared above (0.1 mmol) in CH₂Cl₂ (1 mL) was treated with 4N HCl/dioxane (0.5 mmol). The reaction mixture was stirred at room temperature, monitoring by TLC. After the reaction was completed, the reaction mixture was concentrated and separated by HPLC to give piperidin-3-ylmethyl 5-(dimethylamino)-2-(7-hydroxy-4,5,6,7-tetrahydro-1,3-benzothiazol-2-yl)benzoate and piperidin-3-yl 5-(dimethylamino)-2-(7-hydroxy-4,5,6,7-tetrahydro-1,3-benzothiazol-2-yl)benzoate.

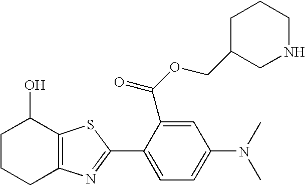

piperidin-3-ylmethyl 5-(dimethylamino)-2-(7-hydroxy-4,5,6,7-tetrahydro-1,3-benzothiazol-2-yl)benzoate, yellow gum, MS: m/z 416 (M+H)

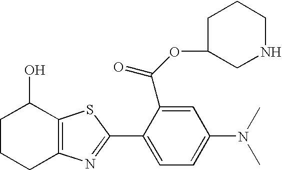

piperidin-3-yl 5-(dimethylamino)-2-(7-hydroxy-4,5,6,7-tetrahydro-1,3-benzothiazol-2-yl)benzoate, yellow gum, MS: m/z 402 (M+H)

### Example 140

### Preparation of 2-[2-bromo-4-(dimethylamino)phenyl]-4,5,6,7-tetrahydro-1,3-benzothiazol-7-ol

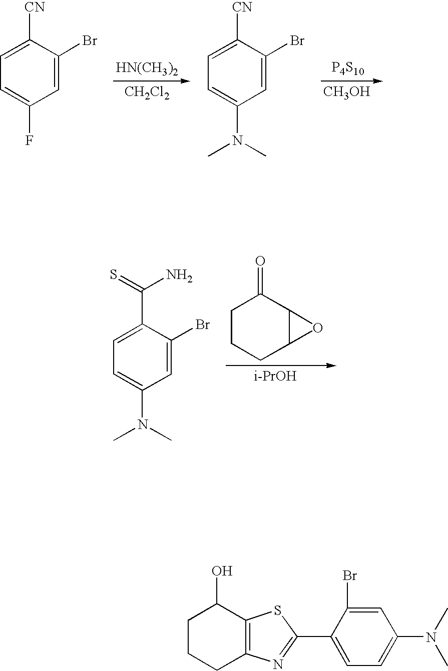

Step A. 4-Dimethylamino-2-bromo-benzonitrile was prepared by following the procedure described in the preparation of Examples 43-44.

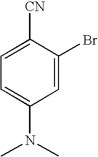

4-Dimethylamino-2-bromo-benzonitrile, MS: m/z 226 (M+H)

Step B. 4-(dimethylamino)-2-bromobenzenecarbothioamide was prepared by following General Procedure II.

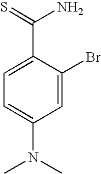

4-(dimethylamino)-2-bromobenzenecarbothioamide, MS: m/z 260 (M+H)

Step C. 2-[2-Bromo-4-(dimethylamino)phenyl]-4,5,6,7-tetrahydro-1,3-benzothiazol-7-ol was prepared by following General Procedure III.

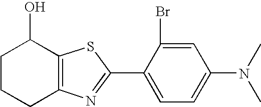

2-[2-bromo-4-(dimethylamino)phenyl]-4,5,6,7-tetrahydro-1,3-benzothiazol-7-ol, yellow solid, MS: m/z 354 (M+H)

### Example 141

### Preparation of 2-{4-[methoxy(methyl)amino]phenyl}-4,5,6,7-tetrahydro-1,3-benzothiazol-7-ol

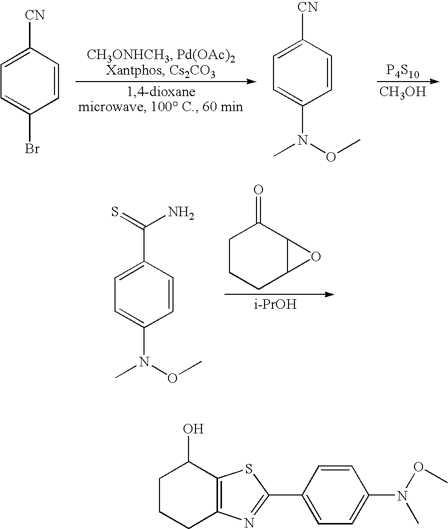

Step A. A mixture of 4-bromobenzonitrile (500 mg, 3.0 mmol), N,N-dimethylhydroxylamine hydrochloride (219 mg, 3.6 mmol), Xantphos (26 mg, 0.045 mmol), Cs₂CO₃ (1.4 g, 4.2 mmol) and Pd(OAc)₂ (4 mg, 0.016 mmol) in 1,4-dioxane (3 mL) was subjected to a microwave-assisted reaction at 10° C. for 1 hour, then filtered and concentrated. The residue was separated by flash chromatography (hexane:EtOAc=1:1) providing 442 mg (92%) of 4-[methoxy(methyl)amino]benzonitrile as a colorless oil.

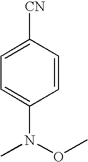

4-[methoxy(methyl)amino]benzonitrile, MS: m/z 163 (M+H)

Step B. 4-[methoxy(methyl)amino]benzenecarbothioamide was prepared by following General Procedure II.

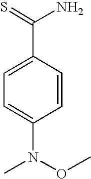

4-[methoxy(methyl)amino]benzenecarbothioamide, MS: m/z 197 (M+H)

Step C. 2-{4-[methoxy(methyl)amino]phenyl}-4,5,6,7-tetrahydro-1,3-benzothiazol-7-ol was prepared by following General Procedure III.

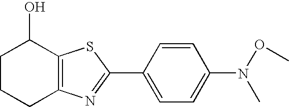

2-{4-[methoxy(methyl)amino]phenyl}-4,5,6,7-tetrahydro-1,3-benzothiazol-7-ol, yellow solid, MS: m/z 291 (M+H)

### Example 142

### Preparation of 2-[4-(trimethylhydrazino)phenyl]-4,5,6,7-tetrahydro-1,3-benzothiazol-7-ol

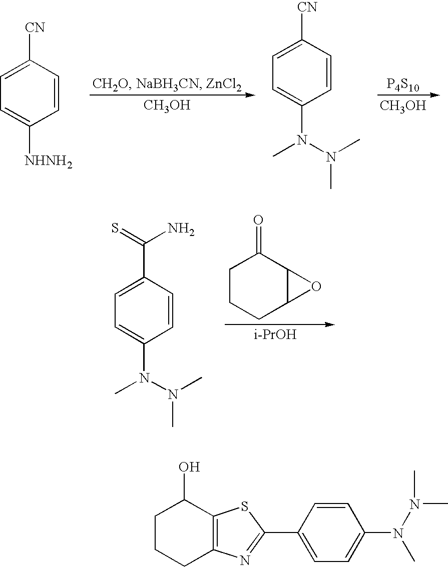

Step A. 4-(Trimethylhydrazino)benzonitrile was prepared as a yellow wax by following the procedure described in Step D, Examples 89-90.

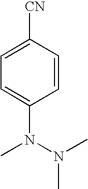

4-(trimethylhydrazino)benzonitrile, MS: m/z 176 (M+H)

Step B. 4-(Trimethylhydrazino)benzenecarbothioamide was prepared by following General Procedure II.

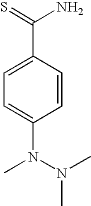

4-(trimethylhydrazino)benzenecarbothioamide, MS: m/z 210 (M+H)

Step C. 2-[4-(Trimethylhydrazino)phenyl]-4,5,6,7-tetrahydro-1,3-benzothiazol-7-ol was prepared by following General Procedure III.

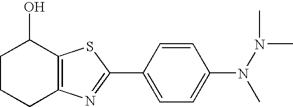

2-[4-(trimethylhydrazino)phenyl]-4,5,6,7-tetrahydro-1,3-benzothiazol-7-ol, yellow solid, MS: m/z 304 (M+H)

### Example 143

### Preparation of 2-(4-azetidin-1-ylphenyl)-4,5,6,7-tetrahydro-1,3-benzothiazol-7-ol, 2-[4-(3-Hydroxypyrrolidin-1-yl)phenyl]-4,5,6,7-tetrahydro-1,3-benzothiazol-7-ol, 2-(4-Piperidin-1-ylphenyl)-4,5,6,7-tetrahydro-1,3-benzothiazol-7-ol and 2-(4-Morpholin-4-ylphenyl)-4,5,6,7-tetrahydro-1,3-benzothiazol-7-ol

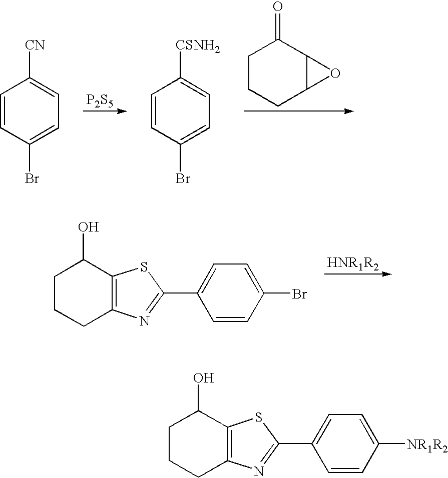

### Preparation of 2-(4-azetidin-1-ylphenyl)-4,5,6,7-tetrahydro-1,3-benzothiazol-7-ol

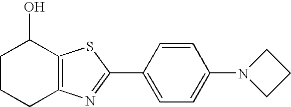

### Step A: 4-Bromobenzenecarbothioamide

To a solution of 4-bromobenzonitrile (3.64 g, 20 mmol) in 10 mL of methyl alcohol is added phosphorus pentasulfide (3.56 g, 8 mmol). The mixture is stirred under nitrogen atmosphere at room temperature for 16 hours and then concentrated. The residue is chromatographed over silica gel, eluting with a gradient of 50% ethyl acetate in hexanes to ethyl acetate. Concentration provides 4-bromobenzenecarbothioamide as a tan solid (2.1 g).

### Step B: 2-(4-Bromophenyl)-4,5,6,7-tetrahydro-1,3-benzothiazol-7-ol

A solution of 4-bromobenzenecarbothioamide (216 mg, 1.0 mmol) and 7-oxabicyclo[4.1.0]heptan-2-one (225 mg, 2.0 mmol) in 4 mL of methyl alcohol is stirred at reflux for 1 hour and then concentrated. The residue is triturated with ethyl acetate to provide 2-(4-bromophenyl)-4,5,6,7-tetrahydro-1,3-benzothiazol-7-ol as a white solid (102 mg). MS: m/z 310.0, 312.0 (M+H).

### Step C: 2-(4-Azetidin-1-ylphenyl)-4,5,6,7-tetrahydro-1,3-benzothiazol-7-ol

A mixture of 2-(4-bromophenyl)-4,5,6,7-tetrahydro-1,3-benzothiazol-7-ol (93 mg, 0.3 mmol), azetidine hydrochloride (37 mg, 0.4 mmol), tris(dibenzylideneacetone)dipalladium(0) (5 mg), rac-2,2′-bis(diphenylphosphino)-1,1′-binaphthyl (5 mg), and sodium tert-butoxide (80 mg, 0.8 mmol) in 2 mL of toluene is stirred at 80° C. for 3 hours and then concentrated. The residue is dissolved in ethyl acetate and filtered through Magnesol. The filtrate is concentrated, then the residue is chromatographed over silica gel, eluting with a gradient of methylene chloride to 5% methyl alcohol in methylene chloride. Concentration provides 2-(4-azetidin-1-ylphenyl)-4,5,6,7-tetrahydro-1,3-benzothiazol-7-ol as a yellow solid (34 mg). MS: m/z 287.1 (M+H).

Examples 144-146 are synthesized analogously to Example 143.

### Example 144

Example 144 is synthesized according to Example 143.

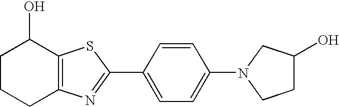

2-[4-(3-Hydroxypyrrolidin-1-yl)phenyl]-4,5,6,7-tetrahydro-1,3-benzothiazol-7-ol, MS: m/z 317.1 (M+H)

### Example 145

Example 145 is synthesized according to Example 143.

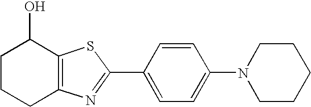

2-(4-Piperidin-1-ylphenyl)-4,5,6,7-tetrahydro-1,3-benzothiazol-7-ol, MS: m/z 315.1 (M+H)

### Example 146

Example 146 is synthesized according to Example 143.

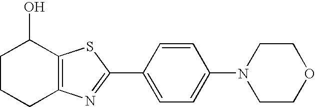

2-(4-Morpholin-4-ylphenyl)-4,5,6,7-tetrahydro-1,3-benzothiazol-7-ol, MS: m/z 317.1 (M+H)

### Example 147

### Preparation of 2-[4-(4-methylpiperazin-1-yl)phenyl]-4,5,6,7-tetrahydro-1,3-benzothiazol-7-ol

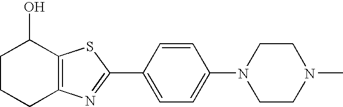

### Step A: 4-(4-Methylpiperazin-1-yl)benzonitrile

A mixture of 4-bromobenzonitrile (1.82 g, 10 mmol), 1-methylpiperazine (2.0 g, 10 mmol), tris(dibenzylideneacetone)dipalladium(0) (50 mg), rac-2,2′-bis(diphenylphosphino)-1,1′-binaphthyl (50 mg), and sodium tert-butoxide (1.92 g, 20 mmol) in 20 mL of toluene is stirred at 80° C. for 18 hours and then concentrated. The residue is chromatographed over silica gel, eluting with a gradient of ethyl acetate to 20% methyl alcohol in ethyl acetate. Concentration provides 4-(4-methylpiperazin-1-yl)benzonitrile as a tan solid (1.6 g).

### Step B: 4-(4-Methylpiperazin-1-yl)benzenecarbothioamide

To a mixture of 4-(4-methylpiperazin-1-yl)benzonitrile (402 mg, 2.0 mmol) in 1.0 mL of methyl alcohol is added phosphorus pentasulfide (356 mg, 0.8 mmol). The mixture is stirred under nitrogen atmosphere at room temperature for 18 hours and then concentrated. The residue is chromatographed over silica gel, eluting with a gradient of ethyl acetate to 20% methyl alcohol in ethyl acetate. Concentration provides 4-(4-methylpiperazin-1-yl)benzenecarbothioamide as a yellow solid (130 mg). MS: m/z 236.1 (M+H).

### Step C: 2-[4-(4-Methylpiperazin-1-yl)phenyl]-4,5,6,7-tetrahydro-1,3-benzothiazol-7-ol

A solution of 4-(4-methylpiperazin-1-yl)benzenecarbothioamide (130 mg, 0.55 mmol) and 7-oxabicyclo[4.1.0]heptan-2-one (100 mg, 0.89 mmol) in 2 mL of methyl alcohol is stirred at 40° C. for 18 hours and then concentrated. The residue is chromatographed over silica gel, eluting with a gradient of ethyl acetate to 50% methyl alcohol in ethyl acetate. Concentration provides 2-[4-(4-methylpiperazin-1-yl)phenyl]-4,5,6,7-tetrahydro-1,3-benzothiazol-7-ol as a white solid (36 mg). MS: m/z 330.1 (M+H).

A synthetic Scheme for preparing compounds 2-[4-(4-methylpiperazin-1-yl)phenyl]-4,5,6,7-tetrahydro-1,3-benzothiazol-7-ol, 2-{4-[(3S)-3-(Dimethylamino)pyrrolidin-1-yl]phenyl}-4,5,6,7-tetrahydro-1,3-benzothiazol-7-ol and 2-[4-(2-Methylpyrrolidin-1-yl)phenyl]-4,5,6,7-tetrahydro-1,3-benzothiazol-7-ol, is provided below.

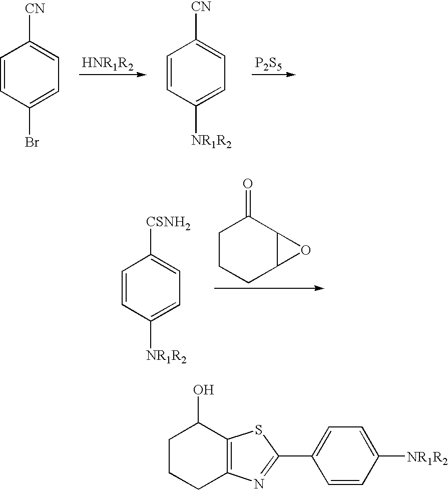

### Examples 148 and 149

### Preparation of 2-{4-[(3S)-3-(Dimethylamino)pyrrolidin-1-yl]phenyl}-4,5,6,7-tetrahydro-1,3-benzothiazol-7-ol and 2-[4-(2-Methylpyrrolidin-1-yl)phenyl]-4,5,6,7-tetrahydro-1,3-benzothiazol-7-ol

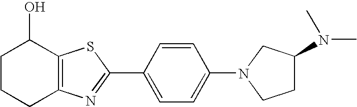

2-{4-[(3S)-3-(Dimethylamino)pyrrolidin-1-yl]phenyl}-4,5,6,7-tetrahydro-1,3-benzothiazol-7-ol, MS: m/z 344.1 (M+H)

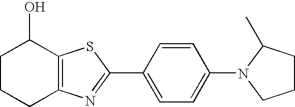

2-[4-(2-Methylpyrrolidin-1-yl)phenyl]-4,5,6,7-tetrahydro-1,3-benzothiazol-7-ol, MS: m/z 315.1 (M+H)

### Example 150

### Preparation of 2-{4-[ethyl(methyl)amino]phenyl}-4,5,6,7-tetrahydro-1,3-benzothiazol-7-ol

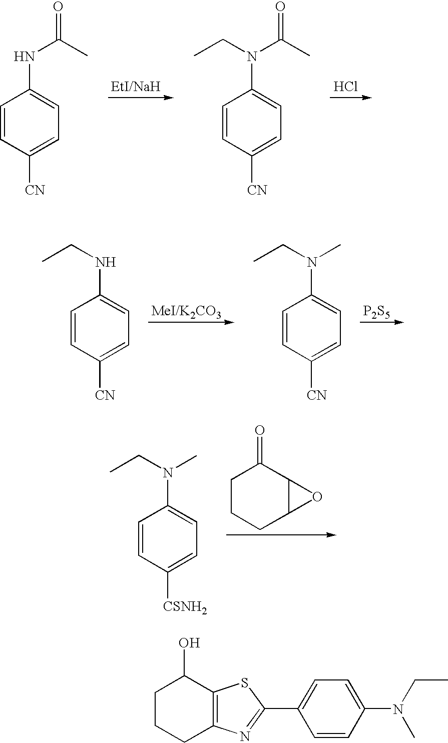

### Preparation of 2-{4-[ethyl(methyl)amino]phenyl}-4,5,6,7-tetrahydro-1,3-benzothiazol-7-ol

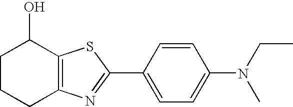

### Step A: N-(4-Cyanophenyl)-N-ethylacetamide

To a solution of 4′-cyanoacetanilide (1.3 g, 8.12 mmol) and iodoethane (1.5 mL, 18.8 mmol) in 10 mL of N,N-dimethylformamide is added sodium hydride (60% in mineral oil, 800 mg, 20 mmol). The mixture is stirred under nitrogen atmosphere at room temperature for 1 hour. The reaction mixture is diluted with ethyl acetate, and then washed with saturated sodium chloride (×3). The organic layer is dried over magnesium sulfate and then concentrated. The residue is chromatographed over silica gel, eluting with a gradient of hexanes to 50% ethyl acetate in hexanes. Concentration provides N-(4-cyanophenyl)-N-ethylacetamide as a yellow solid (760 mg). MS: m/z 189.0 (M+H).

### Step B: 4-(Ethylamino)benzonitrile

A mixture of N-(4-cyanophenyl)-N-ethylacetamide (300 mg, 1.6 mmol) and 1N hydrochloric acid (50 mL) is stirred at reflux for 4 hours then neutralized with potassium carbonate. The product is extracted with ethyl acetate, and the organic layer is washed with saturated sodium chloride, dried over magnesium sulfate, and then concentrated. The residue is chromatographed over silica gel, eluting with a gradient of hexanes to 80% ethyl acetate in hexanes. Concentration provides 4-(ethylamino)benzonitrile as a white solid (128 mg). MS: m/z 147.0 (M+H).

### Step C: 4-[Ethyl(methyl)amino]benzonitrile

A mixture of 4-(ethylamino)benzonitrile (128 mg, 0.88 mmol), iodomethane (0.22 mL, 3.5 mmol), and potassium carbonate (300 mg, 2.2 mmol) in 2 mL of methyl sulfoxide is stirred at 60° C. in a sealed tube for 36 hours. The reaction mixture is diluted with ethyl acetate, and washed with saturated sodium chloride (×3). The organic layer is dried over magnesium sulfate and then concentrated. The residue is chromatographed over silica gel, eluting with a gradient of hexanes to 20% ethyl acetate in hexanes. Concentration provides 4-[ethyl(methyl)amino]benzonitrile as a yellow solid (81 mg). MS: m/z 161.1 (M+H).

### Step D: 4-[Ethyl(methyl)amino]benzenecarbothioamide

To a mixture of 4-[ethyl(methyl)amino]benzonitrile (81 mg, 0.51 mmol) in 1.0 mL of methyl alcohol is added phosphorus pentasulfide (100 mg, 0.22 mmol). The mixture is stirred under nitrogen atmosphere at room temperature for 18 hours, then concentrated. The residue is chromatographed over silica gel, eluting with a gradient of 33% ethyl acetate in hexanes to ethyl acetate. Concentration provides 4-[ethyl(methyl)amino]benzenecarbothioamide as a red oil (78 mg). MS: m/z 195.1 (M+H).

### Step E: 2-{4-[Ethyl(methyl)amino]phenyl}-4,5,6,7-tetrahydro-1,3-benzothiazol-7-ol

A solution of 4-(4-methylpiperazin-1-yl)benzenecarbothioamide (78 mg, 0.40 mmol) and 7-oxabicyclo[4.1.0]heptan-2-one (100 mg, 0.89 mmol) in 2 mL of methyl alcohol is stirred at 40° C. for 18 hours, then concentrated. The residue is chromatographed over silica gel, eluting with a gradient of 50% ethyl acetate in hexanes to ethyl acetate. Concentration provides 2-{4-[ethyl(methyl)amino]phenyl}-4,5,6,7-tetrahydro-1,3-benzothiazol-7-ol as a light tan solid (52 mg). MS: m/z 289.1 (M+H).

### Example 151

### Preparation of 2-(2-nitro-4-pyrrolidin-1-ylphenyl)-4,5,6,7-tetrahydro-1,3-benzothiazol-7-ol and 2-(7-methoxy-4,5,6,7-tetrahydro-1,3-benzothiazol-2-yl)-5-pyrrolidin-1-ylaniline

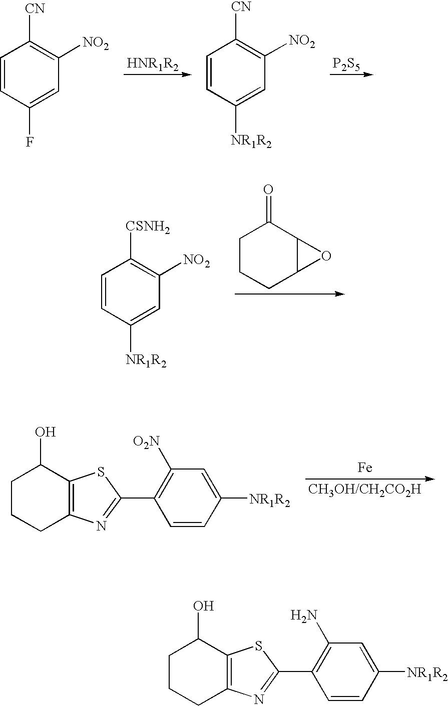

### Preparation of 2-(2-nitro-4-pyrrolidin-1-yl phenyl)-4,5,6,7-tetrahydro-1,3-benzothiazol-7-ol

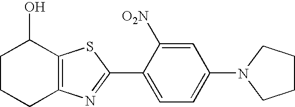

### Step A: 2-Nitro-4-pyrrolidin-1-ylbenzonitrile

A mixture of 4-fluoro-2-nitrobenzonitrile (1.0 g, 6.02 mmol) and 1.5 mL of pyrrolidine in 25 mL of tetrahydrofuran is stirred at room temperature for 2 hours, then concentrated. The residue is diluted with ethyl acetate, and then washed with saturated sodium chloride (×3). The organic layer is dried over magnesium sulfate and then filtered through Magnesol. Concentration provides 2-nitro-4-pyrrolidin-1-ylbenzonitrile as a brick red solid (1.05 g). MS: m/z 218.1 (M+H).

### Step B: 2-Nitro-4-pyrrolidin-1-ylbenzenecarbothioamide

To a mixture of 2-nitro-4-pyrrolidin-1-ylbenzonitrile (1.0 g, 4.61 mmol) in 50 mL of methyl alcohol is added phosphorus pentasulfide (2.0 g, 4.5 mmol). The mixture is stirred under nitrogen atmosphere at room temperature for 3 days and concentrated. The residue is chromatographed over silica gel, eluting with a gradient of 33% ethyl acetate in hexanes to 50% ethyl acetate in hexanes. Concentration provides 2-nitro-4-pyrrolidin-1-ylbenzenecarbothioamide as a red solid (130 mg). MS: m/z 252.1 (M+H)

### Step C: 2-(2-Nitro-4-pyrrolidin-1-ylphenyl)-4,5,6,7-tetrahydro-1,3-benzothiazol-7-ol

A solution of 2-nitro-4-pyrrolidin-1-ylbenzenecarbothioamide (300 mg, 1.20 mmol) and 7-oxabicyclo[4.1.0]heptan-2-one (400 mg, 3.56 mmol) in 50 mL of ethyl alcohol is stirred at 60° C. for 2 hours, then concentrated. The residue is chromatographed over silica gel, eluting with a gradient of 50% ethyl acetate in hexanes to ethyl acetate. Concentration provides 2-(2-nitro-4-pyrrolidin-1-ylphenyl)-4,5,6,7-tetrahydro-1,3-benzothiazol-7-ol as a tan solid (350 mg). MS: m/z 346.1 (M+H).

### Example 152

### Preparation of 2-(7-methoxy-4,5,6,7-tetrahydro-1,3-benzothiazol-2-yl)-5-pyrrolidin-1-ylaniline

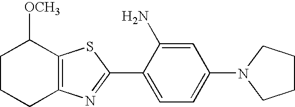

A mixture of 2-(2-nitro-4-pyrrolidin-1-ylphenyl)-4,5,6,7-tetrahydro-1,3-benzothiazol-7-ol (300 mg, 0.87 mmol) and iron powder (500 mg, 8.93 mmol) in 50 mL of methyl alcohol and 10 mL of acetic acid is refluxed for 30 minutes, then concentrated. The residue is chromatographed over silica gel, eluting with a gradient of 50% ethyl acetate in hexanes to ethyl acetate. Concentration provides 2-(7-methoxy-4,5,6,7-tetrahydro-1,3-benzothiazol-2-yl)-5-pyrrolidin-1-ylaniline as a tan solid (42 mg). MS: m/z 330.1 (M+H).

### Example 153

### Preparation of 2-(2-amino-4-pyrrolidin-1-ylphenyl)-4,5,6,7-tetrahydro-1,3-benzothiazol-7-ol

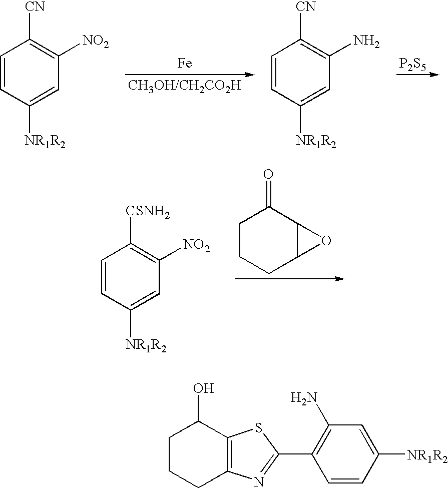

### Preparation of 2-(2-amino-4-pyrrolidin-1-ylphenyl)-4,5,6,7-tetrahydro-1,3-benzothiazol-7-ol

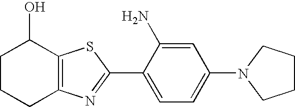

### Step A: 2-Amino-4-pyrrolidin-1-ylbenzonitrile

A mixture of 2-nitro-4-pyrrolidin-1-ylbenzonitrile (1.0 g, 4.61 mmol) and iron powder (2.0 g, 35.7 mmol) in 30 mL of methyl alcohol and 10 mL of acetic acid is refluxed for 1 hour, then concentrated. The residue is diluted with ethyl acetate, and washed with water. The organic layer is dried over magnesium sulfate, and then filtered through Magnesol. Concentration provides 2-amino-4-pyrrolidin-1-ylbenzonitrile as a brick red solid (820 mg). MS: m/z 188.1 (M+H).

### Step B: 2-(2-Amino-4-pyrrolidin-1-ylphenyl)-4,5,6,7-tetrahydro-1,3-benzothiazol-7-ol

To a mixture of 2-amino-4-pyrrolidin-1-ylbenzonitrile (187 mg, 1.0 mmol) in 5 mL of methyl alcohol is added phosphorus pentasulfide (500 mg, 1.12 mmol). The mixture is stirred under nitrogen atmosphere at 60° C. for 18 hours, then concentrated. The residue is chromatographed over silica gel, eluting with a gradient of hexanes to 33% ethyl acetate in hexanes. Concentration provides 2-amino-4-pyrrolidin-1-ylbenzenecarbothioamide as a yellow solid (32 mg). A solution of 2-amino-4-pyrrolidin-1-ylbenzenecarbothioamide (32 mg, 0.14 mmol) and 7-oxabicyclo[4.1.0]heptan-2-one (100 mg, 0.89 mmol) in 2 mL of methyl alcohol is stirred at room temperature for 24 hours, then concentrated. The residue is chromatographed over silica gel, eluting with a gradient of 20% ethyl acetate in hexanes to 50% ethyl acetate in hexanes. Concentration provides 2-(2-amino-4-pyrrolidin-1-ylphenyl)-4,5,6,7-tetrahydro-1,3-benzothiazol-7-ol as a yellow solid (28 mg). MS: m/z 316.3 (M+H).

### Example 154

### Preparation of 2-[2-(methylthio)-4-pyrrolidin-1-ylphenyl]-4,5,6,7-tetrahydro-1,3-benzothiazol-7-ol, 2-[2-(Methylsulfinyl)-4-pyrrolidin-1-yl phenyl]-4,5,6,7-tetrahydro-1,3-benzothiazol-7-ol and 2-[2-(Methylsulfonyl)-4-pyrrolidin-1-ylphenyl]-4,5,6,7-tetrahydro-1,3-benzothiazol-7-ol

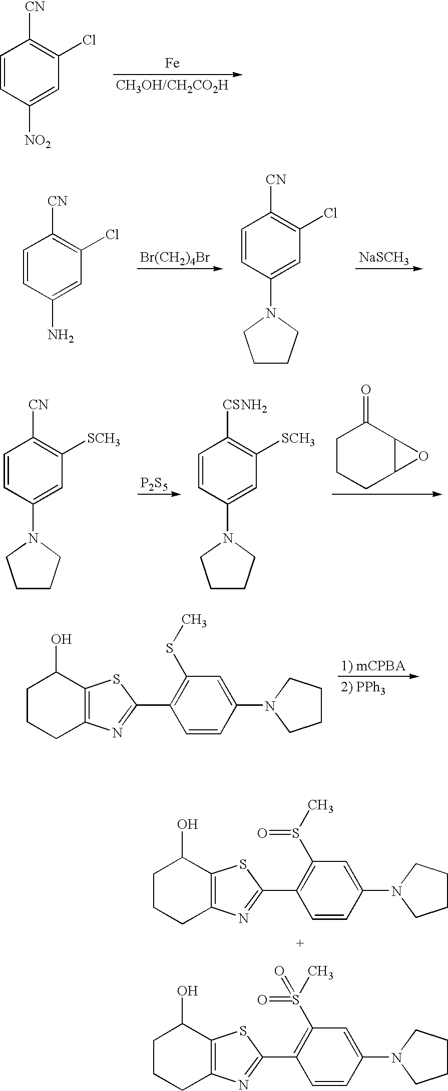

### Preparation of 2-[2-(methylthio)-4-pyrrolidin-1-ylphenyl]-4,5,6,7-tetrahydro-1,3-benzothiazol-7-ol

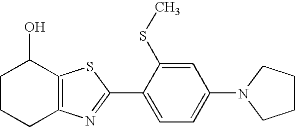

### Step A: 2-Chloro-4-pyrrolidin-1-ylbenzonitrile

A mixture of 4-amino-2-chlorobenzonitrile (810 mg, 5.31 mmol) and 1,4-dibromobutane (2.0 g, 9.26 mmol) in 30 mL of toluene and 2 mL of triethylamine is refluxed for 16 hours, then concentrated. The residue is diluted with ethyl acetate, and washed with saturated sodium chloride (×3). The organic layer is dried over magnesium sulfate and concentrated. The residue is chromatographed over silica gel, eluting with 50% ethyl acetate in hexanes. Concentration provides 2-chloro-4-pyrrolidin-1-ylbenzonitrile as a yellow solid (260 mg). MS: m/z 207.1 (M+H).

### Step B: 2-(Methylthio)-4-pyrrolidin-1-ylbenzonitrile

To a mixture of 2-chloro-4-pyrrolidin-1-ylbenzonitrile (206 mg, 1.0 mmol) in 10 mL of N,N-dimethylformamide is added sodium thiomethoxide (140 mg, 2.0 mmol). The mixture is stirred under nitrogen atmosphere at 70° C. until 2-chloro-4-pyrrolidin-1-ylbenzonitrile totally disappears. The mixture is concentrated and the residue is diluted with ethyl acetate, and filtered through Magnesol. Concentration provides 2-(methylthio)-4-pyrrolidin-1-ylbenzonitrile as a white solid (161 mg). MS: m/z 219.2 (M+H).

### Step C: 2-(Methylthio)-4-pyrrolidin-1-ylbenzenecarbothioamide

To a mixture of 2-(methylthio)-4-pyrrolidin-1-ylbenzonitrile (100 mg, 0.46 mmol) in 10 mL of ethyl alcohol is added phosphorus pentasulfide (500 g, 1.12 mmol). The mixture is stirred under nitrogen atmosphere at 40° C. for 18 hours, then concentrated. The residue is chromatographed over silica gel, eluting with a gradient of 20% ethyl acetate in hexanes to 50% ethyl acetate in hexanes. Concentration provides 2-(methylthio)-4-pyrrolidin-1-ylbenzenecarbothioamide as a yellow solid (32 mg). MS: m/z 253.2 (M+H).

### Step D: 2-[2-(Methylthio)-4-pyrrolidin-1-ylphenyl]-4,5,6,7-tetrahydro-1,3-benzothiazol-7-ol

A solution of 2-(methylthio)-4-pyrrolidin-1-ylbenzenecarbothioamide (51 mg, 0.20 mmol) and 7-oxabicyclo[4.1.0]heptan-2-one (200 mg, 1.78 mmol) in 10 mL of ethyl alcohol is stirred at room temperature for 2 hours, then concentrated. The residue is chromatographed over silica gel, eluting with a gradient of 20% ethyl acetate in hexanes to 50% ethyl acetate in hexanes. Concentration provides 2-[2-(methylthio)-4-pyrrolidin-1-ylphenyl]-4,5,6,7-tetrahydro-1,3-benzothiazol-7-ol as a yellow solid (61 mg). MS: m/z 347.3 (M+H).

### Example 155

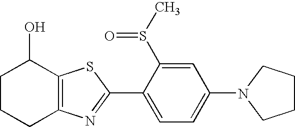

### 2-[2-(Methylsulfinyl)-4-pyrrolidin-1-yl phenyl]-4,5,6,7-tetrahydro-1,3-benzothiazol-7-ol

### Example 156

### Preparation of 2-[2-(Methylsulfonyl)-4-pyrrolidin-1-ylphenyl]-4,5,6,7-tetrahydro-1,3-benzothiazol-7-ol

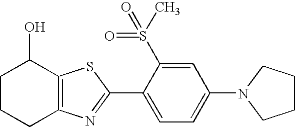

To a mixture of 2-[2-(methylthio)-4-pyrrolidin-1-ylphenyl]-4,5,6,7-tetrahydro-1,3-benzothiazol-7-ol (40 mg, 0.116 mmol) in 5 mL of dichloromethane is added 3-chloroperbenzoic acid in small portions until 2-[2-(methylthio)-4-pyrrolidin-1-ylphenyl]-4,5,6,7-tetrahydro-1,3-benzothiazol-7-ol totally disappears. Triphenylphosphine (50 mg, 0.19 mmol) is added, and the mixture is concentrated. The residue is chromatographed over silica gel, eluting with a gradient of 20% ethyl acetate in hexanes to 50% ethyl acetate in hexanes. Concentration provides 2-[2-(methylsulfinyl)-4-pyrrolidin-1-ylphenyl]-4,5,6,7-tetrahydro-1,3-benzothiazol-7-ol as a yellow solid (15 mg), MS: m/z 363.3 (M+H), and 2-[2-(methylsulfonyl)-4-pyrrolidin-1-ylphenyl]-4,5,6,7-tetrahydro-1,3-benzothiazol-7-ol as a yellow oil (13 mg), MS: m/z 379.3 (M+H).

### Example 157

### Preparation of 2-[4-(3-hydroxy-azetidin-1-yl)-phenyl]-4,5,6,7-tetrahydro-benzothiazol-7-ol

Example 157 was synthesized analogously to Example 143.

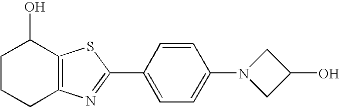

2-[4-(3-hydroxy-azetidin-1-yl)-phenyl]-4,5,6,7-tetrahydro-benzothiazol-7-ol, MS: m/z=303.1 (M+H)

### Example 158

### Preparation of 2-(4-pyrrolidin-1-yl-phenyl)-4,5,6,7-tetrahydro-benzothiazol-7-ol

The following reaction sequence was used to prepare 2-(4-pyrrolidin-1-yl-phenyl)-4,5,6,7-tetrahydro-benzothiazol-7-ol and mass spectral analysis was performed.

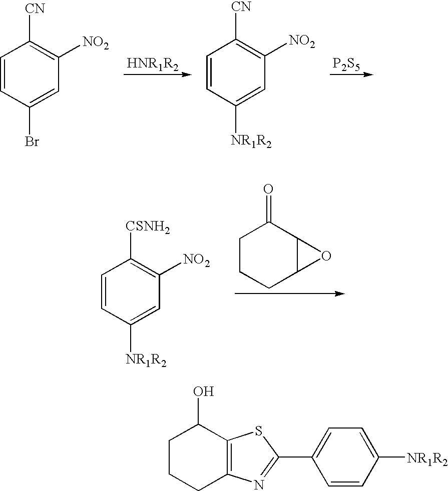

### Step A: 4-pyrrolidin-1-yl-benzonitrile

A mixture of 4-bromobenzonitrile (2.0 g, 11.6 mmol), pyrrolidine (1.84 ml, 21.7 mmol), Pd(OAc)₂ (99 mg, 0.44 mmol), 2,8,9-triisobytul-2,5,8,9-tetraaza-1-phosphabicyclo[3.3.3]undecane (313 ul, 0.88 mmol), and sodium tert-butoxide (1.6 g, 16.6 mmol) in 20 ml of DMF was stirred at 120° C. for 2 hours. The resulting reaction mixture was diluted with ethyl acetate, washed with water and saturated NaCl aqueous solution, dried over MgSO₄, filtered and then concentrated. The residue was chromatographed over silica gel, eluting with 40% ethyl acetate in hexane to provide 1.5 g of 4-pyrrolidin-1-yl-benzonitrile as a tan solid.

Steps B and C of Example 158 were carried out as in steps B and C of Example 154.

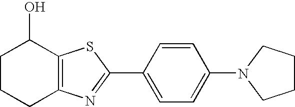

2-(4-pyrrolidin-1-yl-phenyl)-4,5,6,7-tetrahydro-benzothiazol-7-ol, MS: m/z=301.1 (M+H)

### Example 159

### Preparation of 2-[4-(3-dimethylamino-pyrrolidin-1-yl)-phenyl]-4,5,6,7-tetrahydro-benzothiazol-7-ol

Example 159 was synthesized analogously to Example 158.

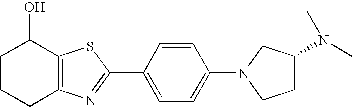

2-[4-(3-dimethylamino-pyrrolidin-1-yl)-phenyl]-4,5,6,7-tetrahydro-benzothiazol-7-ol, MS: m/z=344.1 (M+H)

### Example 160

### Preparation of 2-[4-(2-dimethylaminomethyl-pyrrolidin-1-yl)-phenyl]-4,5,6,7-tetrahydro-benzothiazol-7-ol

Example 160 was synthesized analogously to Example 158.

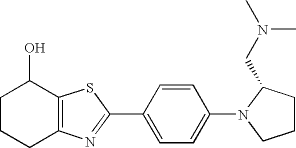

2-[4-(2-dimethylaminomethyl-pyrrolidin-1-yl)-phenyl]-4,5,6,7-tetrahydro-benzothiazol-7-ol, MS: m/z=358.2 (M+H)

### Example 161

### Preparation of 2-(7-Hydroxy-4,5,6,7-tetrahydro-benzothiazol-2-yl)-5-pyrrolidin-1-yl-benzoic acid methyl ester

The following reaction sequence was used to prepare 2-(7-hydroxy-4,5,6,7-tetrahydrobenzothiazol-2-yl)-5-pyrrolidin-1-yl-benzoic acid methyl ester:

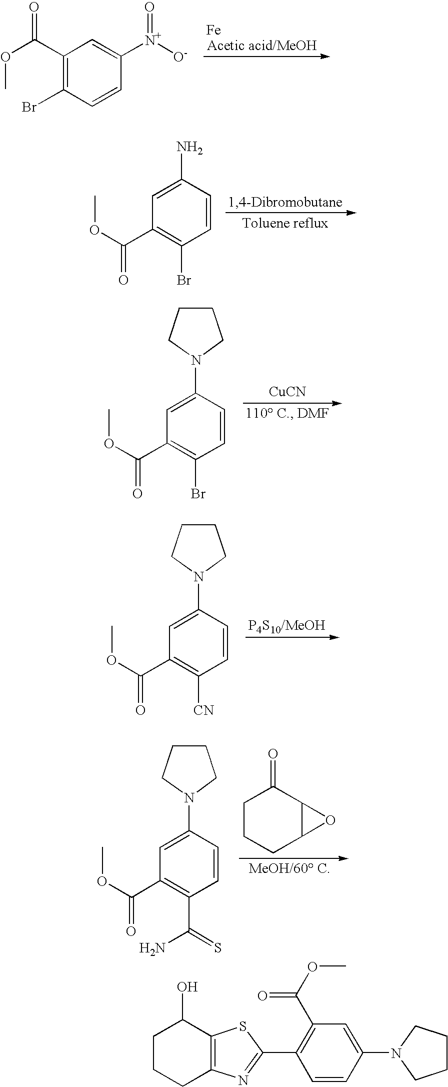

Step 1: 5-Amino-2-bromo-benzoic acid methyl ester (CL-426403). A reaction mixture of 2-bromo-5-nitro-benzoic acid methyl ester (10 g=38.46 mmol), iron (3.5 g=62.5 mmol), 50 ml of acetic acid and 50 ml of methanol was stirred at 110° C. for 1 hour, than evaporated. The residue was stirred with 50 ml of ethyl acetate and then filtered. The filtrated was washed with Na₂CO₃ aqueous solution (PH=9-10) and brine solution, dried over MgSO₄, filtered, concentrated and then dried in-vacuo to give 9.31 g of 5-amino-2-bromo-benzoic acid methyl ester as light yellow oil. MS: m/z=230.0, 232.0 (M+H).

Step 2: 2-Bromo-5-pyrrolidin-1-yl-benzoic acid methyl ester (WAY-284799). A reaction mixture of 5-amino-2-bromo-benzoic acid methyl ester (5.0 g=21.73 mmol), 4-dibromobutane (5.21 ml=43.47 mmol), triethylamine (5.77 ml=43.47 mmol) and toluene (50 ml) was stirred at 100° C. for 2 hours, then evaporated. The residue was dissolved in 50 ml of dichloromethane, washed with NaHCO₃ aqueous solution and brine solution, dried over MgSO₄, filtered, concentrated and chromatographed over a 120 g silica column, eluting with 30% ethylacetate in hexane to provide 2.18 g of 2-bromo-5-pyrrolidin-1-yl-benzoic acid methyl ester as a yellow solid. MS: m/z=284.1, 286.1 (M+H).

Step 3: 2-Cyano-5-pyrrolidin-1-yl-benzoic acide methyl ester. A reaction mixture of 2-bromo-5-pyrrolidin-1-yl-benzoic acid methyl ester (7.1 g=24.9 mmol), copper(I)cyanide (7.2 g=49.97 mmol) and DMF (100 ml) was stirred at 110° C. for 3 hours. The resulting reaction mixture was diluted with ethyl acetate (200 ml), washed with NaHCO₃ aqueous solution and brine solution, dried over MgSO₄, filtered, then concentrated and chromatographed over a 120 g silica column, eluting with 50% ethyl acetate in hexane to provide 4.9 g of 2-cyano-5-pyrrolidin-1-yl-benzoic acid methyl ester as a light yellow solid. MS: m/z=231.2 (M+H).

Step 4: 5-Pyrrolidin-1-yl-2-thiocarbamoyl-benzoic acid methyl ester. Into a solution of 2-cyano-5-pyrrolidin-1-yl-benzoic acid methyl ester (4.89 g=21.26 mg) in 15 ml of methanol at 0° C., P₄S₁₀ (9.47 g=21.26 mmol) was added in portion. The reaction was then stirred at room temperature for 12 hours. The precipitate was filtered, washed with 50% of dichloromethane in hexane and dried in vacuo to obtain 4.2 g of 5-pyrrolidin-1-yl-2-thiocarbamoyl-benzoic acid methyl ester as a red solid. This product was used in the next step without further purification.

Step 5: 2-(7-Hydroxy-4,5,6,7-tetrahydro-benzothiazol-2-yl)-5-pyrrolidin-1-yl-benzoic acid methyl ester. A solution mixture of 5-pyrrolidin-1-yl-2-thiocarbamoyl-benzoic acid methyl ester (1.0 g=3.94 mmol) and 7-oxabicyclo[4.1.0]heptan-2-one (1.57 ml=15.8 mmol) and methanol (10 ml) was stirred at 60° C. for 14 hours, then concentrated. The residue was chromatographed over a 40 g silica column, eluting with 20% acetonitrile in dichloromethane to provide 529.5 mg of 2-(7-hydroxy-4,5,6,7-tetrahydro-benzothiazol-2-yl)-5-pyrrolidin-1-yl-benzoic acid methyl ester as a yellow solid. MS: m/z=359.1 (M+H).

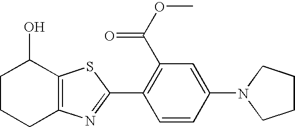

2-(7-Hydroxy-4,5,6,7-tetrahydro-benzothiazol-2-yl)-5-pyrrolidin-1-yl-benzoic acid methyl ester, MS: m/z=359.1 (M+H)

### Example 162

### Preparation of 2-(2-Hydroxymethyl-4-pyrrolidin-1-yl-phenyl)-4,5,6,7-tetrahydro-benzothiazol-7-ol

The following reaction sequence was used to prepare 2-(2-hydroxymethyl-4-pyrrolidin-1-yl-phenyl)-4,5,6,7-tetrahydro-benzothiazol-7-ol

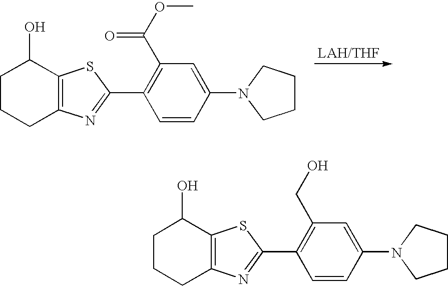

Into a solution of 2-(7-hydroxy-4,5,6,7-tetrahydro-benzothiazol-2-yl)-5-pyrrolidin-1-yl-benzoic acid methyl ester (30 mg=0.08 mmol) in 2 ml of THF, LAH (6.3 mg=0.16 mmol) was added in portions. The reaction was stirred at room temperature for 6 hours, then quenched with water, diluted with ethyl acetate, washed with brine solution, dried over Na₂SO₄, filtered, concentrated and chromatographed over a 12 g silica column (eluting with 10% acetonitrile in dichloromethane) to provide 13 mg of 2-(2-hydroxymethyl-4-pyrrolidin-1-yl-phenyl)-4,5,6,7-tetrahydro-benzothiazol-7-ol as a light yellow solid. MS: m/z=331.1 (M+H).

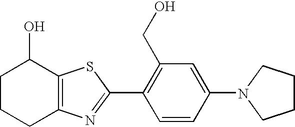

2-(2-Hydroxymethyl-4-pyrrolidin-1-yl-phenyl)-4,5,6,7-tetrahydro-benzothiazol-7-ol,

### Example 163

### Preparation of 2-(4-piperazin-1-yl-phenyl)-4,5,6,7-tetrahydrobenzothiazol-7-ol

The following reaction sequence was used to prepare 2-(4-piperazin-1-yl-phenyl)-4,5,6,7-tetrahydrobenzothiazol-7-ol

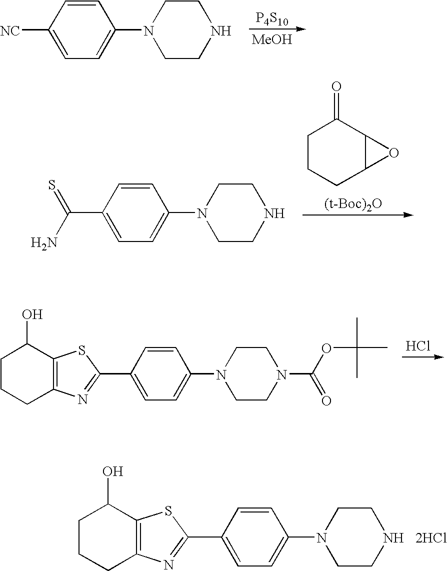

Step 1: 4-piperazin-1-yl-thiobenzamide. Into a solution of 1.(4-cyanophenyl)piperazin (550 mg=2.9 mmol) in methanol at 0° C., P₄S₁₀ (1.29 g=2.9 mmol) was added in portion. The reaction was stirred at room temperature for 14 hours. The precipitate was filtered, washed with 50% dichloromethane in hexane and then dried in-vacuo to provide 800.0 mg of 4-piperazin-1-yl-thiobenzamide as a yellow solid. This product was used in the next step without further purification.

Step 2: 4-[4-(7-Hydroxy-4,5,6,7-tetrahydro-benzothazol-2-yl-phenyl]-piperazine-1-carboxylic acid tert-butyl ester. A solution mixture of 4-piperazin-1-yl-thiobenzamide (400 mg=1.4 mmol), 7-oxalobicyclo[4.1.0]heptane-2-one (556 μL=56 mmol) and methanol (10 ml) was stirred at 60° C. for 14 hours, then concentrated. Into the solution of the above residue with 10 ml of dichloromethane, (t-Boc)₂O (480=2.2 mmol) was added in portion. The reaction was stirred at room temperature for 3 hours, concentrated, and then chromatographed over a 40 g silica column (eluting with 20% acetonitrile in dichloromethane) to provide 138.3 mg of 4-[4-(7-hydroxy-4,5,6,7-tetrahydro-benzothazol-2-yl-phenyl]-piperazine-1-carboxylic acid tert-butyl ester as a light yellow solid. MS: m/z=416.4 (M+H).

Step 3: 2-(4-piperazin-1-yl-phenyl)-4,5,6,7-tetrahydrobenzothiazol-7-ol. Into a solution of 4-[4-(7-hydroxy-4,5,6,7-tetrahydro-benzothazol-2-yl)-phenyl]-piperazine-1-carboxylic acid tert-butyl ester (110 mg=0.26 mmol) in 10 ml of dichloromethane, 4N HCl in dioxane (600 μL) was added dropwise. The reaction was stirred at room temperature for 14 hours, then filtered, washed with dichloromethane, then dried in-vacuo to obtain 90 mg of 2-(4-piperazin-1-yl-phenyl)-4,5,6,7-tetrahydrobenzothiazol-7-ol HCl salt as a yellow solid. MS: m/z=316.2 (M+H).

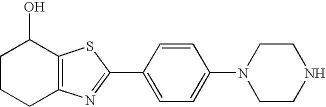

2-(4-piperazin-1-yl-phenyl)-4,5,6,7-tetrahydrobenzothiazol-7-ol, MS: m/z=316.2 (M+H)

### Example 164

### Chiral synthesis of (7R)-2-[4-(dimethylamino)phenyl]-4,5,6,7-tetrahydro-1,3-benzothiazol-7-ol

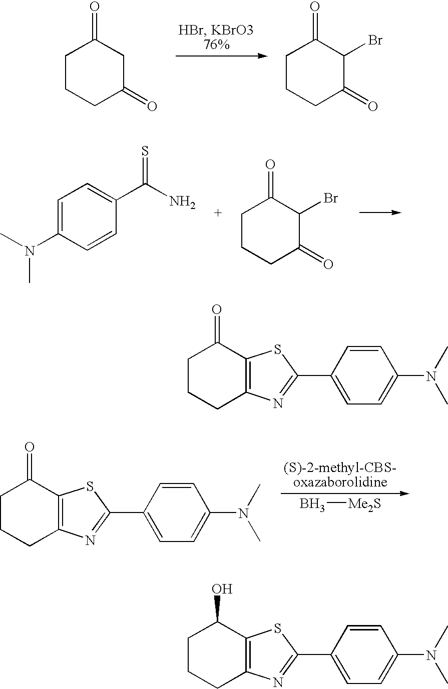

### Step A. 2-Bromo-cyclohexane-1,3-dione

To a suspension of cyclohexane-1,3-dione (11.2 g, 0.1 mol) in 48% aq. HBr (15 mL) and water (50 mL) was added a solution of potassium bromate (5.7 g, 0.034 mol) in water (50 mL) at room temperature. After stirring for 1 hour, the suspension was filtered and then washed with water to give 14.5 g (76%) of 2-bromo-cyclohexane-1,3-dione as a white solid. MS: m/z 191 (M+H)⁺.

### Step B. 2-(4-Dimethylaminophenyl)-5,6-dihydro-7(4H)-benzothiazolone

To a solution of 4-dimethylaminophenylthioamide (0.354 g, 1.966 mol) and THF (25 mL) was added 2-bromo-cyclohexane-1,3-dione (0.3756 g, 1.966 mol). The mixture was heated at 60° C. for 3 hours. After it was cooled, it was filtered and washed with THF to yield 416 g of the crude product. It was dissolved in ethyl acetate and washed with water. The organic was dried to yield 369 mg (69%) of 2-(4-dimethylaminophenyl)-5,6-dihydro-7(4H)-benzothiazolone as a yellow solid. MS: m/z 273.2 (M+H)⁺.

### Step C. (7R)-2-[4-(dimethylamino)phenyl]-4,5,6,7-tetrahydro-1,3-benzothiazol-7-ol

To a solution of (S)-2-methyl-CBS-oxazaborolidine (1 mL, 1.16 mmol, 1M in toluene) and borane-dimethylsulfide (1.5 mL, 3.5 mmol, 2M in THF) was added a solution of 2-(4-dimethylaminophenyl)-5,6-dihydro-7(4H)-benzothiazolone (232 mg, 0.85 mmol) and THF (47 mL) over 25 min. After it was stirred for 1 hour, the solution was cooled to 0° C. Then methanol (25 mL) was added, then the solution was stirred at room temperature overnight. It was evaporated to dryness, and then ether was added. The precipitate was filtered to yield 220 mg (95% yield) of the product as a white solid. MS: m/z 275.1 (M+H)⁺. Enantiomeric purity was determined by chiral HPLC (HP1100-6, Chiralpak™ ADH, 0.46×25 cm, mobile phase: heptane/isopropanol (80/20), flow rate: 1.0 mL/min, detected at 340 nm). The desired R-enantiomer was eluted at 7.6 min (99.1%), whereas the corresponding S-enantiomer was eluted at 11.9 min (0.9%). The percent enantiomeric excess (% ee) of the product was 98.2%.

### Example 165

### Chiral Synthesis of methyl 5-(dimethylamino)-2-[(7R)-7-hydroxy-4,5,6,7-tetrahydro-1,3-benzothiazol-2-yl]benzoate

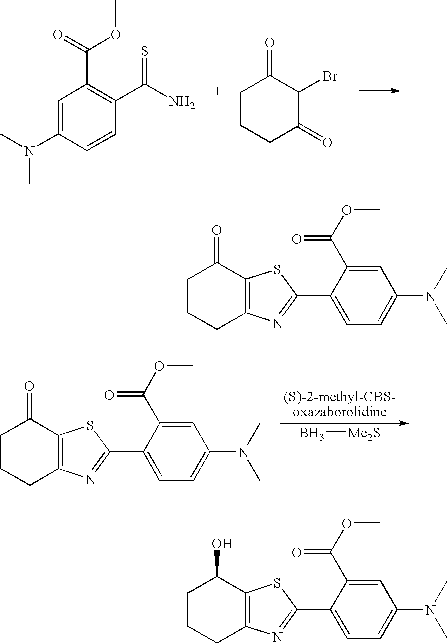

### Step A. Methyl 5-(dimethylamino)-2-(7-oxo-4,5,6,7-tetrahydro-1,3-benzothiazol-2-yl)benzoate

To a solution of 2-bromo-cyclohexane-1,3-dione (100 mg, 0.52 mmol) and THF (2.6 mL) was added methyl 2-(aminocarbonothioyl)-5-(dimethylamino)benzoate (0.224 g, 0.94 mol). After the reaction mixture was stirred at room temperature overnight, it was filtered to yield 139 mg of crude product as a light yellow solid. The compound was purified by preparative TLC (developed with 40% ethyl acetate in hexane) to yield 40 mg (25% yield) of the product as a yellow solid. MS: m/z 331.1 (M+H)⁺.

### Step B. Methyl 5-(dimethylamino)-2-[(7R)-7-hydroxy-4,5,6,7-tetrahydro-1,3-benzothiazol-2-yl]benzoate

To a solution of (S)-2-methyl-CBS-oxazaborolidine (0.035 mL, 0.035 mmol, 1M in toluene) and borane-dimethylsulfide (0.052 mL, 0.105 mmol, 2M in THF) was added a solution of methyl 5-(dimethylamino)-2-(7-oxo-4,5,6,7-tetrahydro-1,3-benzothiazol-2-yl)benzoate (8.7 mg, 0.026 mmol) and THF (1.45 mL) over 18 minutes. After the solution was stirred for 35 minutes, it was cooled to 0° C. Then methanol (1 mL) was added, then the solution was stirred at room temperature overnight. It was evaporated to dryness, and purified by preparative TLC (developed with 5% methanol in chloroform) to yield 4 mg (46% yield) of the product as a yellow solid. MS: m/z 333.2 (M+H)⁺. Enantiomeric purity was determined by chiral HPLC (HP1100-6, Chiralpak ADH, 0.46×25 cm, mobile phase: heptane/isopropanol (80/20), flow rate: 1.0 mL/min, detected at 340 nm). The desired R-enantiomer was eluted at 14.3 min (100%), and showed CD(−) at 220 nm. The % ee of the product was 100%.

Examples 164, 166 and 167 were prepared using analogous procedures described in the preparation of Examples 5 and 6.

### Examples 166 and 167

### Preparation of 2-[4-(dimethylamino)phenyl]-4,5,6,7-tetrahydro-1,3-benzothiazol-7-ol and (7S)-2-[4-(dimethylamino)phenyl]-4,5,6,7-tetrahydro-1,3-benzothiazol-7-ol

### Examples 168 and 169

### Preparation of 2-[4-(dimethylamino)-1-naphthyl]-4,5,6,7-tetrahydro-1,3-benzothiazol-7-ol and [4-(7-ethoxy-4,5,6,7-tetrahydro-1,3-benzothiazol-2-yl)-1-naphthyl]dimethylamine

The following reaction sequence was used to prepare Examples 168-169 and mass spectral analysis was performed on each of the compounds.

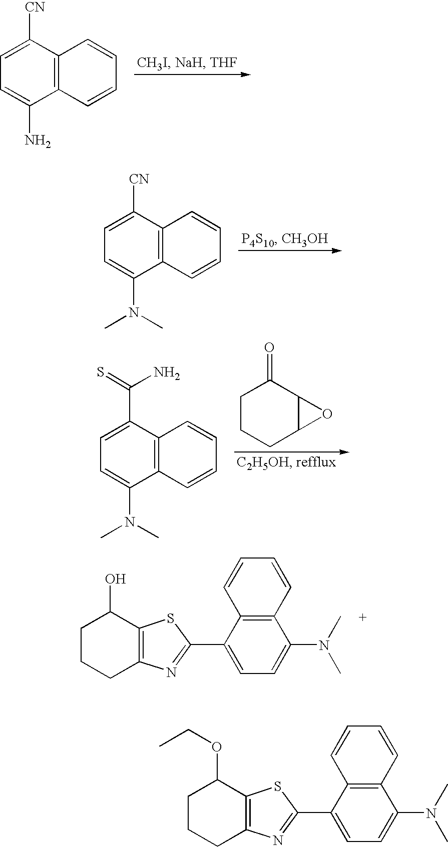

Step A. The compound 4-(dimethylamino)-1-naphthonitrile was prepared by following General Procedure I.

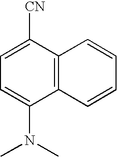

4-(dimethylamino)-1-naphthonitrile, MS: m/z=197 (M+H)

Step B. 4-(dimethylamino)naphthalene-1-carbothioamide was prepared by following General Procedure II.

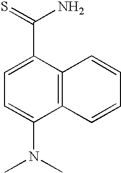

4-(dimethylamino)naphthalene-1-carbothioamide, MS: m/z=231 (M+H)

Step C. 2-[4-(dimethylamino)-1-naphthyl]-4,5,6,7-tetrahydro-1,3-benzothiazol-7-ol and [4-(7-ethoxy-4,5,6,7-tetrahydro-1,3-benzothiazol-2-yl)-1-naphthyl]dimethylamine was prepared by following General Procedure I.

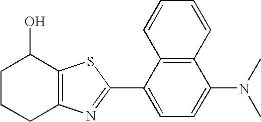

2-[4-(dimethylamino)-1-naphthyl]-4,5,6,7-tetrahydro-1,3-benzothiazol-7-ol, MS: m/z=325 (M+H)

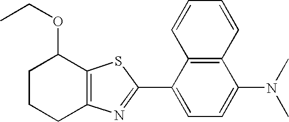

[4-(7-ethoxy-4,5,6,7-tetrahydro-1,3-benzothiazol-2-yl)-1-naphthyl]dimethylamine, MS: m/z=353 (M+H)

### Pharmacological Testing

IC₅₀ analysis was performed for selected fused bicyclic 2-aryl or 2-heteroarylthiazolyl compounds that were tested on HCT116 cells and Securin “knock out” cell lines. A securin screen was performed on selected fused bicyclic 2-aryl- or 2-heteroarylthiazolyl compounds to find compounds that preferentially inhibited the growth of the securin knockout cells, but did not inhibit the growth of securin parental cells (HCT116 FB).

Cell Culture—HCT116 and securin “knock out” cells. HCT116 and securin “knock out” cells (D8 and F3) were grown in RPMI+5% fetal bovine serum and gentamycin. They were maintained in a humidified 37° C. incubator with 5% CO₂. Since the securin “knock out” cells have active chromosomal instability, cells were used at a low passage number of 2-13 passages. At or before passage 13 a new vial of cells was thawed and used for the screen. Cell seeding—HCT116 cells and securin “knock out” cells were trypsinized and suspended in RPMI+5% FBS at a density of 27,000 cells/mL. HCT116 and securin “knock out” cells were seeded separately in wells of a 96-well plate (Falcon Cat # 35-3872) at a cell density of 4000 cells per well. Cells were seeded in 150 μL of RPMI media containing 5% FBS and gentamycin. Cell seeding was performed under aseptic conditions. Cells were allowed to attach and grow overnight in a humidified 37° C. incubator.

Compound Dilution and Dispensation. A series of stock solutions, 10 mg/mL of fused bicyclic 2-aryl- or 2-heteroarylthiazolyl compounds to be tested, were brought to room temperature. The compounds were diluted 1:50 in RPMI+5% FBS (Highest concentration 200 μg/mL). The compounds were then serially diluted 1:3 across a deep-well titration plate resulting in final concentrations in the plate of 200, 66.7, 22.2, 7.4, 2.4, 0.8, 0.27, 0.091, 0.03, 0.01, and 0.0034 μg/mL with one well left without a compound. 50 μL of this dilution series was added to the 150 μL of cells to result in a final concentration of compound in with the cells of 50, 16.6, 5.5, 1.8, 0.6, 0.2, 0.068, 0.023, 0.0076, 0.0025, and 0.00084 μg/mL. The dilution and addition of compounds to the plates is performed using a Multimek 96 Robot (Beckman Coulter).

Incubation and analysis. After dosing, the cells in the 96 well plates were returned to the humidified 37° C. incubator. The compounds remained on the cells for 5 days. After 5 days, the cells were fixed to the plates with the addition of 50 μL of 50% (vol/vol) Trichloro Acetic Acid (1% final concentration). The cells were fixed at 4° C. for 1 hour. The plates were washed 5 times in distilled water and allowed to dry. The cells were stained with 0.4% sulforhodamine B (Sigma) in 1% acetic acid. The cells were stained for 15 minutes at room temperature. The plates were then washed 3 times in 1% acetic acid. The plates were dried and the dye was solubilized in 10 mM Tris at pH 8.0. The plates were read on the Absorbence 560 setting on a Victor² V Model 1420 Multilabel HTS counter (Perkin Elmer).

Analysis of Results—IC₅₀ data. Absorbence data was read off of the Victor and was converted to a percentage of the control (untreated) values. IC₅₀ values were graphed and the point at which a 50% inhibition of the control level was determined. A hyperbolic model that forces a curve between 0% inhibition and 100% inhibition was used. If the model was not able to determine an IC₅₀, the IC₅₀ was assigned to the lowest concentration that gives a 50% or greater decrease in absorbence compared to control levels. If the calculated IC₅₀ value for a cell line exceeds the top concentration for the assay (typically 50 μg/mL), the assay was scored as >50.

Selectivity Ratio. The Selectivity Ratio is the ratio of IC₅₀s between the parent cell line and the knock out cell line and is always a positive number and is typically an integer.

$$Selectivity Ratio = \frac{{IC}_{50} HCT 116 FB ( µg \text{/} mL )}{{IC}_{50} Securin knockout ( µg \text{/} mL )}$$

IC₅₀ data and selectivity ratios of selected fused bicyclic 2-aryl- and 2-heteroarylthiazolyl compounds are summarized in Table 1.

TABLE 1
IC₅₀ data and selectivity ratios of selected fused bicyclic 2-aryl- and 2-
heteroarylthiazolyl compounds of formula I.

| | IC₅₀ (μM) | IC₅₀ (μM) | IC₅₀ (μM) | Selectivity | |
|---|---|---|---|---|---|
| Example | Securin^{+/+} | Securin^{-/-} | Securin^{-/-} | Ratio | |
| No. | HCT116 | D8 | F3 | D8 | F3 |
| 5 | 54.3 | 1.3 | 1.8 | 43 | 30 |
| 6 | 43.4 | 0.46 | 0.84 | 95 | 52 |
| 25 | 161 | 2.6 | 6.7 | 62 | 24 |
| 45 | 133 | 0.54 | 1.1 | 245 | 121 |
| 47 | 76.4 | 0.43 | 3.0 | 179 | 26 |
| 55 | 105 | 0.65 | 1.2 | 160 | 90 |
| 60 | 97.5 | 0.091 | 0.12 | 1076 | 821 |
| 67 | 31.0 | 0.059 | 0.15 | 525 | 206 |
| 72 | 54.0 | 1.0 | 2.0 | 52 | 27 |
| 73 | 99.5 | 0.44 | 0.79 | 226 | 126 |
| 74 | 100 | 0.15 | 0.18 | 650 | 558 |
| 81 | 58.9 | 0.13 | 0.35 | 459 | 170 |
| 85 | 48.4 | 0.19 | 0.31 | 253 | 159 |
| 99 | 173 | 0.24 | 0.21 | 709 | 810 |
| 111 | 100 | 1.3 | 1.4 | 75 | 71 |
| 112 | 97.3 | 0.54 | 0.72 | 182 | 135 |
| 116 | 85.2 | 0.59 | 0.82 | 145 | 104 |
| 121 | 77.3 | 0.69 | 1.7 | 112 | 44 |
| 122 | 42.2 | 0.76 | 2.1 | 55 | 20 |
| 123 | 90.0 | 1.7 | 4.4 | 52 | 20 |
| 126 | 100 | 1.9 | 2.5 | 54 | 40 |
| 127 | 100 | 4.9 | 6.3 | 20 | 16 |
| 128 | 86.7 | 1.9 | 2.6 | 45 | 33 |
| 129 | 77.8 | 0.36 | 0.45 | 216 | 172 |
| 130 | 77.6 | 2.0 | 2.4 | 38 | 32 |
| 133 | 96.5 | 1.1 | 1.8 | 87 | 54 |
| 140 | 37.2 | 0.57 | 1.8 | 66 | 21 |
| 151 | 38.0 | 0.046 | 0.053 | 832 | 724 |
| 155 | 38.8 | 0.099 | 0.17 | 390 | 224 |
| 158 | 154 | 1.7 | 2.9 | 91 | 54 |
| 161 | 66.3 | 0.40 | 0.52 | 165 | 128 |
| 162 | 15.3 | 0.050 | 0.046 | 304 | 333 |
| 163 | 31.6 | 25.9 | 22.9 | 1 | 1 |
| 164 | 101 | 0.81 | 1.3 | 124 | 78 |
| 165 | 100 | 0.29 | 0.74 | 348 | 136 |
| 166 | 52.5 | 1.7 | 3.5 | 30 | 15 |
| 167 | 100 | 60.0 | 70.7 | 2 | 1 |

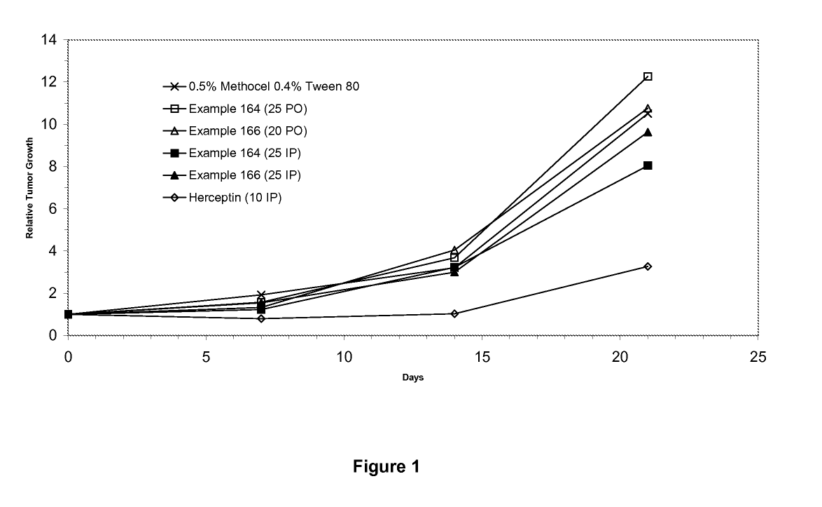

In-vivo Activity in Tumor Xenografts. The effectiveness of Examples 164, 166 and 167 versus Herceptin at controlling tumor growth was studied and the results are summarized in Table 2 and FIG. 1. The fused bicyclic 2-arylthiazolyl compounds were formulated using 0.5% Methocel™ (methylcellulose) and 0.4% Tween 80™ (polysorbate-80 and oleic acid) and administered daily orally (PO), by gavage or by syringe. Tumor mass [(length×width²)/2] was determined every 7 days. Inhibition of tumor growth was calculated relative to conventional (vehicle treated) controls.

TABLE 2
Effectiveness of Selected Fused Bicyclic 2-Arylthiazolyl Compounds versus
Herceptin in Xenografts of a Human Breast Carcinoma Cell Line (MDA-MB-361)

| a | | | | b | | | | c | | |
|---|---|---|---|---|---|---|---|---|---|---|
| Drug Treatment | b | c | d | Day | c | d | b | % | d | e |
| mg/kg/dose | Day 7 | % T/C | (p) | 14 | % T/C | (p) | Day 21 | T/C | (p) | S/T |
| 0.5% Methocel | 1.93 | | | 3.21 | | | 10.51 | | | 15/15 |
| 0.4% Tween 80 ™ | | | | | | | | | | |
| EXAMPLE 164 (25 PO) | 1.57 | 81 | 0.13 | 3.68 | 115 | 0.76 | 12.27 | 117 | 0.74 | 5/5 |
| EXAMPLE 167 (20 PO) | 0.89 | 46 | <0.01 | 1.96 | 61 | 0.12 | 6.39 | 61 | 0.15 | 5/5 |
| EXAMPLE 166 (20 PO) | 1.33 | 69 | 0.01 | 4.05 | 126 | 0.95 | 10.76 | 102 | 0.68 | 10/10 |
| EXAMPLE 164 (25 IP) | 1.23 | 64 | <0.01 | 3.23 | 101 | 0.44 | 8.04 | 74 | 0.19 | 4/5 |
| EXAMPLE 166 (25 IP) | 1.55 | 80 | 0.10 | 3.00 | 93 | 0.47 | 9.63 | 92 | 0.31 | 8/10 |
| HKI-272 (40 PO) | 1.19 | 62 | <0.01 | 2.86 | 89 | 0.33 | 8.03 | 76 | 0.21 | 10/10 |
| EKB-569 (40 PO) | 1.28 | 66 | 0.01 | 2.57 | 80 | 0.24 | 8.24 | 78 | 0.16 | 10/10 |
| Herceptin (10 IP) | 0.80 | 41 | <0.01 | 1.03 | 32 | <0.01 | 3.27 | 31 | <0.01 | 10/10 |

a) Herceptin administered on days 1, 5, 9 and 13. EXAMPLE 164 (25 IP) and EXAMPLE 166 (25 IP) administered on days 1, 3, 5 . . . 19. All other compounds administered on days 1 through 20.
b) Relative Tumor Growth = Mean Tumor Mass on Day 7, 14, 21/Mean Tumor Mass on Day 0
c) % T/C = Relative Tumor Growth of Treated Group/Relative Tumor Growth of Placebo Group (×100)
d) Statistical Analysis (Student's T-test) of Log Relative Tumor Growth. A p-value (p ≦ 0.05) indicates a statistically significant reduction in Relative Tumor Growth of Treated Group compared to the Placebo Control.
e) S/T = # of Survivors/# of Treated on Day +21 post tumor staging.

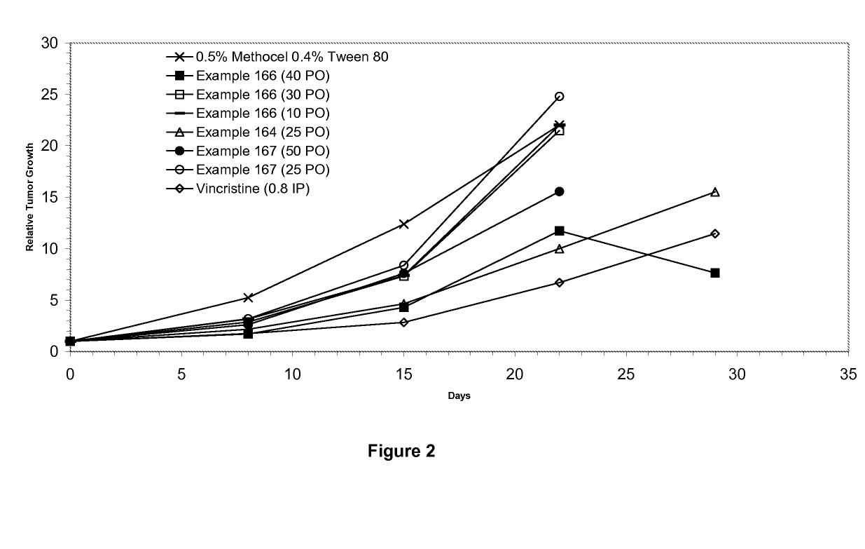

The effectiveness of Examples 164, 166 and 167 versus Vincristine at controlling tumor growth was studied and the results are summarized in Table 3 and FIG. 2.

TABLE 3
Effectiveness of Selected Fused Bicyclic 2-Arylthiazolyl Compounds versus
Vincristine in Xenografts of a Human Breast Carcinoma Cell Line (MDA-MB-361)

| a | | c | | | c | | | c | | | b | |
|---|---|---|---|---|---|---|---|---|---|---|---|---|
| Drug Treatment | b | % | d | b | % | d | b | % | d | b | Day | e |
| mg/kg/dose | Day 8 | T/C | (p) | Day 15 | T/C | (p) | Day 22 | T/C | (p) | Day 29 | 36 | S/T |
| 0.5% Methocel | 5.24 | | | 12.39 | | | 22.03 | | | — | — | 0/15* |
| 0.4% Tween 80 | | | | | | | | | | | | |
| EXAMPLE 166 (50 PO) | 3.04 | 58 | <0.01 | 7.67 | 62 | 0.04 | Toxic | — | — | — | — | 0/5 |
| EXAMPLE 166 (40 PO) | 1.72 | 33 | <0.01 | 4.29 | 35 | <0.01 | 11.74 | 53 | 0.01 | 7.65 | — | 0/5* |
| EXAMPLE 166 (30 PO) | 2.87 | 55 | <0.01 | 7.34 | 59 | 0.01 | 21.48 | 98 | 0.53 | — | — | 0/5* |
| EXAMPLE 166 (10 PO) | 3.16 | 60 | <0.01 | 7.40 | 60 | 0.01 | 22.01 | 100 | 0.48 | — | — | 0/5* |
| EXAMPLE 164 (50 PO) | 2.37 | 45 | <0.01 | Toxic | — | — | — | — | — | — | — | 0/5 |
| EXAMPLE 164 (25 PO) | 2.17 | 41 | <0.01 | 4.64 | 37 | <0.01 | 10.02 | 45 | 0.01 | 15.52 | — | 0/5* |
| EXAMPLE 167 (50 PO) | 2.62 | 50 | <0.01 | 7.61 | 62 | <0.01 | 15.56 | 71 | 0.09 | — | — | 0/5* |
| EXAMPLE 167 (25 PO) | 3.18 | 61 | <0.01 | 8.38 | 68 | 0.04 | 24.80 | 113 | 0.75 | — | — | 0/5* |
| SKI-606 20 (150 PO) | 2.09 | 40 | <0.01 | 2.77 | 22 | <0.01 | 3.62 | 16 | <0.01 | 7.22 | 10.19 | 10/10 |
| SKI-606 20 | 1.97 | 52 | <0.01 | 2.44 | 27 | <0.01 | 3.91 | 18 | <0.01 | 5.73 | 10.26 | 8/10 |
| (100 PO, BID) | | | | | | | | | | | | |
| 0.5% Methocel | 3.72 | | | 8.92 | | | 21.98 | | | — | — | 0/10* |
| 0.4% Tween 80 (BID) | | | | | | | | | | | | |
| Vincristine (0.8 IP) | 1.75 | 33 | <0.01 | 2.84 | 23 | <0.01 | 6.71 | 30 | <0.01 | 11.47 | | 0/5* |

a) Vincristine administered on days 2, 5 and 10. All other compounds administered on days 2 through 21.
b) Relative Tumor Growth = Mean Tumor Mass on Day 8, 15, 22, 29, 36/Mean Tumor Mass on Day 0
c) % T/C = Relative Tumor Growth of Treated Group/Relative Tumor Growth of Placebo Group × 100
d) Statistical Analysis (Student's T-test) of Log Relative Tumor Growth. A p-value (p ≦ 0.05) indicates a statistically significant reduction in Relative Tumor Growth of Treated Group compared to the Placebo Control.
e) S/T = # of Survivors/# of Treated on Day +36 post tumor staging. *Sacrificed earlier due to tumor size

What is claimed is:

1. A compound of formula I: 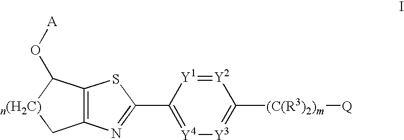
and pharmaceutically acceptable salts thereof,
wherein
A is H, C₁-C₃ alkyl, or acetyl;
Q is —NR¹R², —NR³N(R³)₂, —NR³OR³, or —OH;
R¹ and R² are each independently H or C₁-C₃ alkyl, or R¹ and R² join together with the nitrogen atom to which each is attached, forming a 4 to 6 membered saturated heterocyclic ring comprising heteroatoms selected from 1-2 nitrogen atoms, 0-1 oxygen atom and 0-1 sulfur atom, said ring optionally substituted with one or more of R⁴;
R³ at each occurrence, is independently H or C₁-C₃ alkyl;
R⁴ is C₁-C₃ alkyl, —N(R³)₂, or —OH;
Y¹, Y², Y³, and Y⁴ are the same or different, and are each independently N or CR⁵, or two R⁵ groups on adjacent carbon atoms join together, with the carbon atoms which they are bonded, to form a 9 to 10 membered bicyclic aryl ring or bicyclic heteroaryl ring, said ring comprising members selected from CR⁵ and N;
R⁵ is independently H or is independently selected from C₁-C₃ alkyl, F, Cl, Br, I, CF₃, NO₂, —NR¹R², —CHO, —CONHAr, —C(R³)₂OR³, —C(R³)₂O[C(R³)₂]Ar, —C(R³)₂NR¹R², —C(R³)₂NR³[C(R³)₂]₂NR¹R², —CO₂R⁶, —SOR⁶, and —SO₂R⁶, where Ar is phenyl, 2-pyridyl, 3-pyridyl, 4-pyridyl, 2-thienyl, 3-thienyl, 2-furyl, or 3-furyl, optionally substituted with one or more of R⁴;
R⁶ is independently H or is independently selected from C₁-C₈ alkyl, C₂-C₈ alkenyl, and C₂-C₈ alkynyl, each optionally substituted with —NR¹R², —OR³, C₄-C₆ cycloalkyl, a saturated heterocyclic ring comprising heteroatoms selected from 0-1 nitrogen atom, 0-1 oxygen atom and 0-1 sulfur atom, or —COCH₃, said C₄-C₆ cycloalkyl, optionally substituted with R⁴; or a saturated heterocyclic ring comprising heteroatoms selected from 0-1 nitrogen atom, 0-1 oxygen atom and 0-1 sulfur atom, and optionally substituted with one or more of R⁴;
m is 0 or 1; and n is an integer from 1 to 3.

2. The compound of claim 1, wherein m is 0 and n is 1.

3. The compound of claim 1, wherein m is 0 and n is 2.

4. The compound of claim 1, wherein m is 0 and n is 3.

5. The compound of one of claims 1-4, wherein the ring formed by Y¹, Y², Y³, and Y⁴ is selected from: phenyl, pyridinyl, pyrimidinyl, and pyrazinyl.

6. The compound of one of claims 1-4, wherein Y¹ and Y² or Y³ and Y⁴ are CR⁵ and the two R⁵ groups on adjacent carbon atoms join together, with the carbon atoms which they are bonded, to form a 9 to 10 membered bicyclic aryl ring or bicyclic heteroaryl ring selected from: napthyl, indenyl, indolyl, benzoxazolyl, benzothiazolyl, benzofuranyl, benzisoxazolyl, benzimidazolyl, N-methylbenzimidazolyl, azabenzimidazolyl, indazolyl, quinazolinyl, quinolinyl and isoquinolinyl.

7. A compound selected from: 2-[4-(dimethylamino)phenyl]-5,6-dihydro-4H-cyclopenta[d][1,3]thiazol-6-ol, (6R)-2-[4-(dimethylamino)phenyl]-5,6-dihydro-4H-cyclopenta[d][1,3]thiazol-6-ol, N-[4-(7-methoxy-4,5,6,7-tetrahydro-1,3-benzothiazol-2-yl)phenyl]-N,N-dimethylamine, 2-[4-(dimethylamino)phenyl]-4,5,6,7-tetrahydro-1,3-benzothiazol-7-yl acetate, N-[4-(7-ethoxy-4,5,6,7-tetrahydro-1,3-benzothiazol-2-yl)phenyl]-N,N-diethylamine, 2-[4-(d iethylamino)phenyl]-4,5,6,7-tetrahydro-1,3-benzothiazol-7-ol, N-{4-[(7R)-7-methoxy-4,5,6,7-tetrahydro-1,3-benzothiazol-2-yl]phenyl}-N,N-dimethylamine, 2-(4-aminophenyl)-4,5,6,7-tetrahydro-1,3-benzothiazol-7-ol, 2-[4-(methylamino)phenyl]-4,5,6,7-tetrahydro-1,3-benzothiazol-7-ol, 2-[4-(dimethylamino)-3-fluorophenyl]-4,5,6,7-tetrahydro-1,3-benzothiazol-7-ol, N-[2-fluoro-4-(7-methoxy-4,5,6,7-tetrahydro-1,3-benzothiazol-2-yl)phenyl]-N,N-dimethylamine, 2-[4-(dimethylamino)-2,5-difluorophenyl]-4,5,6,7-tetrahydro-1,3-benzothiazol-7-ol, N-[2,5-difluoro-4-(7-methoxy-4,5,6,7-tetrahydro-1,3-benzothiazol-2-yl)phenyl]-N,N-dimethylamine, 2-[4-(dimethylamino)-2,3,5,6-tetrafluorophenyl]-4,5,6,7-tetrahydro-1,3-benzothiazol-7-ol, N,N-dimethyl-N-[2,3,5,6-tetrafluoro-4-(7-methoxy-4,5,6,7-tetrahydro-1,3-benzo-thiazol-2-yl)phenyl]amine, 2-[3,5-dichloro-4-(dimethylamino)phenyl]-4,5,6,7-tetrahydro-1,3-benzothiazol-7-ol, N-[2,6-dichloro-4-(7-methoxy-4,5,6,7-tetrahydro-1,3-benzothiazol-2-yl)phenyl]-N,N-dimethylamine, 2-[3-bromo-4-(dimethylamino)-phenyl]-4,5,6,7-tetrahydro-1,3-benzothiazol-7-ol, N-[2-bromo-4-(7-methoxy-4,5,6,7-tetrahydro-1,3-benzothiazol-2-yl)phenyl]-N,N-dimethylamine, 2-[3-chloro-4-(dimethylamino)-5-methylphenyl]-4,5,6,7-tetrahydro-1,3-benzothiazol-7-ol, N-[2-chloro-4-(7-methoxy-4,5,6,7-tetrahydro-1,3-benzothiazol-2-yl)-6-methylphenyl]-N,N-dimethylamine, 2-[4-(dimethylamino)-3-nitrophenyl]-4,5,6,7-tetrahydro-1,3-benzo-thiazol-7-ol, N-[4-(7-methoxy-4,5,6,7-tetrahydro-1,3-benzothiazol-2-yl)-2-nitrophenyl]-N,N-dimethylamine, 2-[4-(dimethylamino)-2,6-difluorophenyl]-4,5,6,7-tetrahydro-1,3-benzothiazol-7-ol, [3,5-difluoro-4-(7-methoxy-4,5,6,7-tetrahydro-1,3-benzothiazol-2-yl)phenyl]dimethylamine, 2-[4-(dimethylamino)-3,5-difluorophenyl]-4,5,6,7-tetrahydro-1,3-benzothiazol-7-ol, [2,6-difluoro-4-(7-methoxy-4,5,6,7-tetrahydro-1,3-benzothiazol-2-yl)phenyl]dimethylamine, 2-[3,5-Dichloro-4-(methylamino)phenyl]-4,5,6,7-tetrahydro-1,3-benzothiazol-7-ol, [6-(7-methoxy-4,5,6,7-tetrahedro-benzothiazol-2-yl)-pyridin-3-yl]-dimethylamine, 2-(5-Dimethylamino-pyridine-2-yl)-4,5,6,7-tetrahedro-benzothiazol-7-ol, [4-(7-Methoxy-4,5,6,7-tetrahydro-1,3-benzothiazol-2-yl)phenyl]-amine, 2-[4-(methylamino)-3-nitrophenyl]-4,5,6,7-tetrahydro-1,3-benzothiazol-7-ol, N-[4-(7-methoxy-4,5,6,7-tetrahydro-1,3-benzothiazol-2-yl)-2-nitrophenyl]-N-methyl-amine, [4-(7-ethoxy-4,5,6,7-tetrahydro-1,3-benzothiazol-2-yl)-2-nitrophenyl]-dimethylamine, [4-(7-ethoxy-4,5,6,7-tetrahydro-1,3-benzothiazol-2-yl)-2-nitrophenyl]-methylamine, N-[4-(7-methoxy-4,5,6,7-tetrahydro-1,3-benzothiazol-2-yl)phenyl]-N-methylamine, 5-(7-Methoxy-4,5,6,7-tetrahydro-1,3-benzothiazol-2-yl)-N,N-dimethyl-pyridin-2-amine, 2-[6-(dimethylamino)pyridin-3-yl]-4,5,6,7-tetrahydro-1,3-benzo-thiazol-7-ol, 5-(7-Ethoxy-4,5,6,7-tetrahydro-1,3-benzothiazol-2-yl)-N,N-dimethyl-pyridin-2-amine, 2-[4-(dimethylamino)-2-fluorophenyl]-4,5,6,7-tetrahydro-1,3-benzothiazol-7-ol, [4-(7-ethoxy-4,5,6,7-tetrahydro-1,3-benzothiazol-2-yl)-3-fluorophenyl]dimethylamine, 2-[4-(dimethylamino)-2,3-difluorophenyl]-4,5,6,7-tetrahydro-1,3-benzothiazol-7-ol, [4-(7-ethoxy-4,5,6,7-tetrahydro-1,3-benzothiazol-2-yl)-2,3-difluorophenyl]dimethylamine, 2-[4-(dimethylamino)-2-methylphenyl]-4,5,6,7-tetrahydro-1,3-benzothiazol-7-ol, [4-(7-ethoxy-4,5,6,7-tetrahydro-1,3-benzothiazol-2-yl)-3-methylphenyl]dimethylamine, 2-[4-(dimethylamino)-2-ethylphenyl]-4,5,6,7-tetrahydro-1,3-benzothiazol-7-ol, [4-(7-ethoxy-4,5,6,7-tetrahydro-1,3-benzothiazol-2-yl)-3-ethylphenyl]dimethylamine, 2-[3-amino-4-(dimethylamino)phenyl]-4,5,6,7-tetrahydro-1,3-benzothiazol-7-ol, 4-(7-methoxy-4,5,6,7-tetrahydro-1,3-benzothiazol-2-yl)-N1,N1-dimethylbenzene-1,2-diamine, 2-[3,4-bis(dimethylamino)phenyl]-4,5,6,7-tetrahydro-1,3-benzothiazol-7-ol, 2-[3-amino-4-(methylamino)phenyl]-4,5,6,7-tetrahydro-1,3-benzothiazol-7-ol, 2-[2-(dimethylamino)pyrimidin-5-yl]-4,5,6,7-tetrahydro-1,3-benzothiazol-7-ol, 5-(7-ethoxy-4,5,6,7-tetrahydro-1,3-benzothiazol-2-yl)-N,N-dimethylpyrimidin-2-amine, methyl 5-(dimethylamino)-2-(7-hydroxy-4,5,6,7-tetrahydro-1,3-benzothiazol-2-yl)benzoate, methyl 5-(dimethylamino)-2-(7-ethoxy-4,5,6,7-tetrahydro-1,3-benzothiazol-2-yl)benzoate, methyl 5-(dimethylamino)-2-(7-methoxy-4,5,6,7-tetrahydro-1,3-benzothiazol-2-yl)benzoate, methyl 5-(dimethylamino)-2-(7-isopropoxy-4,5,6,7-tetrahydro-1,3-benzothiazol-2-yl)benzoate, 5-(dimethylamino)-2-(7-hydroxy-4,5,6,7-tetrahydro-1,3-benzothiazol-2-yl)benzoic acid, 2-[4-(dimethylamino)-2-(hydroxymethyl)phenyl]-4,5,6,7-tetrahydro-1,3-benzothiazol-7-ol, 2-[4-(dimethylamino)-2-(pyrrolidin-1-ylmethyl)phenyl]-4,5,6,7-tetrahydro-1,3-benzothiazol-7-ol, 2-[4-(dimethylamino)-2-(morpholin-4-ylmethyl)phenyl]-4,5,6,7-tetrahydro-1,3-benzothiazol-7-ol, 2-{4-(dimethylamino)-2-[(dimethylamino)methyl]phenyl}-4,5,6,7-tetrahydro-1,3-benzothiazol-7-ol, 2-(4-(dimethylamino)-2-{[[2-(dimethylamino)ethyl]-(ethyl)amino]methyl}phenyl)-4,5,6,7-tetrahydro-1,3-benzothiazol-7-ol, 1-methylpyrrolidin-3-yl 5-(dimethylamino)-2-(7-hydroxy-4,5,6,7-tetrahydro-1,3-benzothiazol-2-yl)benzoate, 2-[4-(Dimethylamino)-2-(methoxymethyl)phenyl]-4,5,6,7-tetrahydro-1,3-benzothiazol-7-ol, 2-[2-[(Benzyloxy)-methyl]-4-(dimethylamino)phenyl]-4,5,6,7-tetrahydro-1,3-benzothiazol-7-ol, 3-[(benzyloxy)methyl]-4-(7-isopropoxy-4,5,6,7-tetrahydro-1,3-benzothiazol-2-yl)-N,N-dimethylaniline, 2-[4-(dimethylamino)-2-(1-hydroxy-1-methylethyl)phenyl]-4,5,6,7-tetrahydro-1,3-benzothiazol-7-ol, 2-[4-(dimethylamino)-2-(1-hydroxyethyl)phenyl]-4,5,6,7-tetrahydro-1,3-benzothiazol-7-ol, 1-[5-(Dimethylamino)-2-(7-hydroxy-4,5,6,7-tetrahydro-1,3-benzothiazol-2-yl)phenyl]ethanone, 2-[2-(azetidin-1-ylmethyl)-4-(dimethylamino)phenyl]-4,5,6,7-tetrahydro-1,3-benzothiazol-7-ol, 2-{4-(dimethyl-amino)-2-[(methylamino)methyl]phenyl}-4,5,6,7-tetrahydro-1,3-benzothiazol-7-ol, 2-{4-(dimethylamino)-2-[(3-hydroxyazetidin-1-yl)methyl]phenyl}-4,5,6,7-tetrahydro-1,3-benzothiazol-7-ol, 2-[2-chloro-4-(dimethylamino)phenyl]-4,5,6,7-tetrahydro-1,3-benzothiazol-7-yl acetate, 2-[4-(dimethylamino)-2-fluorophenyl]-4,5,6,7-tetrahydro-1,3-benzothiazol-7-yl acetate, 1-[4-bromo-5-(dimethylamino)-2-(7-hydroxy-4,5,6,7-tetrahydro-1,3-benzothiazol-2-yl)phenyl]ethanone, 1-[2-bromo-3-(dimethylamino)-6-(7-hydroxy-4,5,6,7-tetrahydro-1,3-benzothiazol-2-yl)phenyl]ethanone, 2-[2-(1-hydroxyethyl)-4-(methylamino)phenyl]-4,5,6,7-tetrahydro-1,3-benzothiazol-7-ol, 2-(2-bromo-4-pyrrolidin-1-ylphenyl)-4,5,6,7-tetrahydro-1,3-benzothiazol-7-ol, 2-(2-bromo-4-pyrrolidin-1-ylphenyl)-7-isopropoxy-4,5,6,7-tetrahydro-1,3-benzothiazole, 2-(7-hydroxy-4,5,6,7-tetrahydro-1,3-benzothiazol-2-yl)-5-pyrrolidin-1-ylbenzonitrile, 2-[2-(aminomethyl)-4-pyrrolidin-1-yl phenyl]-4,5,6,7-tetrahydro-1,3-benzothiazol-7-ol, 2-{4-[(dimethylamino)methyl]phenyl}-4,5,6,7-tetrahydro-1,3-benzothiazol-7-ol, 2-[4-(hydroxylmethyl)phenyl]-4,5,6,7-tetrahydro-1,3-benzothiazol-7-ol, 2-(4-amino-3-methylphenyl)-4,5,6,7-tetrahydro-1,3-benzothiazol-7-ol, 2-[4-(dimethylamino)-3-methylphenyl]-4,5,6,7-tetrahydro-1,3-benzothiazol-7-ol, [2-chloro-4-(7-methoxy-4,5,6,7-tetrahydro-1,3-benzothiazol-2-yl)phenyl]-dimethylamine, 2-[3-chloro-4-(dimethylamino)phenyl]-4,5,6,7-tetrahydro-1,3-benzothiazol-7-ol, 3-chloro-4-(7-methoxy-4,5,6,7-tetrahydro-1,3-benzothiazol-2-yl)phenyl]dimethylamine, 2-[2-chloro-4-(dimethylamino)phenyl]-4,5,6,7-tetrahydro-1,3-benzothiazol-7-ol, 2-[4-(dimethylamino)-2-methylphenyl]-4,5,6,7-tetrahydro-1,3-benzothiazol-7-ol, 2-[4-(dimethylamino)-2-methoxyphenyl]-4,5,6,7-tetrahydro-1,3-benzothiazol-7-ol, 2-[4-(dimethylamino)-3-iodophenyl]-4,5,6,7-tetrahydro-1,3-benzothiazol-7-ol, 2-[4-(dimethylamino)-2-(trifluoromethyl)phenyl]-4,5,6,7-tetrahydro-1,3-benzothiazol-7-ol, 2-[4-(dimethylamino)-3-(trifluoromethyl)phenyl]-4,5,6,7-tetrahydro-1,3-benzothiazol-7-ol, (7R)-2-[4-(dimethylamino)-2-methyl phenyl]-4,5,6,7-tetrahydro-1,3-benzothiazol-7-ol, (7S)-2-[4-(dimethylamino)-2-methylphenyl]-4,5,6,7-tetrahydro-1,3-benzothiazol-7-ol, 2-[4-(dimethylamino)-2-hydroxyphenyl]-4,5,6,7-tetrahydro-1,3-benzothiazol-7-ol, 2-[4-(dimethylamino)-3,5-dimethylphenyl]-4,5,6,7-tetrahydro-1,3-benzothiazol-7-ol, methyl 2-(dimethylamino)-5-(7-hydroxy-4,5,6,7-tetrahydro-1,3-benzothiazol-2-yl)benzoate, methyl 2-(dimethylamino)-5-(7-methoxy-4,5,6,7-tetrahydro-1,3-benzothiazol-2-yl)benzoate, 2-(dimethylamino)-5-(7-hydroxy-4,5,6,7-tetrahydro-1,3-benzothiazol-2-yl)benzoic acid, 2-[4-(dimethylamino)-3-(hydroxymethyl)phenyl]-4,5,6,7-tetrahydro-1,3-benzothiazol-7-ol, 2-{4-(dimethylamino)-3-[(dimethylamino)methyl]phenyl}-4,5,6,7-tetrahydro-1,3-benzothiazol-7-ol, 2-[4-(dimethylamino)-3-(pyrrolidin-1-ylmethyl)phenyl]-4,5,6,7-tetrahydro-1,3-benzothiazol-7-ol, 2-[4-(dimethylamino)-3-(piperidin-1-ylmethyl)phenyl]-4,5,6,7-tetrahydro-1,3-benzothiazol-7-ol, 2-(dimethylamino)-5-(7-hydroxy-4,5,6,7-tetrahydro-1,3-benzothiazol-2-yl)benzaldehyde, isopropyl 5-(dimethylamino)-2-(7-hydroxy-4,5,6,7-tetrahydro-1,3-benzothiazol-2-yl)benzoate, allyl 5-(dimethylamino)-2-(7-hydroxy-4,5,6,7-tetrahydro-1,3-benzothiazol-2-yl)benzoate, 2-methoxyethyl 5-(dimethyl-amino)-2-(7-hydroxy-4,5,6,7-tetrahydro-1,3-benzothiazol-2-yl)benzoate, benzyl 5-(dimethylamino)-2-(7-hydroxy-4,5,6,7-tetrahydro-1,3-benzothiazol-2-yl)benzoate, 2-furylmethyl 5-(dimethylamino)-2-(7-hydroxy-4,5,6,7-tetrahydro-1,3-benzothiazol-2-yl)benzoate, 3-furylmethyl 5-(dimethylamino)-2-(7-hydroxy-4,5,6,7-tetrahydro-1,3-benzothiazol-2-yl)benzoate, cyclopentyl 5-(dimethylamino)-2-(7-hydroxy-4,5,6,7-tetrahydro-1,3-benzothiazol-2-yl)benzoate, butyl 5-(dimethylamino)-2-(7-hydroxy-4,5,6,7-tetrahydro-1,3-benzothiazol-2-yl)benzoate, cyclobutylmethyl 5-(dimethylamino)-2-(7-hydroxy-4,5,6,7-tetrahydro-1,3-benzothiazol-2-yl)benzoate, cyclohexyl methyl 5-(dimethylamino)-2-(7-hydroxy-4,5,6,7-tetrahydro-1,3-benzo-thiazol-2-yl)benzoate, propyl 5-(dimethylamino)-2-(7-hydroxy-4,5,6,7-tetrahydro-1,3-benzothiazol-2-yl)benzoate, 3-thienylmethyl 5-(dimethylamino)-2-(7-hydroxy-4,5,6,7-tetrahydro-1,3-benzothiazol-2-yl)benzoate, 2-thienylmethyl 5-(dimethylamino)-2-(7-hydroxy-4,5,6,7-tetrahydro-1,3-benzothiazol-2-yl)benzoate, 2-pyrrolidin-1-ylethyl 5-(dimethylamino)-2-(7-hydroxy-4,5,6,7-tetrahydro-1,3-benzothiazol-2-yl)benzoate, 2-(dimethylamino)ethyl 5-(dimethylamino)-2-(7-hydroxy-4,5,6,7-tetrahydro-1,3-benzothiazol-2-yl)benzoate, pyridin-3-ylmethyl 5-(dimethylamino)-2-(7-hydroxy-4,5,6,7-tetrahydro-1,3-benzothiazol-2-yl)benzoate, pyridin-4-ylmethyl 5-(dimethylamino)-2-(7-hydroxy-4,5,6,7-tetrahydro-1,3-benzothiazol-2-yl)benzoate, pyridin-2-ylmethyl 5-(dimethylamino)-2-(7-hydroxy-4,5,6,7-tetrahydro-1,3-benzo-thiazol-2-yl)benzoate, prop-2-yn-1-yl 5-(dimethylamino)-2-(7-hydroxy-4,5,6,7-tetrahydro-1,3-benzothiazol-2-yl)benzoate, (2E)-but-2-en-1-yl 5-(dimethylamino)-2-(7-hydroxy-4,5,6,7-tetrahydro-1,3-benzothiazol-2-yl)benzoate, 2-oxopropyl 5-(dimethylamino)-2-(7-hydroxy-4,5,6,7-tetrahydro-1,3-benzothiazol-2-yl)benzoate, tetrahydrofuran-2-ylmethyl 5-(dimethylamino)-2-(7-hydroxy-4,5,6,7-tetrahydro-1,3-benzothiazol-2-yl)benzoate, but-2-yn-1-yl 5-(dimethylamino)-2-(7-hydroxy-4,5,6,7-tetrahydro-1,3-benzothiazol-2-yl)benzoate, tetrahydrofuran-3-ylmethyl 5-(dimethylamino)-2-(7-hydroxy-4,5,6,7-tetrahydro-1,3-benzothiazol-2-yl)benzoate, 2-(acetyloxy)ethyl 5-(dimethylamino)-2-(7-hydroxy-4,5,6,7-tetrahydro-1,3-benzothiazol-2-yl)benzoate, 3-methyl but-2-en-1-yl 5-(dimethylamino)-2-(7-hydroxy-4,5,6,7-tetrahydro-1,3-benzothiazol-2-yl)benzoate and 2,2-dimethylpropyl 5-(dimethylamino)-2-(7-hydroxy-4,5,6,7-tetrahydro-1,3-benzothiazol-2-yl)benzoate, piperidin-3-ylmethyl 5-(dimethylamino)-2-(7-hydroxy-4,5,6,7-tetrahydro-1,3-benzothiazol-2-yl)benzoate, piperidin-3-yl 5-(dimethylamino)-2-(7-hydroxy-4,5,6,7-tetrahydro-1,3-benzothiazol-2-yl)benzoate, 2-[2-bromo-4-(dimethylamino)phenyl]-4,5,6,7-tetrahydro-1,3-benzothiazol-7-ol, 2-{4-[methoxy(methyl)amino]phenyl}-4,5,6,7-tetrahydro-1,3-benzothiazol-7-ol, 2-[4-(trimethylhydrazino)phenyl]-4,5,6,7-tetrahydro-1,3-benzothiazol-7-ol, 2-(4-azetidin-1-ylphenyl)-4,5,6,7-tetrahydro-1,3-benzothiazol-7-ol, 2-[4-(3-Hydroxypyrrolidin-1-yl)phenyl]-4,5,6,7-tetrahydro-1,3-benzothiazol-7-ol, 2-(4-Piperidin-1-ylphenyl)-4,5,6,7-tetrahydro-1,3-benzothiazol-7-ol, 2-(4-Morpholin-4-ylphenyl)-4,5,6,7-tetrahydro-1,3-benzothiazol-7-ol, 2-[4-(4-methylpiperazin-1-yl)phenyl]-4,5,6,7-tetrahydro-1,3-benzothiazol-7-ol, 2-{4-[(3S)-3-(Dimethyl-amino)pyrrolidin-1-yl]phenyl}-4,5,6,7-tetrahydro-1,3-benzothiazol-7-ol, 2-[4-(2-Methylpyrrolidin-1-yl)phenyl]-4,5,6,7-tetrahydro-1,3-benzothiazol-7-ol, 2-{4-[ethyl(methyl)amino]phenyl}-4,5,6,7-tetrahydro-1,3-benzothiazol-7-ol, 2-(2-nitro-4-pyrrolidin-1-ylphenyl)-4,5,6,7-tetrahydro-1,3-benzothiazol-7-ol, 2-(7-methoxy-4,5,6,7-tetrahydro-1,3-benzothiazol-2-yl)-5-pyrrolidin-1-ylaniline, 2-(7-methoxy-4,5,6,7-tetrahydro-1,3-benzothiazol-2-yl)-5-pyrrolidin-1-ylaniline, 2-(2-amino-4-pyrrolidin-1-ylphenyl)-4,5,6,7-tetrahydro-1,3-benzothiazol-7-ol, 2-[2-(methylthio)-4-pyrrolidin-1-ylphenyl]-4,5,6,7-tetrahydro-1,3-benzothiazol-7-ol, 2-[2-(Methylsulfinyl)-4-pyrrolidin-1-ylphenyl]-4,5,6,7-tetrahydro-1,3-benzothiazol-7-ol and 2-[2-(Methylsulfonyl)-4-pyrrolidin-1-ylphenyl]-4,5,6,7-tetrahydro-1,3-benzothiazol-7-ol, 2-[4-(3-hydroxy-azetidin-1-yl)-phenyl]-4,5,6,7-tetrahydro-benzothiazol-7-ol, 2-(4-pyrrolidin-1-yl-phenyl)-4,5,6,7-tetrahydro-benzothiazol-7-ol, 2-[4-(3-dimethylamino-pyrrolidin-1-yl)-phenyl]-4,5,6,7-tetrahydro-benzothiazol-7-ol, 2-[4-(2-dimethylaminomethyl-pyrrolidin-1-yl)-phenyl]-4,5,6,7-tetrahydro-benzothiazol-7-ol, 2-(7-Hydroxy-4,5,6,7-tetrahydro-benzothiazol-2-yl)-5-pyrrolidin-1-yl-benzoic acid methyl ester, 2-(2-Hydroxymethyl-4-pyrrolidin-1-yl-phenyl)-4,5,6,7-tetrahydro-benzothiazol-7-ol, 2-(4-Piperazin-1-yl-phenyl)-4,5,6,7-tetrahydrobenzothiazol-7-ol, (7R)-2-[4-(dimethylamino)phenyl]-4,5,6,7-tetrahydro-1,3-benzothiazol-7-ol, methyl 5-(dimethylamino)-2-[(7R)-7-hydroxy-4,5,6,7-tetrahydro-1,3-benzothiazol-2-yl]benzoate, 2-[4-(dimethylamino)phenyl]-4,5,6,7-tetrahydro-1,3-benzothiazol-7-ol, (7S)-2-[4-(dimethylamino)phenyl]-4,5,6,7-tetrahydro-1,3-benzothiazol-7-ol, 2-[4-(dimethylamino)-1-naphthyl]-4,5,6,7-tetrahydro-1,3-benzothiazol-7-ol, [4-(7-ethoxy-4,5,6,7-tetrahydro-1,3-benzothiazol-2-yl)-1-naphthyl]dimethylamine and pharmaceutically acceptable salts thereof.

8. A pharmaceutically acceptable ester of any one of a compound of claims 1-7.

9. A pharmaceutical composition comprising: a compound of any one of claims 1-8 and a pharmaceutically acceptable carrier.

10. A method for preparing a compound of claim 1, comprising the steps of:
(a) reacting a substituted arylnitrile compound of formula 1: 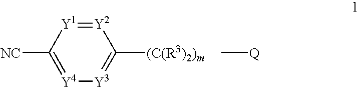
with phosphorous pentasulfide, thereby forming a compound of formula 2: 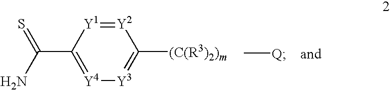
(c) reacting the compound of formula 2 formed in step (b) with a compound of formula 3:

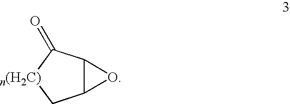

11. A method for preparing a compound of claim 1, comprising the steps of:
(a) reacting a 4-bromoarylnitrile compound of formula 4: 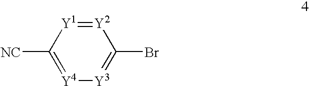
with phosphorous pentasulfide, thereby forming a compound of formula 5: 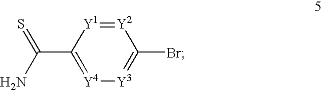
(b) reacting the compound of formula 5, formed in step (a) with a compound of formula 3: 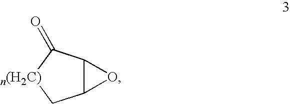
thereby forming a compound of formula 6: 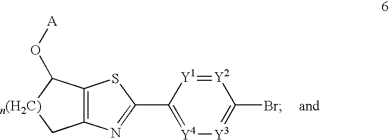
(c) treating the compound of formula 6, formed in step (b) with alkyl or cyclic alkyl amines.

12. A method for treating a disease associated with securin comprising administering to a subject in need a therapeutically effective amount of any one of a compound of claims 1-8.

13. The method of claim 12, wherein the therapeutically effective amount is from 0.1 mg/kg/day to 1000 mg/kg/day.

14. The method of claim 12, wherein the therapeutically effective amount is from 10 mg/kg/day to 600 mg/kg/day.

15. The method of any one of claims 12-14, wherein the disease associated with securin is a cancer selected from: breast, colon, lung, prostate, melanoma, epidermal, leukemia, kidney, bladder, mouth, larynx, esophagus, stomach, ovary, pancreas, liver, skin, thyroid, prostate and brain cancer.

16. The method of claim 15, wherein the disease associated with securin is breast cancer.

17. A method for inhibiting tumor growth in a subject comprising administering to a subject in need a therapeutically effective amount of a compound of any one of a compound of claims 1-8.

18. The method of claim 17, wherein tumor growth is associated with a cancer selected from: breast, colon, lung, prostate, melanoma, epidermal, leukemia, kidney, bladder, mouth, larynx, esophagus, stomach, ovary, pancreas, liver, skin, thyroid, prostate and brain cancer.

19. The method of claim 18, wherein the therapeutically effective amount is from 0.1 mg/kg/day to 1000 mg/kg/day.

20. The method of claims 18 or 19, wherein tumor growth is associated with a human breast carcinoma.

21. A method for treating cancer in a subject comprising administering to a subject in need a therapeutically effective amount of any one of a compound of claims 1-8.

22. The method of claim 21, wherein the cancer treated is selected from: breast, colon, lung, prostate, melanoma, epidermal, leukemia, kidney, bladder, mouth, larynx, esophagus, stomach, ovary, pancreas, liver, skin, thyroid, prostate and brain cancer.

23. The method of claim 21, wherein a chemotherapeutic agent is used in combination with the therapeutically effective amount of any one of a compound of claims 1-8.

24. A pharmaceutical composition comprising any one of a compound of claims 1-7 in combination with other kinase-inhibiting compounds or chemotherapeutic agents and a pharmaceutically acceptable carrier.
